SEQUENCE LISTING (411 sequences)

SEQ ID NO: 1 - DNA, 600 bp, Homo sapiens
  source 1..600: mol_type = genomic DNA
gtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctcca

SEQ ID NO: 2 - DNA, 500 bp, Homo sapiens
  source 1..500: mol_type = other DNA
gatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctcca

SEQ ID NO: 3 - DNA, 400 bp, Homo sapiens
  source 1..400: mol_type = other DNA
gttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctcca

SEQ ID NO: 4 - DNA, 300 bp, Homo sapiens
  source 1..300: mol_type = other DNA
gttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctcca

SEQ ID NO: 5 - DNA, 590 bp, Homo sapiens
  source 1..590: mol_type = other DNA
agacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctcca

SEQ ID NO: 6 - DNA, 580 bp, Homo sapiens
  source 1..580: mol_type = other DNA
tgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctcca

SEQ ID NO: 7 - DNA, 570 bp, Homo sapiens
  source 1..570: mol_type = other DNA
ctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctcca

SEQ ID NO: 8 - DNA, 560 bp, Homo sapiens
  source 1..560: mol_type = other DNA
ctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctcca

SEQ ID NO: 9 - DNA, 550 bp, Homo sapiens
  source 1..550: mol_type = other DNA
cggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctcca

SEQ ID NO: 10 - DNA, 540 bp, Homo sapiens
  source 1..540: mol_type = other DNA
cctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctcca

SEQ ID NO: 11 - DNA, 530 bp, Homo sapiens
  source 1..530: mol_type = other DNA
ctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctcca

SEQ ID NO: 12 - DNA, 520 bp, Homo sapiens
  source 1..520: mol_type = other DNA
taggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctcca

SEQ ID NO: 13 - DNA, 510 bp, Homo sapiens
  source 1..510: mol_type = other DNA
tagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctcca

SEQ ID NO: 14 - DNA, 490 bp, Homo sapiens
  source 1..490: mol_type = other DNA
cccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctcca

SEQ ID NO: 15 - DNA, 480 bp, Homo sapiens
  source 1..480: mol_type = other DNA
cagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctcca

SEQ ID NO: 16 - DNA, 470 bp, Homo sapiens
  source 1..470: mol_type = other DNA
ggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctcca

SEQ ID NO: 17 - DNA, 460 bp, Homo sapiens
  source 1..460: mol_type = other DNA
ataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctcca

SEQ ID NO: 18 - DNA, 450 bp, Homo sapiens
  source 1..450: mol_type = other DNA
ccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctcca

SEQ ID NO: 19 - DNA, 440 bp, Homo sapiens
  source 1..440: mol_type = other DNA
tccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctcca

SEQ ID NO: 20 - DNA, 430 bp, Homo sapiens
  source 1..430: mol_type = other DNA
tggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctcca

SEQ ID NO: 21 - DNA, 420 bp, Homo sapiens
  source 1..420: mol_type = other DNA
acttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctcca

SEQ ID NO: 22 - DNA, 410 bp, Homo sapiens
  source 1..410: mol_type = other DNA
tgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctcca

SEQ ID NO: 23 - DNA, 390 bp, Homo sapiens
  source 1..390: mol_type = other DNA
gccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctcca

SEQ ID NO: 24 - DNA, 380 bp, Homo sapiens
  source 1..380: mol_type = other DNA
cctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctcca

SEQ ID NO: 25 - DNA, 370 bp, Homo sapiens
  source 1..370: mol_type = other DNA
gctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctcca

SEQ ID NO: 26 - DNA, 360 bp, Homo sapiens
  source 1..360: mol_type = other DNA
ccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctcca

SEQ ID NO: 27 - DNA, 350 bp, Homo sapiens
  source 1..350: mol_type = other DNA
gttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctcca

SEQ ID NO: 28 - DNA, 340 bp, Homo sapiens
  source 1..340: mol_type = other DNA
tctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctcca

SEQ ID NO: 29 - DNA, 330 bp, Homo sapiens
  source 1..330: mol_type = other DNA
caggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctcca

SEQ ID NO: 30 - DNA, 320 bp, Homo sapiens
  source 1..320: mol_type = other DNA
aagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctcca

SEQ ID NO: 31 - DNA, 310 bp, Homo sapiens
  source 1..310: mol_type = other DNA
gctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctcca

SEQ ID NO: 32 - DNA, 290 bp, Homo sapiens
  source 1..290: mol_type = other DNA
tgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctcca

SEQ ID NO: 33 - DNA, 280 bp, Homo sapiens
  source 1..280: mol_type = other DNA
atatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctcca

SEQ ID NO: 34 - DNA, 270 bp, Homo sapiens
  source 1..270: mol_type = other DNA
tggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctcca

SEQ ID NO: 35 - DNA, 260 bp, Homo sapiens
  source 1..260: mol_type = other DNA
tagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctcca

SEQ ID NO: 36 - DNA, 250 bp, Homo sapiens
  source 1..250: mol_type = other DNA
gagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctcca

SEQ ID NO: 37 - DNA, 240 bp, Homo sapiens
  source 1..240: mol_type = other DNA
aggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctcca

SEQ ID NO: 38 - DNA, 230 bp, Homo sapiens
  source 1..230: mol_type = other DNA
gggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctcca

SEQ ID NO: 39 - DNA, 220 bp, Homo sapiens
  source 1..220: mol_type = other DNA
ggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctcca

SEQ ID NO: 40 - DNA, 210 bp, Homo sapiens
  source 1..210: mol_type = other DNA
acatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctcca

SEQ ID NO: 41 - DNA, 200 bp, Homo sapiens
  source 1..200: mol_type = other DNA
gtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctcca

SEQ ID NO: 42 - DNA, 550 bp, Homo sapiens
  source 1..550: mol_type = other DNA
gtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctc

SEQ ID NO: 43 - DNA, 540 bp, Homo sapiens
  source 1..540: mol_type = other DNA
agacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctc

SEQ ID NO: 44 - DNA, 530 bp, Homo sapiens
  source 1..530: mol_type = other DNA
tgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctc

SEQ ID NO: 45 - DNA, 520 bp, Homo sapiens
  source 1..520: mol_type = other DNA
ctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctc

SEQ ID NO: 46 - DNA, 510 bp, Homo sapiens
  source 1..510: mol_type = other DNA
ctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctc

SEQ ID NO: 47 - DNA, 500 bp, Homo sapiens
  source 1..500: mol_type = other DNA
cggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctc

SEQ ID NO: 48 - DNA, 490 bp, Homo sapiens
  source 1..490: mol_type = other DNA
cctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctc

SEQ ID NO: 49 - DNA, 480 bp, Homo sapiens
  source 1..480: mol_type = other DNA
ctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctc

SEQ ID NO: 50 - DNA, 470 bp, Homo sapiens
  source 1..470: mol_type = other DNA
taggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctc

SEQ ID NO: 51 - DNA, 460 bp, Homo sapiens
  source 1..460: mol_type = other DNA
tagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctc

SEQ ID NO: 52 - DNA, 450 bp, Homo sapiens
  source 1..450: mol_type = other DNA
gatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctc

SEQ ID NO: 53 - DNA, 440 bp, Homo sapiens
  source 1..440: mol_type = other DNA
cccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctc

SEQ ID NO: 54 - DNA, 430 bp, Homo sapiens
  source 1..430: mol_type = other DNA
cagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctc

SEQ ID NO: 55 - DNA, 420 bp, Homo sapiens
  source 1..420: mol_type = other DNA
ggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctc

SEQ ID NO: 56 - DNA, 410 bp, Homo sapiens
  source 1..410: mol_type = other DNA
ataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctc

SEQ ID NO: 57 - DNA, 400 bp, Homo sapiens
  source 1..400: mol_type = other DNA
ccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctc

SEQ ID NO: 58 - DNA, 390 bp, Homo sapiens
  source 1..390: mol_type = other DNA
tccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctc

SEQ ID NO: 59 - DNA, 380 bp, Homo sapiens
  source 1..380: mol_type = other DNA
tggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctc

SEQ ID NO: 60 - DNA, 370 bp, Homo sapiens
  source 1..370: mol_type = other DNA
acttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctc

SEQ ID NO: 61 - DNA, 360 bp, Homo sapiens
  source 1..360: mol_type = other DNA
tgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctc

SEQ ID NO: 62 - DNA, 350 bp, Homo sapiens
  source 1..350: mol_type = other DNA
gttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctc

SEQ ID NO: 63 - DNA, 340 bp, Homo sapiens
  source 1..340: mol_type = other DNA
gccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctc

SEQ ID NO: 64 - DNA, 330 bp, Homo sapiens
  source 1..330: mol_type = other DNA
cctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctc

SEQ ID NO: 65 - DNA, 320 bp, Homo sapiens
  source 1..320: mol_type = other DNA
gctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctc

SEQ ID NO: 66 - DNA, 310 bp, Homo sapiens
  source 1..310: mol_type = other DNA
ccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctc

SEQ ID NO: 67 - DNA, 300 bp, Homo sapiens
  source 1..300: mol_type = other DNA
gttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctc

SEQ ID NO: 68 - DNA, 290 bp, Homo sapiens
  source 1..290: mol_type = other DNA
tctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctc

SEQ ID NO: 69 - DNA, 280 bp, Homo sapiens
  source 1..280: mol_type = other DNA
caggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctc

SEQ ID NO: 70 - DNA, 270 bp, Homo sapiens
  source 1..270: mol_type = other DNA
aagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctc

SEQ ID NO: 71 - DNA, 260 bp, Homo sapiens
  source 1..260: mol_type = other DNA
gctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctc

SEQ ID NO: 72 - DNA, 250 bp, Homo sapiens
  source 1..250: mol_type = other DNA
gttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctc

SEQ ID NO: 73 - DNA, 240 bp, Homo sapiens
  source 1..240: mol_type = other DNA
tgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctc

SEQ ID NO: 74 - DNA, 230 bp, Homo sapiens
  source 1..230: mol_type = other DNA
atatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctc

SEQ ID NO: 75 - DNA, 220 bp, Homo sapiens
  source 1..220: mol_type = other DNA
tggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctc

SEQ ID NO: 76 - DNA, 210 bp, Homo sapiens
  source 1..210: mol_type = other DNA
tagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctc

SEQ ID NO: 77 - DNA, 200 bp, Homo sapiens
  source 1..200: mol_type = other DNA
gagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctc

SEQ ID NO: 78 - DNA, 550 bp, Homo sapiens; identical to SEQ ID NO: 42
  source 1..550: mol_type = other DNA

SEQ ID NO: 79 - DNA, 500 bp, Homo sapiens
  source 1..500: mol_type = other DNA
gtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctc

SEQ ID NO: 80 - DNA, 450 bp, Homo sapiens
  source 1..450: mol_type = other DNA
gtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccaccc

SEQ ID NO: 81 - DNA, 400 bp, Homo sapiens
  source 1..400: mol_type = other DNA
gtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcct

SEQ ID NO: 82 - DNA, 350 bp, Homo sapiens
  source 1..350: mol_type = other DNA
gtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatct

SEQ ID NO: 83 - DNA, 300 bp, Homo sapiens
  source 1..300: mol_type = other DNA
gtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttat

SEQ ID NO: 84 - DNA, 250 bp, Homo sapiens
  source 1..250: mol_type = other DNA
gtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgg

SEQ ID NO: 85 - DNA, 200 bp, Homo sapiens
  source 1..200: mol_type = other DNA
gtcatggagaagacccaccttgcagatgtcctcactggggctggcagagccggcaacctgcctaaggctgctcagtccattaggagccagtagcctggaagatgtctttacccccagcatcagttcaagtggagcagcacataactcttgccctctgccttccaagattctggtgctgagacttatggagtgtcttggag

SEQ ID NO: 86 - DNA, 1464 bp, Homo sapiens
  source 1..1464: mol_type = genomic DNA
atggctccggccgcctggctccgcagcgcggccgcgcgcgccctcctgcccccgatgctgctgctgctgctccagccgccgccgctgctggcccgggctctgccgccggacgcccaccacctccatgccgagaggagggggccacagccctggcatgcagccctgcccagtagcccggcacctgcccctgccacgcaggaagccccccggcctgccagcagcctcaggcctccccgctgtggcgtgcccgacccatctgatgggctgagtgcccgcaaccgacagaagaggttcgtgctttctggcgggcgctgggagaagacggacctcacctacaggatccttcggttcccatggcagttggtgcaggagcaggtgcggcagacgatggcagaggccctaaaggtatggagcgatgtgacgccactcacctttactgaggtgcacgagggccgtgctgacatcatgatcgacttcgccaggtactggcatggggacgacctgccgtttgatgggcctgggggcatcctggcccatgccttcttccccaagactcaccgagaaggggatgtccacttcgactatgatgagacctggactatcggggatgaccagggcacagacctgctgcaggtggcagcccatgaatttggccacgtgctggggctgcagcacacaacagcagccaaggccctgatgtccgccttctacacctttcgctacccactgagtctcagcccagatgactgcaggggcgttcaacacctatatggccagccctggcccactgtcacctccaggaccccagccctgggcccccaggctgggatagacaccaatgagattgcaccgctggagccagacgccccgccagatgcctgtgaggcctcctttgacgcggtctccaccatccgaggcgagctctttttcttcaaagcgggctttgtgtggcgcctccgtgggggccagctgcagcccggctacccagcattggcctctcgccactggcagggactgcccagccctgtggacgctgccttcgaggatgcccagggccacatttggttcttccaaggtgctcagtactgggtgtacgacggtgaaaagccagtcctgggccccgcacccctcaccgagctgggcctggtgaggttcccggtccatgctgccttggtctggggtcccgagaagaacaagatctacttcttccgaggcagggactactggcgtttccaccccagcacccggcgtgtagacagtcccgtgccccgcagggccactgactggagaggggtgccctctgagatcgacgctgccttccaggatgctgatggctatgcctacttcctgcgcggccgcctctactggaagtttgaccctgtgaaggtgaaggctctggaaggcttcccccgtctcgtgggtcctgacttctttggctgtgccgagcctgccaacactttcctc

SEQ ID NO: 87 - protein, 488 aa, Homo sapiens
MAPAAWLRSAAARALLPPMLLLLLQPPPLLARALPPDAHHLHAERRGPQPWHAALPSSPAPAPATQEAPRPASSLRPPRCGVPDPSDGLSARNRQKRFVLSGGRWEKTDLTYRILRFPWQLVQEQVRQTMAEALKVWSDVTPLTFTEVHEGRADIMIDFARYWHGDDLPFDGPGGILAHAFFPKTHREGDVHFDYDETWTIGDDQGTDLLQVAAHEFGHVLGLQHTTAAKALMSAFYTFRYPLSLSPDDCRGVQHLYGQPWPTVTSRTPALGPQAGIDTNEIAPLEPDAPPDACEASFDAVSTIRGELFFFKAGFVWRLRGGQLQPGYPALASRHWQGLPSPVDAAFEDAQGHIWFFQGAQYWVYDGEKPVLGPAPLTELGLVRFPVHAALVWGPEKNKIYFFRGRDYWRFHPSTRRVDSPVPRRATDWRGVPSEIDAAFQDADGYAYFLRGRLYWKFDPVKVKALEGFPRLVGPDFFGCAEPANTFL

SEQ ID NO: 88 - DNA, 2930 bp, Homo sapiens
  source 1..2930: mol_type = genomic DNA
atgggtgctgaggaggaggtgctggtcacactatcagggggagccccctggggcttccgacttcatgggggggccgagcagaggaaaccgttacaggtgtctaagattcgaagacggagccaggctggcagagcaggactccgagagagggaccagctcttggcaatcaatggggtctcttgcaccaacctctcccatgccagtgccatgagcctcatcgatgcctcaggaaatcagcttgtcctcactgtgcagcggttagcagacgagggtcctgtgcaatctccatctccccatgagcttcaggtgctgtcacccttatctccactaagtcctgagccccctggtgctccagttcctcagcctcttcagcctgggagccttcgttcacctcctgatagtgaggcttactacggagagactgacagtgatgctgatggccctgccacccaggagaagccccgtcgacctcgccgccgaggccccacaaggcccacccctccgggtgccccacctgatgaggtctacctgtctgacagccctgcagagccagcacctactatccctggccctcccagccagggtgacagcgtgtgagctccccgtcttgggaggatggggcagcccttcagccacccccagctgaggctctgctgttaccccatggccccctccgacctggtcctcatctcatccctatggtggggcctgttccccacccagtggcagaagatcttactaccacctacacccagaaggccaagcaagccaaactgcaacgtgcagagagcctccaagagaagagcataaaagaggccaagaccaaatgcaggacaattgcatccctgctcactgcagcccccaacccccactccaaaggggtacttatgtttaagaaacggcggcagagagccaagaagtacaccctggtgagcttcggggctgctgctgggacaggcgctgaggaggaggacggcgttccccccacgagtgagtccgagctggacgaagaagccttctctgacgcccgcagcctcaccaatcaatctgactgggacagtccctatctggacatggagcttgccagggcgggctcaagagcatcagagggccagggctctgggctgggagggcagctgagtgaggtctctgggcgaggggtgcagctctttgaacagcagcgccagcgcgcagactccagcacccaggaactggcacgggtcgaaccagcagccatgctcaacggggaaggcctgcagtcaccacctcgggcccagagtgctcccccagaggcagctgtgctcccacccagccccttgccggcgcctgtagccagccccagacccttccaaccaggtggtggagccccgaccccagctccaagcatctttaaccggtcagccaggccctttaccccgggcctacaagggcagcggccaactaccacctcggttattttccggcctttagcccccaaaagggcgaacgacagcctggggggcctcagccccgccccaccccccttcttgtcttcgcaggggcccacccctctgcccagcttcacttcaggggttcccagccacgcgccagtctctggttcccccagcaccccacgctcctcgggccctgtgacagccaccagctccctgtacatcccagcccctagtcggcctgtcaccccaggtggagctccagagccccccgctcctcctagcgcagctgccatgacctccaccgcttctatcttcctatctgcgcctttgcgaccctctgcgcgcccagaggcgcctgccccaggcccaggggctcctgagccccccagcgctcgcgagcagcgcatctctgtgccagctgcccgcacgggtatcctgcaggaggcccggcgccgggggacccggaagcagatgttccggccgggaaaggaggagacgaagaactcgcccaaccccgagctgctatcgctggtacagaacctggatgaaaagcctcgggccgggggtgcagaatctggtcctgaagaagatgctctgagcctcggggctgaagcctgcaacttcatgcagccagtaggggccaggagttacaagaccctgcctcacgtgacacctaagaccccccctccaatggctcccaagaccccgccccctatgactcctaagactccacccccagtggctcctaagcccccatctcgagggctccttgatgggctcgtgaatggggcagcctcttcggctggaatccctgagccaccaaggctgcagggcaggggtggggagctgtttgctaagcggcagagccgtgcggacaggtatgtggtggaaggtacacctggtcctggtcttggccctcggcctagaagtccttctcctaccccgtctctgcccccttcctggaaatattcacccaacatccgtgccccgcctcctattgcttacaacccactgctctctccctttttcccccaggcggcccgaactctccctaaggcccaatcccaggggcctcgggcaacacccaagcagggcatcaaggctctagattttatgcggcatcagccctatcaacttaaaactgccatgttctgttttgatgaggttcccccgactcctggccctatcgcctcagggtcccccaaaactgcccgagtccaggagattcgccggttttccactccggcaccccagcccactgcagaacccctggctcccactgtgcttgccccccgagcagccactacactggatgagcccatctggagaacagaactggcctcagcccctgttcctagcccagcccctcctccagaggctcccaggggccttggggcttctcccagctcctgcggtttccaggtagccaggccccgattttcagccaccagaacaggattgcaagctcatgtgtggaggcctggggcagggcaccag

SEQ ID NO: 89 - protein, 977 aa, Homo sapiens
MGAEEEVLVTLSGGAPWGFRLHGGAEQRKPLQVSKIRRRSQAGRAGLRERDQLLAINGVSCTNLSHASAMSLIDASGNQLVLTVQRLADEGPVQSPSPHELQVLSPLSPLSPEPPGAPVPQPLQPGSLRSPPDSEAYYGETDSDADGPATQEKPRRPRRRGPTRPTPPGAPPDEVYLSDSPAEPAPTIPGPPSQGDSRVSSPSWEDGAALQPPPAEALLLPHGPLRPGPHLIPMVGPVPHPVAEDLTTTYTQKAKQAKLQRAESLQEKSIKEAKTKCRTIASLLTAAPNPHSKGVLMFKKRRQRAKKYTLVSFGAAAGTGAEEEDGVPPTSESELDEEAFSDARSLTNQSDWDSPYLDMELARAGSRASEGQGSGLGGQLSEVSGRGVQLFEQQRQRADSSTQELARVEPAAMLNGEGLQSPPRAQSAPPEAAVLPPSPLPAPVASPRPFQPGGGAPTPAPSIFNRSARPFTPGLQGQRPTTTSVIFRPLAPKRANDSLGGLSPAPPPFLSSQGPTPLPSFTSGVPSHAPVSGSPSTPRSSGPVTATSSLYIPAPSRPVTPGGAPEPPAPPSAAAMTSTASIFLSAPLRPSARPEAPAPGPGAPEPPSAREQRISVPAARTGILQEARRRGTRKQMFRPGKEETKNSPNPELLSLVQNLDEKPRAGGAESGPEEDALSLGAEACNFMQPVGARSYKTLPHVTPKTPPPMAPKTPPPMTPKTPPPVAPKPPSRGLLDGLVNGAASSAGIPEPPRLQGRGGELFAKRQSRADRYVVEGTPGPGLGPRPRSPSPTPSLPPSWKYSPNIRAPPPIAYNPLLSPFFPQAARTLPKAQSQGPRATPKQGIKALDFMRHQPYQLKTAMFCFDEVPPTPGPIASGSPKTARVQEIRRFSTPAPQPTAEPLAPTVLAPRAATTLDEPIWRTELASAPVPSPAPPPEAPRGLGASPSSCGFQVARPRFSATRTGLQAHVWRPGAGHQ

SEQ ID NO: 90 - DNA, 2259 bp, Homo sapiens
  source 1..2259: mol_type = genomic DNA
atggagacctttgagcccatcagccaagagcccctcagccaagccagctatgacaaagccccagacccagttcctgagctccaagactcgttctatgcagaactgcaacgtgcagagagcctccaagagaagagcataaaagaggccaagaccaaatgcaggacaattgcatccctgctcactgcagcccccaacccccactccaaaggggtacttatgtttaagaaacggcggcagagagccaagaagtacaccctggtgagcttcggggctgctgctgggacaggcgctgaggaggaggacggcgttccccccacgagtgagtccgagctggacgaagaagccttctctgacgcccgcagcctcaccaatcaatctgactgggacagtccctatctggacatggagcttgccagggcgggctcaagagcatcagagggccagggctctgggctgggagggcagctgagtgaggtctctgggcgaggggtgcagctctttgaacagcagcgccagcgcgcagactccagcacccaggaactggcacgggtcgaaccagcagccatgctcaacggggaaggcctgcagtcaccacctcgggcccagagtgctcccccagaggcagctgtgctcccacccagccccttgccggcgcctgtagccagccccagacccttccaaccaggtggtggagccccgaccccagctccaagcatctttaaccggtcagccaggccctttaccccgggcctacaagggcagcggccaactaccacctcggttattttccggcctttagcccccaaaagggcgaacgacagcctggggggcctcagccccgccccaccccccttcttgtcttcgcaggggcccacccctctgcccagcttcacttcaggggttcccagccacgcgccagtctctggttcccccagcaccccacgctcctcgggccctgtgacagccaccagctccctgtacatcccagcccctagtcggcctgtcaccccaggtggagctccagagccccccgctcctcctagcgcagctgccatgacctccaccgcttctatcttcctatctgcgcctttgcgaccctctgcgcgcccagaggcgcctgccccaggcccaggggctcctgagccccccagcgctcgcgagcagcgcatctctgtgccagctgcccgcacgggtatcctgcaggaggcccggcgccgggggacccggaagcagatgttccggccgggaaaggaggagacgaagaactcgcccaaccccgagctgctatcgctggtacagaacctggatgaaaagcctcgggccgggggtgcagaatctggtcctgaagaagatgctctgagcctcggggctgaagcctgcaacttcatgcagccagtaggggccaggagttacaagaccctgcctcacgtgacacctaagaccccccctccaatggctcccaagaccccgccccctatgactcctaagactccacccccagtggctcctaagcccccatctcgagggctccttgatgggctcgtgaatggggcagcctcttcggctggaatccctgagccaccaaggctgcagggcaggggtggggagctgtttgctaagcggcagagccgtgcggacaggtatgtggtggaaggtacacctggtcctggtcttggccctcggcctagaagtccttctcctaccccgtctctgcccccttcctggaaatattcacccaacatccgtgccccgcctcctattgcttacaacccactgctctctccctttttcccccaggcggcccgaactctccctaaggcccaatcccaggggcctcgggcaacacccaagcagggcatcaaggctctagattttatgcggcatcagccctatcaacttaaaactgccatgttctgttttgatgaggttcccccgactcctggccctatcgcctcagggtcccccaaaactgcccgagtccaggagattcgccggttttccactccggcaccccagcccactgcagaacccctggctcccactgtgcttgccccccgagcagccactacactggatgagcccatctggagaacagaactggcctcagcccctgttcctagcccagcccctcctccagaggctcccaggggccttggggcttctcccagctcctgcggtttccaggtagccaggccccgattttcagccaccagaacaggattgcaagctcatgtgtggaggcctggggcagggcaccag

SEQ ID NO: 91 - protein, 753 aa, Homo sapiens
METFEPISQEPLSQASYDKAPDPVPELQDSFYAELQRAESLQEKSIKEAKTKCRTIASLLTAAPNPHSKGVLMFKKRRQRAKKYTLVSFGAAAGTGAEEEDGVPPTSESELDEEAFSDARSLTNQSDWDSPYLDMELARAGSRASEGQGSGLGGQLSEVSGRGVQLFEQQRQRADSSTQELARVEPAAMLNGEGLQSPPRAQSAPPEAAVLPPSPLPAPVASPRPFQPGGGAPTPAPSIFNRSARPFTPGLQGQRPTTTSVIFRPLAPKRANDSLGGLSPAPPPFLSSQGPTPLPSFTSGVPSHAPVSGSPSTPRSSGPVTATSSLYIPAPSRPVTPGGAPEPPAPPSAAAMTSTASIFLSAPLRPSARPEAPAPGPGAPEPPSAREQRISVPAARTGILQEARRRGTRKQMFRPGKEETKNSPNPELLSLVQNLDEKPRAGGAESGPEEDALSLGAEACNFMQPVGARSYKTLPHVTPKTPPPMAPKTPPPMTPKTPPPVAPKPPSRGLLDGLVNGAASSAGIPEPPRLQGRGGELFAKRQSRADRYVVEGTPGPGLGPRPRSPSPTPSLPPSWKYSPNIRAPPPIAYNPLLSPFFPQAARTLPKAQSQGPRATPKQGIKALDFMRHQPYQLKTAMFCFDEVPPTPGPIASGSPKTARVQEIRRFSTPAPQPTAEPLAPTVLAPRAATTLDEPIWRTELASAPVPSPAPPPEAPRGLGASPSSCGFQVARPRFSATRTGLQAHVWRPGAGHQ

SEQ ID NO: 92 - RNA, 4994 nt, Homo sapiens
  source 1..4994: mol_type = mRNA
agagcgagcgccggggccgggcgcgcaggagtgaaaaggaggcggcggccgcagctgcgagcaacagatccggacgccgcgagctgacccgctctgctgttgggcgatttttttttaattgcagaaaaatttattaaattggaaaatcttgcgtttttcaatggcgctggccccgggtcagcgggcgattttctctgcatcaagatgggctttgccgtttccgtagtgggcaccagtggtggcctgattgtcagtcttctcccggcatttttaaggccaggagccgagcgctgcttgtaggcgaataccctacagagcggtttggctttttaaattactgttattattttgggcagagaacagtcggtctggtgcaccccgtcctcgctgcagaagaggctgcgagtccgaggtgggtctctcggaaggtgaaattccttctggggtgagcgagccccggccccgcgcgcagtccagcggccccgcgtgtgtgccctcgccctgccggagccgggaaaatggaggctgtgattgagaaggaatgcagcgcgctcggaggcctcttccagaccatcatcagcgacatgaaggggagctatccagtttgggaagatttcataaacaaagcaggaaagctgcagtcccagcttcggacaacagtagtagcagcagctgccttcttggacgcctttcagaaagtggctgacatggccaccaacacacgtggtgggaccagggagattggatctgctctcaccaggatgtgcatgaggcacagaagcattgaagccaagctgaggcagttttcgagcgctttaattgattgtctgataaacccacttcaagaacagatggaagaatggaagaaagtggccaaccagctggataaagaccacgcaaaagaatataagaaagcccgccaagagataaaaaagaagtcctcggatacgctgaaactgcagaagaaagcaaaaaaagggagaggtgatatccagcctcagttggacagtgctctccaagatgtcaatgataagtatctcttattggaagaaacagaaaagcaggctgtccggaaggctttgattgaagaacgtggccgattctgtaccttcatctctatgctgcggccagtgattgaagaagaaatctcaatgctaggggaaataacccaccttcagaccatctcggaagatctaaaaagcctgaccatggaccctcacaaactgccctcctcaagtgaacaggtgattctggacttgaaaggttctgattacagctggtcgtatcagacgccaccctcttcccccagcaccaccatgtccagaaagtccagtgtctgcagcagcctgaacagtgtcaacagcagtgactcccggtccagcggctcccactcgcattcccccagctcacattaccgctaccgcagctccaacctggcccagcaggctcctgtgaggctgtccagcgtgtcctcccatgactcaggattcatatcccaggatgccttccagtccaagtcaccatcccccatgccgccagaggcccccaaccagttgtctaacgggttttctcactatagtttatcaagtgagtcccacgtggggcccacgggtgcaggccttttccctcattgcctgcctgcctcccgcctgctccctcgggtcacctctgtccaccttccagactacgctcattattacaccattgggcccggcatgttcccgtcatctcagatccctagctggaaggactgggctaagcctgggccctatgaccagcctctggtgaacaccctgcagcgccgcaaagagaagcgagaaccggaccccaacgggggaggacccactaccgccagcggcccacctgcagcagctgaggaggctcagagaccacggagcatgactgtatcggctgccaccaggcctggtgaggagatggaggcttgtgaggagctggccctggccctgtctcggggcctgcagctggacacccagaggagcagccgggactcgcttcagtgctccagcggctacagcacccagacaaccaccccctgctgctctgaggacaccatcccttcccaagtttcagattatgattatttctctgtaagtggtgaccaggaggcagatcagcaggagttcgacaagtcctccaccattccaagaaacagcgacatcagccagtcctaccgacggatgttccaagccaagcgtccagcctcaactgctggcctccccaccaccctgggacctgctatggtcactccaggggttgcaactatccgacggaccccttccaccaagccttctgtccgccggggaaccattggagctggtcccatccccatcaagacacccgtgatccctgtcaagaccccaaccgtcccagacctcccaggggtgttgccagcccctccagatgggccagaagagcggggggagcacagccctgagtcgccatctgtgggtgagggcccccaaggtgtcaccagcatgccctcctcaatgtggagcggccaagcttccgttaaccctccacttccaggcccgaagcccagtatccctgaggagcacagacaggcaattccagaaagtgaagctgaagaccaggaacgggaacccccaagtgccactgtctccccaggccagattccagagagtgaccctgcagacctgagcccaagggatactccacaaggagaagacatgctgaacgccatccgaaggggcgtgaaactgaagaagaccacgacaaacgatcgctcagcccctcgcttttcttaggttcacaagaaatgcgccggtggggaatgaactgtttcattaataaaacctaatttgtcttgatccattccactctataataaaacaaaagattttgtaggcaactcggaatatagctcttttgaaagtactcgacacctttagataagaattaaaaccaacctatgtaactgacataatcttgatcttttaatttgtaaatattgacaattttctttctgcacattttaatcttagtttcccttttgatttttctgaaggtgccaaattccatttaacttttttacaagtctttgtaaaattttaaatgcataaagggggttggggcaggggaaccacgaagtagttaattttagaaaaggatttactatacttcactcttctttttttttccccacaagcttttgtagatgcattgtagtagtctagcttagaagcaaatgcaagttattttaatgtacaaactaaatgggtaagaggtaaaatcttcatttaaatatactatgttctggatgaaaagagcaggagtaacaattgatgagcaatattcagagtgaagtaaatctggaaatggtagactgtgttgggattggggggagggccatgggaggggtacatcgtcaacatagccgatcctgttacatttaagagtagcctcgtaggttgaatttcttctggtagcttcatggtaaatgcatccgaataagccatactggattgcagtgtttgtttctgtagggtgtttaaggacttgacttcctttctcccatgattcctctggactgcacacagcacccacaaccagccccatgcatgctgctgcctctgggcagtcgtagaatctcccacttcagtttctcgttgattgtactcacctttatggaatccaaatacatccaaaagggtaaggcagttttaaaaatgtgaaaacatttaaaaatgataatagcagggaattcttagattatagtaaatgccttttacttaactgtgcccagcaggctgggtgcgttaaaaagcccaagtattttgaaaaaactcgaacagatttgacaagggtagccagcttggagtctagcaacttgccaatgtgtttaccaatctgggggcttgtttttcttttcttctttcaaataaatggcagttaactggctttacagtaaacattgaagagaggaggatttgtttattgtcactgggaatctgaccactatactgtcctttttttgtattctgggtaaatgttttttggaaaagatttgtcttttctaagtggaagttaaatttgttatactgcccatcccctaaagccaacagagatttgtagatttaaagggatcacatttgaagacaatagtgtttaagaaagcaagcaagtcccttagcagtcaggtcataacagggcacatttctgaccgaaccctctcaaggcagaggaggagtttggtgggtttcatacaccctgcagattcctgttggctctaaccctcaattacctaatcttatgctttaacacataactgcattggatgtgagagtaacgtaccgtatggtcattgttctatatattaacattgaacactgctgcgattgctcaaggacattttatgttacggctttaaagcaaaggcatgattattagaaactatttaagcttttttctttgaaaaacaagctccttttacagaatataaacaacagtagtgcctgtggtttagcccaccaatcttgatgactaaaagtagctgatgcattgtgcatatgatgcttgagatggtttttgcaaaagcagaaatcgctgcaaggtaatcacaatagataaaagtggtattttaaacctttgaaataaatggatgtaactgtaccttggtacagcttttcacttgtttagtttttaaacgttagtataatctgaataaataaaatgttgccaaattcaatgtagaaagaatgtgacaacacaccttgggtagttctgcttgtgtttttgcatattgtaaaagcagtgtcacagctaaaaagaaagaaatcgtttctaacagtaaattattgtgctttagttgctagtttgtactgagagttgacctctccctgtgcagttttttgttctaaacttgtataaataacaattgtgtaatgtgtctccctcctacattgtaacaattgcttcagcctacgttataaataaagaaccactagattaaaaaa

SEQ ID NO: 93 - protein, 755 aa, Homo sapiens
MEAVIEKECSALGGLFQTIISDMKGSYPVWEDFINKAGKLQSQLRTTVVAAAAFLDAFQKVADMATNTRGGTREIGSALTRMCMRHRSIEAKLRQFSSALIDCLINPLQEQMEEWKKVANQLDKDHAKEYKKARQEIKKKSSDTLKLQKKAKKGRGDIQPQLDSALQDVNDKYLLLEETEKQAVRKALIEERGRFCTFISMLRPVIEEEISMLGEITHLQTISEDLKSLTMDPHKLPSSSEQVILDLKGSDYSWSYQTPPSSPSTTMSRKSSVCSSLNSVNSSDSRSSGSHSHSPSSHYRYRSSNLAQQAPVRLSSVSSHDSGFISQDAFQSKSPSPMPPEAPNQLSNGFSHYSLSSESHVGPTGAGLFPHCLPASRLLPRVTSVHLPDYAHYYTIGPGMFPSSQIPSWKDWAKPGPYDQPLVNTLQRRKEKREPDPNGGGPTTASGPPAAAEEAQRPRSMTVSAATRPGEEMEACEELALALSRGLQLDTQRSSRDSLQCSSGYSTQTTTPCCSEDTIPSQVSDYDYFSVSGDQEADQQEFDKSSTIPRNSDISQSYRRMFQAKRPASTAGLPTTLGPAMVTPGVATIRRTPSTKPSVRRGTIGAGPIPIKTPVIPVKTPTVPDLPGVLPAPPDGPEERGEHSPESPSVGEGPQGVTSMPSSMWSGQASVNPPLPGPKPSIPEEHRQAIPESEAEDQEREPPSATVSPGQIPESDPADLSPRDTPQGEDMLNAIRRGVKLKKTTTNDRSAPRFS

SEQ ID NO: 94 - RNA, 19 nt, synthetic construct
  source 1..19: mol_type = other RNA
ccatctcggaagatctaaa

SEQ ID NO: 95 - RNA, 19 nt, synthetic construct
  source 1..19: mol_type = other RNA
ccagcttcggacaacagta

SEQ ID NO: 96 - RNA, 19 nt, synthetic construct
  source 1..19: mol_type = other RNA
ccatcatcagcgacatgaa

SEQ ID NO: 97 - RNA, 21 nt, synthetic construct
  source 1..21: mol_type = other RNA
ccgtatggtcattgttctata

SEQ ID NO: 98 - RNA, 21 nt, synthetic construct
  source 1..21: mol_type = other RNA
ccgacggatgttccaagccaa

SEQ ID NO: 99 - RNA, 21 nt, synthetic construct
  source 1..21: mol_type = other RNA
tcccgtcatctcagatcccta

SEQ ID NO: 100 - RNA, 21 nt, synthetic construct
  source 1..21: mol_type = other RNA
ccgtatggtcattgttctata

SEQ ID NO: 101 - DNA, 241 bp, synthetic construct
  source 1..241: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggac

SEQ ID NO: 102 - DNA, 403 bp, synthetic construct
  source 1..403: mol_type = other DNA
aactggcctgcccgagaccaaacgtgcggaacgtagttaagtgttagaggtaggatttgaagcctgtcgatcattctgattctccttttctctacgtctgcttcctgtcaatgggcatcctcactgtcaaatgcagatggtacagcagggcttggtctcagccaggcaggcctctccccagtctccatggctcagctgtccagcagtttcatccctagaccatcccaaacatggttgagaagctctgaggggaggacccagcactgcccggcccctgaagataatcagcagtcctgctcagcatatcaatccaagcccactctagacagagatgccggtgcccagttttctatttttaactggtgtgaactgaaggaaaagcacagcattagaagtccaagca

SEQ ID NO: 103 - DNA, 191 bp, synthetic construct
  source 1..191: mol_type = other DNA
ccttcagattaaaaataactaaggtaagggccatgtgggtaggggaggtggtgtgagacggtcctgtctctcctctatctgcccatcggccctttggggaggaggaatgtgcccaaggactaaaaaaaggccctggagccagaggggcgagggcagcagacctttcatgggcaaacctcagggctgctgtc

SEQ ID NO: 104 - DNA, 96 bp, synthetic construct
  source 1..96: mol_type = other DNA
gtaagtaccgcctatagactctataggcacacccctttggctcttatgcatgctgacagactaacagactgttcctttcctgggtcttttctgcag

SEQ ID NO: 105 - DNA, 133 bp, synthetic construct
  source 1..133: mol_type = other DNA
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 106 - DNA, 540 bp, synthetic construct
  source 1..540: mol_type = other DNA
tcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcg

SEQ ID NO: 107 - DNA, 49 bp, synthetic construct
  source 1..49: mol_type = other DNA
aataaaagatctttattttcattagatctgtgtgttggttttttgtgtg

SEQ ID NO: 108 - DNA, 215 bp, synthetic construct
  source 1..215: mol_type = other DNA
gcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 109 - DNA, 134 bp, synthetic construct
  source 1..134: mol_type = other DNA
gatccagacatgataagatacattgatgagtttggacaaaccacaactagaatgcagtgaaaaaaatgctttatttgtgaaatttgtgatgctattgctttatttgtaaccattataagctgcaataaacaagt

SEQ ID NO: 110 - DNA, 130 bp, synthetic construct
  source 1..130: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 111 - DNA, 130 bp, synthetic construct
  source 1..130: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 112 - protein, 736 aa, unidentified
  source 1..736: note = Unclassified Dependoparvovirus adeno-associated virus
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 113 - protein, 599 aa, unidentified
  source 1..599: note = Unclassified Dependoparvovirus adeno-associated virus
TAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 114 - protein, 534 aa, unidentified
  source 1..534: note = Unclassified Dependoparvovirus adeno-associated virus
MASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 115 - protein, 7 aa, unidentified
  source 1..7: note = Unclassified Dependoparvovirus adeno-associated virus
NGSGQNQ

SEQ ID NO: 116 - protein, 6 aa, unidentified
  source 1..6: note = Unclassified Dependoparvovirus adeno-associated virus
NNSEFA

SEQ ID NO: 117 - protein, 5 aa, unidentified
  source 1..5: note = Unclassified Dependoparvovirus adeno-associated virus
GRDNV

SEQ ID NO: 118 - protein, 14 aa, unidentified
  source 1..14: note = Unclassified Dependoparvovirus adeno-associated virus
ATNHQSAQAQAQTG

SEQ ID NO: 119 - protein, 736 aa, synthetic construct
  REGION 1..736: note = Recombinant AAV Capsid Protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTISQVNGRPRDLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 120 - protein, 736 aa, synthetic construct
  REGION 1..736: note = Recombinant AAV Capsid Protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGYHKSGAAQLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 121 - protein, 736 aa, synthetic construct
  REGION 1..736: note = Recombinant AAV Capsid Protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNVSYQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 122 - protein, 736 aa, synthetic construct
  REGION 1..736: note = Recombinant AAV Capsid Protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSMVNQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 123 - protein, 736 aa, synthetic construct
  REGION 1..736: note = Recombinant AAV Capsid Protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQANYGQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 124 - protein, 736 aa, synthetic construct
  REGION 1..736: note = Recombinant AAV Capsid Protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNVGTQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 125 - protein, 736 aa, synthetic construct
  REGION 1..736: note = Recombinant AAV Capsid Protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGYHKSGAAQLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNVSYQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 126 - protein, 736 aa, synthetic construct
  REGION 1..736: note = Recombinant AAV Capsid Protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNLNSMLIWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 127 - protein, 736 aa, synthetic construct
  REGION 1..736: note = Recombinant AAV Capsid Protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNQDASLLWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 128 - protein, 736 aa, synthetic construct
  REGION 1..736: note = Recombinant AAV Capsid Protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNGMSFTWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 129 - protein, 736 aa, synthetic construct
  REGION 1..736: note = Recombinant AAV Capsid Protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGYHKSGAAQLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQANYGQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 130 - protein, 736 aa, synthetic construct
  REGION 1..736: note = Recombinant AAV Capsid Protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGYHKSGAAQLKFSVAGPSNMAVQGRNYIPGPSYRQQRVSTTVTQNQDASLLWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 131 - protein, 736 aa, synthetic construct
  REGION 1..736: note = Recombinant AAV Capsid Protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGYHKSGAAQLKFSVAGPSNMAVQGRNYIPGPSYRQQRVSTTVTQNNGMSFTWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 132 - protein, 736 aa, synthetic construct
  REGION 1..736: note = Recombinant AAV Capsid Protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNLNSMLIWPGASSWALNGRNSLMNPGPAMASHKEGEDRFFPLSGSLIFGKQGTGRDNVDADKVMITNEEEIKTTNPVATESYGQVATNHQANYGQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 133 - protein, 726 aa, synthetic construct
  REGION 1..726: note = Recombinant AAV Capsid Protein
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTNNYNDPQFVDFAPDSTGEYRTTRPIGTRYLTRPL

SEQ ID NO: 134 - protein, 725 aa, synthetic construct
  REGION 1..725: note = Recombinant AAV Capsid Protein
MAADGYLPDWLEDNLSEGIREWWALKPGAPQPKANQQHQDNARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTSNYYKSNNVEFAVNTEGVYSEPRPIGTRYLTRNL

SEQ ID NO: 135 - protein, 725 aa, synthetic construct
  REGION 1..725: note = Recombinant AAV Capsid Protein
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEAAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 136 - protein, 727 aa, synthetic construct
  REGION 1..727: note = Recombinant AAV Capsid Protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 137 - protein, 736 aa, synthetic construct
  REGION 1..736: note = Recombinant AAV Capsid Protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVTVEMEWELKKENSKRWNPEIQYTNNYNDPQFVDFAPDSTGEYRTTRPIGTRYLTRPL

SEQ ID NO: 138 - protein, 726 aa, synthetic construct
  REGION 1..726: note = Recombinant AAV Capsid Protein
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSFAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 139 - protein, 726 aa, synthetic construct
  REGION 1..726: note = Recombinant AAV Capsid Protein
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 140 - protein, 726 aa, synthetic construct
  REGION 1..726: note = Recombinant AAV Capsid Protein
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTNNYNDPQFVDFAPDSTGEYRTTRPIGTRYLTRPL

SEQ ID NO: 141 - protein, 727 aa, synthetic construct
  REGION 1..727: note = Recombinant AAV Capsid Protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVTVEMEWELKKENSKRWNPEIQYTSNYYKSNNVEFAVNTEGVYSEPRPIGTRYLTRNL

SEQ ID NO: 142 - protein, 726 aa, synthetic construct
  REGION 1..726: note = Recombinant AAV Capsid Protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 143 - protein, 726 aa, synthetic construct
  REGION 1..726: note = Recombinant AAV Capsid Protein
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 144 - protein, 736 aa, synthetic construct
  REGION 1..736: note = Recombinant AAV Capsid Protein
MAADGYLPDWLEDNLSEGIREWWALKPGAPQPKANQQHQDNARGLVLPGYNYLGPGNGLDRGEPVNRADEVAREHDISYNEQLEAGDNPYLKYNHADAEFQERLKEDTSFGGNLGKAVFQAKKRVLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGNASGDWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSKMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVTVEMEWELKKENSKRWNPEIQYTNNYNDPQFVDFAPDSTGEYRTTRPIGTRYLTRNL

SEQ ID NO: 145 - protein, 735 aa, synthetic construct
  REGION 1..735: note = Recombinant AAV Capsid Protein
MSFVDHPPDWLEEVGEGLREFLGLEAGPPKPKPNQQHQDQARGLVLPGYNYLGPGNGLDRGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTNNYNDPQFVDFAPDSTGEYRTTRPIGTRYLTRPL

SEQ ID NO: 146 - protein, 735 aa, synthetic construct
  REGION 1..735: note = Recombinant AAV Capsid Protein
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELKKENSKRWNPEIQYTSNYYKSNNVEFAVNTEGVYSEPRPIGTRYLTRNL

SEQ ID NO: 147 - protein, 726 aa, synthetic construct; identical to SEQ ID NO: 143
  REGION 1..726: note = Recombinant AAV Capsid Protein

SEQ ID NO: 148 - protein, 726 aa, synthetic construct
  REGION 1..726: note = Recombinant AAV Capsid Protein
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 149 - protein, 726 aa, synthetic construct
  REGION 1..726: note = Recombinant AAV Capsid Protein
MSFVDHPPDWLEEVGEGLREFLGLEAGPPKPKPNQQHQDQARGLVLPGYKYLGPGNGLDKGEPVNAADAAALEHDKAYDQQLKAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 150 - protein, 727 aa, synthetic construct
  REGION 1..727: note = Recombinant AAV Capsid Protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPFGLVEEGAKTAPTGKRIDDHFPKRKKARTEEDSKPSTSSDAEAGPSGSQQLQIPAQPASSLGADTMSAGGGGPLGDNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 151 - protein, 727 aa, synthetic construct
  REGION 1..727: note = Recombinant AAV Capsid Protein
MAADGYLPDWLEDNLSEGIREWWALKPGAPQPKANQQHQDNARGLVLPGYKYLGPGNGLDKGEPVNAADAAALEHDKAYDQQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 152 - protein, 735 aa, synthetic construct
  REGION 1..735: note = Recombinant AAV Capsid Protein
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 153 - protein, 736 aa, synthetic construct
  REGION 1..736: note = Recombinant AAV Capsid Protein
MAADGYLPDWLEDNLSEGIREWWALKPGAPQPKANQQHQDNARGLVLPGYNYLGPGNGLDRGEPVNRADEVAREHDISYNEQLEAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 154 - protein, 736 aa, synthetic construct
  REGION 1..736: note = Recombinant AAV Capsid Protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQEKLADDTSFGGNLGKAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 155 - protein, 736 aa, synthetic construct
  REGION 1..736: note = Recombinant AAV Capsid Protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQEKLADDTSFGGNLGKAVFQAKKRVLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 156 - protein, 735 aa, synthetic construct; identical to SEQ ID NO: 145
  REGION 1..735: note = Recombinant AAV Capsid Protein

SEQ ID NO: 157 - protein, 736 aa, synthetic construct
  REGION 1..736: note = Recombinant AAV Capsid Protein
MAADGYLPDWLEDNLSEGIREWWALKPGAPQPKANQQHQDNARGLVLPGYNYLGPGNGLDRGEPVNRADEVAREHDISYNEQLEAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGYHKSGAAQLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 158 - protein, 736 aa, synthetic construct
  REGION 1..736: note = Recombinant AAV Capsid Protein
MAADGYLPDWLEDNLSEGIREWWALKPGAPQPKANQQHQDNARGLVLPGYNYLGPGNGLDRGEPVNRADEVAREHDISYNEQLEAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQANYGQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 159 - protein, 736 aa, synthetic construct
  REGION 1..736: note = Recombinant AAV Capsid Protein
MAADGYLPDWLEDNLSEGIREWWALKPGAPQPKANQQHQDNARGLVLPGYNYLGPGNGLDRGEPVNRADEVAREHDISYNEQLEAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGYHKSGAAQLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNVSYQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 160 - protein, 736 aa, synthetic construct
  REGION 1..736: note = Recombinant AAV Capsid Protein
MAADGYLPDWLEDNLSEGIREWWALKPGAPQPKANQQHQDNARGLVLPGYNYLGPGNGLDRGEPVNRADEVAREHDISYNEQLEAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNLNSMLIWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 161 - protein, 736 aa, synthetic construct
  REGION 1..736: note = Recombinant AAV Capsid Protein
MAADGYLPDWLEDNLSEGIREWWALKPGAPQPKANQQHQDNARGLVLPGYNYLGPGNGLDRGEPVNRADEVAREHDISYNEQLEAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGYHKSGAAQLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQANYGQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 162 - protein, 736 aa, synthetic construct
  REGION 1..736: note = Recombinant AAV Capsid Protein
MAADGYLPDWLEDNLSEGIREWWALKPGAPQPKANQQHQDNARGLVLPGYNYLGPGNGLDRGEPVNRADEVAREHDISYNEQLEAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNLNSMLIWPGASSWALNGRNSLMNPGPAMASHKEGEDRFFPLSGSLIFGKQGTGRDNVDADKVMITNEEEIKTTNPVATESYGQVATNHQANYGQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 163 - protein, 735 aa, synthetic construct
  REGION 1..735: note = Recombinant AAV Capsid Protein
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGYHKSGAAQLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELKKENSKRWNPEIQYTSNYYKSNNVEFAVNTEGVYSEPRPIGTRYLTRNL

SEQ ID NO: 164 - protein, 735 aa, synthetic construct
  REGION 1..735: note = Recombinant AAV Capsid Protein
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQANYGQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELKKENSKRWNPEIQYTSNYYKSNNVEFAVNTEGVYSEPRPIGTRYLTRNL

SEQ ID NO: 165 - protein, 735 aa, synthetic construct
  REGION 1..735: note = Recombinant AAV Capsid Protein
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGYHKSGAAQLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNVSYQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELKKENSKRWNPEIQYTSNYYKSNNVEFAVNTEGVYSEPRPIGTRYLTRNL

SEQ ID NO: 166 - protein, 735 aa, synthetic construct
  REGION 1..735: note = Recombinant AAV Capsid Protein
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNLNSMLIWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELKKENSKRWNPEIQYTSNYYKSNNVEFAVNTEGVYSEPRPIGTRYLTRNL

SEQ ID NO: 167 - protein, 735 aa, synthetic construct
  REGION 1..735: note = Recombinant AAV Capsid Protein
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGYHKSGAAQLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQANYGQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELKKENSKRWNPEIQYTSNYYKSNNVEFAVNTEGVYSEPRPIGTRYLTRNL

SEQ ID NO: 168 - protein, 735 aa, synthetic construct
  REGION 1..735: note = Recombinant AAV Capsid Protein
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNLNSMLIWPGASSWALNGRNSLMNPGPAMASHKEGEDRFFPLSGSLIFGKQGTGRDNVDADKVMITNEEEIKTTNPVATESYGQVATNHQANYGQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELKKENSKRWNPEIQYTSNYYKSNNVEFAVNTEGVYSEPRPIGTRYLTRNL

SEQ ID NO: 169 - protein, 727 aa, synthetic construct
  REGION 1..727: note = Recombinant AAV Capsid Protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPFGLVEEGAKTAPTGKRIDDHFPKRKKARTEEDSKPSTSSDAEAGPSGSQQLQIPAQPASSLGADTMSAGGGGPLGDNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGYHKSGAAQLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 170 - protein, 727 aa, synthetic construct; identical to SEQ ID NO: 150
  REGION 1..727: note = Recombinant AAV Capsid Protein

SEQ ID NO: 171 - protein, 727 aa, synthetic construct
  REGION 1..727: note = Recombinant AAV Capsid Protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPFGLVEEGAKTAPTGKRIDDHFPKRKKARTEEDSKPSTSSDAEAGPSGSQQLQIPAQPASSLGADTMSAGGGGPLGDNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGYHKSGAAQLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNVSYQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 172 - protein, 727 aa, synthetic construct
  REGION 1..727: note = Recombinant AAV Capsid Protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPFGLVEEGAKTAPTGKRIDDHFPKRKKARTEEDSKPSTSSDAEAGPSGSQQLQIPAQPASSLGADTMSAGGGGPLGDNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNLNSMLIWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 173 - protein, 727 aa, synthetic construct
  REGION 1..727: note = Recombinant AAV Capsid Protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPFGLVEEGAKTAPTGKRIDDHFPKRKKARTEEDSKPSTSSDAEAGPSGSQQLQIPAQPASSLGADTMSAGGGGPLGDNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGYHKSGAAQLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQANYGQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 174 - protein, 727 aa, synthetic construct
  REGION 1..727: note = Recombinant AAV Capsid Protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPFGLVEEGAKTAPTGKRIDDHFPKRKKARTEEDSKPSTSSDAEAGPSGSQQLQIPAQPASSLGADTMSAGGGGPLGDNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNLNSMLIWPGASSWALNGRNSLMNPGPAMASHKEGEDRFFPLSGSLIFGKQGTGRDNVDADKVMITNEEEIKTTNPVATESYGQVATNHQANYGQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 175 - protein, 7 aa, synthetic construct
KGSGQNQ

SEQ ID NO: 176 - protein, 7 aa, synthetic construct
NASGQNQ

SEQ ID NO: 177 - protein, 7 aa, synthetic construct
NGTGQNQ

SEQ ID NO: 178 - protein, 7 aa, synthetic construct
NGSGLNQ

SEQ ID NO: 179 - protein, 7 aa, synthetic construct
ANDNKLI

SEQ ID NO: 180 - protein, 7 aa, synthetic construct
VNDNKVI

SEQ ID NO: 181 - protein, 7 aa, synthetic construct
NGSGQNH

SEQ ID NO: 182 - protein, 7 aa, synthetic construct
ANDNKVI

SEQ ID NO: 183 - protein, 14 aa, synthetic construct
ATNHENTVSIAQTG

SEQ ID NO: 184 - protein, 14 aa, synthetic construct
ATNHQTLFNSAQTG

SEQ ID NO: 185 - protein, 14 aa, synthetic construct
ATNHNSTYLGAQTG

SEQ ID NO: 186 - protein, 14 aa, synthetic construct
ATNHGSILTHAQTG

SEQ ID NO: 187 - protein, 14 aa, synthetic construct
ATNHMMTTARAQTG

SEQ ID NO: 188 - protein, 14 aa, synthetic construct
ATNHCSTSIRAQTG

SEQ ID NO: 189 - protein, 14 aa, synthetic construct
ATNHEDNIRSAQTG

SEQ ID NO: 190 - protein, 14 aa, synthetic construct
ATNHNNVISGAQTG

SEQ ID NO: 191 - protein, 14 aa, synthetic construct
ATNHQGAYAQAQTG

SEQ ID NO: 192 - protein, 14 aa, synthetic construct
ATNHQANYGQAQTG

SEQ ID NO: 193 - protein, 14 aa, synthetic construct
ATNHNMNRVNAQTG

SEQ ID NO: 194 - protein, 14 aa, synthetic construct
ATNHSNSVQSAQTG

SEQ ID NO: 195 - protein, 14 aa, synthetic construct
ATNHSSTFQGAQTG

SEQ ID NO: 196 - protein, 14 aa, synthetic construct
ATNHVSSFTSAQTG

SEQ ID NO: 197 - protein, 14 aa, synthetic construct
ATNHSTTNFRAQTG

SEQ ID NO: 198 - protein, 14 aa, synthetic construct
ATNHSSIFNSAQTG

SEQ ID NO: 199 - protein, 14 aa, synthetic construct
ATNHAGNYNNAQTG

SEQ ID NO: 200 - protein, 14 aa, synthetic construct
ATNHTSVISIAQTG

SEQ ID NO: 201 - protein, 14 aa, synthetic construct
ATNHHSRVEIAQTG

SEQ ID NO: 202 - protein, 14 aa, synthetic construct
ATNHSSIIYSAQTG

SEQ ID NO: 203 - protein, 14 aa, synthetic construct
ATNHSGRDSYAQTG

SEQ ID NO: 204 - protein, 14 aa, synthetic construct
ATNHSSSYNNAQTG

SEQ ID NO: 205 - protein, 14 aa, synthetic construct
ATNHHNPSINAQTG

SEQ ID NO: 206 - protein, 14 aa, synthetic construct
ATNHNRNGLLAQTG

SEQ ID NO: 207 - protein, 14 aa, synthetic construct
ATNHESTSVRAQTG

SEQ ID NO: 208 - protein, 14 aa, synthetic construct
ATNHNIRTEMAQTG

SEQ ID NO: 209 - protein, 14 aa, synthetic construct
ATNHLSVSSIAQTG

SEQ ID NO: 210 - protein, 14 aa, synthetic construct
ATNHEDIIRSAQTG

SEQ ID NO: 211 - protein, 14 aa, synthetic construct
ATNRQTAQAQAQTG

SEQ ID NO: 212 - protein, 14 aa, synthetic construct
ATNRQIAQAQAQTG

SEQ ID NO: 213 - protein, 14 aa, synthetic construct
ATNHNTKLAIAQTG

SEQ ID NO: 214 - protein, 14 aa, synthetic construct
ATNHTGTSIIAQTG

SEQ ID NO: 215 - protein, 16 aa, synthetic construct
ATNHQWMSAQAQAQTG

SEQ ID NO: 216 - protein, 14 aa, synthetic construct
ATNHQDARAQAQTG

SEQ ID NO: 217 - protein, 16 aa, synthetic construct
ATNHQHYSAQAQAQTG

SEQ ID NO: 218 - protein, 14 aa, synthetic construct
  VARIANT 1: note = any amino acid
  VARIANT 5..6: note = any amino acid
  VARIANT 10..14: note = any amino acid
XDVQXXPGFXXXXX

SEQ ID NO: 219 - protein, 14 aa, synthetic construct
  VARIANT 6: note = any amino acid
  VARIANT 10..12: note = any amino acid
  VARIANT 14: note = any amino acid
ADVQQXPGFXXXTX

SEQ ID NO: 220 - protein, 14 aa, synthetic construct
  VARIANT 1: note = any amino acid
  VARIANT 5..6: note = any amino acid
  VARIANT 10: note = any amino acid
  VARIANT 13..14: note = any amino acid
XDVQXXPGFXAQXX

SEQ ID NO: 221 - protein, 14 aa, synthetic construct
  VARIANT 1: note = any amino acid
  VARIANT 5..6: note = any amino acid
  VARIANT 10: note = any amino acid
  VARIANT 13..14: note = any amino acid
XDVQXXPGFXAEXX

SEQ ID NO: 222 - protein, 14 aa, synthetic construct
  VARIANT 1: note = any amino acid
  VARIANT 5..6: note = any amino acid
  VARIANT 10: note = any amino acid
  VARIANT 13..14: note = any amino acid
XDVQXXPGFXPQXX

SEQ ID NO: 223 - protein, 14 aa, synthetic construct
  VARIANT 1: note = any amino acid
  VARIANT 5..6: note = any amino acid
  VARIANT 10: note = any amino acid
  VARIANT 13..14: note = any amino acid
XDVQXXPGFXPEXX

SEQ ID NO: 224 - protein, 14 aa, synthetic construct
  VARIANT 6: note = any amino acid
  VARIANT 10: note = any amino acid
  VARIANT 14: note = any amino acid
ADVQQXPGFXAQTX

SEQ ID NO: 225 - protein, 14 aa, synthetic construct
  VARIANT 6: note = any amino acid
  VARIANT 10: note = any amino acid
  VARIANT 14: note = any amino acid
ADVQQXPGFXAETX

SEQ ID NO: 226 - protein, 14 aa, synthetic construct
  VARIANT 6: note = any amino acid
  VARIANT 10: note = any amino acid
  VARIANT 14: note = any amino acid
ADVQQXPGFXPQTX

SEQ ID NO: 227 - protein, 14 aa, synthetic construct
  VARIANT 6: note = any amino acid
  VARIANT 10: note = any amino acid
  VARIANT 14: note = any amino acid
ADVQQXPGFXPETX

SEQ ID NO: 228 - protein, 14 aa, synthetic construct
  VARIANT 1: note = any amino acid
  VARIANT 5..6: note = any amino acid
  VARIANT 13..14: note = any amino acid
XDVQXXPGFQAQXX

SEQ ID NO: 229 - protein, 14 aa, synthetic construct
  VARIANT 6: note = any amino acid
  VARIANT 14: note = any amino acid
ADVQQXPGFQAQTX

SEQ ID NO: 230 - protein, 14 aa, synthetic construct
  VARIANT 1: note = any amino acid
  VARIANT 5: note = any amino acid
  VARIANT 10..13: note = any amino acid
XDVQXLPGFXXXXE

SEQ ID NO: 231 - protein, 14 aa, synthetic construct
  VARIANT 10..12: note = any amino acid
ADVQQLPGFXXXTE

SEQ ID NO: 232 - protein, 14 aa, synthetic construct
  VARIANT 1: note = any amino acid
  VARIANT 5..6: note = any amino acid
  VARIANT 11..14: note = any amino acid
XDVQXXPGFQXXXX

SEQ ID NO: 233 - protein, 14 aa, synthetic construct
  VARIANT 6: note = any amino acid
  VARIANT 11..12: note = any amino acid
  VARIANT 14: note = any amino acid
ADVQQXPGFQXXTX

SEQ ID NO: 234 - protein, 14 aa, synthetic construct
ADVQQLPGFQAQTE

SEQ ID NO: 235 - protein, 14 aa, synthetic construct
ADVQQHPGFQAQTE

SEQ ID NO: 236 - protein, 14 aa, synthetic construct
ADVQQLPGFGAQTE

SEQ ID NO: 237 - protein, 14 aa, synthetic construct
ADVQQLPGFRPETE

SEQ ID NO: 238 - protein, 14 aa, synthetic construct
ADVQQLPGFNAQTE

SEQ ID NO: 239 - protein, 14 aa, synthetic construct
ADVQQLPGFQAETE

SEQ ID NO: 240 - protein, 14 aa, synthetic construct
ADVQQLPGFGAETE

SEQ ID NO: 241 - protein, 14 aa, synthetic construct
ADVQQLPGFGPQTE

SEQ ID NO: 242 - protein, 14 aa, synthetic construct
ADVQQLPGFHAQTE

SEQ ID NO: 243 - protein, 14 aa, synthetic construct
ADVQQLPGFRAQTE

SEQ ID NO: 244 - protein, 14 aa, synthetic construct
ADVQQLPGFRAETE

SEQ ID NO: 245 - protein, 14 aa, synthetic construct
ADVQQLPGFMAQTE

SEQ ID NO: 246 - protein, 14 aa, synthetic construct
ADVQQLPGFPAQTE

SEQ ID NO: 247 - protein, 14 aa, synthetic construct
ADVQQLPGFSAQTE

SEQ ID NO: 248 - protein, 14 aa, synthetic construct
ADVQQLPGFSAETE

SEQ ID NO: 249 - protein, 14 aa, synthetic construct
ADVQQLPGFRPQTE

SEQ ID NO: 250 - protein, 14 aa, synthetic construct
ADVQQVPGFQAQTM

SEQ ID NO: 251 - protein, 14 aa, synthetic construct
ADVQQVPGFQAQTQ

SEQ ID NO: 252 - protein, 14 aa, synthetic construct
ADVQQCPGFQAQTE

SEQ ID NO: 253 - protein, 14 aa, synthetic construct
ADVQQQPGFQAQTE

SEQ ID NO: 254 - protein, 14 aa, synthetic construct
ADVQQGPGFQAQTD

SEQ ID NO: 255 - protein, 14 aa, synthetic construct
ADVQQHPGFQAQTD

SEQ ID NO: 256 - protein, 14 aa, synthetic construct
ADVQQHPGFQAQTL

SEQ ID NO: 257 - protein, 14 aa, synthetic construct
ADVQQIPGFQAQTA

SEQ ID NO: 258 - protein, 14 aa, synthetic construct
ADVQQIPGFQAQTC

SEQ ID NO: 259 - protein, 14 aa, synthetic construct
ADVQQIPGFQAQTQ

SEQ ID NO: 260 - protein, 14 aa, synthetic construct
ADVQQIPGFQAQTH

SEQ ID NO: 261 - protein, 14 aa, synthetic construct
ADVQQIPGFQAQTM

SEQ ID NO: 262 - protein, 14 aa, synthetic construct
ADVQQLPGFQAQTD

SEQ ID NO: 263 - protein, 14 aa, synthetic construct
ADVQQLPGFQAQTC

SEQ ID NO: 264 - protein, 14 aa, synthetic construct
ADVQQLPGFQAQTQ

SEQ ID NO: 265 - protein, 14 aa, synthetic construct
ADVQQLPGFQAQTL

SEQ ID NO: 266 - protein, 14 aa, synthetic construct
ADVQQLPGFQAQTM

SEQ ID NO: 267 - protein, 14 aa, synthetic construct
ADVQQMPGFQAQTD

SEQ ID NO: 268 - protein, 14 aa, synthetic construct
ADVQQMPGFQAQTE

SEQ ID NO: 269 - protein, 14 aa, synthetic construct
ADVQQMPGFQAQTL

SEQ ID NO: 270 - protein, 14 aa, synthetic construct
ADVQQMPGFQAQTF

SEQ ID NO: 271 - protein, 14 aa, synthetic construct
ADVQQMPGFQAQTY

SEQ ID NO: 272 - protein, 14 aa, synthetic construct
ADVQQFPGFQAQTA

SEQ ID NO: 273 - protein, 14 aa, synthetic construct
ADVQQFPGFQAQTQ

SEQ ID NO: 274 - protein, 14 aa, synthetic construct
ADVQQFPGFQAQTH

SEQ ID NO: 275 - protein, 14 aa, synthetic construct
ADVQQFPGFQAQTT

SEQ ID NO: 276 - protein, 14 aa, synthetic construct
ADVQQFPGFQAQTV

SEQ ID NO: 277 - protein, 14 aa, synthetic construct
ADVQQPPGFQAQTE

SEQ ID NO: 278 - protein, 14 aa, synthetic construct
ADVQQTPGFQAQTE

SEQ ID NO: 279 - protein, 14 aa, synthetic construct
ADVQQYPGFQAQTA

SEQ ID NO: 280 - protein, 14 aa, synthetic construct
ADVQQYPGFQAQTC

SEQ ID NO: 281 - protein, 14 aa, synthetic construct
ADVQQYPGFQAQTQ

SEQ ID NO: 282 - protein, 14 aa, synthetic construct
ADVQQYPGFQAQTH

SEQ ID NO: 283 - protein, 14 aa, synthetic construct
ADVQQYPGFQAQTI

SEQ ID NO: 284 - protein, 14 aa, synthetic construct
ADVQQYPGFQAQTM

SEQ ID NO: 285 - protein, 14 aa, synthetic construct
ADVQQYPGFQAQTS

SEQ ID NO: 286 - protein, 14 aa, synthetic construct
ADVQQYPGFQAQTT

SEQ ID NO: 287 - protein, 14 aa, synthetic construct
ADVQQYPGFQAQTV

SEQ ID NO: 288 - protein, 14 aa, synthetic construct
ADVQQVPGFQAQTA

SEQ ID NO: 289 - protein, 14 aa, synthetic construct
ADVQQVPGFQAQTN

SEQ ID NO: 290 - protein, 14 aa, synthetic construct
ADVQQVPGFQAQTD

SEQ ID NO: 291 - protein, 14 aa, synthetic construct
ADVQQVPGFQAQTC

SEQ ID NO: 292 - protein, 14 aa, synthetic construct
ADVQQVPGFQAQTH

SEQ ID NO: 293 - protein, 14 aa, synthetic construct
ADVQQVPGFQAQTL

SEQ ID NO: 294 - protein, 724 aa, unidentified
  source 1..724: note = Unclassified Dependoparvovirus adeno-associated virus
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 295 - protein, 588 aa, unidentified
  source 1..588: note = Unclassified Dependoparvovirus adeno-associated virus
TAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 296 - protein, 532 aa, unidentified
  source 1..532: note = Unclassified Dependoparvovirus adeno-associated virus
MSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 297 - protein, 14 aa, unidentified
  source 1..14: note = Unclassified Dependoparvovirus adeno-associated virus
ATNNQSSTTAPATG

SEQ ID NO: 298 - protein, 738 aa, unidentified
  source 1..738: note = Unclassified Dependoparvovirus adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 299 - protein, 601 aa, unidentified
  source 1..601: note = Unclassified Dependoparvovirus adeno-associated virus
TAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 300 - protein, 535 aa, unidentified
  source 1..535: note = Unclassified Dependoparvovirus adeno-associated virus
MAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 301 - protein, 14 aa, unidentified
  source 1..14: note = Unclassified Dependoparvovirus adeno-associated virus
ADNLQQQNAAPIVG

SEQ ID NO: 302 - protein, 738 aa, unidentified
  source 1..738: note = Unclassified Dependoparvovirus adeno-associated virus
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 303 - protein, 601 aa, unidentified
  source 1..601: note = Unclassified Dependoparvovirus adeno-associated virus
TAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 304 - protein, 535 aa, unidentified
  source 1..535: note = Unclassified Dependoparvovirus adeno-associated virus
MAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 305 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQRGDLGLSAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 306 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGASTNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQRGDLLLSAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 307 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQRGDLLLSAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 308 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGASTNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 309 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGVSLGGGQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 310 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEIAWPRASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYCKSNNVEFAVNTEGVYSEPRPIGTRYLTRNL

SEQ ID NO: 311 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQENRRGDFNNTAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 312 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQRGDHASWAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 313 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSNSRGDYNSLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 314 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQASTRGDHGVLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 315 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLANKTTNKDARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSINVDFTVDTNGVYSEPRPIGTRYLTRNL

SEQ ID NO: 316 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLANKIQRTDARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSINVDFTVDTNGVYSEPRPIGTRYLTRNL

SEQ ID NO: 317 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHEDNIRSAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 318 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIKGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHENTVSIAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 319 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIKGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHNSTYLGAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 320 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIKGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQANYGQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 321 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHMMTTARAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 322 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQGAYAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 323 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHNSTYLGAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 324 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHCSTSIRAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 325 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIKGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHEDNIRSAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 326 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIKGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHNNVISGAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 327 - protein, 736 aa, synthetic construct
  REGION 1..736: note = Recombinant AAV capsid protein ZC388
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNRQTAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 328 - protein, 736 aa, synthetic construct
  REGION 1..736: note = Recombinant AAV capsid protein ZC393
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHNMNRVNAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 329 - protein, 736 aa, synthetic construct
  REGION 1..736: note = Recombinant AAV capsid protein ZC394
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHNNVISGAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 330 - protein, 736 aa, synthetic construct
  REGION 1..736: note = Recombinant AAV capsid protein ZC395
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHSNSVQSAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 331 - protein, 736 aa, synthetic construct
  REGION 1..736: note = Recombinant AAV capsid protein ZC399
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHSSTFQGAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 332 - protein, 736 aa, synthetic construct
  REGION 1..736: note = Recombinant AAV capsid protein ZC405
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHVSSFTSAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 333 - protein, 736 aa, synthetic construct
  REGION 1..736: note = Recombinant AAV capsid protein ZC407
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHSTTNFRAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 334 - protein, 736 aa, synthetic construct
  REGION 1..736: note = Recombinant AAV capsid protein ZC410
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHSSIFNSAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 335 - protein, 736 aa, synthetic construct
  REGION 1..736: note = Recombinant AAV capsid protein ZC411
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHAGNYNNAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 336 - protein, 736 aa, synthetic construct
  REGION 1..736: note = Recombinant AAV capsid protein ZC422
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHTSVISIAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 337 - protein, 736 aa, synthetic construct
  REGION 1..736: note = Recombinant AAV capsid protein ZC425
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHHSRVEIAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 338 - protein, 736 aa, synthetic construct
  REGION 1..736: note = Recombinant AAV capsid protein ZC447
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHSSIIYSAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 339 - protein, 736 aa, synthetic construct
  REGION 1..736: note = Recombinant AAV capsid protein ZC451
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHSGRDSYAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 340 - protein, 736 aa, synthetic construct
  REGION 1..736: note = Recombinant AAV capsid protein ZC455
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHSSSYNNAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 341 - protein, 736 aa, synthetic construct
  REGION 1..736: note = Recombinant AAV capsid protein ZC460
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHHNPSINAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 342 - protein, 736 aa, synthetic construct
  REGION 1..736: note = Recombinant AAV capsid protein ZC465
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNRQIAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 343 - protein, 736 aa, synthetic construct
  REGION 1..736: note = Recombinant AAV capsid protein ZC467
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHNRNGLLAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 344 - protein, 736 aa, synthetic construct
  REGION 1..736: note = Recombinant AAV capsid protein ZC468
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHESTSVRAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 345 - protein, 736 aa, synthetic construct
  REGION 1..736: note = Recombinant AAV capsid protein ZC469
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHNIRTEMAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 346 - protein, 736 aa, synthetic construct
  REGION 1..736: note = Recombinant AAV capsid protein ZC470
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQTLFNSAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 347 - protein, 736 aa, synthetic construct
  REGION 1..736: note = Recombinant AAV capsid protein ZC474
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHLSVSSIAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 348 - protein, 736 aa, synthetic construct
  REGION 1..736: note = Recombinant AAV capsid protein ZC477
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHEDIIRSAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 349 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIKGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQTLFNSAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 350 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIKGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHGSILTHAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 351 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVADVQQLPGFQAQTEWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 352 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVADVQQHPGFQAQTEWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 353 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSHVRGDLSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 354 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGVVSSGARSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 355 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSSVRGDAARLAGAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 356 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVADVQQVPGFQAQTMWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 357 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVADVQQVPGFQAQTQWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 358 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVADVQQLPGFGAQTEWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 359 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVADVQQLPGFRPETEWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 360 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVADVQQLPGFNAQTEWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 361 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVADVQQIPGFQAQTAWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 362 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVADVQQIPGFQAQTQWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 363 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVADVQQLPGFQAQTLWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 364 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVADVQQMPGFQAQTEWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 365 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVADVQQMPGFQAQTYWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 366 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVADVQQYPGFQAQTAWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 367 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVADVQQYPGFQAQTVWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 368 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVADVQQVPGFQAQTCWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 369 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVADVQQVPGFQAQTHWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 370 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVADVQQLPGFRAETEWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 371 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVADVQQLPGFPAQTEWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 372 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVADVQQLPGFSAQTEWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 373 - protein, 7 aa, synthetic construct
RGDAARL

SEQ ID NO: 374 - protein, 7 aa, synthetic construct
RTDLKGL

SEQ ID NO: 375 - protein, 7 aa, synthetic construct
YPSTGSG

SEQ ID NO: 376 - protein, 8 aa, synthetic construct
FAGSLTRA

SEQ ID NO: 377 - protein, 7 aa, synthetic construct
DRTLTTR

SEQ ID NO: 378 - protein, 7 aa, synthetic construct
RIAGRDV

SEQ ID NO: 379 - protein, 7 aa, synthetic construct
SLGSGVR

SEQ ID NO: 380 - protein, 7 aa, synthetic construct
SHVRGDL

SEQ ID NO: 381 - protein, 7 aa, synthetic construct
VVSSGAR

SEQ ID NO: 382 - protein, 7 aa, synthetic construct
PQYGRGG

SEQ ID NO: 383 - protein, 7 aa, synthetic construct
LQVSRVS

SEQ ID NO: 384 - protein, 7 aa, synthetic construct
VRSYSSN

SEQ ID NO: 385 - protein, 7 aa, synthetic construct
TMRVGSL

SEQ ID NO: 386 - protein, 7 aa, synthetic construct
GAYSRGV

SEQ ID NO: 387 - protein, 7 aa, synthetic construct
LRGGSLG

SEQ ID NO: 388 - protein, 7 aa, synthetic construct
VYGTGVR

SEQ ID NO: 389 - protein, 7 aa, synthetic construct
DKLIIVS

SEQ ID NO: 390 - protein, 7 aa, synthetic construct
AEDRTKL

SEQ ID NO: 391 - protein, 7 aa, synthetic construct
LSASASL

SEQ ID NO: 392 - protein, 7 aa, synthetic construct
LADQTKL

SEQ ID NO: 393 - protein, 7 aa, synthetic construct
LLLKLQE

SEQ ID NO: 394 - protein, 7 aa, synthetic construct
ELPVKTG

SEQ ID NO: 395 - protein, 7 aa, synthetic construct
LDLKVVG

SEQ ID NO: 396 - protein, 5 aa, synthetic construct
RDAVL

SEQ ID NO: 397 - protein, 4 aa, synthetic construct
VRGD

SEQ ID NO: 398 - DNA, 3258 bp, Staphylococcus aureus
  source 1..3258: mol_type = genomic DNA
atggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagtaa

SEQ ID NO: 399 - protein, 1085 aa, Staphylococcus aureus
MAPKKKRKVGIHGVPAAKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGKRPAATKKAGQAKKKK

SEQ ID NO: 400 - DNA, 90 bp, synthetic construct
  source 1..90: mol_type = other DNA
tatttgcatgtcgctatgtgttctgggaaatcaccataaacgtgaaatgtctttggatttgggaatcttataagttctgtatgagaccac

SEQ ID NO: 401 - DNA, 215 bp, synthetic construct
  source 1..215: mol_type = other DNA
gaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccac

SEQ ID NO: 402 - DNA, 95 bp, synthetic construct
  source 1..95: mol_type = other DNA
atatttgcatgtcgctatgtgttctgggaaatcaccataaacgtgaaatccctatcagtgatagagacttataagttccctatcagtgatagaga

SEQ ID NO: 403 - DNA, 10 bp, synthetic construct
  source 1..10: mol_type = other DNA
gccaccatgg

SEQ ID NO: 404 - DNA, 21 bp, synthetic construct
  source 1..21: mol_type = other DNA
ccaaagaagaagcggaaggtc

SEQ ID NO: 405 - DNA, 48 bp, synthetic construct
  source 1..48: mol_type = other DNA
aaaaggccggcggccacgaaaaaggccggccaggcaaaaaagaaaaag

SEQ ID NO: 406 - DNA, 2268 bp, Homo sapiens
  source 1..2268: mol_type = genomic DNA
atggaggctgtgattgagaaggaatgcagcgcgctcggaggcctcttccagaccatcatcagcgacatgaaggggagctatccagtttgggaagatttcataaacaaagcaggaaagctgcagtcccagcttcggacaacagtagtagcagcagctgccttcttggacgcctttcagaaagtggctgacatggccaccaacacacgtggtgggaccagggagattggatctgctctcaccaggatgtgcatgaggcacagaagcattgaagccaagctgaggcagttttcgagcgctttaattgattgtctgataaacccacttcaagaacagatggaagaatggaagaaagtggccaaccagctggataaagaccacgcaaaagaatataagaaagcccgccaagagataaaaaagaagtcctcggatacgctgaaactgcagaagaaagcaaaaaaagggagaggtgatatccagcctcagttggacagtgctctccaagatgtcaatgataagtatctcttattggaagaaacagaaaagcaggctgtccggaaggctttgattgaagaacgtggccgattctgtaccttcatctctatgctgcggccagtgattgaagaagaaatctcaatgctaggggaaataacccaccttcagaccatctcggaagatctaaaaagcctgaccatggaccctcacaaactgccctcctcaagtgaacaggtgattctggacttgaaaggttctgattacagctggtcgtatcagacgccaccctcttcccccagcaccaccatgtccagaaagtccagtgtctgcagcagcctgaacagtgtcaacagcagtgactcccggtccagcggctcccactcgcattcccccagctcacattaccgctaccgcagctccaacctggcccagcaggctcctgtgaggctgtccagcgtgtcctcccatgactcaggattcatatcccaggatgccttccagtccaagtcaccatcccccatgccgccagaggcccccaaccagttgtctaacgggttttctcactatagtttatcaagtgagtcccacgtggggcccacgggtgcaggccttttccctcattgcctgcctgcctcccgcctgctccctcgggtcacctctgtccaccttccagactacgctcattattacaccattgggcccggcatgttcccgtcatctcagatccctagctggaaggactgggctaagcctgggccctatgaccagcctctggtgaacaccctgcagcgccgcaaagagaagcgagaaccggaccccaacgggggaggacccactaccgccagcggcccacctgcagcagctgaggaggctcagagaccacggagcatgactgtatcggctgccaccaggcctggtgaggagatggaggcttgtgaggagctggccctggccctgtctcggggcctgcagctggacacccagaggagcagccgggactcgcttcagtgctccagcggctacagcacccagacaaccaccccctgctgctctgaggacaccatcccttcccaagtttcagattatgattatttctctgtaagtggtgaccaggaggcagatcagcaggagttcgacaagtcctccaccattccaagaaacagcgacatcagccagtcctaccgacggatgttccaagccaagcgtccagcctcaactgctggcctccccaccaccctgggacctgctatggtcactccaggggttgcaactatccgacggaccccttccaccaagccttctgtccgccggggaaccattggagctggtcccatccccatcaagacacccgtgatccctgtcaagaccccaaccgtcccagacctcccaggggtgttgccagcccctccagatgggccagaagagcggggggagcacagccctgagtcgccatctgtgggtgagggcccccaaggtgtcaccagcatgccctcctcaatgtggagcggccaagcttccgttaaccctccacttccaggcccgaagcccagtatccctgaggagcacagacaggcaattccagaaagtgaagctgaagaccaggaacgggaacccccaagtgccactgtctccccaggccagattccagagagtgaccctgcagacctgagcccaagggatactccacaaggagaagacatgctgaacgccatccgaaggggcgtgaaactgaagaagaccacgacaaacgatcgctcagcccctcgcttttcttag

SEQ ID NO: 407 - DNA, 72 bp, Homo sapiens
  source 1..72: mol_type = genomic DNA
atggaggctgtgattgagaaggaatgcagcgcgctcggaggcctcttccagaccatcatcagcgacatgaag

SEQ ID NO: 408 - DNA, 201 bp, Homo sapiens
  source 1..201: mol_type = genomic DNA
tgcagtgagccgagatcgcaccactgaactccagcctggcaacagagctagactctgtctcaaaaaaataaagaaataaataaaaataaaaatacagatggttctcatttttcacagtagctatgttttttttttaattatactttaagttctatggtacatgtgcacaacatgcaggtttgttacatatgtatacatgtg

SEQ ID NO: 409 - DNA, 10 bp, Homo sapiens
  source 1..10: mol_type = genomic DNA
tacagatggt

SEQ ID NO: 410 - DNA, 15 bp, Homo sapiens
  source 1..15: mol_type = genomic DNA
aaaataaaaatacag

SEQ ID NO: 411 - DNA, 13 bp, Homo sapiens
  source 1..13: mol_type = genomic DNA
aatacagatggtt

(19) United States
(12) Patent Application Publication
(10) Pub. No.: US 2025/0332288 A1
(43) Pub. Date: Oct. 30, 2025

(54) CARDIOPROTECTIVE HEART DISEASE THERAPIES

(71) Applicant: Tenaya Therapeutics, Inc., South San Francisco, CA (US)

(72) Inventors: James R. PRIEST, Menlo Park, CA (US); Timothy C. HOEY, Hillsborough, CA (US); Jonathan Hung Yuan TSUI, San Mateo, CA (US); Rebecca Elizabeth SLATER, South San Francisco, CA (US); Sylwia M. FIGARSKA, Redwood City, CA (US)

(21) Appl. No.: 19/069,660

(22) Filed: Mar. 4, 2025

Related U.S. Application Data
(63) Continuation of application No. PCT/US2023/073572, filed on Sep. 6, 2023.
(60) Provisional application No. 63/404,176, filed on Sep. 6, 2022.
(60) Provisional application No. 63/492,455, filed on Mar. 27, 2023.
(60) Provisional application No. 63/495,288, filed on Apr. 10, 2023.

(51) Int. Cl. A61K 48/00; A61K 35/34; A61K 35/545; A61K 38/00; A61P 9/04; C07K 14/47; C12N 5/074; C12N 5/077; C12N 15/113; C12N 15/86

(52) U.S. Cl. CPC A61K 48/0058; A61K 35/34; A61K 35/545; A61K 48/0075; A61P 9/04; C07K 14/4705; C12N 5/0657; C12N 5/0696; C12N 15/113; C12N 15/86; A61K 38/00; C12N 2310/14; C12N 2310/531; C12N 2750/14143

(57) ABSTRACT

The present disclosure relates to compositions and methods for the treatment or prevention of heart disease (e.g., cardiomyopathy) in a subject. In some aspects, the present disclosure relates to vectors encoding, and compositions comprising, a therapeutic gene product, such as an MMP11 polypeptide, SYNPO2L polypeptide, or an oligonucleotide for inhibiting expression of MTSS1, that confers a cardioprotective effect, e.g., in a TTN mutant genetic background. The present disclosure also relates to the treatment of heart disease (e.g., cardiomyopathy, heart failure or related disorders) using such vectors or compositions.

### CROSS-REFERENCE TO RELATED APPLICATIONS

This application is a Continuation of International Application No. PCT/US23/73572, filed Sep. 6, 2023, which claims priority to U.S. Provisional Patent Application No. 63/404,176, filed on Sep. 6, 2022; U.S. Provisional Patent Application No. 63/492,455, filed on Mar. 27, 2023; and U.S. Provisional Patent Application No. 63/495,288, filed on Apr. 10, 2023, the contents of each of which are incorporated by reference herein in their entireties.

### REFERENCE TO SEQUENCE LISTING

The Sequence Listing associated with this application is provided electronically in XML file format and is hereby incorporated by reference into the specification in its entirety. The name of the XML file containing the Sequence Listing is TENA_041_03US_SeqList_ST26.xml. The XML file is 564,627 bytes, was created on Mar. 3, 2025, and is being submitted electronically through the USPTO Patent Center.

### TECHNICAL FIELD

The present disclosure relates to compositions and methods for the treatment or prevention of heart disease (e.g., heart failure or cardiomyopathy) in a subject. In some aspects, the present disclosure relates to vectors encoding, and compositions comprising, a therapeutic gene product, such as an MMP11 polypeptide, an SYNPO2L polypeptide, or an oligonucleotide for inhibiting the expression of MTSS1, that confers a cardioprotective effect, e.g., in a cardiac disease-associated mutant genetic background such as a TTN mutant genetic background. The present disclosure also relates to the treatment of heart diseases (e.g., cardiomyopathy, heart failure or related disorders) using such vectors or compositions.

### BACKGROUND

Cardiomyopathy is responsible for about half of cardiac-related deaths. It is estimated that about 1 in 250 to 1 in 10,000 adults are affected by some form of cardiomyopathy (McKenna et al. Circ Res. 121:722-730 (2017)). Despite major efforts in screening, diagnostics, and therapeutic strategies, the prevalence of cardiomyopathies and incidence of cardiomyopathy-related deaths remain high (Brieler et al. Am Fam Physician. 96:640-646 (2017)).

Cardiomyopathy refers to a collection of conditions of the heart that occur when its ability to pump blood is reduced. Reduction in proper functioning, such as a contractile dysfunction, of the heart muscle can lead to myocardial infarction, heart failure, blood clots, valve problems, and cardiac arrest. Cardiomyopathies can be separated into primary and secondary categories that result in varied phenotypes (McKenna et al. Circ Res. 121:722-730 (2017)). Primary cardiomyopathies can be genetic, acquired, or mixed in etiology. Genetic cardiomyopathies are inherited and include arrhythmogenic right ventricular dysplasia, hypertrophic, ion channel disorders, left ventricular compaction, and mitochondrial myopathies. Acquired cardiomyopathies are due primarily to non-secondary, non-genetic causes that lead to cardiac complications and include myocarditis, peripartum, tachycardia-induced cardiomyopathy, and stress-induced cardiomyopathy. Cardiomyopathies with mixed etiology are caused by a combination of non-genetic and genetic factors and include dilated cardiomyopathy and restrictive cardiomyopathy. Secondary cardiomyopathies refer to heart disease resulting from an extra cardiovascular cause. The underlying causes of secondary cardiomyopathies can be endocrine, infection, exposure to toxins, autoimmune related, nutritional, and/or neuromuscular.

Dilated cardiomyopathy (DCM), a disease affecting the ability of the heart muscle to generate sufficient and effective force to circulate blood throughout the body, affects 1:250 individuals worldwide. Genetic mutations are an important cause of dilated cardiomyopathy, and the most common genetic mutation is in the gene Titin (abbreviated TTN). TTN is the largest protein in the human genome and an important component of the sarcomere in cardiac and skeletal muscle. Truncating variants in TTN (TTNtv) account for 15 to 25% of DCM cases (Herman et al., 2012, NEJM 366(7):619-28; Mazzarotto et al., 2020, Circulation 141(5):387-398; Fang et al., 2020, Herz 45 (Supp 1):29-36). Another genetic mutation that is associated with heart failure or DCM is MLP/CSRP3 gene mutation. MLP (or CSRP3) is expressed in cardiac and skeletal muscle, and MLP-deficient mice show sarcomere damage and myofibrillar disarray and develop dilated cardiomyopathy and heart failure (Arber et al., 1997, Cell 88, 393-403, https://doi.org/10.1016/S0092-8674(00)81878-4; Knoll et al., 2010, Circ. Res. 106, 695-704, https://doi.org/10.1161/CIRCRESAHA.109.206243).

Current treatment for DCM is limited to standard heart failure therapies and heart transplantation. No disease modifying treatments are currently available for TTN cardiomyopathy. Because of the large size of TTN (109 kbp) it is well above the size limit for delivery or supplementation using standard AAV capsids (5-6 kbp). Therefore, there remains a need in the art for new approaches to treatment of TTN-associated diseases (e.g., TTN-associated DCM).

Gene therapy approaches for the treatment of heart disease often employ vectors configured to effectively transduce cardiac cells and to express a transgene in a cardiac-tissue specific manner. AAV vectors, cardiac-specific promoters, or both in combination, may be used to deliver a polynucleotide encoding a gene product (e.g., a therapeutic protein) to heart tissue and thereby express the gene product in that tissue to treat the heart disease. Cardiac-specific promoters include desmin (Des), alpha-myosin heavy chain (α-MHC), myosin light chain 2 (MLC-2) and cardiac troponin C (TNNC1 or cTnC) promoters, as well as the 600 base pair cardiac troponin T (TNNT2) promoter. The delivery of polynucleotides encoding large proteins remains challenging, however, due in part to the packaging limit of viral vectors.

Given these challenges, there remains a need in the art for new genetic targets and improved therapies for heart disease.

### SUMMARY

In some aspects, provided is a vector comprising one or more polynucleotides encoding one or more gene products, operably linked to one or more promoters, wherein the one or more gene products are selected from the group consisting of: (a) a SYNPO2LA polypeptide (e.g., a human SYNPO2LA polypeptide), optionally wherein the promoter is cardiac-specific promoter; (b) a SYNPO2LB polypeptide (e.g., a human SYNPO2LB polypeptide), optionally wherein the promoter is cardiac-specific promoter; and (c) an MTSS1 inhibitor, optionally wherein the MTSS1 inhibitor inhibits the expression of MTSS1 (e.g., a human MTSS1). In some embodiments, the cardiac-specific promoter is a TNNT2 promoter, e.g., a human TNNT2 promoter, a promoter of SEQ ID NO:1, or a promoter of SEQ ID NO:3. In some embodiments, the vector is a viral vector. In some embodiments, the vector is an AAV vector. In some embodiments, the vector is a lentiviral vector. In some embodiments, the vector is a non-viral vector.

In some aspects, provided is recombinant AAV (rAAV) virion, comprising a vector expressing one or more gene products selected from the group consisting of: a SYNPO2LA polypeptide, a SYNPO2LB polypeptide, and an MTSS1 inhibitor, and any one of the AAV capsid proteins described herein. In some embodiments, any one of the AAV capsid proteins (such as engineered AAV capsid proteins) disclosed in the section “Recombinant AAV Vectors and Virions” and/or in Tables 5 to 9B can be used in the rAAV virions provided herein. In some embodiments, the AAV capsid protein is a wild type AAV9 or an engineered AAV9 capsid protein described herein. In some embodiments, the AAV capsid protein is a wild type AAV5 or an engineered AAV5 capsid protein described herein.

In some aspects, provided is a method of treating and/or preventing a heart disease in a subject, comprising administering to the subject a vector according to various embodiments disclosed herein expressing one or more gene products selected from the group consisting of: a SYNPO2LA polypeptide, a SYNPO2LB polypeptide, and an MTSS1 inhibitor (e.g., an MTSS1 inhibitor that inhibits the expression of MTSS1). In some embodiments, the subject is a mammal. In some embodiments, the subject is a human.

In some aspects, provided is a method of expressing one or more gene products in a cell, comprising transducing the cell with a vector according to various embodiments disclosed herein, wherein the one or more gene products are selected from the group consisting of: a SYNPO2LA polypeptide, a SYNPO2LB polypeptide, and an MTSS1 inhibitor (e.g., an MTSS1 inhibitor that inhibits the expression of MTSS1). In some embodiments, the cell is an induced pluripotent stem cell or an isolated cardiomyocyte. In some embodiments, the transducing of the cell is in vitro or ex vivo.

In some aspects, provided is a vector comprising a polynucleotide encoding a SYNPO2LA polypeptide operably linked to a promoter and/or a SYNPO2LB polypeptide operably linked to a promoter. In some embodiments, the promoter is a cardiac-specific promoter, optionally wherein the cardiac-specific promoter is a TNNT2 promoter, optionally wherein the SYNPO2LA polypeptide and/or SYNPO2LB polypeptide is human SYNPO2LA polypeptide and/or human SYNPO2LB polypeptide. In some embodiments, the polynucleotide encoding the SYNPO2LA polypeptide shares at least 80%, at least 85%, at least 90%, at least 95%, at least 96%, at least 97%, at least 98%, at least 99%, or 100% identity to SEQ ID NO: 88, and/or the SYNPO2LA polypeptide shares at least 80%, at least 85%, at least 90%, at least 95%, at least 96%, at least 97%, at least 98%, at least 99%, or 100% identity to SEQ ID NO: 89; and/or the polynucleotide encoding the SYNPO2LB polypeptide shares at least 80%, at least 85%, at least 90%, at least 95%, at least 96%, at least 97%, at least 98%, at least 99%, or 100% identity to SEQ ID NO: 90, and/or the SYNPO2LB polypeptide shares at least 80%, at least 85%, at least 90%, at least 95%, at least 96%, at least 97%, at least 98%, at least 99%, or 100% identity to SEQ ID NO: 91.

In some aspects, provided is a vector comprising a polynucleotide encoding a SYNPO2LA polypeptide operably linked to a promoter, optionally wherein the promoter is a cardiac-specific promoter, optionally wherein the cardiac-specific promoter is a TNNT2 promoter, optionally wherein the SYNPO2LA polypeptide is human SYNPO2LA. In some embodiments, the polynucleotide encoding the SYNPO2LA polypeptide shares at least 80%, at least 85%, at least 90%, at least 95%, at least 96%, at least 97%, at least 98%, at least 99%, or 100% identity to SEQ ID NO: 88, and/or the SYNPO2LA polypeptide shares at least 80%, at least 85%, at least 90%, at least 95%, at least 96%, at least 97%, at least 98%, at least 99%, or 100% identity to SEQ ID NO: 89.

In some aspects, provided is a vector comprising a polynucleotide encoding a SYNPO2LB polypeptide operably linked to a promoter, optionally wherein the promoter is a cardiac-specific promoter, optionally wherein the cardiac-specific promoter is a TNNT2 promoter, optionally wherein the SYNPO2LB polypeptide is human SYNPO2LB polypeptide. In some embodiments, the polynucleotide encoding the SYNPO2LB polypeptide shares at least 80%, at least 85%, at least 90%, at least 95%, at least 96%, at least 97%, at least 98%, at least 99%, or 100% identity to SEQ ID NO: 90, and/or the SYNPO2LB polypeptide shares at least 80%, at least 85%, at least 90%, at least 95%, at least 96%, at least 97%, at least 98%, at least 99%, or 100% identity to SEQ ID NO: 91.

In some embodiments, the SYNPO2LA polypeptide and/or SYNPO2LB polypeptide has a cardioprotective effect when the vector is administered to a cell or a mammal having a deleterious mutation in TTN gene, optionally wherein the deleterious mutation in the TTN gene is a truncating variant mutation in the TTN gene. In some embodiments, the cardioprotective effect is protection against and/or amelioration of sarcomere dysfunction or disarray observed in cells having a deleterious mutation in the TTN gene, optionally wherein the cells are cardiomyocytes.

In some embodiments, the vector is a viral vector. In some embodiments, the viral vector is an adeno-associated virus (AAV) vector, optionally wherein the AAV is AAV9 or a variant thereof. In some embodiments, the vector genome has a size equal to or less than 5.8 kB, 5.7 kB or 5.6 kB. In some embodiments, the vector is a lentiviral vector. In some embodiments, the vector is a non-viral vector.

In some aspects, provided is a recombinant AAV (rAAV) virion, comprising a vector according to various embodiments disclosed herein, and in particular a vector or virion encoding SYNPO2LA and/or SYNPO2LB, and an AAV capsid protein. In some embodiments, the rAAV virion is a serotype AAV9 virion or a variant thereof, and/or the AAV capsid protein is an AAV9 capsid protein or a variant thereof. In some embodiments, the rAAV virion comprises any one of the AAV capsid proteins described herein. In some embodiments, any one of the AAV capsid proteins (such as engineered AAV capsid proteins) disclosed in the section “Recombinant AAV Vectors and Virions” and/or in Tables 5 to 9B can be used in the rAAV virions provided herein.

In some aspects, provided is a pharmaceutical composition comprising a vector or virion according to various embodiments disclosed herein, and in particular a vector or virion encoding SYNPO2LA and/or SYNPO2LB, wherein and a pharmaceutically acceptable carrier.

In some aspects, provided is an isolated cell comprising a vector according to various embodiments disclosed herein, and in particular a vector encoding SYNPO2LA and/or SYNPO2LB. In some embodiments, the isolated cell is an induced pluripotent stem cell or an isolated cardiomyocyte. In some aspects, provided is a cell therapy composition comprising a cell according to various embodiments disclosed herein.

In some aspects, provided is a method of treating and/or preventing a heart disease in a subject, comprising administering to the subject a vector, pharmaceutical composition, virion, cell, or cell therapy composition according to various embodiments disclosed herein, and in particular those encoding SYNPO2LA and/or SYNPO2LB. In some embodiments, the subject is a human. In some embodiments, the heart disease is an acquired or genetic form of heart failure or cardiomyopathy. In some embodiments, the subject has a genetic mutation associated with heart failure or cardiomyopathy, optionally wherein the subject has a genetic mutation associated with heart failure with reduced ejection fraction or dilated cardiomyopathy. In some embodiments, the heart disease is cardiomyopathy. In some embodiments, the cardiomyopathy is dilated cardiomyopathy. In some embodiments, the heart disease is an acquired or genetic form of heart failure. In some embodiments, the heart failure is heart failure with reduced ejection fraction. In some embodiments, the heart disease is associated with a deleterious mutation in the TTN gene, and/or the subject has a deleterious mutation in the TTN gene. In some embodiments, the deleterious mutation in the TTN gene is a truncating variant mutation in the TTN gene. In some embodiments, the heart disease is associated with a deleterious mutation in the MLP/CSRP3 gene, and/or the subject has a deleterious mutation in the MLP/CSRP3 gene. In some embodiments, the administering improves cardiac function and/or ameliorates sarcomere dysfunction in cardiac cells of the subject. In some embodiments, the administering is systemic administration or local administration to the heart. In some embodiments, the systemic administration is intravenous administration. In some embodiments, the local administration is by direct injection into the heart or cardiac tissue, intracoronary administration, or retrograde coronary sinus infusion.

In some aspects, provided is a method of expressing a SYNPO2LA polypeptide and/or a SYNPO2LB polypeptide in a cell, comprising transducing the cell with a vector according to various embodiments disclosed herein, optionally wherein the cell is an induced pluripotent stem cell or an isolated cardiomyocyte. In some embodiments, the transducing of the cell is in vitro or ex vivo.

In some aspects, provided is a vector comprising a polynucleotide encoding an MTSS1 inhibitor operably linked to a promoter. In some embodiments, the inhibitor inhibits the expression of MTSS1 (e.g., human MTSS1).

In some embodiments, the MTSS1 inhibitor comprises an inhibitory RNA that inhibits the expression of MTSS1. In some embodiments, the inhibitory RNA is an siRNA or an shRNA, and wherein the promoter is an RNA-specific promoter (e.g., a pol III promoter). In some embodiments, the RNA-specific promoter is a U6 promoter. In some embodiments, the inhibitory RNA, optionally siRNA or shRNA, comprises at least 15, 16, 17, 18, or 19 contiguous nucleotides, with 0, 1, 2, or 3 mismatches, optionally with 0 mismatches, of any one of SEQ ID NOs: 94-100. In some embodiments, the inhibitory RNA, optionally siRNA or shRNA, targets any region of MTSS1 mRNA of SEQ ID NO: 92. In some embodiments, the inhibitory RNA, optionally siRNA or shRNA, inhibits the expression of MTSS1 protein of SEQ ID NO: 93.

In some embodiments, the MTSS1 inhibitor comprises (i) a Cas endonuclease protein operably linked to a cardiac-specific promoter, optionally wherein the cardiac-specific promoter is a TNNT2 promoter, and/or (ii) a guide RNA (gRNA) operably linked to an RNA-specific promoter (e.g., a pol III promoter), optionally wherein the RNA-specific promoter is a U6 promoter. In some embodiments, the Cas endonuclease and the gRNA are encoded by a single vector. In other embodiments, the Cas endonuclease and the gRNA are encoded by separate vectors.

In some embodiments, the gRNA is complementary to a sequence of the MTSS1 gene. In some embodiments, the sequence of the MTSS1 gene is a coding sequence of the MTSS1 gene or any region of SEQ ID NO: 406. In some embodiments, the coding sequence of the MTSS1 gene is the first exon of MTSS1 or SEQ ID NO: 407. In some embodiments, the gRNA is complementary to any region of SEQ ID NO: 406. In some embodiments, the gRNA is complementary to a region within any one or more of the exons of SEQ ID NO: 406. In some embodiments, the gRNA is complementary to any region of SEQ ID NO: 407.

In some embodiments, the gRNA is complementary to a non-coding DNA sequence promoting the expression of the MTSS1 gene. In some embodiments, the non-coding DNA sequence promoting the expression of the MTSS1 gene is an enhancer of the MTSS1 gene. In some embodiments, the non-coding DNA sequence is muscle-specific. In some embodiments, the non-coding DNA sequence is cardiac-specific. In some embodiments, the non-coding DNA sequence is within hg38 coordinates chr8:124,845,017-124,845,217. In some embodiments, the non-coding DNA sequence is or comprises SEQ ID NO: 408. In some embodiments, the non-coding DNA sequence is or comprises HAND2 binding site sequence, MEF2A/B/C binding site sequence, and/or a TWIST1 binding site sequence. In some embodiments, the non-coding DNA sequence is or comprises SEQ ID NO: 409, SEQ ID NO: 410, and/or SEQ ID NO: 411. In some embodiments, the gRNA is complementary to any region of SEQ ID NO: 408. In some embodiments, the gRNA is complementary to any region of SEQ ID NO: 409, SEQ ID NO: 410, and/or SEQ ID NO: 411. In some embodiments, the gRNA is complementary to any region of SEQ ID NO: 409. In some embodiments, the gRNA is complementary to any region of SEQ ID NO: 410. In some embodiments, the gRNA is complementary to any region of SEQ ID NO: 411.

In some embodiments, the MTSS1 inhibitor has a cardioprotective effect when the vector is administered to a cell or a mammal having a deleterious mutation in TTN gene, optionally wherein the deleterious mutation in the TTN gene is a truncating variant mutation in the TTN gene. In some embodiments, the cardioprotective effect is protection against and/or amelioration of sarcomere dysfunction or disarray observed in cells having a deleterious mutation in the TTN gene, optionally wherein the cells are cardiomyocytes.

In some embodiments, the vector is a viral vector. In some embodiments, the viral vector is an adeno-associated virus (AAV) vector, optionally wherein the AAV is AAV9 or a variant thereof. In some embodiments, the vector genome has a size equal to or less than 5.8 kB, 5.7 kB or 5.6 kB. In some embodiments, the vector is a lentiviral vector. In some embodiments, the vector is a non-viral vector.

In some aspects, provided is a recombinant AAV (rAAV) virion, comprising a vector according to various embodiments disclosed herein, and in particular a vector or virion encoding an MTSS1 inhibitor, and an AAV capsid protein. In some embodiments, the rAAV virion is a serotype AAV9 virion or a variant thereof, and/or the AAV capsid protein is an AAV9 capsid protein or a variant thereof. In some embodiments, the rAAV virion comprises any one of the AAV capsid proteins described herein. In some embodiments, any one of the AAV capsid proteins (such as engineered AAV capsid proteins) disclosed in the section “Recombinant AAV Vectors and Virions” and/or in Tables 5 to 9B can be used in the rAAV virions provided herein.

In some aspects, provided is a pharmaceutical composition comprising a vector or virion according to various embodiments disclosed herein, and in particular a vector or virion encoding an MTSS1 inhibitor, and a pharmaceutically acceptable carrier.

In some aspects, provided is an isolated cell comprising a vector according to various embodiments disclosed herein, and in particular a vector encoding an MTSS1 inhibitor. In some embodiments, the isolated cell is an induced pluripotent stem cell or an isolated cardiomyocyte. In some aspects, provided is a cell therapy composition comprising a cell according to various embodiments disclosed herein.

In some aspects, provided is a method of treating and/or preventing a heart disease in a subject, comprising administering to the subject a vector, virion, pharmaceutical composition, cell, or cell therapy composition according to various embodiments disclosed herein, and in particular those encoding an MTSS1 inhibitor. In some embodiments, the subject is a human. In some embodiments, the heart disease is an acquired or genetic form of heart failure or cardiomyopathy. In some embodiments, the subject has a genetic mutation associated with heart failure or cardiomyopathy, optionally wherein the subject has a genetic mutation associated with heart failure with reduced ejection fraction or dilated cardiomyopathy. In some embodiments, the heart disease is cardiomyopathy. In some embodiments, the cardiomyopathy is dilated cardiomyopathy. In some embodiments, the heart disease is an acquired or genetic form of heart failure. In some embodiments, the heart failure is heart failure with reduced ejection fraction. In some embodiments, the heart disease is associated with a deleterious mutation in the TTN gene, and/or the subject has a deleterious mutation in the TTN gene. In some embodiments, the deleterious mutation in the TTN gene is a truncating variant mutation in the TTN gene. In some embodiments, the heart disease is associated with a deleterious mutation in the MLP/CSRP3 gene, and/or the subject has a deleterious mutation in the MLP/CSRP3 gene. In some embodiments, the administering improves cardiac function and/or ameliorates sarcomere dysfunction in cardiac cells of the subject. In some embodiments, the administering is systemic administration or local administration to the heart. In some embodiments, the systemic administration is intravenous administration. In some embodiments, the local administration is by direct injection into the heart or cardiac tissue, intracoronary administration, or retrograde coronary sinus infusion.

In some aspects, provided is a method of inhibiting the expression of MTSS1 in a cell, comprising transducing the cell with a vector according to various embodiments disclosed herein. In some embodiments, the cell is an induced pluripotent stem cell or an isolated cardiomyocyte. In some embodiments, the transducing of the cell is in vitro or ex vivo.

In some aspects, provided is an inhibitory RNA or RNAi (e.g., an inhibitory siRNA or shRNA) inhibiting the expression of MTSS1 (e.g., human MTSS1). In some embodiments, the RNAi (e.g., inhibitory siRNA or shRNA) comprises at least 15, 16, 17, 18, or 19 contiguous nucleotides, with 0, 1, 2, or 3 mismatches, optionally with 0 mismatches, of any one of SEQ ID NOs: 94-100. In some embodiments, the RNAi (e.g., inhibitory siRNA or shRNA) targets any region of MTSS1 mRNA of SEQ ID NO: 92. In some embodiments, the RNAi (e.g., siRNA or shRNA) inhibits the expression of MTSS1 protein of SEQ ID NO: 93. In some embodiments, the RNAi is an siRNA. In some embodiments, the RNAi is an shRNA.

In some aspects, provided is a pharmaceutical composition comprising an inhibitory siRNA or shRNA according to various embodiments disclosed herein, and a pharmaceutically acceptable carrier.

In some aspects, provided is a method of treating and/or preventing heart disease in a subject, comprising administering to a subject an inhibitory siRNA or shRNA according to various embodiments disclosed herein, or a pharmaceutical composition comprising the same and a pharmaceutically acceptable carrier. In some embodiments, the subject is a human. In some embodiments, the heart disease is an acquired or genetic form of heart failure or cardiomyopathy. In some embodiments, the subject has a genetic mutation associated with heart failure or cardiomyopathy, optionally wherein the subject has a genetic mutation associated with heart failure with reduced ejection fraction or dilated cardiomyopathy. In some embodiments, the heart disease is cardiomyopathy. In some embodiments, the cardiomyopathy is dilated cardiomyopathy. In some embodiments, the heart disease is an acquired or genetic form of heart failure. In some embodiments, the heart failure is heart failure with reduced ejection fraction. In some embodiments, the heart disease is associated with a deleterious mutation in the TTN gene, and/or the subject has a deleterious mutation in the TTN gene. In some embodiments, the deleterious mutation in the TTN gene is a truncating variant mutation in the TTN gene. In some embodiments, the heart disease is associated with a deleterious mutation in the MLP/CSRP3 gene, and/or the subject has a deleterious mutation in the MLP/CSRP3 gene. In some embodiments, the administering improves cardiac function and/or ameliorates sarcomere dysfunction in cardiac cells of the subject. In some embodiments, the administering is systemic administration or local administration to the heart. In some embodiments, the systemic administration is intravenous administration.

### BRIEF DESCRIPTION OF THE DRAWINGS

FIG. 1A shows a schematic of a plasmid map for an AAV vector comprising AMP]1.

FIG. 1B shows a schematic of a plasmid map for an AAV vector comprising SYNPO2LA.

FIG. 1C shows a schematic of a plasmid map for an AAV vector comprising SYNPO2LB.

FIG. 2 is an illustration of high content screening, using AAV overexpression or siRNA inhibition of human genetic targets, to identify genes and siRNAs that confer a cardioprotective effect in iPSCs with compromised TTN. The screening was performed using software which outputs nuclear count, sarcomere count, sarcomere length, sarcomere angle, and sarcomere fit score for each image.

FIG. 3 shows that the sarcomere structure is disturbed by siRNA knockdown of TTN gene.

FIG. 4 shows sarcomere count distribution obtained using high-content microscopy of an induced pluripotent stem cell (iPSC) line treated with a) siTTN, and with b) either AAV delivery of the longer isoform of SYNPO2L_A (p=0.002), or AAV delivery of the shorter isoform of SYNPO2L_B (p<0.001).

FIG. 5A shows sarcomere count distribution obtained using high-content microscopy of an induced pluripotent stem cell (iPSC) line which carries a heterozygous TTN truncating mutation (P22353X+/−) when treated with siMTSS1 relative to untreated cells.

FIG. 5B shows a plot of the twitch force in engineered heart tissues derived from the TTN P22353X^{+/-} iPSC line when treated with siMTSS1 relative to a scrambled control (siSCR, which served as negative control).

FIG. 6 shows the impact of MTSS1 inhibition on cardiomyocyte health in an siMLP/CSRP3 genetic DCM background. Plotted is the probability of ‘rescue’ of knockdown of MLP/CSRP3 for controls and test samples. SCR is a negative control (scrambled control). The siMLP knockdown alone is classified primarily as abnormal (median probability 0.02). In comparison to the siMLP knockdown alone, the 5 nM siMTSS1+siMLP displayed improved likelihood of normal appearance (median probability 0.16, p=5e-04) and the 10 nM siMTSS1+siMLP displayed even higher likelihood of normal appearance (median probability 0.48, p=2e-16).

FIG. 7 shows a plot of the twitch force in engineered heart tissues constructed from a wild type iPSC line treated with siMLP with and without siMTSS1 at two concentrations or scrambled control (siSCR, which served as negative control). The wild-type iPSCs treated with siMLP revealed a significant and sustained improvement in force produced when treated with siMTSS1 at 5 nM or 10 nM concentration for 48 hours (average 13% improvement over baseline, SE 3.7%, p=0.001) over 7 days.

FIG. 8 shows that a single-nucleotide polymorphism (SNP) mutation of G to T near the MTSS1 gene locus (rs12541595) disrupts the activity of a cardiac enhancer of MTSS1 and results in decreased MTSS1 expression in the heart.

FIG. 9 shows the association of the rs12541595 T allele to cardiac phenotypes including decreased left ventricle (LV) diastolic dimension, increased LV ejection fraction, decreased LV end-systolic volume, decreased global circumferential strain, and decreased dilated cardiomyopathy risks.

FIG. 10 shows that in a study of 89 individuals of European ancestry who carry a TTN protein-truncating variant (PTV) and have been diagnosed with DCM, carriers of the rs12541595 T allele showed significantly improved event-free survival (death or heart transplant) by a Hazard ratio of 0.32 with p-value of 0.007, after adjustment for age and genetic sex.

### DETAILED DESCRIPTION

In some embodiments, described herein are polynucleotides, expression cassettes, vectors, and virions (e.g., AAV such as AAV9 or a variant thereof) comprising one or more cardioprotective genes encoding cardioprotective gene products and/or cardioprotective inhibitory RNA or CRISPR/Cas system. In some embodiments, described herein are polynucleotides, expression cassettes, vectors, and virions (e.g., AAV such as AAV9 or a variant thereof) encoding a SYNPO2LA gene and/or SYNPO2LB gene product. In some embodiments, described herein are polynucleotides, expression cassettes, vectors, and virions (e.g., AAV such as AAV9 or a variant thereof) encoding an MTSS1 inhibitor, for example, wherein the MTSS1 inhibitor inhibits the expression of the MTSS1 gene, or inhibits the level or activity of the MTSS1 gene product. In some embodiments, the MTSS1 inhibitor is an inhibitory RNA, such as siRNAs or shRNAs inhibiting the expression of MTSS1. In some embodiments, the MTSS1 inhibitor is a gene editing system inhibiting the expression of MTSS1 (such as a Cas endonuclease and a guide RNA, wherein the guide RNA is complementary to a coding sequence of MTSS1 gene or a non-coding sequence regulating the expression of MTSS1). In some embodiments, the MTSS1 inhibitor targets a non-coding region promoting or driving the expression of the MTSS1 gene in cardiac cells (e.g., selectively promoting the expression of the MTSS1 gene in cardiac cells). In some embodiments, the targeted non-coding region is an enhancer. In some embodiments, the MTSS1 inhibitor does not inhibit, or does not substantially inhibit, the expression of the MTSS1 gene in non-muscle cells and/or non-cardiac cells (e.g., in liver cells, kidney cells, brain, etc.). In some embodiments, the polynucleotides, expression cassettes, vectors, and virions described herein encode or deliver the cardioprotective gene product, RNA or CRISPR/Cas system in a muscle-specific or cardiac-specific manner. In some embodiments, the specificity of expression of the gene product is achieved by use of a muscle cell-specific or cardiac cell-specific promoter (e.g., a TNNT2 promoter).

In some embodiments, described herein is any MTSS1 inhibitor, for example, wherein the MTSS1 inhibitor inhibits the expression of the MTSS1 gene, or inhibits the level or activity of the MTSS1 gene product. In some embodiments, the MTSS1 inhibitor is an inhibitory RNA (RNAi), e.g., siRNA or shRNA that inhibits the expression of the MTSS1 gene. In some embodiments, the MTSS1 inhibitor is a small molecule. The MTSS1 inhibitor (e.g., RNAi) can be delivered using a viral and non-viral delivery route. In some embodiments, an MTSS1 inhibitor is administered using any viral vector known in the art or described herein. In some embodiments, an MTSS1 inhibitor is administered using an AAV vector as described herein. In some embodiments, an MTSS1 inhibitor is administered using a delivery vehicle such as a liposome or conjugated to a targeting molecule. For example, RNAi encapsulated in a liposome can be administered parenterally or intravenously.

In some embodiments, the inhibitors, polynucleotides, expression cassettes, vectors, and virions described herein are for use in the treatment or prevention of heart disease, e.g., heart failure or dilated cardiomyopathy.

In some embodiments, described herein are polynucleotides, expression cassettes, vectors, and virions (e.g., AAV such as AAV9 or a variant thereof) comprising one or more cardioprotective genes encoding cardioprotective gene products (such as MMP11, SYNPO2LA, or SYNPO2LB) and/or cardioprotective inhibitory RNAs (such siRNAs or shRNAs inhibiting the expression of MTSS1), for use in the treatment or prevention of heart disease, e.g., dilated cardiomyopathy. The heart disease may be an acquired form of heart disease or a genetic or polygenic form of heart disease. In some embodiments, the heart disease is caused by a genetic mutation (e.g., a mutation in a gene associated with cardiac function and/or cardiac disease). In some embodiments, described herein are polynucleotides, expression cassettes, vectors, and virions (e.g., AAV such as AAV9 or a variant thereof) comprising one or more cardioprotective genes encoding cardioprotective gene products (such as MMP11, SYNPO2LA, or SYNPO2LB) and/or cardioprotective inhibitory RNAs (such as siRNAs or shRNAs inhibiting the expression of MTSS1), for use in the treatment or prevention of TTN mutation-associated heart disease, e.g., dilated cardiomyopathy, such as in subjects with a genetic mutation in TTN (e.g., TTNtv). In some embodiments, described herein are polynucleotides, expression cassettes, vectors, and virions (e.g., AAV such as AAV9) comprising one or more cardioprotective genes encoding cardioprotective gene products (such as MMP11, SYNPO2LA, or SYNPO2LB) and/or cardioprotective inhibitory RNAs (such siRNAs or shRNAs inhibiting the expression of MTSS1), for use in the treatment or prevention of MLP/CSRP3 mutation-associated heart disease, e.g., dilated cardiomyopathy, such as in subjects with a genetic mutation in MLP/CSRP3.

In some embodiments, described herein are methods of treatment or prevention of heart disease, e.g., dilated cardiomyopathy, by administering to a subject a vector or virion (e.g., AAV such as AAV9 or a variant thereof) comprising one or more cardioprotective genes encoding cardioprotective gene products (such as MMP11, SYNPO2LA, or SYNPO2LB) and/or cardioprotective inhibitory RNAs (such as siRNAs or shRNAs inhibiting the expression of MTSS1). The heart disease may be an acquired form of heart disease or a genetic or polygenic form of heart disease. In some embodiments, the heart disease is caused by a genetic mutation (e.g., a mutation in a gene associated with cardiac function and/or cardiac disease). In some embodiments, described herein are methods of treatment or prevention of TTN mutation-associated heart disease, e.g., dilated cardiomyopathy, by administering to a subject with a genetic mutation in TTN (e.g., TTNtv) a vector or virion (e.g., AAV such as AAV9 or a variant thereof) comprising one or more cardioprotective genes encoding cardioprotective gene products (such as MMP11, SYNPO2LA, or SYNPO2LB) and/or cardioprotective inhibitory RNAs (such as siRNAs or shRNAs inhibiting the expression of MTSS1). In some embodiments, described herein are methods of treatment or prevention of a mutation-associated heart disease (e.g., MLP/CSRP3 mutation-associated heart disease), by administering to a subject with a genetic mutation (e.g., in MLP/CSRP3) a vector or virion (e.g., AAV such as AAV9 or a variant thereof) comprising one or more cardioprotective genes encoding cardioprotective gene products (such as MMP11, SYNPO2LA, or SYNPO2LB) and/or cardioprotective inhibitory RNAs (such siRNAs or shRNAs inhibiting the expression of MTSS1).

In some embodiments, described herein are methods of treatment or prevention of heart disease, e.g., dilated cardiomyopathy, by administering to a subject a cardioprotective inhibitory RNA (such as an siRNA or shRNA inhibiting the expression of MTSS1). In some embodiments, described herein are methods of treatment or prevention of TTN mutation-associated heart diseases, e.g., dilated cardiomyopathy, by administering to a subject with a genetic mutation in TTN (e.g., TTNtv) a cardioprotective inhibitory RNA (such as an siRNA or shRNA inhibiting the expression of MTSS1). In some embodiments, described herein are methods of treatment or prevention of MLP/CSRP3 mutation-associated heart disease, e.g., dilated cardiomyopathy, by administering to a subject with a genetic mutation in MLP/CSRP3 a cardioprotective inhibitory RNA (such as an siRNA or shRNA inhibiting the expression of MTSS1).

Without being bound by any theory or mechanism of action, provided herein are therapies that compensate for deleterious mutations in one or more genes associated with cardiac function and/or cardiac disease in a subject. In particular, without being bound by any theory or mechanism of action, provided herein are therapies that compensate for TTN mutations in a subject. For example, TTN-associated DCM is characterized by sarcomere disarray and/or dysfunction, and therapies provided herein protect against and/or ameliorate such TTN-associated disarray and/or dysfunction. In some embodiments, without being bound by any theory or mechanism of action, provided herein are therapies that compensate for MLP/CSRP3 mutations in a subject. The therapies provided herein include, without limitation, supplementation or overexpression of a protective gene (e.g., MMP11, SYNPO2LA, or SYNPO2LB), for example using AAV delivery of the gene or another method of delivery. The therapies provided herein also include, without limitation, knocking down, inhibiting, or otherwise decreasing the expression of a risk-inducing gene (e.g., using a small-interfering RNA (siRNA), short-hairpin RNA (shRNA) or another inhibitory RNA), for example using AAV delivery of inhibitory RNA or another method of delivery. The risk-inducing gene can be MTSS1. The therapies provided herein also include, without limitation, a combination of multiple simultaneous manipulations (such as supplementation of multiple genes and/or inhibition of multiple genes). The therapies described herein can achieve a desired and therapeutic clinical benefit. The therapies provided herein can be used for treating or preventing TTN-related heart diseases, e.g., TTN-related cardiomyopathy such as TTN-related DCM. The therapies provided herein can be used for treating or preventing TTN-related heart failure. The therapies provided herein can be used for treating or preventing MLP/CSRP3-related heart diseases, e.g., MLP/CSRP3-related cardiomyopathy such as MLP/CSRP3-related DCM. The therapies provided herein can be used for treating or preventing MLP/CSRP3-related heart failure. The therapies provided herein can also be used for treating or preventing heart disease in any genetic background. For example, the therapies provided herein can also be used for treating or preventing cardiomyopathy, including genetic and non-genetic DCM. In some embodiments, the therapies provided herein can be used for treating or preventing genetic DCM. In some embodiments, the therapies provided herein can be used for treating or preventing non-genetic DCM. In some embodiments, the therapies provided herein can be used for treating or preventing heart failure, e.g., in a patient having any genetic background.

In some aspects, the therapies provided are for treating patients who have a mutation, e.g., a deleterious mutation such as any mutation that confers a risk for cardiac disease.

In some aspects, the therapies provided herein do not comprise supplementation, overexpression or other manipulation of the TTN gene itself. In some aspects, the therapies provided herein may further comprise supplementation, overexpression or other manipulation of the TTN gene itself.

In some aspects, the therapies provided are for treating patients who have a mutation, e.g., a deleterious mutation such as any mutation that confers a risk for cardiac disease, in the TTN gene (e.g., a TTNtv mutation).

In some aspects, the therapies provided herein do not comprise supplementation, overexpression or other manipulation of the MLP/CSRP3 gene itself. In some aspects, the therapies provided herein may further comprise supplementation, overexpression or other manipulation of the MLP/CSRP3 gene itself.

In some aspects, the therapies provided are for treating patients who have a mutation, e.g., a deleterious mutation such as any mutation that confers a risk for cardiac disease, in the MLP/CSRP3 gene.

In some aspects, the therapies provided are for treating patients who do not have a mutation, e.g., a loss of function mutation or other mutation that confers a risk factor for cardiac disease, in the MMP11 gene. In other aspects, the therapies provided are for treating patients who have a mutation, e.g., a loss of function mutation or other mutation that confers a risk factor for cardiac disease, in the MMP11 gene.

In some aspects, the therapies provided are for treating patients who do not have a mutation, e.g., a loss of function mutation or other mutation that confers a risk factor for cardiac disease, in a SYNPO2L gene. In other aspects, the therapies provided are for treating patients who have a mutation, e.g., a loss of function mutation or other mutation that confers a risk factor for cardiac disease, in a SYNPO2L gene.

In some aspects, the therapies provided are for treating patients who do not have a mutation, e.g., a loss of function mutation or other mutation that confers a risk factor for cardiac disease, in a SYNPO2LA gene. In other aspects, the therapies provided are for treating patients who have a mutation, e.g., a loss of function mutation or other mutation that confers a risk factor for cardiac disease, in a SYNPO2LA gene.

In some aspects, the therapies provided are for treating patients who do not have a mutation, e.g., a loss of function mutation or other mutation that confers a risk factor for cardiac disease, in a SYNPO2LB gene. In other aspects, the therapies provided are for treating patients who have a mutation, e.g., a loss of function mutation or other mutation that confers a risk factor for cardiac disease, in a SYNPO2LB gene.

In some aspects, the therapies provided are for treating patients who do not have a mutation in, e.g., a gain of function mutation or other mutation that confers a risk factor for cardiac disease, or overexpression of, a MTSS1 gene. In other aspects, the therapies provided are for treating patients who have a mutation in, e.g., a gain of function mutation or other mutation that confers a risk factor for cardiac disease, or overexpression of, a MTSS1 gene.

In some aspects, provided herein is a method for identification of a cardioprotective therapy by:
(i) providing a cardiac cell (e.g., a cardiomyocyte, such as an iPSC-derived cardiomyocyte), wherein the cardiac cell has a deleterious mutation in a TTN gene and/or a MLPCSRP3 gene or wherein the expression of the TTN gene and/or the MLP/CSRP3 gene is inhibited (e.g., by knockdown, siRNA or shRNA inhibition), wherein said deleterious mutation or inhibition of expression leads to dysfunction in the cardiac cell;
(ii) introducing a test therapy, wherein the test therapy may comprise one, two, three, or more (e.g., a plurality) of gene therapy cassettes encoding a gene product and/or one, two, three, or more (e.g., a plurality) of agents inhibiting expression of a gene product (e.g. siRNA or shRNA); and
(iii) evaluating whether the test therapy ameliorates or rescues the dysfunction in the cardiac cell, optionally wherein the dysfunction is in sarcomere architecture (e.g., sarcomere count, length, angle, and/or fit, as described herein) and/or in contractility (e.g., as measured by twitch force, as described herein),
wherein detection of amelioration or rescue of a dysfunction in the cardiac cell indicates that the test therapy is cardioprotective and thereby identifies a cardioprotective therapy.

### Terminology

As used in this specification and the appended claims, the singular forms “a,” “an” and “the” include plural references unless the content clearly dictates otherwise.

As used in this specification, the term “and/or” is used in this disclosure to mean either “and” or “or” unless indicated otherwise.

Throughout this specification, unless the context requires otherwise, the words “comprise”, or variations such as “comprises” or “comprising”, will be understood to imply the inclusion of a stated element or integer or group of elements or integers but not the exclusion of any other element or integer or group of elements or integers.

As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

The terms “polynucleotide” and “nucleic acid,” used interchangeably herein, refer to a polymeric form of nucleotides of more than about 100 nucleotides, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. “Oligonucleotide” generally refers to polynucleotides of between about 5 and about 100 nucleotides of single- or double-stranded DNA or RNA. However, for the purposes of this disclosure, there is no upper limit to the length of an oligonucleotide. Oligonucleotides are also known as “oligomers” or “oligos” and may be isolated from genes, or chemically synthesized by methods known in the art. The terms “polynucleotide” and “nucleic acid” should be understood to include, as applicable to the embodiments being described, single-stranded (such as sense or antisense) and double-stranded polynucleotides.

The term “promoter” as used herein refers a polynucleotide sequence that has one or more recognition site(s) to which an RNA polymerase binds, such that in a host or target cell, an RNA polymerase may initiate and transcribe a polynucleotide sequence “downstream” of the promoter into an RNA. Similarly stated, a “promoter” is operably linked or operatively linked to a polynucleotide sequence if in a host or target cell in which the promoter is active, an RNA polymerase initiates transcription of the polynucleotide at a transcription state site. Promoters operative in mammalian cells generally comprise an AT-rich region located approximately 25 to 30 bases upstream from the site where transcription is initiated and/or another sequence found 70 to 80 bases upstream from the start of transcription, a CNCAAT region where N may be any nucleotide.

The terms “upstream” and “upstream end” refer to a portion of a polynucleotide that is, with reference to a transcription start site (TSS), 5′ to the TSS on the sense strand (or coding strand) of the polynucleotide; and 3′ to the TSS on the antisense strand of the polynucleotide. The terms “downstream” and “downstream end” refer to a portion of a polynucleotide that is, with reference to a TSS, 3′ to TSS on the sense strand (or coding strand) of the polynucleotide; and 5′ to the TSS on the antisense strand of the polynucleotide. Thus, a deletion from the upstream end of a promoter is a deletion of one or more base pairs in the non-transcribed region of the polynucleotide, 5′ to the TSS on the sense strand (or equivalently, 3′ to the TSS on the antisense strand). A deletion from the downstream end of a promoter is a deletion of one or more base pairs in the transcribed region of the polynucleotide, 3′ to the TSS on the sense strand (or equivalently, 5′ to the TSS on the antisense strand).

As used herein, the term “transgene” refers to a nucleic acid sequence encoding a protein or RNA (e.g., a therapeutic protein), which is partly or entirely heterologous, i.e., foreign, to the transgenic animal or cell into which it is introduced, or, is homologous to an endogenous gene of the transgenic animal or cell into which it is introduced, but which is designed to be inserted, or is inserted, into the animal's genome in such a way as to alter the genome of the cell into which it is inserted (e.g., it is inserted at a location which differs from that of the natural gene or its insertion results in a knockout). A transgene can include one or more transcriptional regulatory sequences and any other nucleic acid, such as introns, that may be necessary for optimal expression of a selected nucleic acid.

The term “sequence identity” refers to the percentage of bases or amino acids between two polynucleotide or polypeptide sequences that are the same, and in the same relative position. As such one polynucleotide or polypeptide sequence has a certain percentage of sequence identity compared to another polynucleotide or polypeptide sequence. For sequence comparison, typically one sequence acts as a reference sequence, to which test sequences are compared. The term “reference sequence” refers to a molecule to which a test sequence is compared.

Methods of sequence alignment for comparison and determination of percent sequence identity is well known in the art. Optimal alignment of sequences for comparison can be conducted, e.g., by the homology alignment algorithm of Needleman and Wunsch, J Mol. Biol. 48:443 (1970), by the search for similarity method of Pearson and Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), by manual alignment and visual inspection (see, e.g., Brent et al., Current Protocols in Molecular Biology (2003)), by use of algorithms know in the art including the BLAST and BLAST 2.0 algorithms, which are described in Altschul et al., Nuc. Acids Res. 25:3389-3402 (1977); and Altschul et al., J. Mol. Biol. 215:403-410 (1990), respectively. Software for performing BLAST analyses is publicly available through the National Center for Biotechnology Information.

In some embodiments, the determination of the percentage of sequence identity may take place after a local alignment. Such alignments are well known in the art, for instance the service EMBOSS Matcher identifies local similarities between two sequences using an algorithm based on the LALIGN application, version 2.0u4. In an example, the identity between two nucleic acid sequences may be calculated using the service Matcher (EMBOSS) set to the default parameters, e.g., matrix (DNAfull), gap open (16), gap extend (4), alternative matches (1).

An “expression cassette” or “expression construct” refers to a DNA polynucleotide sequence operably linked to a promoter. “Operably linked” or “operatively linked” refers to a juxtaposition wherein the components so described are in a relationship permitting them to function in their intended manner. For instance, a promoter is operably linked to a polynucleotide sequence if the promoter affects the transcription or expression of the polynucleotide sequence.

As used herein, the term “delivery”, which is used interchangeably with “transduction,” refers to the process by which exogenous nucleic acid molecules are transferred into a cell such that they are located inside the cell. Delivery of nucleic acids is a distinct process from expression of nucleic acids.

The term “modified” refers to a substance or compound (e.g., a cell, a polynucleotide sequence, and/or a polypeptide sequence) that has been altered or changed as compared to the corresponding unmodified substance or compound.

The term “sample” refers to a biological composition (e.g., a cell or a portion of a tissue) that is subjected to analysis and/or genetic modification. In some embodiments, a sample is a “primary sample” in that it is obtained directly from a subject; in some embodiments, a “sample” is the result of processing of a primary sample, for example to remove certain components and/or to isolate or purify certain components of interest.

The term “transfection” refers to the uptake of foreign DNA by a cell. A cell has been “transfected” when exogenous DNA has been introduced inside the cell membrane. A number of transfection techniques are generally known in the art. See, e.g., Graham et al., Virology 52:456 (1973); Sambrook et al., Molecular Cloning: A Laboratory Manual (1989); Davis et al., Basic Methods in Molecular Biology (1986); Chu et al., Gene 13:197 (1981). Such techniques can be used to introduce one or more exogenous DNA moieties, such as a nucleotide integration vector and other nucleic acid molecules, into suitable host cells. The term captures chemical and electrical transfection procedures.

The term “expression” refers to the process by which a nucleic acid is translated into peptides or is transcribed into RNA, which, for example, can be translated into peptides, polypeptides or proteins. If the nucleic acid is derived from genomic DNA, expression may, if an appropriate eukaryotic host cell or organism is selected, include splicing of the mRNA. For heterologous nucleic acid to be expressed in a host cell, it must initially be delivered into the cell and then, once in the cell, ultimately reside in the nucleus.

As used herein, a “heterologous” polynucleotide or nucleic acid refers to a polynucleotide or portion of a polynucleotide derived from a source other than the host organism or, for a viral vector, the native, non-recombinant virus. Examples of heterologous DNA include, but are not limited to, DNA that encodes traceable marker proteins, such as a protein that confers drug resistance, DNA that encodes therapeutically effective substances, such as anti-cancer agents, enzymes and hormones, and DNA that encodes other types of proteins, such as antibodies.

The term “wild type” refers to the naturally-occurring polynucleotide sequence encoding a protein, or a portion thereof, or protein sequence, or portion thereof, respectively, as it normally exists in vivo in a normal or healthy subject.

The term “variant” refers to a protein or nucleic acid having one or more genetic changes (e.g., insertions, deletions, substitutions, or the like) that returns all or substantially all of the functions of the reference protein or nucleic acid. For example, a variant of a therapeutic protein retains the same or substantially the same activity and/or provides the same or substantially the same therapeutic benefit to a subject in need thereof. A variant of a promoter sequence retains the ability to initiate transcription at the same or substantially the same level as the reference promoter, and retains the same or substantially the same cell type specificity. In particular embodiments, polynucleotides variants have at least or about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a reference sequence. In particular embodiments, protein variants have at least or about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a reference sequence.

The term “subject” includes animals, such as e.g., mammals. In some embodiments, the mammal is a primate. In some embodiments, the mammal is a human. In some embodiments, subjects are livestock such as cattle, sheep, goats, cows, swine, and the like; or domesticated animals such as dogs and cats. In some embodiments (e.g., particularly in research contexts) subjects are rodents (e.g., mice, rats, hamsters), rabbits, primates, or swine such as inbred pigs and the like. The terms “subject” and “patient” are used interchangeably herein.

The term “administering” to a subject is a procedure by which one or more delivery agents, together or separately, are introduced into or applied onto a subject such that target cells which are present in the subject are eventually contacted with the agent.

As used herein, the term “gene product” refers to a protein or nucleic acid produced by the transcription of a polynucleotide and, in the case of a protein gene product, the subsequent translation of transcript into a protein.

As used herein, the term “cardiomyopathy” refers to the deterioration of the function of the myocardium (i.e., the actual heart muscle) for any reason. Subjects with cardiomyopathy are often at risk of arrhythmia or sudden cardiac death or both.

The term “dilated cardiomyopathy” includes, but is not limited to, cardiomyopathy caused by truncating variants in the TTN Gene.

As used herein, the term “hypertrophic cardiomyopathy” refers to a disease of the heart and myocardium in which a portion of the myocardium is hypertrophied.

As used herein, the term “familial hypertrophic cardiomyopathy” refers to a genetic disorder characterized by increased growth (i.e., hypertrophy) in thickness of the wall of the left ventricle.

As used herein, the term “effective amount” refers to the minimum amount of an agent or composition required to result in a particular physiological effect. The effective amount of a particular agent may be represented in a variety of ways based on the nature of the agent, such as mass/volume, # of cells/volume, particles/volume, (mass of the agent)/(mass of the subject), # of cells/(mass of subject), or particles/(mass of subject). The effective amount of a particular agent may also be expressed as the half-maximal effective concentration (ECso), which refers to the concentration of an agent that results in a magnitude of a particular physiological response that is half-way between a reference level and a maximum response level.

### Cardioprotective Genes, Inhibitory RNA, Vectors, and Compositions

In some embodiments, described herein are cardioprotective genes and cardioprotective inhibitory RNAs. In some embodiments, described herein are polynucleotides, expression cassettes, and vectors comprising cardioprotective genes and cardioprotective inhibitory RNAs. In some embodiments, described herein are expression cassettes and vectors comprising a cardioprotective gene encoding a cardioprotective gene product, for use in the treatment of heart disease (e.g., cardiomyopathy). In some embodiments, described herein are cardioprotective inhibitory RNAs and compositions comprising the same, for use in the treatment of heart disease (e.g., cardiomyopathy).

In some embodiments, the cardioprotective genes and inhibitory RNAs described herein, when delivered to a subject, are effective to protect against and/or ameliorate sarcomere dysfunction and/or disarray. In some embodiments, the cardioprotective genes and inhibitory RNAs described herein, when delivered to a subject, are effective to treat a heart disease (e.g., cardiomyopathy). In some embodiments, the cardioprotective genes and inhibitory RNA described herein, when delivered to a subject, are effective to treat dilated cardiomyopathy (DCM). In some embodiments, the cardioprotective genes and inhibitory RNA described herein, when delivered to a subject, are effective to treat heart failure.

In some embodiments, treatment with a vector or composition comprising the cardioprotective gene, gene product or inhibitory RNA described herein provides a therapeutic physiological effect or benefit to a subject with heart disease (e.g., a subject with cardiomyopathy).

### Cardioprotective Genes and Inhibitory RNAs

In some aspects, the present disclosure provides genes, gene products, and inhibitory RNAs suitable for use in a method of treating a heart disease.

In some aspects, the present disclosure provides genes, gene products, and inhibitory RNAs suitable for use in a method of treating a heart disease caused by a genetic mutation (e.g., a mutation in a gene associated with cardiac function and/or cardiac disease) in a subject in need thereof.

In some aspects, the present disclosure provides genes, gene products, and inhibitory RNAs suitable for use in a method of treating a disease caused by a TTN mutation in a subject in need thereof.

In some aspects, the present disclosure provides genes, gene products, and inhibitory RNAs suitable for use in a method of treating a disease caused by a MLP/CSRP3 mutation in a subject in need thereof.

In some embodiments, described herein is any gene or gene product that has a protective effect, such as cardioprotective effect, when delivered to a mammal having a deleterious mutation in the TTN gene (e.g., in a viral vector). In some embodiments, described herein is any inhibitory RNA that has a protective effect, e.g., cardioprotective effect, when delivered to a mammal having a deleterious mutation in the TTN gene (e.g., in a viral vector, a lipid nanoparticle, or as naked RNA).

In some embodiments, described herein is any gene or gene product that has a protective effect, such as cardioprotective effect, when delivered to a mammal having a deleterious mutation in the MLP/CSRP3 gene (e.g., in a viral vector). In some embodiments, described herein is any inhibitory RNA that has a protective effect, e.g., cardioprotective effect, when delivered to a mammal having a deleterious mutation in the MLP/CSRP3 gene (e.g., in a viral vector, a lipid nanoparticle or as naked RNA).

Deleterious mutations in the TTN gene and/or the MLP/CSRP3 gene can be, without limitation, any deleterious mutations known in the art or described herein. In some embodiments, a deleterious mutation is a truncating variant mutation in the TTN gene (TTNtv), e.g., as described in Akhtar et al., 2020, Circ. Heart Fail. 13(10):e006832. DOI:10.1161/CIRCHEARTFAILURE.119.006832.

Polynucleotides, expression cassettes and vectors for expression of such cardioprotective genes and gene products are described herein. Cardioprotective inhibitory RNAs described herein can be used as naked RNA, in a composition (e.g., lipid nanoparticle composition) or as part of a polynucleotide, expression cassette and/or vector, for delivery to a subject.

In some embodiments, the cardioprotective gene is MMP11, or a functional homolog or variant thereof. In some embodiments, the cardioprotective gene product is MMP11 polypeptide, or a functional homolog or variant thereof. In some embodiments, the cardioprotective gene is SYNPO2L, or a functional homolog or variant thereof. In some embodiments, the cardioprotective gene product is SYNPO2L polypeptide, or a functional homolog or variant thereof. In some embodiments, the cardioprotective gene is SYNPO2LA, or a functional homolog or variant thereof. In some embodiments, the cardioprotective gene product is SYNPO2LA polypeptide, or a functional homolog or variant thereof. In some embodiments, the cardioprotective gene is SYNPO2LB, or a functional homolog or variant thereof. In some embodiments, the cardioprotective gene product is SYNPO2LB polypeptide, or a functional homolog or variant thereof. In some embodiments, the cardioprotective gene product is an inhibitor of MTSS1, e.g., an inhibitory RNA that inhibits the expression of MTSS1, a vector comprising an expression cassette encoding a gene editing system (e.g., a CRISPR/Cas system as described herein) that targets the MTSS1 gene, its cardiac-specific non-coding regulatory sequence (e.g., enhancer sequence), or otherwise inhibits the expression of MTSS1, or a functional homolog or variant thereof. In some embodiments, the cardioprotective inhibitory RNA is an siRNA or shRNA, as described herein. See Table 1 and below for more information about these genes and gene products thereof.

TABLE 1
Exemplary genes

| | Gene | NCBI | UniProt |
|---|---|---|---|
| Gene Name | Symbol | Gene ID | ID |
| Matrix | MMP11 | NM_005940.5 | P24347 |
| metallopeptidase 11 | | | |
| Synaptopodin 2 | SYNPO2LA | NM_001114133.3 | Q9H987 |
| like A | | | |
| Synaptopodin 2 | SYNPO2LB | NM_024875.5 | Q9H987 |
| like B | | | |
| Metastasis | MTSS1 | NM_014751.6 | O43312 |
| Suppressor Protein 1 | | | |

### MMP11

MMP11 is a gene expressed in a variety of non-cardiac tissues, as well as cardiomyocytes. Proteins of the matrix metalloproteinase (MMP) family are involved in the breakdown of extracellular matrix in normal physiological processes, such as embryonic development, reproduction, and tissue remodeling, as well as in disease processes, such as arthritis and metastasis. Most MMPs are secreted as inactive proproteins which are activated when cleaved by extracellular proteinases. However, MMP11 is activated intracellularly by furin within the constitutive secretory pathway. Additionally, MMP11 cleaves alpha 1-proteinase inhibitor, α3 chain of collagen VI, α2-macroglobulin, IGFBP1, but weakly degrades structural proteins of the extracellular matrix. See, e.g., https://www.ncbi.nlm.nih.gov/gene/4320; Bassiouni et al., 2021, FEBS J. 288(24):7162-7182; Luo et al., 2002, J. Biol. Chem, Genes: Structure and Regulation 277(28):P25527-25536; Manes et al., 1997, J. Biol. Chem 272(41):25706-12; Aung et al., 2019, Circulation 140(16):1318-1330.

In some embodiments, the disclosure provides a vector comprising a polynucleotide sequence that encodes MMP11 protein (or a mutant, variant, or fragment thereof), operatively linked to a cardiac-specific promoter (e.g., a TNNT2 promoter). In some embodiments, the polynucleotide sequence that encodes MMP11 protein shares at least 80%, at least 85%, at least 90%, at least 95%, at least 96%, at least 97%, at least 98%, at least 99%, or 100% sequence identity to wild-type human MMP11 gene sequence. In some embodiments, the polynucleotide sequence that encodes MMP11 protein shares at least 80%, at least 85%, at least 90%, at least 95%, at least 96%, at least 97%, at least 98%, at least 99%, or 100% sequence identity to SEQ ID NO: 86. In some embodiments, the MMP11 protein shares at least 80%, at least 85%, at least 90%, at least 95%, at least 97%, at least 98%, at least 99%, or 100% sequence identity to wild-type human MMP11 protein sequence. In some embodiments, the MMP11 protein shares at least 80%, at least 85%, at least 90%, at least 95%, at least 96%, at least 97%, at least 98%, at least 99%, or 100% sequence identity to SEQ ID NO: 87.

In some embodiments, a mutant, variant or fragment of MMP11 retains the function of MMP11. In some embodiments, a truncated variant of MMP11 (such as a functional truncated variant of MMP11) is used in the polynucleotides, expression cassettes, vectors and methods described herein.

Illustrative sequences are shown in Table 2 below.

### SYNPO2L

SYNPO2L is expressed in cardiac cells (cardiomyocytes) only. SYNPO2L is a gene encoding an actin-associated protein that may play a role in modulating actin-cytoskeleton mediated cell shape and conformation. There are at least two isoforms of SYNPO2L, the longer isoform of SYNPO2L, known as the adult isoform (SYNPO2LA) and the shorter isoform of SYNPO2L, known as the fetal isoform (SYNPO2LB). See https://alphafold.ebi.ac.uk for structure of SYNPO2LA and SYNPO2LB proteins. Variants of SYNPO2L were reported to confer either risk or protection for atrial fibrillation. See Front Cardiovasc Med. 2021; 8: 650667; UK Biobank https://azphewas.com; Nat Communications. 2020 Jan. 9; 11(1):163.

In some embodiments, the disclosure provides a vector comprising a polynucleotide sequence that encodes a SYNPO2L protein (or a mutant, variant, or fragment thereof), operatively linked to a cardiac-specific promoter (e.g., a TNNT2 promoter). In some embodiments, the polynucleotide sequence that encodes SYNPOP2L protein shares at least 80%, at least 85%, at least 90%, at least 95%, at least 96%, at least 97%, at least 98%, at least 99%, or 100% sequence identity to a wild-type human SYNPO2L gene sequence. In some embodiments, the SYNPO2L protein shares at least 80%, at least 85%, at least 90%, at least 95%, at least 96%, at least 97%, at least 98%, at least 99%, or 100% sequence identity to a wild-type human SYNPO2L protein sequence.

In some embodiments, the disclosure provides a vector comprising a polynucleotide sequence that encodes the SYNPO2LA protein (or a mutant, variant, or fragment thereof), operatively linked to a cardiac-specific promoter (e.g., a TNNT2 promoter). In some embodiments, the polynucleotide sequence that encodes SYNPOP2LA protein shares at least 80%, at least 85%, at least 90%, at least 95%, at least 96%, at least 97%, at least 98%, at least 99%, or 100% sequence identity to human SYNPO2LA gene sequence. In some embodiments, the polynucleotide sequence that encodes SYNPP2LA protein shares at least 80%, at least 85%, at least 90%, at least 95%, at least 96%, at least 97%, at least 98%, at least 99%, or 100% sequence identity to SEQ ID NO: 88. In some embodiments, the SYNPO2LA protein shares at least 80%, at least 85%, at least 90%, at least 95%, at least 96%, at least 97%, at least 98%, at least 99% or 100% sequence identity to human SYNPO2LA protein sequence. In some embodiments, the SYNPO2LA protein shares at least 80%, at least 85%, at least 90%, at least 95%, at least 96%, at least 97%, at least 98%, at least 99%, or 100% sequence identity to SEQ ID NO: 89.

In some embodiments, the disclosure provides a vector comprising a polynucleotide sequence that encodes a SYNPO2LB protein (or a mutant, variant, or fragment thereof), operatively linked to a cardiac-specific promoter (e.g., a TNNT2 promoter). In some embodiments, the polynucleotide sequence that encodes SYNPOP2LB protein shares at least 80%, at least 85%, at least 90%, at least 95%, at least 96%, at least 97%, at least 98%, at least 99%, or 100% sequence identity to human SYNPO2LB gene sequence. In some embodiments, the polynucleotide sequence that encodes SYNPP2LB protein shares at least 80%, at least 85%, at least 90%, at least 95%, at least 96%, at least 97%, at least 98%, at least 99%, or 100% sequence identity to SEQ ID NO: 90. In some embodiments, the SYNPO2LB protein shares at least 80%, at least 85%, at least 90%, at least 95%, at least 96%, at least 97%, at least 98%, at least 99%, or 100% sequence identity to human SYNPO2LB protein sequence. In some embodiments, the SYNPO2LB protein shares at least 80%, at least 85%, at least 90%, at least 95%, at least 96%, at least 97%, at least 98%, at least 99%, or 100% sequence identity to SEQ ID NO: 91.

In some embodiments, it is the longer isoform of SYNPO2L, SYNPO2LA, which is used in the polynucleotides, expression cassettes, vectors and methods described herein. In other embodiments, it is the shorter isoform of SYNPO2L, SYNPO2LB, which is used in the polynucleotides, expression cassettes, vectors and methods described herein.

In some embodiments, a mutant, variant or fragment of SYNPO2LA or SYNPO2LB retains the function of SYNPO2LA or SYNPO2LB, respectively. In some embodiments, a truncated variant of SYNPO2LA or SYNPO2LB (such as a functional truncated variant of SYNPO2LA or SYNPO2LB) is used in the polynucleotides, expression cassettes, vectors and methods described herein.

Illustrative sequences are shown in Table 2 below.

### MTSS1

MTSS1 is a gene expressed in non-cardiac tissues with low tissue specificity, as well as cardiomyocytes with strongest expression in the left ventricle. MTSS1 interacts with the actin cytoskeleton and the cell membrane to regulate cell structure and intercellular junctions. Diseases associated with MTSS1 include Lung Giant Cell Carcinoma and Wiskott-Aldrich Syndrome. Cardioprotective effects of MTSS1 enhancer variants have been reported. See Morley et al. 2019, Circulation, 139:2073-2076.

In some embodiments, the disclosure provides a vector comprising a polynucleotide sequence that comprises or encodes an inhibitory RNA targeting MTSS1 expression, operatively linked to a promoter (e.g., U6 promoter). In some embodiments, the inhibitory RNA is an siRNA. In some embodiments, the inhibitory RNA is an shRNA. In some embodiments, the inhibitory RNA is an antisense RNA. When introduced into a cell or a subject, the inhibitory RNA can decrease the expression of MTSS1 protein.

In some embodiments, the disclosure provides an inhibitory RNA, or a composition comprising an inhibitory RNA, targeting MTSS1 expression. In some embodiments, the inhibitory RNA is naked inhibitory RNA. In some embodiments, the inhibitory RNA is formulated in a lipid nanoparticle composition. In some embodiments, the inhibitory RNA is an siRNA. In some embodiments, the inhibitory RNA is an shRNA. In some embodiments, the inhibitory RNA is an antisense RNA. When introduced into a cell or a subject, the inhibitory RNA can decrease the expression of MTSS1 protein.

In some embodiments, the inhibitory RNA targeting MTSS1 expression comprises at least 15, at least 16, at least 17, at least 18, at least 19, or all contiguous nucleotides of any one of SEQ NOs: 94-100, with 0, 1, 2, or 3 mismatches. In some embodiments, the inhibitory RNA targeting MTSS1 expression comprises at least 15, at least 16, at least 17, at least 18, at least 19, or all contiguous nucleotides of any one of SEQ NOs: 94-100. In some embodiments, the inhibitory RNA targeting MTSS1 expression comprises at least 15, at least 16, at least 17, at least 18, at least 19, or all contiguous nucleotides of SEQ NO: 94. In some embodiments, the inhibitory RNA targeting MTSS1 expression comprises at least 15, at least 16, at least 17, at least 18, at least 19, or all contiguous nucleotides of SEQ NO: 95. In some embodiments, the inhibitory RNA targeting MTSS1 expression comprises at least 15, at least 16, at least 17, at least 18, at least 19, or all contiguous nucleotides of SEQ NO: 96. In some embodiments, the inhibitory RNA targeting MTSS1 expression comprises at least 15, at least 16, at least 17, at least 18, at least 19, or all contiguous nucleotides of SEQ NO: 97. In some embodiments, the inhibitory RNA targeting MTSS1 expression comprises at least 15, at least 16, at least 17, at least 18, at least 19, or all contiguous nucleotides of SEQ NO: 98. In some embodiments, the inhibitory RNA targeting MTSS1 expression comprises at least 15, at least 16, at least 17, at least 18, at least 19, or all contiguous nucleotides of SEQ NO: 99. In some embodiments, the inhibitory RNA targeting MTSS1 expression comprises at least 15, at least 16, at least 17, at least 18, at least 19, or all contiguous nucleotides of SEQ NO: 100.

In some embodiments, the inhibitory RNA targeting MTSS1 expression targets any region of wild-type human MTSS1 mRNA. In some embodiments, the inhibitory RNA targeting MTSS1 expression targets any region of SEQ ID NO: 92.

In some embodiments, the inhibitory RNA targeting MTSS1 expression comprises at least 15, at least 16, at least 17, at least 18, or at least 19 contiguous nucleotides of SEQ ID NO: 92, with 0, 1, 2, or 3 mismatches. In some embodiments, the inhibitory RNA targeting MTSS1 expression comprises at least 15, at least 16, at least 17, at least 18, or at least 19 contiguous nucleotides of SEQ ID NO: 92.

In some embodiments, the inhibitory RNA targeting MTSS1 expression is complementary to at least 15, at least 16, at least 17, at least 18, or at least 19 contiguous nucleotides of SEQ ID NO: 92, with 0, 1, 2, or 3 mismatches. In some embodiments, the inhibitory RNA targeting MTSS1 expression is complementary to at least 15, at least 16, at least 17, at least 18, or at least 19 contiguous nucleotides of SEQ ID NO: 92.

In some embodiments, the inhibitory RNA targeting MTSS1 expression is effective to inhibit the expression of MTSS1 protein, such as wild-type human MTSS1 protein. In some embodiments, the inhibitory RNA targeting MTSS1 expression is effective to inhibit the expression of MTSS1 protein sequence of SEQ ID NO: 93 (e.g., in a cell or cells, such as cardiac cells, e.g., a cardiomyocyte).

In some embodiments, the disclosure provides a vector comprising a polynucleotide sequence that comprises or encodes a gene editing system targeting MTSS1 expression, operatively linked to a promoter. In some embodiments, the inhibition of MTSS1 expression occurs by gene editing approaches well known to a person skilled in the art, e.g., by clustered regularly interspaced short palindromic repeat (CRISPR) and CRISPR-associated (Cas) proteins. CRISPR/Cas systems generally comprise at least two components: one or more non-coding RNAs referred to as guide RNAs (gRNAs) and a Cas protein with nuclease functionality. Cas9, originally derived from S. pyogenes (SpCas9) or S. aureus (SaCas9), is the most widely used Cas protein.

In some embodiments, the present disclosure provides compositions (e.g., polynucleotides, expression cassettes and/or vectors) and methods of targeting any coding or non-coding region of the MTSS1 gene. In some embodiments, the expression cassettes and/or vectors comprise a first polynucleotide encoding a Cas endonuclease protein (e.g., a Cas9 protein such as a SpCas9 or a SaCas9 protein) operably linked to a protein-expression driving promoter (e.g., a cardiac specific promoter such as a TNNT2 promoter), and/or a second polynucleotide encoding a gRNA complementary to a coding or non-coding sequence of the MTSS1 gene, operably linked to an RNA expression-driving promoter (e.g., a U6 promoter). In some embodiments, the gRNA and the Cas endonuclease are delivered via single vector (e.g., a single AAV vector). In some embodiments, dual AAV delivery of a guide RNA and of a Cas9 protein under the control of a cardiac-specific promoter is used. The gRNA can be designed to target any suitable region of the MTSS1 gene locus, including, e.g., an intron, an exon, a gene coding region (also known as a CoDing Sequence, or “CDS”), or a non-coding region. For example, a gRNA targeting a sequence within the first exon of the MTSS1 gene can be used. Alternatively, the gRNA can be designed to target any suitable region of an enhancer, e.g., a cardiac-specific enhancer, of the MTSS1 gene. In some embodiments, the expression cassettes and/or vectors are the form of a viral vector or a virion, e.g., an AAV vector or an AAV virion as described herein.

In some embodiments, the inhibition of MTSS1 RNA expression occurs by CRISPR-mediated disruption of the MTSS1 coding sequence (e.g., the first exon sequence). In some embodiments, the expression cassettes and/or vectors comprise a first polynucleotide encoding a Cas endonuclease protein (e.g., a Cas9 protein such as a SpCas9 or a SaCas9 protein) operably linked to a protein-expression driving promoter (e.g., a cardiac specific promoter such as a TNNT2 promoter), and/or a second polynucleotide encoding a gRNA complementary to a sequence of a coding region of the MTSS1 gene (e.g., the first exon), operably linked to an RNA expression-driving promoter (e.g., a U6 promoter).

In some embodiments, the gRNA is complementary to any region of the coding sequence of the MTSS1 gene. In some embodiments, the gRNA is complementary to any region of SEQ ID NO: 406. In some embodiments, the gRNA is complementary to any region within one or more exons of SEQ ID NO: 406. In some embodiments, the gRNA is complementary to any region of the first exon of MTSS1. In some embodiments, the gRNA is complementary to any region of SEQ ID NO: 407. In some embodiments, the expression cassettes and/or vectors comprise a first polynucleotide encoding a Cas endonuclease protein (e.g., a Cas9 protein such as a SpCas9 or a SaCas9 protein) operably linked to a protein-expression driving promoter (e.g., a cardiac specific promoter such as a TNNT2 promoter), and/or a second polynucleotide encoding a gRNA complementary to any region of SEQ ID NO: 406, operably linked to an RNA expression-driving promoter (e.g., a U6 promoter). In some embodiments, the expression cassettes and/or vectors comprise a first polynucleotide encoding a Cas endonuclease protein (e.g., a Cas9 protein such as a SpCas9 or a SaCas9 protein) operably linked to a protein-expression driving promoter (e.g., a cardiac specific promoter such as a TNNT2 promoter), and/or a second polynucleotide encoding a gRNA complementary to any region of SEQ ID NO: 407, operably linked to an RNA expression-driving promoter (e.g., a U6 promoter).

In some embodiments, the present disclosure provides compositions (e.g., polynucleotides, expression cassettes and/or vectors) and methods of targeting a non-coding region regulating the expression of the MTSS1 gene (e.g., a non-coding region regulating or promoting the expression of the MTSS1 gene in muscle cells and/or cardiac cells). In some embodiments, the expression cassettes and/or vectors comprise a first polynucleotide encoding a Cas endonuclease protein (e.g., a Cas9 protein such as a SpCas9 or a SaCas9 protein) operably linked to a protein-expression driving promoter (e.g., a cardiac specific promoter such as a TNNT2 promoter), and/or a second polynucleotide encoding a gRNA complementary to a sequence of a non-coding region of the MTSS1 gene (e.g., a non-coding region promoting or driving the expression of MTSS1 in cardiac cells), operably linked to an RNA expression-driving promoter (e.g., a U6 promoter). In some embodiments, the non-coding region is an enhancer (such as a native MTSS1 enhancer, e.g., an enhancer selectively regulating MTSS1 expression in cardiac cells). In some embodiments, the non-coding region is a promoter (such as a native MTSS1 promoter, e.g., a promoter selectively regulating MTSS1 expression in cardiac cells). In some embodiments, the targeting of MTSS1 using the CRISPR/Cas system as described herein is muscle-cell specific and/or cardiac cell-specific. In some embodiments, the targeting of MTSS1 using the CRISPR/Cas system as described herein does not substantially affect MTSS1 expression in one or more non-target tissues (e.g., brain, kidney and/or liver tissues).

In some embodiments, the present disclosure provides compositions (e.g., polynucleotides, expression cassettes and/or vectors) and methods of targeting a cardiac-specific enhancer of the MTSS1 gene. In some embodiments, an expression cassette and/or vector comprises a first polynucleotide encoding a Cas endonuclease protein (e.g., a Cas9 protein such as a SpCas9 or a SaCas9 protein) operably linked to a protein-expression driving promoter (e.g., a cardiac specific promoter such as a TNNT2 promoter), and/or a second polynucleotide encoding a gRNA complementary to a sequence of a cardiac-specific enhancer of the MTSS1 gene, operably linked to an RNA expression-driving promoter (e.g., a U6 promoter).

In some embodiments, the present disclosure provides compositions (e.g., polynucleotides, expression cassettes and/or vectors) and methods of targeting a region of conserved transcription factor binding sites upstream to the SNP rs12541595 (e.g., HAND2, TWIST1, and/or MEF2A, B, C or D transcription factor binding sites). In some embodiments, an expression cassette and/or vector comprises a first polynucleotide encoding a Cas endonuclease protein (e.g., a Cas9 protein such as a SpCas9 or a SaCas9 protein) operably linked to a protein-expression driving promoter (e.g., a cardiac specific promoter such as a TNNT2 promoter), and/or a second polynucleotide encoding a gRNA complementary to a sequence of a region of conserved transcription factor binding sites upstream to the SNP rs12541595, operably linked to an RNA expression-driving promoter (e.g., a U6 promoter).

In some embodiments, the present disclosure provides compositions (e.g., polynucleotides, expression cassettes and/or vectors) and methods of targeting one or more of HAND2, TWIST1, and/or MEF2A, B, C or D transcription factor binding site(s) within the enhancer of the MTSS1 gene (e.g., those promoting cardiac-specific expression of MTSS1). In some embodiments, an expression cassette and/or vector comprises a first polynucleotide encoding a Cas endonuclease protein (e.g., a Cas9 protein such as a SpCas9 or a SaCas9 protein) operably linked to a protein-expression driving promoter (e.g., a cardiac specific promoter such as a TNNT2 promoter), and/or a second polynucleotide encoding a gRNA complementary to a sequence of HAND2, TWIST1, and/or MEF2A, B, C or D transcription factor binding site(s) within the enhancer of the MTSS1 gene (e.g., promoting cardiac-specific expression of MTSS1), operably linked to an RNA expression-driving promoter (e.g., a U6 promoter).

In some embodiments, the present disclosure provides compositions (e.g., polynucleotides, expression cassettes and/or vectors) and methods of targeting the MTSS1 gene by CRISPR-mediated disruption of non-coding genomic sequence that regulates (e.g., specifically directs) cardiac expression of MTSS1 (e.g., hg38 coordinates chr8:124,845,017-124,845,217). In some embodiments, an expression cassette and/or vector comprises a first polynucleotide encoding a Cas endonuclease protein (e.g., a Cas9 protein such as a SpCas9 or a SaCas9 protein) operably linked to a protein-expression driving promoter (e.g., a cardiac specific promoter such as a TNNT2 promoter), and/or a second polynucleotide encoding a gRNA complementary to a non-coding genomic sequence that specifically directs cardiac expression of MTSS1 (e.g., hg38 coordinates chr8:124,845,017-124,845,217), operably linked to an RNA expression-driving promoter (e.g., a U6 promoter).

In some embodiments, the present disclosure provides compositions (e.g., polynucleotides, expression cassettes and/or vectors) and methods of targeting the MTSS1 gene by CRISPR-mediated disruption of any region of the sequence identified by hg38 coordinates chr8:124,845,017-124,845,217. In some embodiments, an expression cassette and/or vector comprises a first polynucleotide encoding a Cas endonuclease protein (e.g., a Cas9 protein such as a SpCas9 or a SaCas9 protein) operably linked to a protein-expression driving promoter (e.g., a cardiac specific promoter such as a TNNT2 promoter), and/or a second polynucleotide encoding a gRNA complementary to a sequence within hg38 coordinates chr8:124,845,017-124,845,217, operably linked to an RNA expression-driving promoter (e.g., a U6 promoter).

In some embodiments, the gRNA is complementary (partially or fully) to any region of SEQ ID NO: 408. In some embodiments, the gRNA is complementary (at least partially or fully) to one or more of the following region: SEQ ID NO: 409, SEQ ID NO: 410, and/or SEQ ID NO: 411. In some embodiments, the gRNA is complementary (partially or fully) to any region of SEQ ID NO: 409. In some embodiments, the gRNA is complementary (partially or fully) to any region of SEQ ID NO: 410. In some embodiments, the gRNA is complementary (partially or fully) to any region of SEQ ID NO: 411. In some embodiments, the rs1241595 site, nucleotide G (within SEQ ID NO: 408) is targeted or is within the sequence being targeted by gRNA. In some embodiments, the expression cassettes and/or vectors comprise a first polynucleotide encoding a Cas endonuclease protein (e.g., a Cas9 protein such as a SpCas9 or a SaCas9 protein) operably linked to a protein-expression driving promoter (e.g., a cardiac specific promoter such as a TNNT2 promoter), and/or a second polynucleotide encoding a gRNA complementary to any region of SEQ ID NO: 408, operably linked to an RNA expression-driving promoter (e.g., a U6 promoter). In some embodiments, the expression cassettes and/or vectors comprise a first polynucleotide encoding a Cas endonuclease protein (e.g., a Cas9 protein such as a SpCas9 or a SaCas9 protein) operably linked to a protein-expression driving promoter (e.g., a cardiac specific promoter such as a TNNT2 promoter), and/or a second polynucleotide encoding a gRNA complementary to any region of SEQ ID NO: 409, SEQ ID NO: 410 and/or SEQ ID NO: 411, operably linked to an RNA expression-driving promoter (e.g., a U6 promoter).

In some embodiments, the CRISPR-mediated gene editing systems described herein are effective to inhibit the expression of MTSS1 protein, such as wild-type human MTSS1 protein. In some embodiments, the CRISPR-mediated gene editing systems described herein effective to inhibit the expression of MTSS1 protein sequence of SEQ ID NO: 93 (e.g., in a cell or cells, such as cardiac cells, e.g., a cardiomyocyte).

In some embodiments, the inhibition of MTSS1 expression occurs by administration of a small molecule inhibitor of the MTSS1 protein. In some embodiments, the small molecule inhibitor inhibits the transcription and/or translation of the MTSS1 protein. In some embodiments, the small molecule inhibitor reduces the level of the MTSS1 protein in cells. In some embodiments, the small molecule inhibitor inhibits the folding of the MTSS1 protein. In some embodiments, the small molecule inhibitor inhibits the function of the MTSS1 protein. In some embodiments, the small molecule inhibitor reduces the half-life and/or stability of the MTSS1 protein. In some embodiments, the small molecule inhibitor promotes the degradation and/or destruction of the MTSS1 protein.

Illustrative sequences are shown in Table 2 below.

Combination of Cardioprotective Genes and/or Inhibitory RNA

In some embodiments, the disclosure provides a vector comprising a polynucleotide sequence that comprises at least two cardioprotective genes and/or cardioprotective inhibitory RNA and/or cardioprotective gene editing systems.

In some embodiments, the disclosure provides a vector comprising a polynucleotide sequence that comprises at least two cardioprotective genes and/or cardioprotective inhibitory RNA and/or cardioprotective gene editing systems, which vector had cardioprotective effect when delivered to a mammal having a deleterious mutation in the TTN gene (e.g., a TTNtv mutation).

In some embodiments, the disclosure provides a vector comprising a polynucleotide sequence that comprises at least two cardioprotective genes and/or cardioprotective inhibitory RNA and/or cardioprotective gene editing systems, which vector had cardioprotective effect when delivered to a mammal having a deleterious mutation in the MLP/CSRP3 gene.

In some embodiments, the disclosure provides a vector comprising a polynucleotide sequence that comprises at least two cardioprotective genes and/or cardioprotective inhibitory RNA, wherein at least one gene or RNA is selected from the group consisting of: an MMP11 gene (or a mutant, variant or fragment thereof), a SYNPO2L gene (e.g., SYNPO2LA, SYNPO2B, or a mutant, variant or fragment of any thereof), an inhibitory RNA targeting MTSS1 expression, and a CRISPR/Cas system targeting MTSS1 expression. In some embodiments, the disclosure provides a vector comprising a polynucleotide sequence that comprises at least two cardioprotective genes and/or cardioprotective inhibitory RNA, wherein at least one gene or RNA is selected from the group consisting of: an MMP11 gene (or a mutant, variant or fragment thereof), a SYNPO2L gene (e.g., SYNPO2LA, SYNPO2B, or a mutant, variant or fragment of any thereof), and an inhibitory RNA targeting MTSS1 expression, optionally wherein the MMP11 gene and SYNPO2L gene are to be operatively linked to a cardiac-specific promoter (e.g., a TNNT2 promoter), and the inhibitory RNA is to be linked to a U6 promoter (or another promoter suitable for expression of inhibitory RNA).

In some embodiments, the disclosure provides a vector comprising a polynucleotide sequence that comprises at least two cardioprotective genes and/or cardioprotective inhibitory RNA, which vector has a cardioprotective effect when delivered to a mammal having a deleterious mutation in the TTN gene (e.g., a TTNtv mutation), and wherein at least one gene or RNA is selected from the group consisting of: an MMP11 gene (or a mutant, variant or fragment thereof), a SYNPO2L gene (e.g., SYNPO2LA, SYNPO2B, or a mutant, variant or fragment of any thereof), an inhibitory RNA targeting MTSS1 expression, and a gene editing system targeting MTSS1 expression.

In some embodiments, the disclosure provides a vector comprising a polynucleotide sequence that comprises at least two cardioprotective genes and/or cardioprotective inhibitory RNA, which vector has a cardioprotective effect when delivered to a mammal having a deleterious mutation in the MLP/CSRP3 gene, and wherein at least one gene or RNA is selected from the group consisting of: an MMP11 gene (or a mutant, variant or fragment thereof), a SYNPO2L gene (e.g., SYNPO2LA, SYNPO2B, or a mutant, variant or fragment of any thereof), and an inhibitory RNA targeting MTSS1 expression.

In some embodiments, the disclosure provides a vector comprising a polynucleotide sequence that comprises at least two of: an MMP11 gene (or a mutant, variant or fragment thereof), a SYNPO2L gene (e.g., SYNPO2LA, SYNPO2B, or a mutant, variant or fragment of any thereof), an inhibitory RNA targeting MTSS1 expression, and a gene editing system targeting MTSS1 expression. In some embodiments, the disclosure provides a vector comprising a polynucleotide sequence that comprises at least two of: an MMP11 gene (or a mutant, variant or fragment thereof), a SYNPO2L gene (e.g., SYNPO2LA, SYNPO2B, or a mutant, variant or fragment of any thereof), an inhibitory RNA targeting MTSS1 expression, and a gene editing system targeting MTSS1 expression, optionally wherein the MMP11 gene and SYNPO2L gene are to be operatively linked to a cardiac-specific promoter (e.g., a TNNT2 promoter), and the inhibitory RNA is to be linked to a U6 promoter (or another promoter suitable for expression of inhibitory RNA).

In some embodiments, the disclosure provides a vector comprising a polynucleotide sequence that comprises all three of: an MMP11 gene (or a mutant, variant or fragment thereof), a SYNPO2L gene (e.g., SYNPO2LA, SYNPO2B, or a mutant, variant or fragment of any thereof), and an inhibitory RNA targeting MTSS1 expression or a gene editing system targeting MTSS1 expression. In some embodiments, the disclosure provides a vector comprising a polynucleotide sequence that comprises all three of: an MMP11 gene (or a mutant, variant or fragment thereof), a SYNPO2L gene (e.g., SYNPO2LA, SYNPO2B, or a mutant, variant or fragment of any thereof), and an inhibitory RNA targeting MTSS1 expression, optionally wherein the MMP11 gene and SYNPO2L gene are to be operatively linked to a cardiac-specific promoter (e.g., a TNNT2 promoter), and the inhibitory RNA is to be linked to a U6 promoter (or another promoter suitable for expression of inhibitory RNA).

In some embodiments, the disclosure provides a vector comprising a polynucleotide sequence that encodes at least one gene product selected from: MMP11, SYNPO2LA, SYNPO2LB, and siRNA or shRNA targeting MTSS1, optionally wherein the MMP11 and SYNPO2L sequences are operatively linked to a cardiac-specific promoter (e.g., TNNT2 promoter) and optionally wherein the siRNA or shRNA targeting MTSS1 is operatively linked to a U6 promoter.

In each of the embodiments described herein, two or more cardioprotective genes may be under the control of one, two or more promoters, which may be the same or different from each other.

TABLE 2
Exemplary cardioprotective gene and inhibitory RNA sequences

| | | SEQ |
|---|---|---|
| Name | Sequence | ID NO: |
| MMP11 DNA | atggctccggccgcctggctccgcagcgcggccgcgcgcgccctcctgcccccgatgctgct | 86 |
| sequence | gctgctgctccagccgccgccgctgctggcccgggctctgccgccggacgcccaccacctcc | |
| | atgccgagaggagggggccacagccctggcatgcagccctgcccagtagcccggcacctgc | |
| | ccctgccacgcaggaagccccccggcctgccagcagcctcaggcctccccgctgtggcgtgc | |
| | ccgacccatctgatgggctgagtgcccgcaaccgacagaagaggttcgtgctttctggcgggc | |
| | gctgggagaagacggacctcacctacaggatccttcggttcccatggcagttggtgcaggagca | |
| | ggtgcggcagacgatggcagaggccctaaaggtatggagcgatgtgacgccactcacctttact | |
| | gaggtgcacgagggccgtgctgacatcatgatcgacttcgccaggtactggcatggggacgac | |
| | ctgccgtttgatgggcctgggggcatcctggcccatgccttcttccccaagactcaccgagaagg | |
| | ggatgtccacttcgactatgatgagacctggactatcggggatgaccagggcacagacctgctg | |
| | caggtggcagcccatgaatttggccacgtgctggggctgcagcacacaacagcagccaaggc | |
| | cctgatgtccgccttctacacctttcgctacccactgagtctcagcccagatgactgcaggggcgt | |
| | tcaacacctatatggccagccctggcccactgtcacctccaggaccccagccctgggccccca | |
| | ggctgggatagacaccaatgagattgcaccgctggagccagacgccccgccagatgcctgtga | |
| | ggcctcctttgacgcggtctccaccatccgaggcgagctctttttcttcaaagcgggctttgtgtgg | |
| | cgcctccgtgggggccagctgcagcccggctacccagcattggcctctcgccactggcaggg | |
| | actgcccagccctgtggacgctgccttcgaggatgcccagggccacatttggttcttccaaggtg | |
| | ctcagtactgggtgtacgacggtgaaaagccagtcctgggccccgcacccctcaccgagctgg | |
| | gcctggtgaggttcccggtccatgctgccttggtctggggtcccgagaagaacaagatctacttct | |
| | tccgaggcagggactactggcgtttccaccccagcacccggcgtgtagacagtcccgtgcccc | |
| | gcagggccactgactggagaggggtgccctctgagatcgacgctgccttccaggatgctgatg | |
| | gctatgcctacttcctgcgcggccgcctctactggaagtttgaccctgtgaaggtgaaggctctgg | |
| | aaggcttcccccgtctcgtgggtcctgacttctttggctgtgccgagcctgccaacactttcctc | |
| MMP11 protein | MAPAAWLRSAAARALLPPMLLLLLQPPPLLARALPPDAHHL | 87 |
| sequence | HAERRGPQPWHAALPSSPAPAPATQEAPRPASSLRPPRCGVP | |
| | DPSDGLSARNRQKRFVLSGGRWEKTDLTYRILRFPWQLVQE | |
| | QVRQTMAEALKVWSDVTPLTFTEVHEGRADIMIDFARYWH | |
| | GDDLPFDGPGGILAHAFFPKTHREGDVHFDYDETWTIGDDQ | |
| | GTDLLQVAAHEFGHVLGLQHTTAAKALMSAFYTFRYPLSLS | |
| | PDDCRGVQHLYGQPWPTVTSRTPALGPQAGIDTNEIAPLEPD | |
| | APPDACEASFDAVSTIRGELFFFKAGFVWRLRGGQLQPGYPA | |
| | LASRHWQGLPSPVDAAFEDAQGHIWFFQGAQYWVYDGEKP | |
| | VLGPAPLTELGLVRFPVHAALVWGPEKNKIYFFRGRDYWRF | |
| | HPSTRRVDSPVPRRATDWRGVPSEIDAAFQDADGYAYFLRG | |
| | RLYWKFDPVKVKALEGFPRLVGPDFFGCAEPANTFL | |
| SYNPO2LA | atgggtgctgaggaggaggtgctggtcacactatcagggggagccccctggggcttccgactt | 88 |
| DNA sequence | catgggggggccgagcagaggaaaccgttacaggtgtctaagattcgaagacggagccaggc | |
| | tggcagagcaggactccgagagagggaccagctcttggcaatcaatggggtctcttgcaccaa | |
| | cctctcccatgccagtgccatgagcctcatcgatgcctcaggaaatcagcttgtcctcactgtgca | |
| | gcggttagcagacgagggtcctgtgcaatctccatctccccatgagcttcaggtgctgtcaccctt | |
| | atctccactaagtcctgagccccctggtgctccagttcctcagcctcttcagcctgggagccttcgt | |
| | tcacctcctgatagtgaggcttactacggagagactgacagtgatgctgatggccctgccaccca | |
| | ggagaagccccgtcgacctcgccgccgaggccccacaaggcccacccctccgggtgcccca | |
| | cctgatgaggtctacctgtctgacagccctgcagagccagcacctactatccctggccctcccag | |
| | ccagggtgacagcgtgtgagctccccgtcttgggaggatggggcagcccttcagccaccccca | |
| | gctgaggctctgctgttaccccatggccccctccgacctggtcctcatctcatccctatggtgggg | |
| | cctgttccccacccagtggcagaagatcttactaccacctacacccagaaggccaagcaagcca | |
| | aactgcaacgtgcagagagcctccaagagaagagcataaaagaggccaagaccaaatgcag | |
| | gacaattgcatccctgctcactgcagcccccaacccccactccaaaggggtacttatgtttaagaa | |
| | acggcggcagagagccaagaagtacaccctggtgagcttcggggctgctgctgggacaggcg | |
| | ctgaggaggaggacggcgttccccccacgagtgagtccgagctggacgaagaagccttctctg | |
| | acgcccgcagcctcaccaatcaatctgactgggacagtccctatctggacatggagcttgccag | |
| | ggcgggctcaagagcatcagagggccagggctctgggctgggagggcagctgagtgaggtc | |
| | tctgggcgaggggtgcagctctttgaacagcagcgccagcgcgcagactccagcacccagga | |
| | actggcacgggtcgaaccagcagccatgctcaacggggaaggcctgcagtcaccacctcggg | |
| | cccagagtgctcccccagaggcagctgtgctcccacccagccccttgccggcgcctgtagcca | |
| | gccccagacccttccaaccaggtggtggagccccgaccccagctccaagcatctttaaccggtc | |
| | agccaggccctttaccccgggcctacaagggcagcggccaactaccacctcggttattttccgg | |
| | cctttagcccccaaaagggcgaacgacagcctggggggcctcagccccgccccacccccctt | |
| | cttgtcttcgcaggggcccacccctctgcccagcttcacttcaggggttcccagccacgcgccag | |
| | tctctggttcccccagcaccccacgctcctcgggccctgtgacagccaccagctccctgtacatc | |
| | ccagcccctagtcggcctgtcaccccaggtggagctccagagccccccgctcctcctagcgca | |
| | gctgccatgacctccaccgcttctatcttcctatctgcgcctttgcgaccctctgcgcgcccagag | |
| | gcgcctgccccaggcccaggggctcctgagccccccagcgctcgcgagcagcgcatctctgt | |
| | gccagctgcccgcacgggtatcctgcaggaggcccggcgccgggggacccggaagcagat | |
| | gttccggccgggaaaggaggagacgaagaactcgcccaaccccgagctgctatcgctggtac | |
| | agaacctggatgaaaagcctcgggccgggggtgcagaatctggtcctgaagaagatgctctga | |
| | gcctcggggctgaagcctgcaacttcatgcagccagtaggggccaggagttacaagaccctgc | |
| | ctcacgtgacacctaagaccccccctccaatggctcccaagaccccgccccctatgactcctaa | |
| | gactccacccccagtggctcctaagcccccatctcgagggctccttgatgggctcgtgaatggg | |
| | gcagcctcttcggctggaatccctgagccaccaaggctgcagggcaggggggggagctgttt | |
| | gctaagcggcagagccgtgcggacaggtatgtggtggaaggtacacctggtcctggtcttggc | |
| | cctcggcctagaagtccttctcctaccccgtctctgcccccttcctggaaatattcacccaacatcc | |
| | gtgccccgcctcctattgcttacaacccactgctctctccctttttcccccaggcggcccgaactct | |
| | ccctaaggcccaatcccaggggcctcgggcaacacccaagcagggcatcaaggctctagattt | |
| | tatgcggcatcagccctatcaacttaaaactgccatgttctgttttgatgaggttcccccgactcctg | |
| | gccctatcgcctcagggtcccccaaaactgcccgagtccaggagattcgccggttttccactccg | |
| | gcaccccagcccactgcagaacccctggctcccactgtgcttgccccccgagcagccactaca | |
| | ctggatgagcccatctggagaacagaactggcctcagcccctgttcctagcccagcccctcctc | |
| | cagaggctcccaggggccttggggcttctcccagctcctgcggtttccaggtagccaggccccg | |
| | attttcagccaccagaacaggattgcaagctcatgtgtggaggcctggggcagggcaccag | |
| SYNPO2LA | MGAEEEVLVTLSGGAPWGFRLHGGAEQRKPLQVSKIRRRSQ | 89 |
| protein | AGRAGLRERDQLLAINGVSCTNLSHASAMSLIDASGNQLVLT | |
| sequence | VQRLADEGPVQSPSPHELQVLSPLSPLSPEPPGAPVPQPLQPG | |
| | SLRSPPDSEAYYGETDSDADGPATQEKPRRPRRRGPTRPTPP | |
| | GAPPDEVYLSDSPAEPAPTIPGPPSQGDSRVSSPSWEDGAALQ | |
| | PPPAEALLLPHGPLRPGPHLIPMVGPVPHPVAEDLTTTYTQK | |
| | AKQAKLQRAESLQEKSIKEAKTKCRTIASLLTAAPNPHSKGV | |
| | LMFKKRRQRAKKYTLVSFGAAAGTGAEEEDGVPPTSESELD | |
| | EEAFSDARSLTNQSDWDSPYLDMELARAGSRASEGQGSGLG | |
| | GQLSEVSGRGVQLFEQQRQRADSSTQELARVEPAAMLNGEG | |
| | LQSPPRAQSAPPEAAVLPPSPLPAPVASPRPFQPGGGAPTPAP | |
| | SIFNRSARPFTPGLQGQRPTTTSVIFRPLAPKRANDSLGGLSPA | |
| | PPPFLSSQGPTPLPSFTSGVPSHAPVSGSPSTPRSSGPVTATSSL | |
| | YIPAPSRPVTPGGAPEPPAPPSAAAMTSTASIFLSAPLRPSARP | |
| | EAPAPGPGAPEPPSAREQRISVPAARTGILQEARRRGTRKQM | |
| | FRPGKEETKNSPNPELLSLVQNLDEKPRAGGAESGPEEDALS | |
| | LGAEACNFMQPVGARSYKTLPHVTPKTPPPMAPKTPPPMTP | |
| | KTPPPVAPKPPSRGLLDGLVNGAASSAGIPEPPRLQGRGGELF | |
| | AKRQSRADRYVVEGTPGPGLGPRPRSPSPTPSLPPSWKYSPNI | |
| | RAPPPIAYNPLLSPFFPQAARTLPKAQSQGPRATPKQGIKALD | |
| | FMRHQPYQLKTAMFCFDEVPPTPGPIASGSPKTARVQEIRRFS | |
| | TPAPQPTAEPLAPTVLAPRAATTLDEPIWRTELASAPVPSPAP | |
| | PPEAPRGLGASPSSCGFQVARPRFSATRTGLQAHVWRPGAG | |
| | HQ | |
| SYNPO2LB | atggagacctttgagcccatcagccaagagcccctcagccaagccagctatgacaaagcccca | 90 |
| DNA sequence | gacccagttcctgagctccaagactcgttctatgcagaactgcaacgtgcagagagcctccaag | |
| | agaagagcataaaagaggccaagaccaaatgcaggacaattgcatccctgctcactgcagccc | |
| | ccaacccccactccaaaggggtacttatgtttaagaaacggcggcagagagccaagaagtaca | |
| | ccctggtgagcttcggggctgctgctgggacaggcgctgaggaggaggacggcgttcccccc | |
| | acgagtgagtccgagctggacgaagaagccttctctgacgcccgcagcctcaccaatcaatctg | |
| | actgggacagtccctatctggacatggagcttgccaggggggctcaagagcatcagagggcc | |
| | agggctctgggctgggagggcagctgagtgaggtctctgggcgaggggtgcagctctttgaac | |
| | agcagcgccagcgcgcagactccagcacccaggaactggcacgggtcgaaccagcagccat | |
| | gctcaacggggaaggcctgcagtcaccacctcgggcccagagtgctcccccagaggcagctg | |
| | tgctcccacccagccccttgccggcgcctgtagccagccccagacccttccaaccaggtggtg | |
| | gagccccgaccccagctccaagcatctttaaccggtcagccaggccctttaccccgggcctaca | |
| | agggcagcggccaactaccacctcggttattttccggcctttagcccccaaaagggcgaacgac | |
| | agcctggggggcctcagccccgccccaccccccttcttgtcttcgcaggggcccacccctctgc | |
| | ccagcttcacttcaggggttcccagccacgcgccagtctctggttcccccagcaccccacgctcc | |
| | tcgggccctgtgacagccaccagctccctgtacatcccagcccctagtcggcctgtcaccccag | |
| | gtggagctccagagccccccgctcctcctagcgcagctgccatgacctccaccgcttctatcttc | |
| | ctatctgcgcctttgcgaccctctgcgcgcccagaggcgcctgccccaggcccaggggctcct | |
| | gagccccccagcgctcgcgagcagcgcatctctgtgccagctgcccgcacgggtatcctgcag | |
| | gaggcccggcgccgggggacccggaagcagatgttccggccgggaaaggaggagacgaa | |
| | gaactcgcccaaccccgagctgctatcgctggtacagaacctggatgaaaagcctcgggccgg | |
| | gggtgcagaatctggtcctgaagaagatgctctgagcctcggggctgaagcctgcaacttcatg | |
| | cagccagtaggggccaggagttacaagaccctgcctcacgtgacacctaagaccccccctcca | |
| | atggctcccaagaccccgccccctatgactcctaagactccacccccagtggctcctaagcccc | |
| | catctcgagggctccttgatgggctcgtgaatggggcagcctcttcggctggaatccctgagcca | |
| | ccaaggctgcagggcaggggggggagctgtttgctaagcggcagagccgtgcggacaggta | |
| | tgtggtggaaggtacacctggtcctggtcttggccctcggcctagaagtccttctcctaccccgtct | |
| | ctgcccccttcctggaaatattcacccaacatccgtgccccgcctcctattgcttacaacccactgc | |
| | tctctccctttttcccccaggcggcccgaactctccctaaggcccaatcccaggggcctcgggca | |
| | acacccaagcagggcatcaaggctctagattttatgcggcatcagccctatcaacttaaaactgc | |
| | catgttctgttttgatgaggttcccccgactcctggccctatcgcctcagggtcccccaaaactgcc | |
| | cgagtccaggagattcgccggttttccactccggcaccccagcccactgcagaacccctggctc | |
| | ccactgtgcttgccccccgagcagccactacactggatgagcccatctggagaacagaactgg | |
| | cctcagcccctgttcctagcccagcccctcctccagaggctcccaggggccttggggcttctccc | |
| | agctcctgcggtttccaggtagccaggccccgattttcagccaccagaacaggattgcaagctca | |
| | tgtgtggaggcctggggcagggcaccag | |
| SYNPOP2LB | METFEPISQEPLSQASYDKAPDPVPELQDSFYAELQRAESLQE | 91 |
| protein | KSIKEAKTKCRTIASLLTAAPNPHSKGVLMFKKRRQRAKKYT | |
| sequence | LVSFGAAAGTGAEEEDGVPPTSESELDEEAFSDARSLTNQSD | |
| | WDSPYLDMELARAGSRASEGQGSGLGGQLSEVSGRGVQLFE | |
| | QQRQRADSSTQELARVEPAAMLNGEGLQSPPRAQSAPPEAA | |
| | VLPPSPLPAPVASPRPFQPGGGAPTPAPSIFNRSARPFTPGLQG | |
| | QRPTTTSVIFRPLAPKRANDSLGGLSPAPPPFLSSQGPTPLPSF | |
| | TSGVPSHAPVSGSPSTPRSSGPVTATSSLYIPAPSRPVTPGGAP | |
| | EPPAPPSAAAMTSTASIFLSAPLRPSARPEAPAPGPGAPEPPSA | |
| | REQRISVPAARTGILQEARRRGTRKQMFRPGKEETKNSPNPE | |
| | LLSLVQNLDEKPRAGGAESGPEEDALSLGAEACNFMQPVGA | |
| | RSYKTLPHVTPKTPPPMAPKTPPPMTPKTPPPVAPKPPSRGLL | |
| | DGLVNGAASSAGIPEPPRLQGRGGELFAKRQSRADRYVVEG | |
| | TPGPGLGPRPRSPSPTPSLPPSWKYSPNIRAPPPIAYNPLLSPFF | |
| | PQAARTLPKAQSQGPRATPKQGIKALDFMRHQPYQLKTAMF | |
| | CFDEVPPTPGPIASGSPKTARVQEIRRFSTPAPQPTAEPLAPTV | |
| | LAPRAATTLDEPIWRTELASAPVPSPAPPPEAPRGLGASPSSC | |
| | GFQVARPRFSATRTGLQAHVWRPGAGHQ | |
| MTSS1 cDNA | atggaggctgtgattgagaaggaatgcagcgcgctcggaggcctcttccagaccatcatcagcg | 406 |
| sequence (with | acatgaaggggagctatccagtttgggaagatttcataaacaaagcaggaaagctgcagt | |
| first exon | cccagcttcggacaacagtagtagcagcagctgccttcttggacgcctttcagaaagtggctga | |
| underlined, and | catggccaccaacacacgtggtgggaccagggagattggatctgctctcaccaggatgtgc | |
| “alternative” | atgaggcacagaagcattgaagccaagctgaggcagttttcgagcgctttaattgattgtctg | |
| exons (that may | ataaacccacttcaagaacagatggaagaatggaagaaagtggccaaccagctggataaagac | |
| or may not be | cacgcaaaagaatataagaaagcccgccaagagataaaaaagaagtcctcggatacgct | |
| spliced out in | gaaactgcagaagaaagcaaaaaaagggagaggtgatatccagcctcagttggacagtgct | |
| MTSS1 | ctccaagatgtcaatgataagtatctcttattggaagaaacagaaaagcaggctgtccggaaggc | |
| variants) | tttgattgaagaacgtggccgattctgtaccttcatctctatgctgcggccagtgattgaagaaga | |
| emphasized in | aatctcaatgctaggggaaataacccaccttcagaccatctcggaagatctaaaaagcctg | |
| bold font) | accatggaccctcacaaactgccctcctcaagtgaacaggtgattctggacttgaaaggttct | |
| | gattacagctggtcgtatcagacgccaccctcttcccccagcaccaccatgtccagaaagtccag | |
| | tgtctgcagcagcctgaacagtgtcaacagcagtgactcccggtccagcggctcccactcg | |
| | cattcccccagctcacattaccgctaccgcagctccaacctggcccagcaggctcctgtga | |
| | ggctgtccagcgtgtcctcccatgactcaggattcatatcccaggatgccttccagtccaag | |
| | tcaccatcccccatgccgccagaggcccccaaccagttgtctaacgggttttctcactatagttt | |
| | atcaagtgagtcccacgtggggcccacgggtgcaggccttttccctcattgcctgcctgcctccc | |
| | gcctgctccctcgggtcacctctgtccaccttccagactacgctcattattacaccattgggcccg | |
| | gcatgttcccgtcatctcagatccctagctggaaggactgggctaagcctgggccctatgacc | |
| | agcctctggtgaacaccctgcagcgccgcaaagagaagcgagaaccggaccccaacgg | |
| | gggaggacccactaccgccagcggcccacctgcagcagctgaggaggctcagagaccac | |
| | ggagcatgactgtatcggctgccaccaggcctggtgaggagatggaggcttgtgaggagctg | |
| | gccctggccctgtctcggggcctgcagctggacacccagaggagcagccgggactcgcttca | |
| | gtgctccagcggctacagcacccagacaaccaccccctgctgctctgaggacaccatcccttcc | |
| | caagtttcagattatgattatttctctgtaagtggtgaccaggaggcagatcagcaggagtt | |
| | cgacaagtcctccaccattccaagaaacagcgacatcagccagtcctaccgacggatgttc | |
| | caagccaagcgtccagcctcaactgctggcctccccaccaccctgggacctgctatggtca | |
| | ctccaggggttgcaactatccgacggaccccttccaccaagccttctgtccgccggggaac | |
| | cattggagctggtcccatccccatcaagacacccgtgatccctgtcaagaccccaaccgtc | |
| | ccagacctcccaggggtgttgccagcccctccagatgggccagaagagcggggggagca | |
| | cagccctgagtcgccatctgtgggtgagggcccccaaggtgtcaccagcatgccctcctca | |
| | atgtggagcggccaagcttccgttaaccctccacttccaggcccgaagcccagtatccctg | |
| | aggagcacagacaggcaattccagaaagtgaagctgaagaccaggaacgggaaccccc | |
| | aagtgccactgtctccccaggccagattccagagagtgaccctgcagacctgagcccaag | |
| | ggatactccacaaggagaagacatgctgaacgccatccgaaggggcgtgaaactgaag | |
| | aagaccacgacaaacgatcgctcagcccctcgcttttcttag | |
| MTSS1 first | atggaggctgtgattgagaaggaatgcagcgcgctcggaggcctcttccagaccatcatcagcg | 407 |
| exon DNA | acatgaag | |
| sequence | | |
| hg 38 | tgcagtgagccgagatcgcaccactgaactccagcctggcaacagagctagactctgtctcaaa | 408 |
| chr8: | aaaataaagaaataaataaaaataaaaATACAGATGGTTCTCATTTTTCAC | |
| 124,845,017- | AGTAGCTATGttttttttttaattatactttaagttctatggtacatgtgcacaacatgcaggtt | |
| 124,845,217 | tgttacatatgtatacatgtg | |
| sequence (with | | |
| rs1241595 site | | |
| ″G″ in capital | | |
| letter, bold | | |
| and underlined; | | |
| HAND2 | | |
| binding site in | | |
| capital letter | | |
| and bold, | | |
| MEF2A/B/C | | |
| binding site is | | |
| underlined, and | | |
| TWIST1 | | |
| binding site is | | |
| in italics; | | |
| repeat | | |
| regions are | | |
| indicated in | | |
| lower case and | | |
| non-repeat | | |
| regions in | | |
| capital letters) | | |
| HAND2 | tacagatggt | 409 |
| binding site | | |
| sequence | | |
| MEF2A/B/C | aaaataaaaatacag | 410 |
| binding site | | |
| sequence | | |
| TWIST1 | aatacagatggtt | 411 |
| binding site | | |
| sequence | | |
| MTSS1 mRNA | agagcgagcgccggggccgggcgcgcaggagugaaaaggaggcggcggccgcagcugc | 92 |
| sequence | gagcaacagauccggacgccgcgagcugacccgcucugcuguugggcgauuuuuuuuu | |
| | aauugcagaaaaauuuauuaaauuggaaaaucuugcguuuuucaauggcgcuggcccc | |
| | gggucagcgggcgauuuucucugcaucaagaugggcuuugccguuuccguagugggc | |
| | accagugguggccugauugucagucuucucccggcauuuuuaaggccaggagccgagc | |
| | gcugcuuguaggcgaauacccuacagagcgguuuggcuuuuuaaauuacuguuauua | |
| | uuuugggcagagaacagucggucuggugcaccccguccucgcugcagaagaggcugcg | |
| | aguccgaggugggucucucggaaggugaaauuccuucuggggugagcgagccccggc | |
| | cccgcgcgcaguccagcggccccgcgugugugcccucgcccugccggagccgggaaaa | |
| | uggaggcugugauugagaaggaaugcagcgcgcucggaggccucuuccagaccaucau | |
| | cagcgacaugaaggggagcuauccaguuugggaagauuucauaaacaaagcaggaaag | |
| | cugcagucccagcuucggacaacaguaguagcagcagcugccuucuuggacgccuuuc | |
| | agaaaguggcugacauggccaccaacacacguggugggaccagggagauuggaucugc | |
| | ucucaccaggaugugcaugaggcacagaagcauugaagccaagcugaggcaguuuucg | |
| | agcgcuuuaauugauugucugauaaacccacuucaagaacagauggaagaauggaaga | |
| | aaguggccaaccagcuggauaaagaccacgcaaaagaauauaagaaagcccgccaagag | |
| | auaaaaaagaaguccucggauacgcugaaacugcagaagaaagcaaaaaaagggagagg | |
| | ugauauccagccucaguuggacagugcucuccaagaugucaaugauaaguaucucuua | |
| | uuggaagaaacagaaaagcaggcuguccggaaggcuuugauugaagaacguggccgau | |
| | ucuguaccuucaucucuaugcugcggccagugauugaagaagaaaucucaaugcuagg | |
| | ggaaauaacccaccuucagaccaucucggaagaucuaaaaagccugaccauggacccuc | |
| | acaaacugcccuccucaagugaacaggugauucuggacuugaaagguucugauuacag | |
| | cuggucguaucagacgccacccucuucccccagcaccaccauguccagaaaguccagug | |
| | ucugcagcagccugaacagugucaacagcagugacucccgguccagcggcucccacucg | |
| | cauucccccagcucacauuaccgcuaccgcagcuccaaccuggcccagcaggcuccugu | |
| | gaggcuguccagcguguccucccaugacucaggauucauaucccaggaugccuuccag | |
| | uccaagucaccaucccccaugccgccagaggcccccaaccaguugucaacggguuuuc | |
| | ucacuauaguuuaucaagugagucccacguggggcccacgggugcaggccuuuucccu | |
| | cauugccugccugccucccgccugcucccucgggucaccucuguccaccuuccagacua | |
| | cgcucauuauuacaccauugggcccggcauguucccgucaucucagaucccuagcugg | |
| | aaggacugggcuaagccugggcccuaugaccagccucuggugaacacccugcagcgcc | |
| | gcaaagagaagcgagaaccggaccccaacgggggaggacccacuaccgccagcggccca | |
| | ccugcagcagcugaggaggcucagagaccacggagcaugacuguaucggcugccacca | |
| | ggccuggugaggagauggaggcuugugaggagcuggcccuggcccugucucggggcc | |
| | ugcagcuggacacccagaggagcagccgggacucgcuucagugcuccagcggcuacag | |
| | cacccagacaaccacccccugcugcucugaggacaccaucccuucccaaguuucagauu | |
| | augauuauuucucuguaaguggugaccaggaggcagaucagcaggaguucgacaaguc | |
| | cuccaccauuccaagaaacagcgacaucagccaguccuaccgacggauguuccaagcca | |
| | agcguccagccucaacugcuggccuccccaccacccugggaccugcuauggucacucca | |
| | gggguugcaacuauccgacggaccccuuccaccaagccuucuguccgccggggaacca | |
| | uuggagcuggucccauccccaucaagacacccgugaucccugucaagaccccaaccguc | |
| | ccagaccucccagggguguugccagccccuccagaugggccagaagagcggggggagc | |
| | acagcccugagucgccaucugugggugagggcccccaaggugucaccagcaugcccuc | |
| | cucaauguggagcggccaagcuuccguuaacccuccacuuccaggcccgaagcccagua | |
| | ucccugaggagcacagacaggcaauuccagaaagugaagcugaagaccaggaacgggaa | |
| | cccccaagugccacugucuccccaggccagauuccagagagugacccugcagaccugag | |
| | cccaagggauacuccacaaggagaagacaugcugaacgccauccgaaggggcgugaaac | |
| | ugaagaagaccacgacaaacgaucgcucagccccucgcuuuucuuagguucacaagaaa | |
| | ugcgccgguggggaaugaacuguuucauuaauaaaaccuaauuugucuugauccauuc | |
| | cacucuauaauaaaacaaaagauuuuguaggcaacucggaauauagcucuuuugaaag | |
| | uacucgacaccuuuagauaagaauuaaaaccaaccuauguaacugacauaaucuugauc | |
| | uuugauuuuucugaaggugccaaauuccauuuaacuuuuuuacaagucuuuguaaaa | |
| | uuuuaaaugcauaaaggggguuggggcaggggaaccacgaaguaguuaauuuuagaaa | |
| | aggauuuacuauacuucacucuucuuuuuuuuuccccacaagcuuuuguagaugcauu | |
| | guaguagucuagcuuagaagcaaaugcaaguuauuuuaauguacaaacuaaaugggua | |
| | agagguaaaaucuucauuuaaauauacuauguucuggaugaaaagagcaggaguaaca | |
| | auugaugagcaauauucagagugaaguaaaucuggaaaugguagacuguguugggau | |
| | uggggggagggccaugggagggguacaucgucaacauagccgauccuguuacauuuaa | |
| | gaguagccucguagguugaauuucuucugguagcuucaugguaaaugcauccgaauaa | |
| | gccauacuggauugcaguguuuguuucuguaggguguuuaaggacuugacuuccuuu | |
| | cucccaugauuccucuggacugcacacagcacccacaaccagccccaugcaugcugcug | |
| | ccucugggcagucguagaaucucccaacuucaguuucucguugaugguacucaccuuua | |
| | uggaauccaaauacauccaaaaggguaaggcaguuuuaaaaaugugaaaacauuuaaaa | |
| | augauaauagcagggaauucuuagauuauaguaaaugccuuuuacuuaacugugccca | |
| | gcaggcugggugcguuaaaaagcccaaguauuuugaaaaaacucgaacagauuugaca | |
| | aggguagccagcuuggagucuagcaacuugccaauguguuuaccaaucugggggcuu | |
| | guuuuucuuuucuucuuucaaauaaauggcaguuaacuggcuuuacaguaaacauuga | |
| | agagaggaggauuuguuuauugucacugggaaucugaccacuauacuguccuuuuuu | |
| | uguauucuggguaaauguuuuuuggaaaagauuugucuuuucuaaguggaaguuaaa | |
| | uuuguuauacugcccauccccuaaagccaacagagauuuguagauuuaaagggaucac | |
| | auuugaagacaauaguguuuaagaaagcaagcaagucccuuagcagucaggucauaac | |
| | agggcacauuucugaccgaacccucucaaggcagaggaggaguuugguggguuucaua | |
| | cacccugcagauuccuguuggcucuaacccucaauuaccuaaucunaugcuuuaacaca | |
| | uaacugcauuggaugugagaguaacguaccguauggucauuguucuauauauuaacau | |
| | ugaacacugcugcgauugcucaaggacauuuuauguuacggcuuuaaagcaaaggcau | |
| | gauuauuagaaacuauuuaagcuuuuuuuuugaaaaacaagcuccuuuuacagaaua | |
| | uaaacaacaguagugccugugguuuagcccaccaaucuugaugacuaaaaguagcuga | |
| | ugcauugugcauaugaugcuugagaugguuuuugcaaaagcagaaaucgcugcaagg | |
| | uaaucacaauagauaaaagugguauuuuaaaccuuugaaaaaauggauguaacugua | |
| | ccuugguacagcuuuucacuuguuuaguuuuuaaacguuaguauaaucugaaaaaa | |
| | aaauguugccaaauucaauguagaaagaaugugacaacacaccuuggguaguucugcu | |
| | uguguuuuugcauauuguaaaagcagugucacagcuaaaaagaaagaaaucguuucua | |
| | acaguaaauuauugugcuuuaguugcuaguuuguacugagaguugaccucucccugu | |
| | gcaguuuuuuguucuaaacuuguauaaauaacaauuguguaaugugucucccuccuac | |
| | auuguaacaauugcuucagccuacguuauaaauaaagaaccacuagauuaaaaaa | |
| MTSS1 protein | MEAVIEKECSALGGLFQTIISDMKGSYPVWEDFINKAGKLQS | 93 |
| sequence | QLRTTVVAAAAFLDAFQKVADMATNTRGGTREIGSALTRM | |
| | CMRHRSIEAKLRQFSSALIDCLINPLQEQMEEWKKVANQLD | |
| | KDHAKEYKKARQEIKKKSSDTLKLOKKAKKGRGDIQPQLDS | |
| | ALQDVNDKYLLLEETEKQAVRKALIEERGRFCTFISMLRPVIE | |
| | EEISMLGEITHLQTISEDLKSLTMDPHKLPSSSEQVILDLKGSD | |
| | YSWSYQTPPSSPSTTMSRKSSVCSSLNSVNSSDSRSSGSHSHS | |
| | PSSHYRYRSSNLAQQAPVRLSSVSSHDSGFISQDAFQSKSPSP | |
| | MPPEAPNQLSNGFSHYSLSSESHVGPTGAGLFPHCLPASRLLP | |
| | RVTSVHLPDYAHYYTIGPGMFPSSQIPSWKDWAKPGPYDQP | |
| | LVNTLQRRKEKREPDPNGGGPTTASGPPAAAEEAQRPRSMT | |
| | VSAATRPGEEMEACEELALALSRGLQLDTQRSSRDSLQCSSG | |
| | YSTQTTTPCCSEDTIPSQVSDYDYFSVSGDQEADQQEFDKSST | |
| | IPRNSDISQSYRRMFQAKRPASTAGLPTTLGPAMVTPGVATIR | |
| | RTPSTKPSVRRGTIGAGPIPIKTPVIPVKTPTVPDLPGVLPAPPD | |
| | GPEERGEHSPESPSVGEGPQGVTSMPSSMWSGQASVNPPLPG | |
| | PKPSIPEEHRQAIPESEAEDQEREPPSATVSPGQIPESDPADLSP | |
| | RDTPQGEDMLNAIRRGVKLKKTTTNDRSAPRFS | |
| MTSS1 siRNA | ccaucucggaagaucuaaa | 94 |
| (ThermoFisher) | | |
| MTSS1 siRNA | ccagcuucggacaacagua | 95 |
| (ThermoFisher) | | |
| MTSS1 siRNA | ccaucaucagcgacaugaa | 96 |
| (ThermoFisher) | | |
| MTSS1 siRNA | ccgtatggtcattgttctata | 97 |
| (Qiagen) | | |
| MTSS1 siRNA | ccgacggatgttccaagccaa | 98 |
| (Qiagen) | | |
| MTSS1 siRNA | tcccgtcatctcagatcccta | 99 |
| (Qiagen) | | |
| MTSS1 siRNA | ccgtatggtcattgttctata | 100 |
| (Qiagen) | | |

### Polynucleotides and Expression Cassettes

In some embodiments, the present disclosure provides polynucleotide sequences, expression cassettes, and/or vectors for the treatment and/or prevention of heart disease (e.g., cardiomyopathy). In some embodiments, a polynucleotide sequence, expression cassette, and/or vector comprises a promoter (e.g., a cardiac-specific promoter or a U6 promoter, as appropriate) operatively linked to a cardioprotective gene (e.g., encoding an MMPI 1 protein, a SYNPO2L protein) or an MTSS1 inhibitor (e.g., an inhibitory RNA or a gene-editing system inhibiting the expression of MTSS1). In some embodiments, a polynucleotide sequence, expression cassette, and/or vector comprises one, two, or more promoters (e.g., a cardiac-specific promoter or a U6 promoter, as appropriate) operatively linked to one, two, or more cardioprotective genes (e.g., encoding an MMP11 protein, a SYNPO2L protein) or an MTSS1 inhibitor (e.g., an inhibitory RNA or a gene-editing system inhibiting the expression of MTSS1).

The polynucleotides, expression cassettes, and/or vectors contemplated herein may be combined with other sequences, such as promoters and/or enhancers, untranslated regions (UTRs), signal sequences, Kozak sequences, polyadenylation signals, additional restriction enzyme sites, multiple cloning sites, internal ribosomal entry sites (IRES), recombinase recognition sites (e.g., LoxP, FRT, and Att sites), termination codons, transcriptional termination signals, and polynucleotides encoding self-cleaving polypeptides, epitope tags, as disclosed elsewhere herein or as known in the art. In some embodiments, the polynucleotides, expression cassettes, and/or vectors described herein may also contain a ribosome binding site for translation initiation, a transcription terminator, and/or polynucleotide sequences for amplifying expression.

In some embodiments, the polynucleotide sequence comprises a promoter. In some embodiments, the polynucleotide sequence comprises a promoter operatively linked to a cardioprotective gene, inhibitory RNA, Cas endonuclease, or guide RNA. In some embodiments, the promoter is a promoter suitable for expression of proteins. In some embodiments, the promoter is a cardiac-specific promoter. In some embodiments, the promoter is suitable for expression of RNA.

In some embodiments, the present disclosure provides polynucleotide sequences, expression cassettes, and/or vectors comprising one or more cardiac-specific promoters operably linked to a gene encoding MMP11 protein (or a variant, mutant or fragment thereof), a gene encoding a SYNPO2L protein (e.g., SYNPO2LA, SYNPO2LB, or a variant, mutant or fragment of any thereof), and/or a gene encoding a Cas endonuclease, and/or an RNA-specific promoter (e.g., a U6 promoter) operably linked to an inhibitory RNA inhibiting the expression of MTSS1 protein or a guide RNA targeting MTSS1 gene.

As a result of the degeneracy of the genetic code, there are many nucleotide sequences that encode a polypeptide, or fragment of variant thereof, as described herein. Some of these polynucleotides bear minimal homology to the nucleotide sequence of any native gene. Nonetheless, polynucleotides that vary due to differences in codon usage are specifically contemplated in particular embodiments, for example polynucleotides that are optimized for human and/or primate codon selection. Further, alleles of the genes comprising the polynucleotide sequences provided herein may also be used. Alleles are endogenous genes that are altered as a result of one or more mutations, such as deletions, additions and/or substitutions of nucleotides.

Polynucleotides can be prepared, manipulated and/or expressed using any of a variety of well-established techniques known and available in the art.

The disclosure provides an expression cassette comprising a transgene encoding a cardioprotective gene product (e.g., MMP11, SYNPO2LA, and/or SYNPO2LB polypeptide, or functional variant thereof), an inhibitory RNA (e.g., siRNA or shRNA inhibiting expression of MTSS1), a Cas endonuclease, and/or a guide RNA (e.g., a Cas endonuclease and guide RNA system for disrupting the expression of MTSS1).

The transgene polynucleotide sequence in an expression cassette can be, for example, an open reading frame encoding a protein. The expression cassette may comprise, optionally, a promoter operatively linked to the transgene, optionally an intron region, optionally a polyadenylation (poly(A)) signal, optionally a woodchuck hepatitis virus post-transcriptional element (WPRE), and optionally a transcription termination signal.

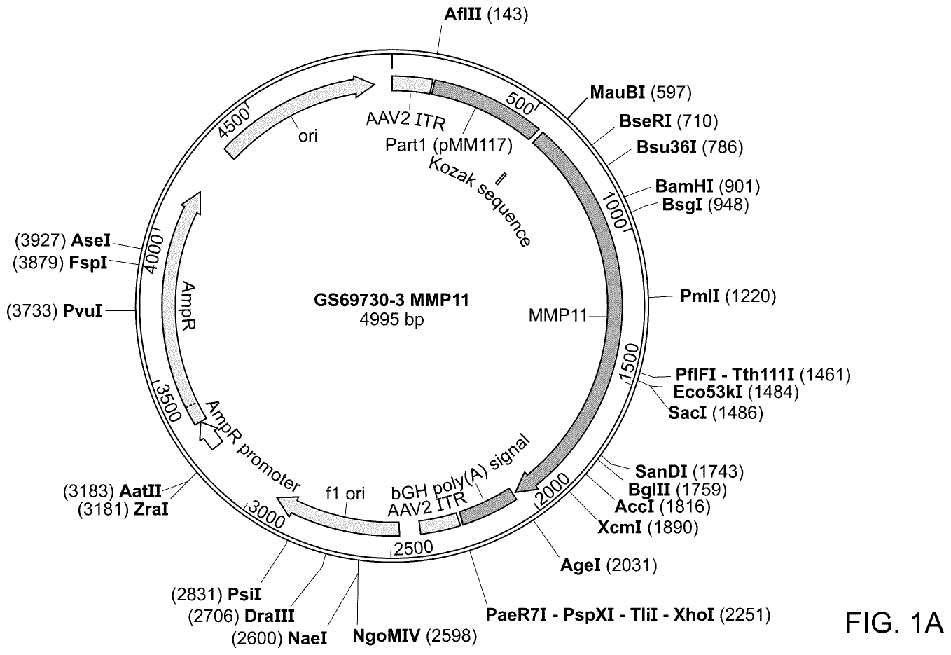

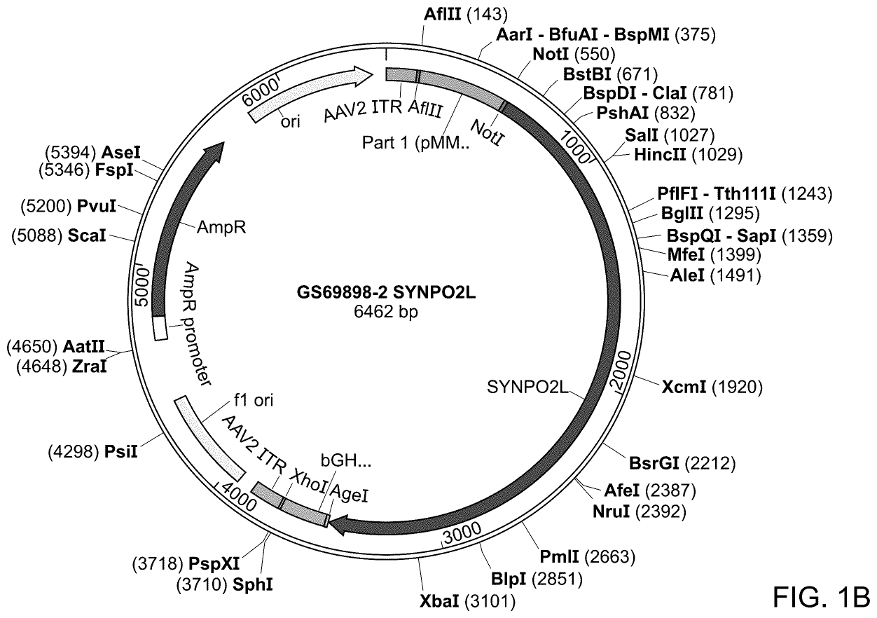

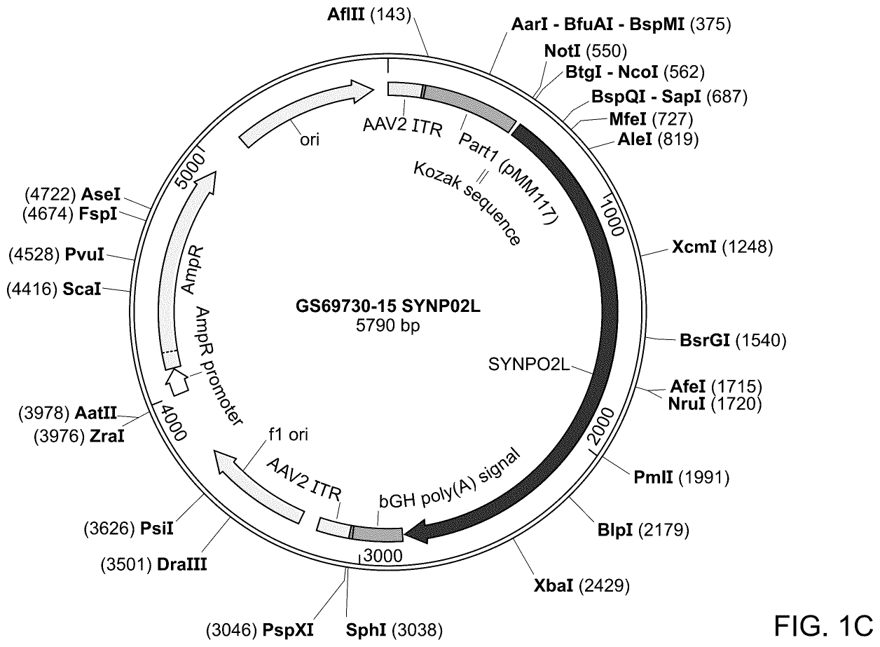

The expression cassette may be flanked by one or more inverted terminal repeats (ITRs). An expression cassette flanked by one or more ITRs is herein referred to as a “viral genome.” The ITRs in an expression cassette serve as markers used for viral packaging of the expression cassette (Clark et al. Hum Gene Ther. 6:1329-41 (1995)). Illustrative and non-limiting embodiments of viral genomes of the disclosure are shown in FIGS. 1A-1C. The expression cassette can be integrated into the host cell genome by, for example, infecting the host cell with an rAAV virion comprising capsid protein and a viral genome comprising an expression cassette, thereby expressing the transgene within a host cell.

The regulatory elements described herein can be applied to the polynucleotides, expression cassettes, and/or vectors for expressing a gene-editing system (e.g., a CRISPR/Cas system targeting the MTSS1 gene), for example, in a single vector system as described; or be applied to the polynucleotides, expression cassettes, and/or vectors expressing a cardioprotective gene product (e.g., MMP11, SYNPO2LA, and/or SYNPO2LB polypeptide, or functional variant thereof) and/or an inhibitory RNA (e.g., siRNA or shRNA inhibiting expression of MTSS1) as described.

### Illustrative Expression Cassettes

The disclosure provides expression cassettes comprising a polynucleotide comprising a 5′ to 3′ arrangement (sometimes referred to as an orientation) of elements. In some embodiments, the elements comprise one or more promoters; optionally one or more enhancers; optionally one or more introns; one or more transgenes; optionally one or more WPRE sequences; and optionally one or more polyadenylation sequences (p(A)). In some embodiments, the 5′ to 3′ arrangement of elements is selected from:
5′-promoter-transgene-WPRE-p(A)-3′;
5′-promoter-intron-transgene-WPRE-p(A)-3′;
5′-promoter-transgene-WPRE-p(A)-promoter-transgene-WPRE-p(A);
5′-enhancer-promoter-transgene-WPRE-p(A)-3′;
5′-enhancer-promoter-intron-transgene-WPRE-p(A)-3′;
5′-enhancer-enhancer-promoter-transgene-WPRE-p(A)-3′;
5′-enhancer-enhancer-promoter-intron-transgene-WPRE-p(A)-3′;
5′-enhancer-promoter-intron-transgene-WPRE-p(A)-p(A)-transgene-intron-promoter-enhancer-3′;
5′-enhancer-promoter-intron-transgene-WPRE-p(A)-enhancer-promoter-intron-transgene-p(A)-3′;
5′-p(A)-WPRE-transgene-intron-promoter-enhancer-enhancer-promoter-intron-transgene-p(A)-3′;
5′-promoter-intron-transgene-WPRE-p(A)-p(A)-transgene-intron-promoter-3′;
5′-promoter-intron-transgene-WPRE-p(A)-promoter-intron-transgene-p(A)-3′;
5′-p(A)-WPRE-transgene-intron-promoter-promoter-intron-transgene-p(A)-3′; and
5′-RNA-specific promoter-RNAi-3′.
5′-promoter-Cas protein-p(A)-RNA-specific promoter-gRNA-3′.

In some embodiments of the expression cassettes provided herein, the WPRE element is replaced by any other post-transcriptional regulatory element known in the art. In some embodiments, the expression cassettes provided herein comprise any post-transcriptional regulatory element known in the art. In some embodiments, the expression cassettes provided herein do not comprise a post-transcriptional regulatory element (e.g., do not comprise the WPRE element). In some embodiments, the expression cassettes provided herein comprise WPRE.

In the expression cassettes described herein (such as those listed above), the orientation of the promoter, enhancer, transgene and poly(A) elements can be forward or reverse (e.g., in cases where there are more than one promoter, one promoter, optionally enhancer, and operably linked transgene can be oriented in a forward direction, and another promoter, optionally enhancer, and operably linked transgene can be oriented in a reverse direction).

### Regulatory Elements

As used herein, the term “regulatory element” refers those non-translated regions of the vector (e.g., origin of replication, selection cassettes, promoters, enhancers, translation initiation signals (Shine Dalgarno sequence or Kozak sequence) introns, a polyadenylation sequence, 5′ and 3′ untranslated regions) which interact with host cellular proteins to carry out transcription and translation. Such elements may vary in their strength and specificity. The transcriptional regulatory element may be functional in either a eukaryotic cell (e.g., a mammalian cell) or a prokaryotic cell (e.g., bacterial or archaeal cell). In some embodiments, a polynucleotide sequence encoding a cardioprotective gene, gene product, Cas endonuclease, inhibitory RNA or guide RNA described herein is operably linked to multiple control elements that allow expression of the polynucleotide in both prokaryotic and eukaryotic cells.

As used herein, the term “transcription start site” or “TSS” refers to the first base pair transcribed by an RNA polymerase when the RNA polymerase initiates transcription. A TSS is different from the start codon (canonically, ATG), which must be downstream of the TSS in the transcribed region of the polynucleotide. The location of a transcription start site can be determined experimentally or by prediction using any of various prediction algorithms. Annotated TSSs are available from the Eukaryotic Promoter Database and the UCSC Genome Browser. Multiple TSSs for TNNT2 are identified in the UCSC Genome Browser.

As used herein, the TSS for TNNT2 is defined to be the sequence identified by the C at the 5′ end of the motif identified by dbTSS: CTCCATC.

### Promoters

The term “promoter” as used herein refers to a DNA sequence that directs the binding of RNA polymerase and thereby promotes RNA synthesis. Promoters and corresponding protein or polypeptide expression may be ubiquitous, meaning strongly active in a wide range of cells, tissues and species or cell-type specific, tissue-specific, or species specific. Examples of ubiquitous promoters include the CAG promoter and CMV promoter (Yue et al. BioTechniques 33:672-678 (2002)). Promoters may be “constitutive,” meaning continually active, or “inducible,” meaning the promoter can be activated or deactivated by the presence or absence of biotic or abiotic factors. Also included in the nucleic acid constructs or vectors of the invention are enhancer sequences that may or may not be contiguous with the promoter sequence. Enhancer sequences influence promoter-dependent gene expression and may be located in the 5′ or 3′ regions of the native gene.

In some embodiments, the expression cassette comprises a single promoter. In some embodiments, the expression cassette comprises at least one promoter. In some embodiments, the expression cassette comprises two promoters. In some embodiments, the expression cassette comprises a ubiquitous promoter. In some embodiments, the expression cassette comprises an inducible promoter. In some embodiments, the expression cassette comprises a cell-type specific promoter. In some embodiments, the promoter specifically promotes expression of the polynucleotide encoding a polypeptide, or functional variant thereof, in a cardiac cell (e.g., a cardiomyocyte).

In some embodiments, the polynucleotides, expression cassettes and vectors described herein comprise a CMV promoter. In some embodiments, the polynucleotides, expression cassettes and vectors described herein comprise a CAG promoter. In some embodiments, the polynucleotides, expression cassettes and vectors described herein comprise a SV40 promoter.

In some embodiments, the polynucleotides, expression cassettes and vectors described herein comprise a muscle cell-specific promoter. In some embodiments, the polynucleotides, expression cassettes and vectors described herein comprise a cardiac cell-specific promoter.

In some embodiments, the polynucleotides, expression cassettes and vectors described herein comprise a cardiomyocyte-specific promoter. In some embodiment, the polynucleotides, expression cassettes and vectors described herein designed to express a Cas endonuclease, or one or more cardioprotective gene products, such as MMP11, SYNPO2LA or SYNPO2LB, comprise a cardiomyocyte-specific promoter.

A “cardiomyocyte-specific promoter”, as used herein, specifies a promoter whose activity in cardiomyocytes is at least 2-fold higher than in any other non-cardiac cell type or cardiac cell which is not a cardiomyocyte. Preferably, a cardiomyocyte-specific promoter suitable for being used in the vector of the present disclosure has an activity in cardiomyocytes which is at least 5-fold, at least 10-fold, at least 15-fold, at least 20-fold, at least 25-fold, or at least 50-fold higher compared to its activity in a non-cardiac cell type or a cardiac cell type which is not a cardiomyocyte.

In some embodiments, the cardiac-specific or cardiomyocyte-specific promoter is a human promoter. Examples of cardiac-specific or cardiomyocyte-specific promoter include, but are not limited to, the alpha myosin heavy chain promoter, the myosin light chain 2v promoter, the alpha myosin heavy chain promoter, the alpha-cardiac actin promoter, the alpha-tropomyosin promoter, the cardiac troponin C promoter, the cardiac troponin I promoter, the cardiac myosin-binding protein C promoter, and the sarco/endoplasmic reticulum Ca²⁺ ATPase (SERCA) promoter (e.g. isoform 2 of SERCA2).

In some embodiments, the cardiac-specific promoter is the cardiac TNNT2 promoter. In some embodiments, the cardiac TNNT2 promoter is modified, e.g., by the deletion, insertion, or substitution of polynucleotides. Illustrative polynucleotide sequences of the cardiac TNNT2 promoter are shown in Table 3A below. The transcription start site (TSS) of the TNNT2 promoters are bolded and underlined.

TABLE 3A
Exemplary TNNT2 promoter sequences

| | | SEQ |
|---|---|---|
| | | ID |
| Name | Sequence | NO: |
| TNNT2p- | gtcatggagaagacccaccttgcagatgtcctcac | 1 |
| 600 | tggggctggcagagccggcaacctgcctaaggctg | |
| | ctcagtccattaggagccagtagcctggaagatgt | |
| | ctttacccccagcatcagttcaagtggagcagcac | |
| | ataactcttgccctctgccttccaagattctggtg | |
| | ctgagacttatggagtgtcttggaggttgccttct | |
| | gccccccaaccctgctcccagctggccctcccagg | |
| | cctgggttgctggcctctgctttatcaggattctc | |
| | aagagggacagctggtttatgttgcatgactgttc | |
| | cctgcatatctgctctggttttaaatagcttatct | |
| | gagcagctggaggaccacatgggcttatatggcgt | |
| | ggggtacatgttcctgtagccttgtccctggcacc | |
| | tgccaaaatagcagccaacaccccccacccccacc | |
| | gccatccccctgccccacccgtcccctgtcgcaca | |
| | ttcctccctccgcagggctggctcaccaggcccca | |
| | gcccacatgcctgcttaaagccctctccatcctct | |
| | gcctcacccagtccccgctgagactgagcagacgc | |
| | ctcca | |
| TNNT2p- | gatgtctttacccccagcatcagttcaagtggagc | 2 |
| 500 | agcacataactcttgccctctgccttccaagattc | |
| | tggtgctgagacttatggagtgtcttggaggttgc | |
| | cttctgccccccaaccctgctcccagctggccctc | |
| | ccaggcctgggttgctggcctctgctttatcagga | |
| | ttctcaagagggacagctggtttatgttgcatgac | |
| | tgttccctgcatatctgctctggttttaaatagct | |
| | tatctgagcagctggaggaccacatgggcttatat | |
| | ggcgtggggtacatgttcctgtagccttgtccctg | |
| | gcacctgccaaaatagcagccaacaccccccaccc | |
| | ccaccgccatccccctgccccacccgtcccctgtc | |
| | gcacattcctccctccgcagggctggctcaccagg | |
| | ccccagcccacatgcctgcttaaagccctctccat | |
| | cctctgcctcacccagtccccgctgagactgagca | |
| | gacgcctcca | |
| TNNT2p- | gttgccttctgccccccaaccctgctcccagctgg | 3 |
| 400 | ccctcccaggcctgggttgctggcctctgctttat | |
| | caggattctcaagagggacagctggtttatgttgc | |
| | atgactgttccctgcatatctgctctggttttaaa | |
| | tagcttatctgagcagctggaggaccacatgggct | |
| | tatatggcgtggggtacatgttcctgtagccttgt | |
| | ccctggcacctgccaaaatagcagccaacaccccc | |
| | cacccccaccgccatccccctgccccacccgtccc | |
| | ctgtcgcacattcctccctccgcagggctggctca | |
| | ccaggccccagcccacatgcctgcttaaagccctc | |
| | tccatcctctgcctcacccagtccccgctgagact | |
| | gagcagacgcctcca | |
| TNNT2p- | gttgcatgactgttccctgcatatctgctctggtt | 4 |
| 300 | ttaaatagcttatctgagcagctggaggaccacat | |
| | gggcttatatggcgtggggtacatgttcctgtagc | |
| | cttgtccctggcacctgccaaaatagcagccaaca | |
| | ccccccacccccaccgccatccccctgccccaccc | |
| | gtcccctgtcgcacattcctccctccgcagggctg | |
| | gctcaccaggccccagcccacatgcctgcttaaag | |
| | ccctctccatcctctgcctcacccagtccccgctg | |
| | agactgagcagacgcctcca | |

In some aspects, the present disclosure provides a cardiac troponin T promoter, comprising a polynucleotide having between 300 bp and 500 bp. In some embodiments, the polynucleotide comprises a sequence that shares at least 80%, at least 90%, or at least 100% identity to any one of SEQ ID NOs: 1-85. In some embodiments, the polynucleotide comprises a sequence that shares at least 80%, at least 90%, or at least 100% identity to SEQ ID NO: 1. In some embodiments, the polynucleotide comprises a sequence that shares at least 80%, at least 90%, or at least 100% identity to SEQ ID NO: 3. In some embodiments, the polynucleotide shares at least 80%, 90%, 95%, 96%, 97%, 98%, 99%, or and 100% sequence identity with a genomic polynucleotide sequence upstream of and including the transcription start site of a troponin T gene. In some embodiments, the polynucleotide shares at least 80%, 90%, 95%, 96%, 97%, 98%, 99%, or and 100% sequence identity with a genomic polynucleotide sequence −450 bp to +1 bp relative to the transcription start site of a troponin T gene. In some embodiments, the polynucleotide shares at least 80%, 90%, 95%, 96%, 97%, 98%, 99%, or and 100% sequence identity with a genomic polynucleotide sequence −350 bp to +1 bp relative to the transcription start site of a troponin T gene. In some embodiments, the polynucleotide shares at least 80%, 90%, 95%, 96%, 97%, 98%, 99%, or and 100% sequence identity with a genomic polynucleotide sequence −250 bp to +1 bp relative to the transcription start site of a troponin T gene. In some embodiments, the polynucleotide shares at least 80%, 90%, 95%, 96%, 97%, 98%, 99%, or and 100% sequence identity with a genomic polynucleotide sequence −450 bp to +50 bp relative to the transcription start site of a troponin T gene. In some embodiments, the polynucleotide shares at least 80%, 90%, 95%, 96%, 97%, 98%, 99%, or and 100% sequence identity with a genomic polynucleotide sequence −350 bp to +50 bp relative to the transcription start site of a troponin T gene. In some embodiments, the polynucleotide shares at least 80%, 90%, 95%, 96%, 97%, 98%, 99%, or and 100% sequence identity with a genomic polynucleotide sequence −250 bp to +50 bp relative to the transcription start site of a troponin T gene. In some embodiments, the troponin T gene is a human troponin T gene.

In some embodiments, the promoter is a muscle-specific promoter. In some embodiments, the promoter is a cardiac cell-specific promoter. In some embodiments, the promoter is a cardiomyocyte-specific promoter. In some embodiments, the promoter has the same cell-type specificity as a native troponin T promoter of about 600 bp. In some embodiments, the promoter described herein has the same cell-type specificity as a reference promoter comprising SEQ ID NO: 1. In some embodiments, the promoter expresses a gene product operatively linked thereto at least about 10%, at least about 20%, at least about 30% more than a native troponin T promoter. In some embodiments, the promoter described herein expresses a gene product operatively linked thereto at least about 10%, at least about 20%, at least about 30% more than a reference promoter comprising SEQ ID NO: 1.

The term “modified cardiac TNNT2 promoter” as used herein refers to a promoter that comprises a polynucleotide sequence of at least 200 base pairs that comprises one or more continuous or discontinuous polynucleotide segments each sharing 70%, 80%, 90%, 95%, 96%, 97%, 98%, 99%, or 100% identity to a corresponding segment of the TNNT2p-600 segment provided in Table 3A as SEQ ID NO: 1. As it is a “promoter,” a modified cardiac TNNT2 promoter must be capable of promoting initiation of transcription by an RNA polymerase in a host or target cell at or near a TSS within the promoter (i.e. at or near the TTS of TNNT2 as defined herein) or, if the endogenous TSS of TNNT2 is not present in the modified cardiac TNNT2 promoter then at a heterologous TSS at most 100 base pairs downstream (3′ on the sense strand) to the downstream (3′) end of the modified cardiac TNNT2 promoter. Similarly stated, a modified cardiac TNNT2 promoter may comprise only sequences upstream of the TSS of TNNT2 or more comprise the TSS of TNNT2.

In some embodiments, the cardiac TNNT2 promoter is modified to comprise a polynucleotide sequence of between about 200 and 500 base pairs, between about 250 and 500 base pairs, between about 300 to 500 base pairs, between about 350 to 500 base pairs, between about 400 to 500 base pairs, between about 450 to 500 base pairs, between about 200 and 450 base pairs, between about 200 and 400 base pairs, between about 200 and 350 base pairs, between about 200 and 300 base pairs, and between about 200 and 250 base pairs in length. In some embodiments, the modified cardiac TNNT2 promoter comprises a polynucleotide sequence of between about 350 base pairs to about 450 base pairs, between about 375 base pairs to about 425 base pairs, between about 375 base pairs to about 400 base pairs, between about 375 base pairs to about 425 base pairs, between about 400 base pairs to about 425 base pairs, or between about 400 base pairs to about 450 base pairs. In some embodiments, the cardiac TNNT2 promoter comprises a polynucleotide sequence of about 400 base pairs.

In a particular embodiment, the modified cardiac troponin T promoter comprises between 300 bp and 500 bp of SEQ ID NO: 1. For instance, the modified cardiac troponin T promoter may comprise SEQ ID NO: 3. In some examples, the 300 bp-500 bp sequence may be linked to further polynucleotide sequences but may not be linked to additional sequences derived from SEQ ID NO: 1. For example, in an embodiment, the modified cardiac troponin T promoter may include no more than 500 bp of SEQ ID NO: 1 but may include additional unrelated polynucleotide sequences. In another example, the modified cardiac troponin T promoter may include SEQ ID NO: 3, no additional sequences derived from SEQ ID NO: 1, but may include additional unrelated polynucleotide sequences.

In some embodiments, the cardiac TNNT2 promoter is modified by the deletion of polynucleotides. A modification may include one, two, three or more internal deletions. Each deletion may be a deletion of 1 base pair, 2 base pairs, 3 base pairs, 4 base pairs, 5 base pairs, 10 base pairs, 15 base pairs, 20 base pairs, 25 base pairs, 30 base pairs, 40 base pairs, 50 base pairs, 60 base pairs, 70 base pairs, 80 base pairs, 90 base pairs, 100 base pairs, 125 base pairs, 150 base pairs, 175 base pairs, 200 base pairs, 225 base pairs, 250 base pairs, 275 base pairs, or 300 base pairs with respect to a reference cardiac TNNT2 promoter (SEQ ID NO: 1) having about 600 base pairs.

In some embodiments, the TNNT2 promoter is modified by the deletion of polynucleotides from the upstream end of the promoter with respect to a reference cardiac TNNT2 promoter (SEQ ID NO: 1) having about 600 base pairs. A modification may include the deletion of 1 base pair, 2 base pairs, 3 base pairs, 4 base pairs, 5 base pairs, 10 base pairs, 15 base pairs, 20 base pairs, 25 base pairs, 30 base pairs, 40 base pairs, 50 base pairs, 60 base pairs, 70 base pairs, 80 base pairs, 90 base pairs, 100 base pairs, 125 base pairs, 150 base pairs, 175 base pairs, 200 base pairs, 225 base pairs, 250 base pairs, 275 base pairs, or 300 base pairs from the upstream end of the promoter with respect to a reference cardiac TNNT2 promoter (SEQ ID NO: 1) having about 600 base pairs. In some embodiments, the modification is a 200 base pair deletion from the upstream end of the promoter with respect to a reference cardiac TNNT2 promoter (SEQ ID NO: 1) having about 600 base pairs.

In some embodiments, the cardiac TNNT2 promoter is modified by the deletion of polynucleotides from the downstream end of the promoter with respect to a reference cardiac TNNT2 promoter (SEQ ID NO: 1) having about 600 base pairs. A modification may include the deletion of 1 base pair, 2 base pairs, 3 base pairs, 4 base pairs, 5 base pairs, 10 base pairs, 15 base pairs, 20 base pairs, 25 base pairs, 30 base pairs, 40 base pairs, 50 base pairs, 60 base pairs, 70 base pairs, 80 base pairs, 90 base pairs, 100 base pairs, 125 base pairs, 150 base pairs, 175 base pairs, 200 base pairs, 225 base pairs, 250 base pairs, 275 base pairs, or 300 base pairs from the downstream end of the promoter with respect to a reference cardiac TNNT2 promoter (SEQ ID NO: 1) having about 600 base pairs.

In some embodiments, the cardiac TNNT2 promoter is modified by an internal deletion of polynucleotides. A modification may include the internal deletion of 1 base pair, 2 base pairs, 3 base pairs, 4 base pairs, 5 base pairs, 10 base pairs, 15 base pairs, 20 base pairs, 30 base pairs, 40 base pairs, 50 base pairs, 60 base pairs, 70 base pairs, 80 base pairs, 90 base pairs, 100 base pairs, 125 base pairs, 150 base pairs, 175 base pairs, 200 base pairs, 225 base pairs, 250 base pairs, 275 base pairs, or 300 base pairs with respect to a reference cardiac TNNT2 promoter (SEQ ID NO: 1).

In some embodiments, the cardiac TNNT2 promoter is modified by the insertion of polynucleotides. A modification may include the insertion of 1 base pair, 2 base pairs, 3 base pairs, 4 base pairs, 5 base pairs, 10 base pairs, 15 base pairs, 20 base pairs, 25 base pairs, 30 base pairs, 35 base pairs, 40 base pairs, 45 base pairs, 50 base pairs, 55 base pairs, 60 base pairs, 65 base pairs, 70 base pairs, 75 base pairs, 80, base pairs, 85 base pairs, 90 base pairs, 100 base pairs, 125 base pairs, 150 base pairs, 175 base pairs, 200 base pairs, 225 base pairs, 250 base pairs, 275 base pairs, or 300 base pairs with respect to a reference cardiac TNNT2 promoter (SEQ ID NO: 1).

In some embodiments, the cardiac TNNT2 promoter is modified by the substitution of polynucleotides. A modification may include the substitution of 1 base pair, 2 base pairs, 3 base pairs, 4 base pairs, 5 base pairs, 6 base pairs, 7 base pairs, 8 base pairs, 9 base pairs, or 10 base pairs with respect to a reference cardiac TNNT2 promoter (SEQ ID NO: 1).

In some embodiments, the polynucleotide sequence of the TNNT2 promoter shares at least 80%, 90%, 95%, 96%, 97%, 98%, 99%, or 100% sequence identity with the polynucleotide sequence −450 base pairs to +1 base pairs relative to the transcription start site of the human TNNT2 gene. In some embodiments, the polynucleotide sequence of the TNNT2 promoter shares at least 80%, 90%, 95%, 96%, 97%, 98%, 99%, or and 100% sequence identity with the polynucleotide sequence −350 base pairs to +1 base pairs relative to the transcription start site of the human TNNT2 gene. In some embodiments, the polynucleotide sequence of the TNNT2 promoter shares at least 80%, 90%, 95%, 96%, 97%, 98%, 99%, or and 100% sequence identity with the polynucleotide sequence −250 base pairs to +1 base pairs relative to the transcription start site of the human TNNT2 gene.

In some embodiments, the polynucleotide sequence of the cardiac TNNT2 promoter shares at least 80%, 90%, 95%, 96%, 97%, 98%, 99%, or and 100% sequence identity with the polynucleotide sequence −450 base pairs to +50 base pairs relative to the transcription start site of the TNNT2 gene. In some embodiments, the polynucleotide sequence of the cardiac TNNT2 promoter shares at least 80%, 90%, 95%, 96%, 97%, 98%, 99%, or and 100% sequence identity with the polynucleotide sequence −350 base pairs to +50 base pairs relative to the transcription start site of the TNNT2 gene. In some embodiments, the polynucleotide sequence of the cardiac TNNT2 promoter shares at least 80%, 90%, 95%, 96%, 97%, 98%, 99%, or and 100% sequence identity with the polynucleotide sequence −250 base pairs to +5 base pairs relative to the transcription start site of the TNNT2 gene.

In some embodiments, the cardiac TNNT2 promoter comprises a polynucleotide comprising a sequence that shares at least 80%, 90%, 95%, 96%, 97%, 98%, 99%, or and 100% identity to any one of SEQ ID NOs: 1-85. In some embodiments, the polynucleotide comprises a sequence that shares at least 80% identity to any one of SEQ ID NOS: 1-85. In some embodiments, the polynucleotide comprises a sequence that shares at least 90% identity to any one of SEQ ID NOS: 1-85. In some embodiments, the polynucleotide comprises a sequence that shares at least 100% identity to any one of SEQ ID NOS: 1-85. In some embodiments, the polynucleotide comprises a sequence that shares at least 80% identity to SEQ ID NO: 1. In some embodiments, the polynucleotide comprises a sequence that shares at least 90% identity to SEQ ID NO: 1. In some embodiments, the polynucleotide comprises a sequence that shares at least 100% identity to SEQ ID NO: 1. In some embodiments, the polynucleotide comprises a sequence that shares at least 80% identity to SEQ ID NO: 3. In some embodiments, the polynucleotide comprises a sequence that shares at least 90% identity to SEQ ID NO: 3. In some embodiments, the polynucleotide comprises a sequence that shares at least 100% identity to SEQ ID NO: 3.

In some embodiments of the expression cassettes and vectors described herein, the TNNT2 promoter of SEQ ID NO: 1 or the TNNT2 promoter of SEQ ID NO: 3 is used for cardiac-specific protein expression.

In addition to or instead of a modified cardiac TNNT2 promoter, some embodiments employ other eukaryotic promoters, including but not limited to: cytomegalovirus (CMV) immediate early, herpes simplex virus (HSV) thymidine kinase, a viral simian virus 40 (SV40) (e.g., early and late SV40), a spleen focus forming virus (SFFV) promoter, long terminal repeats (LTRs) from retrovirus (e.g., a Moloney murine leukemia virus (MoMLV) LTR promoter or a Rous sarcoma virus (RSV) LTR), a herpes simplex virus (HSV) (thymidine kinase) promoter, H5, P7.5, and P11 promoters from vaccinia virus, an elongation factor 1-alpha (EF1α) promoter, early growth response 1 (EGR1) promoter, a ferritin H (FerH) promoter, a ferritin L (FerL) promoter, a Glyceraldehyde 3-phosphate dehydrogenase (GAPDH) promoter, a eukaryotic translation initiation factor 4A1 (EIF4A1) promoter, a heat shock 70 kDa protein 5 (HSPA5) promoter, a heat shock protein 90 kDa beta, member 1 (HSP90B1) promoter, a heat shock protein 70 kDa (HSP70) promoter, a β-kinesin (β-KIN) promoter, the human ROSA 26 locus (Irions et al., Nature Biotechnology 25, 1477-1482 (2007)), a Ubiquitin C (UBC) promoter, a phosphoglycerate kinase-1 (PGK) promoter, a cytomegalovirus enhancer/chicken j-actin (CAG) promoter, a β-actin promoter and a myeloproliferative sarcoma virus enhancer, negative control region deleted, d1587rev primer-binding site substituted (MND) promoter, and mouse metallothionein-1.

In some embodiments, the promoters provided herein for expression of a gRNA or an inhibitory RNA are promoter sequences known in the art for expressing RNA (e.g. siRNA or shRNA). In some aspects, the present disclosure provides an RNA polymerase III (Pol III) promoter, such as a U6 promoter. Any natural or synthetic promoter for expression of short RNAs can also be used for expressing inhibitory RNA (e.g., siRNA or shRNA). In some embodiments, the promoter for expressing inhibitory RNA described herein is any natural promoter for expression of short RNA described herein or known in the art. In some embodiments, the promoter for expressing a gRNA or an inhibitory RNA described herein is any synthetic promoter for expression of short RNA described herein or known in the art. In some embodiments, the RNA expression-driving promoter is a human Pol III promoter.

In some embodiments, the polynucleotides, expression cassettes and vectors described herein comprise a U6 promoter. In some embodiments, the polynucleotides, expression cassettes and vectors described herein comprise a human U6 promoter. In some embodiment, the polynucleotides, expression cassettes and vectors described herein comprising or encoding one or more inhibitory RNA or guide RNA, such as inhibitory or guide RNA inhibiting expression of MTSS1 protein, comprise a U6 promoter. In some embodiments, the U6 promoter comprises SEQ ID NO: 101. In some embodiments, the promoter comprises a sequence that shares at least 60%, at least 65%, at least 70%, at least 75%, at least 80%, at least 85%, at least 90%, at least 95%, at least 96%, at least 97%, at least 98%, at least 99%, or 100% identity to SEQ ID NO: 101.

In some embodiments, the polynucleotides, expression cassettes and vectors described herein comprise a modified U6 promoter. In some embodiment, the polynucleotides, expression cassettes and vectors described herein comprising or encoding one or more inhibitory RNA, such as an inhibitory RNA inhibiting expression of MTSS1 protein, comprise a modified U6 promoter. In some embodiments, the modified U6 promoter shares at least 80%, 85%, 90%, 95%, 96%, 97%, 98%, or 99% sequence identity to SEQ ID NO: 101.

In some embodiments, the polynucleotides, expression cassettes and vectors described herein comprise a human H1 promoter, for example, a human H1 promoter 1 of SEQ ID NO: 400, or human H1 promoter 2 of SEQ ID NO: 401. In some embodiment, the polynucleotides, expression cassettes and vectors described herein comprising or encoding one or more inhibitory RNA or guide RNA, such as inhibitory or guide RNA inhibiting expression of MTSS1 protein, comprise human H1 promoter 1 of SEQ ID NO: 400 or human H1 promoter 2 of SEQ ID NO: 401. In some embodiments, the RNA expression-driving promoter comprises a sequence that shares at least 60%, at least 65%, at least 70%, at least 75%, at least 80%, at least 85%, at least 90%, at least 95%, at least 96%, at least 97%, at least 98%, at least 99%, or 100% identity to SEQ ID NO: 400 or SEQ ID NO: 401.

In some embodiments, the RNA expression-driving promoter is doxycycline-inducible variant of the human H1 RNA promoter. In some embodiments, the promoter is doxycycline-inducible variant of the human H1 RNA promoter of SEQ ID NO: 402. In some embodiments, the RNA expression-driving promoter comprises a sequence that shares at least 60%, at least 65%, at least 70%, at least 75%, at least 80%, at least 85%, at least 90%, at least 95%, at least 96%, at least 97%, at least 98%, at least 99%, or 100% identity to SEQ ID NO: 402.

TABLE 3B
Exemplary RNA promoter sequences

| | | SEQ |
|---|---|---|
| | | ID |
| Name | Sequence | NO: |
| Human U6 | gagggcctatttcccatgattccttcatat | 101 |
| Promoter | ttgcatatacgatacaaggctgttagagag | |
| | ataattggaattaatttgactgtaaacaca | |
| | aagatattagtacaaaatacgtgacgtaga | |
| | aagtaataatttcttgggtagtttgcagtt | |
| | ttaaaattatgttttaaaatggactatcat | |
| | atgcttaccgtaacttgaaagtatttcgat | |
| | ttcttggctttatatatcttgtggaaagga | |
| | c | |
| Human H1 | tatttgcatgtcgctatgtgttctgggaaa | 400 |
| promoter | tcaccataaacgtgaaatgtctttggattt | |
| 1 | gggaatcttataagttctgtatgagaccac | |
| Human H1 | gaacgctgacgtcatcaacccgctccaagg | 401 |
| promoter | aatcgcgggcccagtgtcactaggcgggaa | |
| 2 | cacccagcgcgcgtgcgccctggcaggaag | |
| | atggctgtgagggacaggggagtggcgccc | |
| | tgcaatatttgcatgtcgctatgtgttctg | |
| | ggaaatcaccataaacgtgaaatgtctttg | |
| | gatttgggaatcttataagttctgtatgag | |
| | accac | |
| Doxycycline | atatttgcatgtcgctatgtgttctgggaa | 402 |
| -inducible | atcaccataaacgtgaaatccctatcagtg | |
| variant | atagagacttataagttccctatcagtgat | |
| of the | agaga | |
| human H1 | | |
| RNA | | |
| promoter | | |

### Enhancers

The term “enhancer” refers to a segment of DNA which contains sequences capable of providing enhanced transcription and in some instances can function independent of their orientation relative to another control sequence. An enhancer can function cooperatively or additively with promoters and/or other enhancer elements. The term “enhancer” further refers to a DNA sequence that directs the binding of transcriptional regulatory proteins (e.g., transcriptional machinery) and RNA polymerase, and thereby promotes RNA synthesis. An enhancer may overlap with a promoter or be upstream or downstream of the promoter.

The expression cassette can include one or more enhancers. The enhancer can be operably linked to a promoter and modulate the expression of a transgene operably linked to a promoter. The presence of an enhancer can modulate transgene expression by, for example, increasing expression or decreasing expression. An enhancer can modulate transgene expression by, for example, increasing expression levels in a desired cell type, for example, a cardiac cell. An enhancer can modulate transgene expression by, for example, decreasing expression levels in an “off-target” cell type, or a cell type in which expression is not desired.

In some embodiments, the expression cassette comprises a single enhancer. In some embodiments, the expression cassette comprises at least one enhancer. In some embodiments, the expression cassette comprises two enhancers. In some embodiments, the expression cassette comprises three enhancers. In some embodiments, the expression cassette comprises four enhancers. In some embodiments, the expression cassette comprises an enhancer that is operably linked to a promoter. In some embodiments, the modified cardiac TNNT2 promoter comprises one or more enhancers. In some embodiments, the modified cardiac TNNT2 promoter comprises no enhancer. For example, a ACTC1 cardiac enhancer can be linked to a human cTnT promoter. In some embodiments, the expression cassette comprises an enhancer that is operably linked to another enhancer. For example, a ACTC1 cardiac enhancer can be operably linked to an uMHC enhancer. In some embodiments, the expression cassette comprises an enhancer that is operably linked to a promoter and operably linked to another enhancer.

In some embodiments, the enhancer comprises an ACTC1 cardiac enhancer (ACTC1e). In some embodiments, the ACTC1 cardiac enhancer shares at least 90%, 95%, 96%, 97%, 98%, 99%, or 100% identity to SEQ ID NO: 102. In some embodiments, the ACTC1 cardiac enhancer comprises SEQ ID NO: 102. In some embodiments, the enhancer comprises an uMHC enhancer (αMHCe). In some embodiments, the uMHC enhancer shares at least 90%, 95%, 96%, 97%, 98%, 99%, or 100% identity to SEQ ID NO: 103. In some embodiments, the uMHC enhancer comprises SEQ ID NO: 103.

TABLE 4A
Exemplary enhancer sequences

| | | SEQ |
|---|---|---|
| | | ID |
| Name | Sequence | NO: |
| ACTC1 | aactggcctgcccgagaccaaacgtgcgga | 102 |
| cardiac | acgtagttaagtgttagaggtaggatttga | |
| enhancer | agcctgtcgatcattctgattctccttttc | |
| (ACTC1e) | tctacgtctgcttcctgtcaatgggcatcc | |
| | tcactgtcaaatgcagatggtacagcaggg | |
| | cttggtctcagccaggcaggcctctcccca | |
| | gtctccatggctcagctgtccagcagtttc | |
| | atccctagaccatcccaaacatggttgaga | |
| | agctctgaggggaggacccagcactgcccg | |
| | gcccctgaagataatcagcagtcctgctca | |
| | gcatatcaatccaagcccactctagacaga | |
| | gatgccggtgcccagttttctatttttaac | |
| | tggtgtgaactgaaggaaaagcacagcatt | |
| | agaagtccaagca | |
| αMHC | ccttcagattaaaaataactaaggtaaggg | 103 |
| cardiac | ccatgtgggtaggggaggtggtgtgagacg | |
| enhancer | gtcctgtctctcctctatctgcccatcggc | |
| (αMHCe) | cctttggggaggaggaatgtgcccaaggac | |
| | taaaaaaaggccctggagccagaggggcga | |
| | gggcagcagacctttcatgggcaaacctca | |
| | gggctgctgtc | |

### Introns

The expression cassette can include an intron sequence, for example, a synthetic or chimeric intron sequence. The intron sequence can be used to adjust the length (i.e., size) of the expression cassette for improving recombinant AAV packaging. The intron sequence can be used to improve the efficiency of transgene expression (i.e., mRNA production or transcription) in a host cell containing the expression cassette. In some embodiments, the expression cassette comprises an intron. In some embodiments, the intron comprises the CMV intron (CMVint). In some embodiments, the CMV intron shares at least 90%, 95%, 96%, 97%, 98%, 99%, or 100% identity to SEQ ID NO: 104. In some embodiments, the CMV intron comprises SEQ ID NO: 104. In some embodiments, the intron comprises a chimeric intron. In some embodiments, the chimeric intron shares at least 90%, 95%, 96%, 97%, 98%, 99%, or 100% identity to SEQ ID NO: 105. In some embodiments, the chimeric intron comprises SEQ ID NO: 105.

TABLE 4B
Exemplary intron sequences

| | | SEQ ID |
|---|---|---|
| Name | Sequence | NO: |
| CMV | gtaagtaccgcctatagactctataggcac | 104 |
| intron | acccctttggctcttatgcatgctgacaga | |
| | ctaacagactgttcctttcctgggtctttt | |
| | ctgcag | |
| Chimeric | gtaagtatcaaggttacaagacaggtttaa | 105 |
| intron | ggagaccaatagaaactgggcttgtcgaga | |
| (Chimint) | cagagaagactcttgcgtttctgataggca | |
| | cctattggtcttactgacatccactttgcc | |
| | tttctctccacag | |

### WPRE Sequences and Other Post-Transcriptional Elements

In some embodiments, the expression cassette comprises a posttranscriptional regulatory element.

In some embodiments, the expression cassette comprises a woodchuck hepatitis virus post-transcriptional element (WPRE). The WPRE sequence can be inserted, for example, proximal to on the 3′ end of a transgene in a viral vector to, for example, optimize gene expression in a viral vector (Lee et al. Exp Physiol. 90:33-37 (2005)). In some embodiments, the WPRE comprises a polynucleotide sequence that shares at least 90%, 95%, 96%, 97%, 98%, 99%, or 100% identity to SEQ ID NO: 106. In some embodiments, the WPRE comprises SEQ ID NO: 106.

In some embodiments, the expression cassette provided herein does not comprise a posttranscriptional regulatory element.

In some embodiments, the expression cassette provided herein does not comprise a WPRE.

TABLE 4C
Exemplary WPRE sequence

| | | SEQ ID |
|---|---|---|
| Name | Sequence | NO: |
| WPRE | tcaacctctggattacaaaatttgtgaaag | 106 |
| | attgactggtattcttaactatgttgctcc | |
| | ttttacgctatgtggatacgctgctttaat | |
| | gcctttgtatcatgctattgcttcccgtat | |
| | ggctttcattttctcctccttgtataaatc | |
| | ctggttgctgtctctttatgaggagttgtg | |
| | gcccgttgtcaggcaacgtggcgtggtgtg | |
| | cactgtgtttgctgacgcaacccccactgg | |
| | ttggggcattgccaccacctgtcagctcct | |
| | ttccgggactttcgctttccccctccctat | |
| | tgccacggeggaactcategccgcctgcct | |
| | tgcccgctgctggacaggggctcggctgtt | |
| | gggcactgacaattccgtggtgttgtcggg | |
| | gaaatcatcgtcctttccttggctgctcgc | |
| | ctgtgttgccacctggattctgcgcgggac | |
| | gtccttctgctacgtcccttcggccctcaa | |
| | tccageggacettecttcccgeggcctgct | |
| | gccggctctgeggcctcttccgcgtcttcg | |

### Polyadenylation Sequences

In some embodiments, the expression cassette comprises a poly(A) signal sequence. In some embodiments, the poly(A) signal comprises, consists essentially of, or consists of a sequence that shares at least 90%, 95%, 96%, 97%, 98%, 99%, or 100% identity to SEQ ID NO: 107. In some embodiments, the poly(A) signal comprises, consists essentially of, or consists of a sequence that shares at least 95% identity to SEQ ID NO: 107. In some embodiments, the poly(A) signal is SEQ ID NO: 107.

In some embodiments, the poly(A) signal is a BGH poly(A) sequence. In some embodiments, the BGH poly(A) signal sequence comprises, consists essentially of, or consists of the polynucleotide sequence that shares at least 90%, 95%, 96%, 97%, 98%, 99%, or 100% identity to SEQ ID NO: 108. In some embodiments, the poly(A) signal is an SV40 poly(A) signal. In some embodiments, the SV40 poly(A) signal sequence comprises, consists essentially of, or consists of the polynucleotide sequence that shares at least 90%, 95%, 96%, 97%, 98%, 99%, or 100% identity to SEQ ID NO: 109.

In some embodiments, vectors comprise a polyadenylation sequence 3′ of a polynucleotide encoding a polypeptide to be expressed. The term “poly(A) site” or “poly(A) sequence” as used herein denotes a DNA sequence which directs both the termination and polyadenylation of the nascent RNA transcript by RNA polymerase II. Polyadenylation sequences can promote mRNA stability by addition of a poly(A) tail to the 3′ end of the coding sequence and thus, contribute to increased translational efficiency. Cleavage and polyadenylation are directed by a poly(A) sequence in the RNA. The core poly(A) sequence for mammalian pre-mRNAs has two recognition elements flanking a cleavage-polyadenylation site. Typically, an almost invariant AAUAAA hexamer lies 20-50 nucleotides upstream of a more variable element rich in U or GU residues. Cleavage of the nascent transcript occurs between these two elements and is coupled to the addition of up to 250 adenosines to the 5′ cleavage product. In particular embodiments, the core poly(A) sequence is an ideal poly(A) sequence (e.g., AATAAA, ATTAAA, AGTAAA). In particular embodiments, the poly(A) sequence is an SV40 poly(A) sequence, a bovine growth hormone poly(A) sequence (BGHpA), a rabbit β-globin poly(A) sequence (rogpA), variants thereof, or another suitable heterologous or endogenous poly(A) sequence known in the art

TABLE 4D
Exemplary poly(A) sequences

| | | SEQ ID |
|---|---|---|
| Name | Sequence | NO: |
| Synthetic | aataaaagatctttattttcattagatctg | 107 |
| poly(A) | tgtgttggttttttgtgtg | |
| BGH | gcctcgactgtgccttctagttgccagcca | 108 |
| poly(A) | tctgttgtttgcccctcccccgtgccttcc | |
| | ttgaccctggaaggtgccactcccactgtc | |
| | ctttcctaataaaatgaggaaattgcatcg | |
| | cattgtctgagtaggtgtcattctattctg | |
| | gggggtggggggggcaggacagcaaggggg | |
| | aggattgggaagacaatagcaggcatgctg | |
| | ggga | |
| SV40 | gatccagacatgataagatacattgatgag | 109 |
| poly(A) | tttggacaaaccacaactagaatgcagtga | |
| | aaaaaatgctttatttgtgaaatttgtgat | |
| | gctattgctttatttgtaaccattataagc | |
| | tgcaataaacaagt | |

### Inverted Terminal Repeat Sequences

In some embodiments, the expression cassette is flanked by AAV inverted terminal repeats (ITRs). In some embodiments, the ITRs comprise the polynucleotide sequence that shares at least 90%, 95%, 96%, 97%, 98%, 99%, or 100% identity to SEQ ID NO: 110 and/or SEQ ID NO: 111.

TABLE 4E
Exemplary ITR sequences

| | | SEQ ID |
|---|---|---|
| Name | Sequence | NO: |
| 5′ ITR | ctgcgcgctcgctcgctcactgaggccgcc | 110 |
| | cgggcaaagcccgggcgtcgggcgaccttt | |
| | ggtcgcccggcctcagtgagcgagcgagcg | |
| | cgcagagagggagtggccaactccatcact | |
| | aggggttcct | |
| 3′ ITR | aggaacccctagtgatggagttggccactc | 111 |
| | cctctctgcgcgctcgctcgctcactgagg | |
| | ccgggcgaccaaaggtcgcccgacgcccgg | |
| | gctttgcccgggcggcctcagtgagcgagc | |
| | gagcgcgcag | |

In some embodiments, rAAV genome comprises an expression cassette that is flanked by one or both of a 5′ inverted terminal repeat (ITR) and a 3′ ITR. In some embodiments, the 5′ ITR comprises a sequence that shares 95% identity to SEQ ID NO: 110. In some embodiments, the 3′ ITR comprises a sequence that shares at least 95% identity to SEQ ID NO: 111.

### Additional Elements

In some embodiments of an expression cassette expressing a Cas endonuclease, the Cas protein coding sequence is preceded by a Kozak sequence. Suitable Kozak sequences are known in the art and include, but are not limited to, the Kozak sequence of SEQ ID NO: 403.

In some embodiments, the coding sequence of the Cas protein is linked to one or more nuclear localization sequences (NLS). Suitable NLS sequences are known in the art and include, but are not limited to, the sequence encoded by the SV40 NLS (SEQ ID NO: 404) and the nucleoplasmin NLS (SEQ ID NO: 405). In some embodiments, a Cas protein coding sequence comprises an NLS at 5′ end (or an N-terminal end of Cas) and/or 3′ end (or a C-terminal end of Cas).

TABLE 4F
Exemplary additional element sequences

| | | SEQ |
|---|---|---|
| | | ID |
| Name | Sequence | NO: |
| Kozak | gccaccatgg | 403 |
| Sequence | | |
| SV40 NLS | ccaaagaagaagcggaaggtc | 404 |
| Nucleoplasmin | aaaaggccggcggccacgaaa | 405 |
| NLS | aaggccggccaggcaaaaaag | |
| | aaaaag | |

In some embodiments, the expression cassettes described herein comprise a transcription termination signal. Elements directing the efficient termination and polyadenylation of the heterologous nucleic acid transcripts increases heterologous gene expression. Transcription termination signals are generally found downstream of the polyadenylation signal.

In some embodiments, the expression cassette described herein comprises a termination signal for producing a gRNA. In some embodiments, the polynucleotide encoding the gRNA described herein, and/or the expression cassette encoding the gRNA described herein, comprise a pol III termination signal. In some embodiments, the pol III termination signal is TTTTTG.

### Vectors

In some aspects, the disclosure provides vectors comprising the expression cassettes provided herein. The vector can be any viral vector, or any non-viral vector known in the art or described herein.

In some embodiments, the vector is a viral vector. In some embodiments the viral vector is an adeno-associated virus vector (AAV), an adenoviral vector (AV), a lentiviral vector (LV), a retroviral vector (RV), a herpes simplex virus vector (HSV), or a poxvirus vector.

In some embodiments, provided herein is an AAV comprising any expression cassette described herein. In some embodiments, provided herein is an AV comprising any expression cassette described herein. In some embodiments, provided herein is an LV comprising any expression cassette described herein. In some embodiments, provided herein is an RV comprising any expression cassette described herein. In some embodiments, provided herein is an HSV comprising any expression cassette described herein. In some embodiments, provided herein is a poxvirus-based vector comprising any expression cassette described herein.

In some embodiments, the viral vector is a retroviral vector, e.g., a lentiviral vector. As used herein, the term “retrovirus” or “retroviral” refers an RNA virus that reverse transcribes its genomic RNA into a linear double-stranded DNA copy and subsequently covalently integrates its genomic DNA into a host genome. Retrovirus vectors are a common tool for gene delivery (Miller, Nature. 357: 455-460 (2000)). Once the virus is integrated into the host genome, it is referred to as a “provirus.” The provirus serves as a template for RNA polymerase II and directs the expression of RNA molecules encoded by the virus. In some embodiments, a retroviral vector is altered so that it does not integrate into the host cell genome.

Illustrative retroviruses (family Retroviridae) include but are not limited to: (1) genus gammaretrovirus, such as, Moloney murine leukemia virus (M-MuLV), Moloney murine sarcoma virus (MoMSV), murine mammary tumor virus (MuMTV), gibbon ape leukemia virus (GaLV), and feline leukemia virus (FLV), (2) genus spumavirus, such as, simian foamy virus, (3) genus lentivirus, such as, human immunodeficiency virus-1 and simian immunodeficiency virus.

As used herein, the term “lentiviral” or “lentivirus” refers to a group (or genus) of complex retroviruses. Illustrative lentiviruses include but are not limited to HIV (human immunodeficiency virus; including HIV type 1, and HIV type 2; visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV).

In some embodiments, the viral vector is an adenoviral vector. The genetic organization of adenovirus includes an approximate 36 kb, linear, double-stranded DNA virus, which allows substitution of large pieces of adenoviral DNA with foreign sequences up to 7 kb (Grunhaus et al., Seminar in Virology 200(2):535-546, 1992)).

In some embodiments, the viral vector is an adeno-associated viral (AVV) vector, such as an AAV vector selected from the group consisting of serotype 1, 2, 3, 4, 5, 6, 7, 8, 9, and 10 or chimeric AAV derived thereof.

In some embodiments, the AAV expression vector is pseudotyped to enhance targeting. A pseudotyping strategy can promote gene transfer and sustain expression in a target cell type. For example, the AAV2 genome can be packaged into the capsid of another AAV serotype such as AAV5, AAV7, or AAV8, producing pseudotyped vectors such as AAV2/5, AAV2/7, and AAV2/8 respectively, as described in Balaji et al. J Surg Res. September; 184(1): 691-698 (2013). In some embodiments, an AAV9 may be used to target expression in myofibroblast-like lineages, as described in Piras et al. Gene Therapy 23:469-478 (2016). In some embodiments, AAV1, AAV6, or AAV9 is used, and in some embodiments, the AAV is engineered, as described in Asokari et al. Hum Gene Ther. November; 24(11): 906-913 (2013); Pozsgai et al. Mol Ther. April 5; 25(4): 855-869 (2017); Kotterman, M. A. and D. V. Schaffer Engineering Adeno-Associated Viruses for Clinical Gene Therapy. Nature Reviews Genetics, 15:445-451 (2014); and US20160340393A1 to Schaffer et al. In some embodiments, the viral vector is AAV engineered to increase target cell infectivity as described in US20180066285A1. In some embodiments, the vector is an AAV9 vector.

In some embodiments, the vector is a non-viral vector. In some embodiments, the non-viral vector is a naked DNA (e.g., a DNA plasmid). In some embodiments, the non-viral vector is a plasmid. In some embodiments, the non-viral vector is a liposome or lipid vector comprising plasmid DNA and a lipid solution.

For example, viral and non-viral vectors and delivery systems are described in Sung & Kim 2019, Biomaterials Research 23:8; Mali, 2013, Indian Journal of Human Genetics, 19(1):3-8; Hardee et al., 2017, Genes 8:65; Bulcha et al., 2020, Signal Transduction and Targeted Therapy; Ghosh et al., 2020, Applied Biosafety: Journal of ABSA International 25(1):7-18, the disclosures of each of which are hereby incorporated by reference herein in their entireties.

In some embodiments, the vectors are recombinant vectors.

In some embodiments, the vectors described herein comprise an expression cassette comprising a polynucleotide encoding any gene product or comprising any inhibitory RNA described herein.

In some aspects of the disclosure, a vector is used to deliver the expression cassettes described herein to cardiac cells of a subject, e.g., to treat cardiomyopathy. In some embodiments, the disclosure provides a viral vector comprising an expression cassette comprising a polynucleotide encoding a gene product, an inhibitory siRNA or a gene editing system, operably linked to a promoter, and a pharmaceutically acceptable carrier. In some embodiments, the disclosure provides a virion comprising a capsid and an expression cassette comprising a polynucleotide encoding a gene product, an inhibitory RNA, or a gene editing system, operably linked to a promoter, and a pharmaceutically acceptable carrier. In some embodiments, the disclosure provides a plasmid comprising an expression cassette comprising a polynucleotide encoding a gene product, an inhibitory RNA, or a gene editing system, operably linked to a promoter and a pharmaceutically acceptable carrier.

In some embodiments, the viral vectors described herein are replication incompetent, in that it cannot independently further replicate and package its genome. For example, when a cardiac cell is targeted with a virion, the transgene is expressed in the targeted cardiac cell, however, due to the fact that the targeted cardiac cell lacks packaging and accessory function genes, the virion is not able to replicate. In some embodiments, the viral vectors described herein are replication-competent.

In some embodiments, the vectors described herein are capable of being delivered to both dividing and non-dividing cells. In some embodiments, the vectors described herein are capable of being delivered to non-dividing cells. In some embodiments, the vectors described herein are capable of being delivered to dividing cells.

In some embodiments, the vectors comprising the expression cassettes described herein lead to cardiac cell-specific expression of a transgene (such as MMP11, SYNPO2LA, or SYNPO2LB). In some embodiments, the vectors comprising the expression cassettes described herein lead to cardiomyocyte-specific expression of a transgene. In some embodiments, the vectors comprising the expression cassettes described herein allow high expression of a transgene in a cardiac cell (e.g., a cardiomyocyte) and low or no expression in other cells (e.g., low or no expression in liver cells, low or no expression in muscle cells except for muscle cells of the heart, low or no expression in cardiac fibroblasts). In some embodiments, the vectors comprising the expression cassettes described herein allow high expression of a transgene in heart tissue of a subject (e.g., in human heart). In some embodiments, the vectors comprising the expression cassettes described herein allow no or low expression of a transgene in tissues of a subject other than the heart (e.g., in liver or in muscles except those of the heart). “High” and “low” can be relative to each other, for example, the expression of a transgene in cardiac cells (e.g., cardiomyocytes) and/or heart tissue can be at least 2-fold, 5-fold, 10-fold, 15-fold, 20-fold, 50-fold, 100-fold, 150-fold, or 200-fold higher than its expression in other cells and tissues (e.g., liver, muscle except for the heart).

In some embodiments, the vectors comprising the expression cassettes described herein comprising inhibitory RNA lead to cardiac cell-specific inhibition of expression of a gene (such as MTSS1). In some embodiments, the vectors comprising the expression cassettes described herein lead to cardiomyocyte-specific inhibition of MTSS1. In some embodiments, the vectors comprising the expression cassettes described herein lead to at least 30%, 40%, 50%, 60%, 70%, 80%, 90%, 95%, or 98% inhibition of expression of MTSS1, e.g., in a cardiomyocyte and/or heart tissue of a subject (e.g., in human heart). In some embodiments, the vectors comprising the expression cassettes described herein inhibit MTSS1 expression by less than 50%, 40%, 30%, 20%, 10%, 5%, or 3% in other cells (e.g., liver cells) and/or tissues of a subject other than the heart (e.g., in liver).

In some embodiments, the vectors comprising the expression cassettes described herein comprising a gene editing system lead to cardiac cell-specific inhibition of expression of a gene (such as MTSS1). In some embodiments, the vectors comprising the expression cassettes described herein lead to cardiomyocyte-specific inhibition of MTSS1. In some embodiments, the vectors comprising the expression cassettes described herein lead to at least 30%, 40%, 50%, 60%, 70%, 80%, 90%, 95%, or 98% inhibition of expression of MTSS1, e.g., in a cardiomyocyte and/or heart tissue of a subject (e.g., in human heart). In some embodiments, the vectors comprising the expression cassettes described herein inhibit MTSS1 expression by less than 50%, 40%, 30%, 20%, 10%, 5%, or 3% in other cells (e.g., liver cells) and/or tissues of a subject other than the heart (e.g., in liver).

In some embodiments, the vector genome has a size of less than 6 kilobases. In some embodiments, the vector genome has a size of less than 5.8 kilobases. In some embodiments, the vector genome has a size of less than 5.7 kilobases. In some embodiments, the vector genome has a size of less than 5.6 kilobases. In some embodiments, the vector genome has a size of about or at most 4.0 kilobases, 4.5 kilobases, 4.8 kilobases, 5 kilobases, 5.1 kilobases, 5.2 kilobases, 5.3 kilobases, 5.4 kilobases, or 5.5 kilobases. In some embodiments, the vector genome has a size of 4 kilobases to 5.6 kilobases. In some embodiments, the vector genome has a size of equal to or less than 4.8 kilobases. In some of these embodiments, the vector is an AAV vector, e.g., AAV9.

Methods of introducing polynucleotides into a host cell are known in the art, and any known method can be used to introduce the polynucleotides described herein into a cell. Suitable methods include e.g., viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery, microfluidics delivery methods, and the like.

### Recombinant Aav Vectors and Virions

In some aspects of the disclosure, a recombinant adeno-associated virus (rAAV) virion is used to deliver the expression cassettes described herein to cardiac cells of a subject, e.g., to treat cardiomyopathy. Accordingly, the disclosure provides an rAAV virion, the rAAV virion comprising an AAV capsid and an expression cassette comprising a polynucleotide encoding a cardioprotective gene, gene product, an inhibitory RNA, a Cas endonuclease, and/or a guide RNA, described herein operatively linked to a promoter.

In some embodiments, an AAV referenced herein is any AAV known in the art or described herein. In some embodiments, an AAV is an AAV selected from the group consisting of serotype 1, 2, 3, 4, 5, 6, 7, 8, 9, 10, 11, 12, rh.10, rh.20, rh.74, and a chimeric AAV derived therefrom. In some embodiments, the AAV is AAV1, AAV2, AAV3, AAV4, AAV5, AAV6, AAV7, AAV8, AAV9, AAV10, AAV11, AAV12, AAVrh.10, AAVrh.20, AAVrh.74, or a variant thereof.

In some embodiments, the rAAV virions of the disclosure comprise a capsid protein. Capsid proteins are structural proteins that make up the assembled icosahedral packaging of the rAAV virion that contains the expression cassette. Capsid proteins largely determine the immunogenicity and tropism of AAVs and are classified by the serotype. Wild type capsid serotypes in rAAV virions can be, for example, AAV1, AAV2, AAV3, AAV4, AAV5, AAV6, AAV7, AAV8, AAV9, AAV10, AAV11, AAV12, AAVrh.10, AAVrh.20, or AAVrh.74 (Naso et al. BioDrugs 31:317-334 (2017)). Engineered capsid types include chimeric capsids and mosaic capsids (Choi et al. Curr Gene Ther. 5: 299-310 (2005)). Capsids are selected for rAAV virions based on their ability to transduce specific tissue or cell types (Liu et al. Curr Pharm Des. 21:3248-56 (2015)).

Any capsid protein that can facilitate rAAV virion transduction into cardiac cells for delivery of a transgene, as described herein, can be used. Capsid proteins used in rAAV virions for transgene delivery to cardiac cells that result in high expression can be, for example, AAV4, AAV6, AAV7, AAV8, and AAV9 (Zincarelli et al. Mol. Ther. 16:P1073-1080 (2008)).

In some embodiments, the disclosure provides an rAAV virion comprising an AAV capsid protein (e.g., AAV9 capsid protein) and an expression cassette described herein. In some embodiments, the disclosure provides an rAAV virion comprising a modified AAV capsid protein (e.g., a modified AAV9 capsid protein comprising one or more substitutions or insertions) and an expression cassette described herein.

In some embodiments, the rAAV virions of the disclosure comprise an AAV9 capsid protein or variant thereof. In some embodiments, the rAAV virions of the disclosure comprise wild type AAV9 capsid protein. In some embodiments, the rAAV virions of the disclosure comprise an engineered AAV9 capsid protein. In some embodiments, the rAAV virions comprise wild type AAV5 capsid protein or variant thereof. In some embodiments, the rAAV virions comprise a chimeric AAV5/AAV9 capsid protein, e.g., wherein the chimeric capsid protein comprises at least 1, 2, 3, 4, 5 or more polypeptide segments that are derived from AAV5 capsid protein and/or at least 1, 2, 3, 4, 5 or more polypeptide segments that are derived from AAV9 capsid protein. In certain of these embodiments, at least one polypeptide segment is derived from the AAV5 capsid protein and at least one polypeptide segment is derived from the AAV9 capsid protein. In some embodiments, the rAAV virions comprise wild type AAVrh.10 capsid protein or variant thereof. In some embodiments, the rAAV virions comprise wild type AAVrh.74 capsid protein or variant thereof.

Artificial capsids, such as chimeric capsids generated through combinatorial libraries, can also be used for transgene delivery to cardiac cells that results in high expression (see U.S. 63/012,703, the contents of which are herein incorporated by reference). Other capsid proteins with various features can also be used in the rAAV virions of the disclosure. AAV vectors and capsids are provided in U.S. Pat. Pub. Nos. U.S. Ser. No. 10/011,640B2; U.S. Pat. No. 7,892,809B2, U.S. Pat. No. 8,632,764B2, U.S. Pat. No. 8,889,641B2, U.S. Pat. No. 9,475,845B2, U.S. Ser. No. 10/889,833B2, U.S. Ser. No. 10/480,011B2, and U.S. Ser. No. 10/894,949B2, the contents of which are herein incorporated by reference; and Int'l Pat. Pub. Nos. WO2020198737A1, WO2019028306A2, WO2016054554A1, WO2018152333A1, WO2017106236A1, WO2008124724A1, WO2017212019A1, WO2020117898A1, WO2017192750A1, WO2020191300A1, and WO2017100671A1, the contents of which are herein incorporated by reference.

In some embodiments, the rAAV virions described herein comprise any capsid protein or variant capsid protein, e.g., any AAV9 variant capsid protein (e.g., comprising one or more substitutions or insertions), described in WO2021/163357A2 and/or U.S. Pat. No. 11,129,908, both of which are incorporated by reference herein in their entirety. In some embodiments, the rAAV virions comprise an engineered capsid protein selected from Table 5.

TABLE 5
Engineered capsid proteins

| Engineered Capsid | SEQ ID NO: |
|---|---|
| CR9-01 | 119 |
| CR9-07 | 120 |
| CR9-08 | 121 |
| CR9-09 | 122 |
| CR9-10 | 123 |
| CR9-11 | 124 |
| CR9-13 | 125 |
| CR9-14 | 126 |
| CR9-15 | 127 |
| CR9-16 | 128 |
| CR9-17 | 129 |
| CR9-20 | 130 |
| CR9-21 | 131 |
| CR9-22 | 132 |
| ZC23 | 133 |
| ZC24 | 134 |
| ZC25 | 135 |
| ZC26 | 136 |
| ZC27 | 137 |
| ZC28 | 138 |
| ZC29 | 139 |
| ZC30 | 140 |
| ZC31 | 141 |
| ZC32 | 142 |
| ZC33 | 143 |
| ZC34 | 144 |
| ZC35 | 145 |
| ZC40 | 146 |
| ZC41 | 147 |
| ZC42 | 148 |
| ZC43 | 149 |
| ZC44 | 150 |
| ZC45 | 151 |
| ZC46 | 152 |
| ZC47 | 153 |
| ZC48 | 154 |
| ZC49 | 155 |
| ZC50 | 156 |
| TN47-07 | 157 |
| TN47-10 | 158 |
| TN47-13 | 159 |
| TN47-14 | 160 |
| TN47-17 | 161 |
| TN47-22 | 162 |
| TN40-07 | 163 |
| TN40-10 | 164 |
| TN40-13 | 165 |
| TN40-14 | 166 |
| TN40-17 | 167 |
| TN40-22 | 168 |
| TN44-07 | 169 |
| TN44-10 | 270 |
| TN44-13 | 271 |
| TN44-14 | 272 |
| TN44-17 | 273 |
| TN44-22 | 274 |

In some embodiments, the engineered capsid proteins and the rAAV virions comprising the engineered capsid proteins (e.g., any AAV9 variant capsid protein, e.g., comprising one or more substitutions or insertions of the wild type AAV9 capsid protein) are any of those disclosed in WO2021/26456A2, which is incorporated by reference herein in its entirety.

The wild type AAV9 VP1 has the amino acid sequence of SEQ ID NO: 112. The wild type AAV9 VP2 has the amino acid sequence of SEQ ID NO: 113. The wild type AAV9 VP3 has the amino acid sequence of SEQ ID NO: 114. In some embodiments, the engineered AAV9 capsid protein comprises a sequence that shares at least about 80%, 85%, 90%, 95%, 96%, 97%, 98%, 99%, 99.5%, or 100% identity to SEQ ID NO: 112, as shown below. In some embodiments, the engineered AAV9 capsid protein described herein comprises a sequence that shares at least about 80%, 85%, 90%, 95%, 96%, 97%, 98%, 99%, 99.5%, or 100% identity to SEQ ID NO: 113. In some embodiments, the engineered AAV9 capsid protein described herein comprises a sequence that shares at least about 80%, 85%, 90%, 95%, 96%, 97%, 98%, 99%, 99.5%, or 100% identity to SEQ ID NO: 114. The N-terminal residue of VP1, VP2, and VP3, as well as the VR sites (VR-IV, VR-V, VR-VII and VR-VIII), are indicated (in bold, and underlined) in the sequence of full-length VP1 (SEQ ID NO: 112) below.

```
(SEQ ID NO: 112)
VP1-->
MAADGYLPDWLEDNLSEGIREWWALKPGAPQPKANQQHQDNARGLVLPGYKYLGPGNGLDKG
EPVNAADAAALEHDKAYDQQLKAGDNPYLKYNHADAEFQERLKEDTSFGGNLGRAVFQAKKR
LL.
(SEQ ID NO: 113)
                      VP2-->
EPLGLVEEAAKTAPGKKRPVEQSPQEPDSSAGIGKSGAQPAKKRLNFGQTGDTESVPDPQPI
G
(SEQ ID NO: 114)
                        VP3-->
EPPAAPSGVGSLTMASGGGAPVADNNEGADGVGSSSGNWHCDSQWLGDRVITTSTRTWALPT
YNNHLYKQISNSTSGGSSNDNAYFGYSTPWGYFDFNRFHCHESPRDWORLINNNWGFRPKRL
NFKLFNIQVKEVTDNNGVKTIANNLTSTVQVFTDSDYQLPYVLGSAHEGCLPPFPADVEMIP
QYGYLTLNDGSQAVGRSSFYCLEYFPSQMLRTGNNFQFSYEFENVPFHSSYAHSQSLDRLMN
PLID
                      VR-IV VR-V
QYLYYLSKTINGSGQNQQTLKFSVAGPSNMAVQGRNYIPGPSYRQQRVSTTVTQNNNSEFAW
P
                                                                                        VR-VII
GASSWALNGRNSLMNPGPAMASHKEGEDRFFPLSGSLIFGKQGTGRDNVDADKVMITNEEEI
K
                              VR-VIII
TTNPVATESYGQVATNHQSAQAQAQTGWVQNQGILPGMVWQDRDVYLQGPIWAKIPHTDGNE
HPSPLMGGFGMKHPPPQILIKNTPVPADPPTAFNKDKLNSFITQYSTGQVSVEIEWELQKEN
SKRWNPEIQYTSNYYKSNNVEFAVNTEGVYSEPRPIGTRYLTRNL
```

TABLE 6
Exemplary AAV9 capsid protein sequences

| | | SEQ |
|---|---|---|
| Name | Sequence | ID NO: |
| AAV9 | MAADGYLPDWLEDNLSEGIREWWALKPGAP | 112 |
| capsid | QPKANQQHQDNARGLVLPGYKYLGPGNGLD | |
| VP1 | KGEPVNAADAAALEHDKAYDQQLKAGDNPY | |
| | LKYNHADAEFQERLKEDTSFGGNLGRAVFQ | |
| | AKKRLLEPLGLVEEAAKTAPGKKRPVEQSP | |
| | QEPDSSAGIGKSGAQPAKKRLNFGQTGDTE | |
| | SVPDPQPIGEPPAAPSGVGSLTMASGGGAP | |
| | VADNNEGADGVGSSSGNWHCDSQWLGDRVI | |
| | TTSTRTWALPTYNNHLYKQISNSTSGGSSN | |
| | DNAYFGYSTPWGYFDFNRFHCHFSPRDWQR | |
| | LINNNWGFRPKRLNFKLFNIQVKEVTDNNG | |
| | VKTIANNLTSTVQVFTDSDYQLPYVLGSAH | |
| | EGCLPPFPADVFMIPQYGYLTLNDGSQAVG | |
| | RSSFYCLEYFPSQMLRTGNNFQFSYEFENV | |
| | PFHSSYAHSQSLDRLMNPLIDQYLYYLSKT | |
| | INGSGQNQQTLKFSVAGPSNMAVQGRNYIP | |
| | GPSYRQQRVSTTVTQNNNSEFAWPGASSWA | |
| | LNGRNSLMNPGPAMASHKEGEDRFFPLSGS | |
| | LIFGKQGTGRDNVDADKVMITNEEEIKTTN | |
| | PVATESYGQVATNHQSAQAQAQTGWVQNQG | |
| | ILPGMVWQDRDVYLQGPIWAKIPHTDGNFH | |
| | PSPLMGGFGMKHPPPQILIKNTPVPADPPT | |
| | AFNKDKLNSFITQYSTGQVSVEIEWELQKE | |
| | NSKRWNPEIQYTSNYYKSNNVEFAVNTEGV | |
| | YSEPRPIGTRYLTRNL | |
| AAV9 | TAPGKKRPVEQSPQEPDSSAGIGKSGAQPA | 113 |
| capsid | KKRLNFGQTGDTESVPDPQPIGEPPAAPSG | |
| VP2 | VGSLTMASGGGAPVADNNEGADGVGSSSGN | |
| | WHCDSQWLGDRVITTSTRTWALPTYNNHLY | |
| | KQISNSTSGGSSNDNAYFGYSTPWGYFDFN | |
| | RFHCHFSPRDWQRLINNNWGFRPKRLNFKL | |
| | FNIQVKEVTDNNGVKTIANNLTSTVQVFTD | |
| | SDYQLPYVLGSAHEGCLPPFPADVFMIPQY | |
| | GYLTLNDGSQAVGRSSFYCLEYFPSQMLRT | |
| | GNNFQFSYEFENVPFHSSYAHSQSLDRLMN | |
| | PLIDQYLYYLSKTINGSGQNQQTLKFSVAG | |
| | PSNMAVQGRNYIPGPSYRQQRVSTTVTQNN | |
| | NSEFAWPGASSWALNGRNSLMNPGPAMASH | |
| | KEGEDRFFPLSGSLIFGKQGTGRDNVDADK | |
| | VMITNEEEIKTTNPVATESYGQVATNHQSA | |
| | QAQAQTGWVQNQGILPGMVWQDRDVYLQGP | |
| | IWAKIPHTDGNFHPSPLMGGFGMKHPPPQI | |
| | LIKNTPVPADPPTAFNKDKLNSFITQYSTG | |
| | QVSVEIEWELQKENSKRWNPEIQYTSNYYK | |
| | SNNVEFAVNTEGVYSEPRPIGTRYLTRNL | |
| AAV9 | MASGGGAPVADNNEGADGVGSSSGNWHCDS | 114 |
| capsid | QWLGDRVITTSTRTWALPTYNNHLYKQISN | |
| VP3 | STSGGSSNDNAYFGYSTPWGYFDFNRFHCH | |
| | FSPRDWQRLINNNWGFRPKRLNFKLFNIQV | |
| | KEVTDNNGVKTIANNLTSTVQVFTDSDYQL | |
| | PYVLGSAHEGCLPPFPADVFMIPQYGYLTL | |
| | NDGSQAVGRSSFYCLEYFPSQMLRTGNNFQ | |
| | FSYEFENVPFHSSYAHSQSLDRLMNPLIDQ | |
| | YLYYLSKTINGSGQNQQTLKFSVAGPSNMA | |
| | VQGRNYIPGPSYRQQRVSTTVTQNNNSEFA | |
| | WPGASSWALNGRNSLMNPGPAMASHKEGED | |
| | RFFPLSGSLIFGKQGTGRDNVDADKVMITN | |
| | EEEIKTTNPVATESYGQVATNHQSAQAQAQ | |
| | TGWVQNQGILPGMVWQDRDVYLQGPIWAKI | |
| | PHTDGNFHPSPLMGGFGMKHPPPQILIKNT | |
| | PVPADPPTAFNKDKLNSFITQYSTGQVSVE | |
| | IEWELQKENSKRWNPEIQYTSNYYKSNNVE | |
| | FAVNTEGVYSEPRPIGTRYLTRNL | |
| AAV9 VR-IV | NGSGQNQ | 115 |
| AAV0 VR-V | NNSEFA | 116 |
| AAV9 VR-VII | GRDNV | 117 |
| AAV9 VR-VIII | ATNHQSAQAQAQTG | 118 |

In some embodiments, the rAAV virions of the disclosure comprise an engineered capsid protein, e.g., an engineered AAV9 capsid protein. Engineered capsid proteins can be derived from a parental, e.g., wild type, capsid (e.g., wild type AAV9 capsid protein) and include, for example, variant polypeptide sequence with respect to a parental capsid sequence at one or more sites. For example, variant sites of the parental capsid can occur at the VR-IV site, VR-V site, VR-VII site, and/or VR-VIII site (see, e.g., Büning and Srivastava. Mol Ther Methods Clin Dev. 12:248-265 (2019)).

In some embodiments, the variant has a substitution or insertion in the VR-IV region of the capsid protein (e.g., of AAV9). In some embodiments, the variant has a substitution or insertion in the VR-V region of the capsid protein (e.g., of AAV9). In some embodiments, the variant has a substitution or insertion in the VR-VII region of the capsid protein (e.g., of AAV9). In some embodiments, the variant has a substitution or insertion in the VR-VIII region of the capsid protein (e.g., of AAV9). In some embodiments, the variant has a substitution or insertion in the VR-IV region and the VR-VIII region of the capsid protein (e.g., of AAV9).

In some embodiments, the capsid protein is an AAV5/AAV9 chimeric capsid protein. In some embodiments, the chimeric capsid protein comprises at least 1, 2, 3, 4, 5 or more polypeptide segments that are derived from AAV5 capsid protein. In some embodiments, the chimeric capsid protein comprises at least 1, 2, 3, 4, 5 or more polypeptide segments that are derived from AAV9 capsid protein. In some embodiments, at least one polypeptide segment is derived from the AAV5 capsid protein and at least one polypeptide segment is derived from the AAV9 capsid protein.

In some embodiments, the capsid protein is a combinatory capsid protein. As used herein, “combinatory capsid protein” refers to a AAV5/AAV9 chimeric capsid protein, which further comprises amino acid variations with respect to the chimeric parental sequence at one or more sites. In some embodiments, the one or more sites of the chimeric parental sequence are selected from those equivalent to the VR-IV site, the VR-V site, the VR-VII site and the VR-VIII site of the AAV9 capsid protein.

In some embodiments, the rAAV virions described herein comprise any capsid protein or variant capsid protein, e.g., any AAV9 variant capsid protein (e.g., comprising one or more substitutions or insertions) described herein. In some embodiments, the rAAV capsid protein shares at least 80%, at least 85%, at least 90%, or at least 95% polypeptide sequence identity to an AAV9 VP3 reference sequence according to SEQ ID NO: 114, wherein the capsid protein comprises, relative to reference sequence SEQ ID NO: 112, one or more modifications described herein.

In some embodiments, the capsid protein comprises one, two, three, four or more substitutions in the VR-VIII site. In some embodiments, the capsid protein comprises one, two, three, four or more insertions in the VR-VIII site. In some embodiments, the capsid protein comprises, relative to reference SEQ ID NO: 112, one, two, three, four or more substitutions at positions from 584 to 590 in the VR-VIII site, or one, two, three, four or more substitutions at positions from 585 to 590 in the VR-VIII site. In some embodiments, the capsid protein comprises, relative to reference SEQ ID NO: 112, one, two, three, four or more insertions at positions from 584 to 590 in the VR-VIII site, or one, two, three, four or more insertions at positions from 585 to 590 in the VR-VIII site.

In some embodiments, the capsid protein comprises at least two, three, four, five or more substitutions in the VR-VIII site. In some embodiments, the capsid protein comprises at least two, three, four or more insertions in the VR-VIII site. In some embodiments, the capsid protein comprises, relative to reference SEQ ID NO: 112, at least two, three, four, five or more substitutions at positions from 584 to 590 in the VR-VIII site, or at least two, three, four, five or more substitutions at positions from 585 to 590 in the VR-VIII site. In some embodiments, the capsid protein comprises, relative to reference SEQ ID NO: 112, at least two, three, four or more insertions at positions from 584 to 590 in the VR-VIII site, or at least two, three, four or more insertions at positions from 585 to 590 in the VR-VIII site.

In some embodiments, the capsid protein: (i) is cardiotropic, (ii) mediates increased transduction efficiency in cardiac cells compared to the parental sequence, (iii) mediates decreased transduction efficiency in liver cells compared to the parental sequence, and/or (iv) mediates increased selectivity for the cardiac cells over liver cells compared to the parental sequence. These characteristics may be assessed in cells (e.g., iPSC-derived cardiac cells or cardiomyocytes) in vitro, or in mice or primates in vivo, by any methods known in the art.

In some embodiments, the capsid protein may comprise an amino acid insertion at position 584 (relative to reference sequence SEQ ID NO: 112) comprising one or more of an asparagine (N), a threonine (T), a tyrosine (Y), phenylalanine (F), and an alanine (A).

In some embodiments, the capsid protein may comprise an amino acid insertion at position 585 (relative to reference sequence SEQ ID NO: 112) comprising one or more of a histidine (H) and a methionine (M).

In some embodiments, the capsid protein may comprise an amino acid insertion at position 586 (relative to reference sequence SEQ ID NO: 112) comprising one or more of a histidine (H), a tyrosine (Y), a valine (V), a threonine (T), an alanine (A), an isoleucine (I), a tryptophan (W), a methionine (M), and a leucine.

In some embodiments, the capsid protein may comprise an amino acid insertion at position 587 (relative to reference sequence SEQ ID NO: 112) comprising one or more of an isoleucine (I) and a proline (P).

In some embodiments, the capsid protein may comprise an amino acid insertion at position 588 (relative to reference sequence SEQ ID NO: 112) comprising one or more of an isoleucine (I), a threonine (T), and a proline (P).

In some embodiments, the capsid protein may comprise one or more amino acid substitutions selected from the group consisting of N452K, N452A, N452V, G453A, G453N, S454T, S454D, G455N, Q456L, Q456K, N457L, N457V, Q458I, and Q458H (relative to reference sequence SEQ ID NO: 112).

In some embodiments, the capsid protein may comprise one or more amino acid substitutions selected from the group consisting of T582D, T582L, T582E, T582A, T582F, T582R, T582P, N583V, N583T, H584R, H584Q, H584K, H584V, H584Y, H584M, H584T, H584W, H584E, H584D, Q585T, Q585C, Q585V, Q585L, Q585N, Q585S, Q585P, Q585A, Q585M, Q585E, Q585Y, Q585G, Q585H, Q585I, S586D, S586T, S586G, S586K, S586M, S586N, S586I, S586Q, S586L, S586P, S586F, S586R, A587F, A587S, A587T, A587N, A587L, A587P, A587V, A587K, A587I, A587R, A587H, A587G, A587M, A587D, A587W, Q588L, Q588S, Q588F, Q588N, Q588G, Q588R, Q588I, Q588V, Q588T, Q588Y, Q588H, Q588M, Q588K, Q588D, A589R, A589I, A589N, A589S, A589V, A589Q, A589F, A589T, A589K, A589H, A589E, A589W, A589L, A589Y, A589M, Q590I, Q590S, Q590N, Q590G, Q590D, Q590R, Q590H, Q590T, Q590M, Q590F, Q590Y, Q590L, A591I, G594Q, and G594D (relative to reference sequence SEQ ID NO: 112).

In some embodiments, an rAAV capsid protein of the present technology shares, or comprises a sequence sharing, at least 80% (e.g., at least 80%, at least 85%, at least 90%, at least 95%, at least 96%, at least 97%, at least 98%, at least 99%, or 100%) amino acid sequence identity to an AAV9 VP3 reference sequence according to SEQ ID NO: 114, and wherein the capsid protein comprises, relative to reference sequence SEQ ID NO: 112:
an amino acid insertion between position 583 and 584 comprising one or more of an asparagine (N), a threonine (T), a tyrosine (Y), phenylalanine (F), and an alanine (A);
an amino acid insertion between position 584 and 585 comprising one or more of a histidine (H) and a methionine (M);
an amino acid insertion between position 585 and 586 comprising one or more of a histidine (H), a tyrosine (Y), a valine (V), a threonine (T), an alanine (A), an isoleucine (I), a tryptophan (W), a methionine (M), and a leucine (L);
an amino acid insertion between position 586 and 587 comprising one or more of an isoleucine (I) and a proline (P);
an amino acid insertion between position 587 and 588 comprising one or more of an isoleucine (I), a threonine (T), and a proline (P);
an amino acid insertion between position 588 and 589 comprising one or more of a glycine (G) and a glutamine (Q);
one or more amino acid substitutions selected from the group consisting of N452K, N452A, N452V, N452I, G453A, G453N, S454T, S454D, G455N, Q456L, Q456K, N457L, N457V, Q458I, and Q458H; and/or
one or more amino acid substitutions selected from the group consisting of T582D, T582L, T582E, T582A, T582F, T582R, T582P, N583V, N583T, H584R, H584Q, H584K, H584V, H584Y, H584M, H584T, H584W, H584E, H584D, Q585T, Q585C, Q585V, Q585L, Q585N, Q585S, Q585P, Q585A, Q585M, Q585E, Q585Y, Q585G, Q585H, Q585I, S586D, S586T, S586G, S586K, S586M, S586N, S586I, S586Q, S586L, S586P, S586F, S586R, A587F, A587S, A587T, A587N, A587L, A587P, A587V, A587K, A587I, A587R, A587H, A587G, A587M, A587D, A587W, Q588L, Q588S, Q588F, Q588N, Q588G, Q588R, Q588I, Q588V, Q588T, Q588Y, Q588H, Q588M, Q588K, Q588D, A589R, A589I, A589N, A589S, A589V, A589Q, A589F, A589T, A589K, A589H, A589E, A589W, A589L, A589Y, A589M, Q590I, Q590S, Q590N, Q590G, Q590D, Q590R, Q590H, Q590T, Q590M, Q590F, Q590Y, Q590L, A591I, G594Q, and G594D.

In some embodiments, the capsid protein may comprise an amino acid insertion at position 584 (relative to reference sequence SEQ ID NO: 112) consisting of a TY, FN, or AT.

In some embodiments, the capsid protein may comprise an amino acid insertion at position 585 (relative to reference sequence SEQ ID NO: 112) consisting of MH.

In some embodiments, the capsid protein may comprise an amino acid insertion at position 586 (relative to reference sequence SEQ ID NO: 112) consisting of HY, VT, AI, WM, or ML.

In some embodiments, the capsid protein may comprise an amino acid insertion at position 587 (relative to reference sequence SEQ ID NO: 112) consisting of PI.

In some embodiments, the capsid protein may comprise an amino acid insertion at position 588 (relative to reference sequence SEQ ID NO: 112) consisting of IT or PT.

In some embodiments, the capsid protein may comprise one or more amino acid substitutions selected from the group consisting of T582D, T582E, N583V, H584Q, S586K, A587P, A587S, Q588G, Q588M, A589S, A591I, G594Q, and G594D (relative to reference sequence SEQ ID NO: 112).

In some embodiments, the capsid protein may comprise one or more amino acid substitutions selected from the group consisting of T582L, T582A, T582F, T582R, T582P, H584R, H584K, H584V, H584Y, H584M, H584Q, H584W, H584E, H584D, Q585T, Q585N, Q585M, Q585E, Q585V, Q585H, S586T, S586G, S586Q, S586I, S586L, S586F, S586D, S586R, S586M, A587F, A587I, A587H, A587M, A587N, A587W, Q588Y, Q588S, Q588T, and Q588R (relative to reference sequence SEQ ID NO: 112).

In some embodiments, the capsid protein may comprise one or more amino acid substitutions selected from the group consisting of Q585C, Q585S, and S586I (relative to reference sequence SEQ ID NO: 112).

In some embodiments, the capsid protein may comprise one or more amino acid substitutions selected from the group consisting of Q585C, Q585S, S586I, A587V and A587G (relative to reference sequence SEQ ID NO: 112).

In some embodiments, the capsid protein may comprise one or more amino acid substitutions selected from the group consisting of Q585V, Q585T, Q585L, Q585C, Q585N, Q585S, Q585M, Q585E, Q585P, Q585A, Q585G, Q585H, Q585I, S586D, S586G, S586T, S586M, S586N, S586L, S586R, S586I, S586K, A587S, A587T, A587N, A587L, A587V, A587K, A587I, A587F, A587P, A587R, A587D, Q588L, Q588S, Q588F, Q588N, Q588R, Q588I, Q588V, Q588T, Q588H, Q588Y, Q588M, Q588K, Q588D, Q588G, A589R, A589I, A589N, A589S, A589V, A589Q, A589F, A589T, A589K, A589H, A589E, A589W, A589L, A589Y, A589M, Q590I, Q590S, Q590N, Q590G, Q590D, Q590R, Q590H, Q590T, Q590M, Q590F, Q590Y, and Q590L (relative to reference sequence SEQ ID NO: 112).

In some embodiments, the capsid protein may comprise one or more amino acid substitutions selected from the group consisting of A587V and A587G (relative to reference sequence SEQ ID NO: 112).

In some embodiments, the capsid protein may comprise the amino acid sequence ANYG at positions 586-589 or at about positions 586-589 (relative to reference sequence SEQ ID NO: 112).

In some embodiments, the capsid protein may comprise two or more amino acid substitutions selected from the group consisting of N452K, N452A, N452V, G453A, G453N, S454T, S454D, G455N, Q456L, Q456K, N457L, N457V, Q458I, and Q458H (relative to reference sequence SEQ ID NO: 112).

In some embodiments, the capsid protein may comprise the amino acid substitution N452K, N452A, or N452V (relative to reference sequence SEQ ID NO: 112).

In some embodiments, the capsid protein may comprise the amino acid substitution N452K (relative to reference sequence SEQ ID NO: 112).

In some embodiments, the capsid protein may comprise the amino acid substitution G453A or G453N (relative to reference sequence SEQ ID NO: 112).

In some embodiments, the capsid protein may comprise the amino acid substitution S454T or S454D (relative to reference sequence SEQ ID NO: 112).

In some embodiments, the capsid protein may comprise the amino acid substitution G455N (relative to reference sequence SEQ ID NO: 112).

In some embodiments, the capsid protein may comprise the amino acid substitution Q456L or Q456K (relative to reference sequence SEQ ID NO: 112).

In some embodiments, the capsid protein may comprise the amino acid substitution N457L or N457V (relative to reference sequence SEQ ID NO: 112).

In some embodiments, the capsid protein may comprise the amino acid substitution Q458I or Q458H (relative to reference sequence SEQ ID NO: 112).

In some embodiments, the capsid protein may comprise an ammo acid sequence selected from KGSGQNQ (SEQ ID NO: 175), NASGQNQ (SEQ ID NO: 176), NGTGQNQ (SEQ ID NO: 177), NGSGLNQ (SEQ ID NO: 178), ANDNKLI (SEQ ID NO: 179), VNDNKVI (SEQ ID NO: 180), NGSGQNH (SEQ ID NO: 181), and ANDNKVI (SEQ ID NO: 182) at positions 452-458 or at about positions 452-458 (relative to reference sequence SEQ ID NO: 112).

In some embodiments, the capsid protein comprises relative to reference sequence SEQ ID NO: 112, at position 452 an amino acid selected from the group consisting of: K and N. In some embodiments, the capsid protein comprises, relative to reference sequence SEQ ID NO: 112, an amino acid substitution N452K.

In some embodiments, an rAAV capsid protein of the present technology shares, or comprises a sequence sharing, at least 80% amino acid sequence identity to an AAV9 VP3 reference sequence according to SEQ ID NO: 114, and wherein the capsid protein comprises, relative to reference sequence SEQ ID NO: 112, amino acid substitution N452K. In some embodiments, N452K is the only substitution in the capsid protein relative to the parental or wild-type AAV9. In some embodiments, N452K is not the only substitution in the capsid protein relative to the parental or wild-type AAV9.

In some embodiments, the capsid protein comprises, relative to reference sequence SEQ ID NO: 112:
at position 585 an amino acid selected from: E, N, G, M, C, V, T and Q;
at position 586 an amino acid selected from: N, T, M, G, D, and S;
at position 587 an amino acid selected from: T, L, I, K, S, N, V and A;
at position 588 an amino acid selected from: V, F, Y, L, T, S, I, R and Q;
at position 589 an amino acid selected from: S, N, L, T, I, R and A; and/or
at position 590 an amino acid selected from: I, S, G, H, R and Q.

In some embodiments, the capsid protein comprises, relative to reference sequence SEQ ID NO: 112:
at position 585 an amino acid selected from: E, N, G, M, C, V, T and Q;
at position 586 an amino acid selected from: N, T, M, G, D, and S;
at position 587 an amino acid selected from: T, L, I, K, S, N, V and A;
at position 588 an amino acid selected from: V, F, Y, L, T, S, I, R and Q;
at position 589 an amino acid selected from: S, N, L, T, I, R and A; and
at position 590 an amino acid selected from: I, S, G, H, R and Q.

In some embodiments, the capsid protein comprises, relative to reference sequence SEQ ID NO: 112:
at position 585 an amino acid selected from: E, N, G, M, C, V and T;
at position 586 an amino acid selected from: N, T, M, G, and D;
at position 587 an amino acid selected from: T, L, I, K, S, N and V;
at position 588 an amino acid selected from: V, F, Y, L, T, S, I and R;
at position 589 an amino acid selected from: S, N, L, T, I and R; and/or
at position 590 an amino acid selected from: I, S, G, H and R.

In some embodiments, the capsid protein comprises, relative to reference sequence SEQ ID NO: 112:
at position 585 an amino acid selected from: E, N, G, M, C, V and T;
at position 586 an amino acid selected from: N, T, M, G, and D;
at position 587 an amino acid selected from: T, L, I, K, S, N and V;
at position 588 an amino acid selected from: V, F, Y, L, T, S, I and R;
at position 589 an amino acid selected from: S, N, L, T, I and R; and
at position 590 an amino acid selected from: I, S, G, H and R.

In some embodiments, the capsid protein comprises, relative to reference sequence SEQ ID NO: 112:
at position 584 an amino acid selected from the group consisting of: R and H;
at position 585 an amino acid selected from the group consisting of N, M, C, E, G, S, V, A, T, H, L and Q;
at position 586 an amino acid selected from the group consisting of: M, D, N, G, A, T, R, I and S;
at position 587 an amino acid selected from the group consisting of: T, N, V, L, I, S, R, P and A;
at position 588 an amino acid selected from the group consisting of: Y, T, S, I, V, F, L, R, N, D, G and Q;
at position 589 an amino acid selected from the group consisting of: L, I, R, S, G, N, T, V, Q, F, E, Y and A; and/or
at position 590 an amino acid selected from the group consisting of: G, R, S, I, H, N, Y, L, M and Q; and
optionally at position 452 an amino acid selected from the group consisting of: N and K.

In some embodiments, the capsid protein comprises, relative to reference sequence SEQ ID NO: 112:
at position 452 an amino acid selected from the group consisting of: K and N;
at position 584 an amino acid selected from the group consisting of: R and H;
at position 585 an amino acid selected from the group consisting of N, M, C, E, G, S, V, A, T, H, L and Q;
at position 586 an amino acid selected from the group consisting of: M, D, N, G, A, T, R, I and S;
at position 587 an amino acid selected from the group consisting of: T, N, V, L, I, S, R, P and A;
at position 588 an amino acid selected from the group consisting of: Y, T, S, I, V, F, L, R, N, D, G and Q;
at position 589 an amino acid selected from the group consisting of: L, I, R, S, G, N, T, V, Q, F, E, Y and A; and
at position 590 an amino acid selected from the group consisting of: G, R, S, I, H, N, Y, L, M and Q.

In some embodiments, the capsid protein comprises, relative to reference sequence SEQ ID NO: 112:
at position 584 amino acid R;
at position 585 an amino acid selected from the group consisting of: N, M, C, E, G, S, V, A, T, H and, L;
at position 586 an amino acid selected from the group consisting of: M, D, N, G, A, T, R, and I;
at position 587 an amino acid selected from the group consisting of: T, N, V, L, I, S, R, and P;
at position 588 an amino acid selected from the group consisting of: Y, T, S, I, V, F, L, R, N, D, and G;
at position 589 an amino acid selected from the group consisting of: L, I, R, S, G, N, T, V, Q, F, E, and Y; and/or
at position 590 an amino acid selected from the group consisting of: G, R, S, I, H, N, Y, L, and M; and
optionally at position 452 an amino acid selected from the group consisting of: N and K.

In some embodiments, the capsid protein comprises, relative to reference sequence SEQ ID NO: 112, at least two, three, four, five, six, seven or all eight of any of the following:
(i) at position 452 amino acid K;
(ii) at position 584 amino acid R;
(iii) at position 585 an amino acid selected from the group consisting of: N, M, C, E, G, S, V, A, T, H, and L;
(iv) at position 586 an amino acid selected from the group consisting of: M, D, N, G, A, T, R, and I;
(v) at position 587 an amino acid selected from the group consisting of: T, N, V, L, I, S, R, and P;
(vi) at position 588 an amino acid selected from the group consisting of: Y, T, S, I, V, F, L, R, N, D, and G;
(vii) at position 589 an amino acid selected from the group consisting of: L, I, R, S, G, N, T, V, Q, F, E, and Y; and
(viii) at position 590 an amino acid selected from the group consisting of: G, R, S, I, H, N, Y, L, and M.

In some embodiments, the capsid protein comprises, relative to reference sequence SEQ ID NO: 112:
at position 585 an amino acid selected from the group consisting of: E, N, G, M, C, V, T and Q;
at position 586 an amino acid selected from the group consisting of: N, T, M, G, D, and S;
at position 587 an amino acid selected from the group consisting of: T, L, I, K, S, N, V and A;
at position 588 an amino acid selected from the group consisting of: V, F, Y, L, T, S, I, R and Q;
at position 589 an amino acid selected from the group consisting of: S, N, L, T, I, R and A; and/or
at position 590 an amino acid selected from the group consisting of: I, S, G, H, R and Q; and optionally at position 452 an amino acid selected from the group consisting of N and K.

In some embodiments, the capsid protein comprises, relative to reference sequence SEQ ID NO: 112:
at position 452 an amino acid selected from the group consisting of: K and N;
at position 585 an amino acid selected from the group consisting of: E, N, G, M, C, V, T and Q;
at position 586 an amino acid selected from the group consisting of: N, T, M, G, D, and S;
at position 587 an amino acid selected from the group consisting of: T, L, I, K, S, N, V and A;
at position 588 an amino acid selected from the group consisting of: V, F, Y, L, T, S, I, R and Q;
at position 589 an amino acid selected from the group consisting of: S, N, L, T, I, R and A; and
at position 590 an amino acid selected from the group consisting of: I, S, G, H, R and Q.

In some embodiments, the capsid protein comprises, relative to reference sequence SEQ ID NO: 112:
at position 585 an amino acid selected from the group consisting of: E, N, G, M, C, V and T;
at position 586 an amino acid selected from the group consisting of: N, T, M, G, and D;
at position 587 an amino acid selected from the group consisting of: T, L, I, K, S, N and V;
at position 588 an amino acid selected from the group consisting of: V, F, Y, L, T, S, I and R;
at position 589 an amino acid selected from the group consisting of: S, N, L, T, I and R; and/or
at position 590 an amino acid selected from the group consisting of: I, S, G, H and R; and optionally at position 452 an amino acid selected from the group consisting of: N and K.

In some embodiments, the capsid protein comprises, relative to reference sequence SEQ ID NO: 112, at least two, three, four, five, six or all seven of any of the following:
(i) at position 452 amino acid K;
(ii) at position 585 an amino acid selected from the group consisting of E, N, G, M, C, V and T;
(iii) at position 586 an amino acid selected from the group consisting of: N, T, M, G, and D;
(iv) at position 587 an amino acid selected from the group consisting of T, L, I, K, S, NandV;
(v) at position 588 an amino acid selected from the group consisting of V, F, Y, L, T, S, I and R;
(vi) at position 589 an amino acid selected from the group consisting of S, N, L, T, I and R; and
(vii) at position 590 an amino acid selected from the group consisting of: I, S, G, H and R.

In some embodiments, the capsid protein comprises, relative to reference sequence SEQ ID NO: 112:
at position 585 an amino acid selected from the group consisting of: E, N, M, C, and Q;
at position 586 an amino acid selected from the group consisting of A, M, G, D, N and S;
at position 587 an amino acid selected from the group consisting of: T, N, V and A;
at position 588 an amino acid selected from the group consisting of: V, Y, T, S, I and Q;
at position 589 an amino acid selected from the group consisting of: S, G, L, I, R and A; and/or
at position 590 an amino acid selected from the group consisting of: I, S, G, R and Q; and optionally at position 452 an amino acid selected from the group consisting of: N and K.

In some embodiments, the capsid protein comprises, relative to reference sequence SEQ ID NO: 112:
at position 452 an amino acid selected from the group consisting of: K and N;
at position 585 an amino acid selected from the group consisting of: E, N, M, C, and Q;
at position 586 an amino acid selected from the group consisting of: A, M, G, D, N and S;
at position 587 an amino acid selected from the group consisting of: T, N, V and A;
at position 588 an amino acid selected from the group consisting of: V, Y, T, S, I and Q;
at position 589 an amino acid selected from the group consisting of: S, G, L, I, R and A; and
at position 590 an amino acid selected from the group consisting of: I, S, G, R and Q.

In some embodiments, the capsid protein comprises, relative to reference sequence SEQ ID NO: 112:
at position 585 an amino acid selected from the group consisting of: E, N, M, and C;
at position 586 an amino acid selected from the group consisting of: A, M, G, D, and N;
at position 587 an amino acid selected from the group consisting of: T, N, and V;
at position 588 an amino acid selected from the group consisting of: V, Y, T, S, and I;
at position 589 an amino acid selected from the group consisting of: S, G, L, I and R; and/or
at position 590 an amino acid selected from the group consisting of: I, S, G, and R; and optionally at position 452 an amino acid selected from the group consisting of: N and K.

In some embodiments, the capsid protein comprises, relative to reference sequence SEQ ID NO: 112, at least two, three, four, five, six or all seven of any of the following:
(i) at position 452 amino acid K;
(ii) at position 585 an amino acid selected from the group consisting of: E, N, M, and C;
(iii) at position 586 an amino acid selected from the group consisting of: A, M, G, D, and N;
(iv) at position 587 an amino acid selected from the group consisting of: T, N, and V;
(v) at position 588 an amino acid selected from the group consisting of: V, Y, T, S, and I;
(vi) at position 589 an amino acid selected from the group consisting of: S, G, L, I and R; and
(vii) at position 590 an amino acid selected from the group consisting of: I, S, G, and R.

In some embodiments, the capsid protein comprises, relative to reference sequence SEQ ID NO: 112:
at position 452 an amino acid selected from the group consisting of: K and N; and
at position 587 amino acid substitution A587T.

In some embodiments, the capsid protein comprises, relative to reference sequence SEQ ID NO: 112:
at position 452 an amino acid selected from the group consisting of: K and N; and amino acid N or R at one, two or more positions selected from the group consisting of: 584, 585, 586, 588, 589, and 590.

In some embodiments, the capsid protein comprises, relative to reference sequence SEQ ID NO: 112:
at position 452 an amino acid selected from the group consisting of: K and N; and amino acid S at two or more positions selected from the group consisting of: 585, 586, 587, 588, 589 and 590.

In some embodiments, the capsid protein comprises, relative to reference sequence SEQ ID NO: 112:
at position 452 an amino acid selected from the group consisting of: K and N; and
at three, four or more positions in the region 585-590 of the VR-VIII site, an amino acid selected from the group consisting of: N, S, T, R and I.

In some embodiments, the capsid protein comprises, relative to reference sequence SEQ ID NO: 112:
at three, four or more positions in the region 585-590 of the VR-VIII site, an amino acid selected from the group consisting of: N, S, T, and R.

In some embodiments, the capsid protein comprises, relative to reference sequence SEQ ID NO: 112:
at position 452 an amino acid selected from the group consisting of: K and N; and
at three, four or more positions in the region 585-590 of the VR-VIII site, amino acids selected from the group consisting of: N, S, T, R and I (such as any combination and number of each of these amino acids).

In some embodiments, the capsid protein comprises, relative to reference sequence SEQ ID NO: 112:
at three, four or more positions in the region 585-590 of the VR-VIII site, amino acids selected from the group consisting of: N, S, T, and R (such as any combination and number of each of these amino acids).

In some embodiments, the capsid protein comprises, relative to reference sequence SEQ ID NO: 112:
at position 452 an amino acid selected from the group consisting of: K and N; and
at four, five or more positions in the region 585-590 of the VR-VIII site, amino acids selected from the group consisting of: N, S, T, R and I (such as any combination and number of each of these amino acids).

In some embodiments, the capsid protein comprises, relative to reference sequence SEQ ID NO: 112:
at four, five or more positions in the region 585-590 of the VR-VIII site, amino acids selected from the group consisting of: N, S, T, and R (such as any combination and number of each of these amino acids).

In some embodiments, the capsid protein comprises, relative to reference sequence SEQ ID NO: 112, at least two, three, four or more of the amino acid substitutions Q585E, S586N, A587T, Q588V, A589S, Q590I, and/or N452K (or any combination of these substitutions). In some embodiments, the capsid protein comprises, relative to reference sequence SEQ ID NO: 112, amino acid substitutions Q585E, S586N, A587T, Q588V, A589S, Q590I, and N452K.

In some embodiments, the capsid protein comprises, relative to reference sequence SEQ ID NO: 112, at least two, three, four or more of the amino acid substitutions S586T, A587L, Q588F, A589N, Q590S, and/or N452K (or any combination of these substitutions). In some embodiments, the capsid protein comprises, relative to reference sequence SEQ ID NO: 112, amino acid substitutions S586T, A587L, Q588F, A589N, Q590S, and N452K.

In some embodiments, the capsid protein comprises, relative to reference sequence SEQ ID NO: 112, at least two, three, four or more of the amino acid substitutions Q585N, A587T, Q588Y, A589L, Q590G, and/or N452K (or any combination of these substitutions). In some embodiments, the capsid protein comprises, relative to reference sequence SEQ ID NO: 112, amino acid substitutions Q585N, A587T, Q588Y, A589L, Q590G, and N452K.

In some embodiments, the capsid protein comprises, relative to reference sequence SEQ ID NO: 112, at least two, three, four or more of the amino acid substitutions Q585G, A587I, Q588L, A589T, Q590H, and/or 452K (or any combination of these substitutions). In some embodiments, the capsid protein comprises, relative to reference sequence SEQ ID NO: 112, amino acid substitutions Q585G, A587I, Q588L, A589T, Q590H, and N452K.

In some embodiments, the capsid protein comprises, relative to reference sequence SEQ ID NO: 112, at least two, three, four or more of the amino acid substitutions Q585M, S586M, A587T, Q588T, and/or Q590R (or any combination of these substitutions). In some embodiments, the capsid protein comprises, relative to reference sequence SEQ ID NO: 112, amino acid substitutions Q585M, S586M, A587T, Q588T, and Q590R. In some embodiments, the capsid protein comprises, relative to reference sequence SEQ ID NO: 112, amino acid substitutions Q585M, S586M, A587T, Q588T, and Q590R; and amino acid N at position 452.

In some embodiments, the capsid protein comprises, relative to reference sequence SEQ ID NO: 112, at least two, three, four or more of the amino acid substitutions Q585N, A587T, Q588Y, A589L, and/or Q590G (or any combination of these substitutions). In some embodiments, the capsid protein comprises, relative to reference sequence SEQ ID NO: 112, amino acid substitutions Q585N, A587T, Q588Y, A589L, and Q590G. In some embodiments, the capsid protein comprises, relative to reference sequence SEQ ID NO: 112, amino acid substitutions Q585N, A587T, Q588Y, A589L, and Q590G; and amino acid N at position 452.

In some embodiments, the capsid protein comprises, relative to reference sequence SEQ ID NO: 112, at least two, three, four or more of the amino acid substitutions Q585C, A587T, Q588S, A589I, and/or Q590R (or any combination of these substitutions). In some embodiments, the capsid protein comprises, relative to reference sequence SEQ ID NO: 112, amino acid substitutions Q585C, A587T, Q588S, A589I, and Q590R. In some embodiments, the capsid protein comprises, relative to reference sequence SEQ ID NO: 112, amino acid substitutions Q585C, A587T, Q588S, A589I, and Q590R; and amino acid N at position 452.

In some embodiments, the capsid protein comprises, relative to reference sequence SEQ ID NO: 112, at least two, three, four or more of the amino acid substitutions Q585E, S586D, A587N, Q588I, A589R, and/or Q590S (or any combination of these substitutions). In some embodiments, the capsid protein comprises, relative to reference sequence SEQ ID NO: 112, amino acid substitutions Q585E, S586D, A587N, Q588I, A589R, and Q590S. In some embodiments, the capsid protein comprises, relative to reference sequence SEQ ID NO: 112, amino acid substitutions Q585E, S586D, A587N, Q588I, A589R, and Q590S; and amino acid N at position 452.

In some embodiments, the capsid protein comprises, relative to reference sequence SEQ ID NO: 112, at least two, three, four or more of the amino acid substitutions Q585E, S586D, A587N, Q588I, A589R, Q590S, and/or N452K (or any combination of these substitutions). In some embodiments, the capsid protein comprises, relative to reference sequence SEQ ID NO: 112, amino acid substitutions Q585E, S586D, A587N, Q588I, A589R, Q590S, and N452K.

In some embodiments, the capsid protein comprises, relative to reference sequence SEQ ID NO: 112, amino acid S586G and/or Q588Y. In some embodiments, the capsid protein comprises, relative to reference sequence SEQ ID NO: 112, amino acid substitutions S586G and Q588Y; and amino acid N at position 452.

In some embodiments, the capsid protein comprises, relative to reference sequence SEQ ID NO: 112, at least two, three, four or more of the amino acid substitutions S586A, A587N, Q588Y, A589G, and/or N452K (or any combination of these substitutions). In some embodiments, the capsid protein comprises, relative to reference sequence SEQ ID NO: 112, amino acid substitutions S586A, A587N, Q588Y, A589G, and N452K.

In some embodiments, the capsid protein comprises, relative to reference sequence SEQ ID NO: 112, amino acids ATN at positions 581-583, and amino acids AQTG at positions 591-594.

In some embodiments, the capsid protein comprises, relative to reference sequence SEQ ID NO: 112, amino acids ATNH at positions 581-584, and amino acids AQTG at positions 591-594.

In some embodiments, the capsid protein comprises, relative to reference sequence SEQ ID NO: 112, any one of the following:
(i) amino acid sequence ATNHENTVSIAQTG (SEQ ID NO: 183) at the VR-VIII positions 581-594, and amino acid K at the VR-IV position 452;
(ii) amino acid sequence ATNHQTLFNSAQTG (SEQ ID NO: 184) at the VR-VIII positions 581-594, and amino acid K at the VR-IV position 452;
(iii) amino acid sequence ATNHNSTYLGAQTG (SEQ ID NO: 185) at the VR-VIII positions 581-594, and amino acid K at the VR-IV position 452;
(iv) amino acid sequence ATNHGSILTHAQTG (SEQ ID NO: 186) at the VR-VIII positions 581-594, and amino acid K at the VR-IV position 452;
(v) amino acid sequence ATNHMMTTARAQTG (SEQ ID NO: 187) at the VR-VIII positions 581-594, and amino acid N at the VR-IV position 452;
(vi) amino acid sequence ATNHNSTYLGAQTG (SEQ ID NO: 185) at the VR-VIII positions 581-594, and amino acid N at the VR-IV position 452;
(vii) amino acid sequence ATNHCSTSIRAQTG (SEQ ID NO: 188) at the VR-VIII positions 581-594, and amino acid N at the VR-IV position 452;
(viii) amino acid sequence ATNHEDNIRSAQTG (SEQ ID NO: 189) at the VR-VIII positions 581-594, and amino acid N at the VR-IV position 452;
(ix) amino acid sequence ATNHEDNIRSAQTG (SEQ ID NO: 189) at the VR-VIII positions 581-594, and amino acid K at the VR-IV position 452;
(x) amino acid sequence ATNHNNVISGAQTG (SEQ ID NO: 190) at the VR-VIII positions 581-594, and amino acid K at the VR-IV position 452;
(xi) amino acid sequence ATNHQGAYAQAQTG (SEQ ID NO: 191) at the VR-VIII positions 581-594, and amino acid N at the VR-IV position 452;
(xii) amino acid sequence ATNHQANYGQAQTG (SEQ ID NO: 192) at the VR-VIII positions 581-594, and amino acid K at the VR-IV position 452;
(xiii) amino acid sequence ATNHNMNRVNAQTG (SEQ ID NO: 193) at the VR-VIII positions 581-594, and amino acid N at the VR-IV position 452;
(xiv) amino acid sequence ATNHNNVISGAQTG (SEQ ID NO: 190) at the VR-VIII positions 581-594, and amino acid N at the VR-IV position 452;
(xv) amino acid sequence ATNHSNSVQSAQTG (SEQ ID NO: 194) at the VR-VIII positions 581-594, and amino acid N at the VR-IV position 452;
(xvi) amino acid sequence ATNHSSTFQGAQTG (SEQ ID NO: 195) at the VR-VIII positions 581-594, and amino acid N at the VR-IV position 452;
(xvii) amino acid sequence ATNHVSSFTSAQTG (SEQ ID NO: 196) at the VR-VIII positions 581-594, and amino acid N at the VR-IV position 452;
(xviii) amino acid sequence ATNHSTTNFRAQTG (SEQ ID NO: 197) at the VR-VIII positions 581-594, and amino acid N at the VR-IV position 452;
(xix) amino acid sequence ATNHSSIFNSAQTG (SEQ ID NO: 198) at the VR-VIII positions 581-594, and amino acid N at the VR-IV position 452;
(xx) amino acid sequence ATNHAGNYNNAQTG (SEQ ID NO: 199) at the VR-VIII positions 581-594, and amino acid N at the VR-IV position 452;
(xxi) amino acid sequence ATNHTSVISIAQTG (SEQ ID NO: 200) at the VR-VIII positions 581-594, and amino acid N at the VR-IV position 452;
(xxii) amino acid sequence ATNHHSRVEIAQTG (SEQ ID NO: 201) at the VR-VIII positions 581-594, and amino acid N at the VR-IV position 452;
(xxiii) amino acid sequence ATNHSSIIYSAQTG (SEQ ID NO: 202) at the VR-VIII positions 581-594, and amino acid N at the VR-IV position 452;
(xxiv) amino acid sequence ATNHSGRDSYAQTG (SEQ ID NO: 203) at the VR-VIII positions 581-594, and amino acid N at the VR-IV position 452;
(xxv) amino acid sequence ATNHSSSYNNAQTG (SEQ ID NO: 204) at the VR-VIII positions 581-594, and amino acid N at the VR-IV position 452;
(xxvi) amino acid sequence ATNHHNPSINAQTG (SEQ ID NO: 205) at the VR-VIII positions 581-594, and amino acid N at the VR-IV position 452;
(xxvii) amino acid sequence ATNHNRNGLLAQTG (SEQ ID NO: 206) at the VR-VIII positions 581-594, and amino acid N at the VR-IV position 452;
(xxviii) amino acid sequence ATNHESTSVRAQTG (SEQ ID NO: 207) at the VR-VIII positions 581-594, and amino acid N at the VR-IV position 452;
(xxix) amino acid sequence ATNHNIRTEMAQTG (SEQ ID NO: 208) at the VR-VIII positions 581-594, and amino acid N at the VR-IV position 452;
(xxx) amino acid sequence ATNHQTLFNSAQTG (SEQ ID NO: 184) at the VR-VIII positions 581-594, and amino acid N at the VR-IV position 452;
(xxxi) amino acid sequence ATNHLSVSSIAQTG (SEQ ID NO: 209) at the VR-VIII positions 581-594, and amino acid N at the VR-IV position 452;
(xxxii) amino acid sequence ATNHEDIIRSAQTG (SEQ ID NO: 210) at the VR-VIII positions 581-594, and amino acid N at the VR-IV position 452;
(xxxiii) amino acid sequence ATNRQTAQAQAQTG (SEQ ID NO: 211) at the VR-VIII positions 581-594, and amino acid N at the VR-IV position 452; or
(xxxiv) amino acid sequence ATNRQIAQAQAQTG (SEQ ID NO: 212) at the VR-VIII positions 581-594, and amino acid N at the VR-IV position 452.

In some embodiments, the capsid protein comprises a variant polypeptide sequence at the VR-VIII site (e.g., positions 581-594 relative to reference sequence SEQ ID NO: 112), wherein the VR-VIII site (e.g., the entire VR-VIII site) comprises, consists essentially of, or consists of, a sequence having at least about 60%, 65%, 70%, 71%, 74%, 75%, 78%, 78.5%, 79%, 80%, 83%, 85%, 86%, 90%, 92%, 93% or 100% identity to any one of the following sequences provided in Table 7A (e.g., with at most 1, 2, or 3 amino acid substitutions relative to any one of the following sequences):

TABLE 7A
Exemplary VR-VIII variant sequences

| VR-VIII Alignment | SEQ ID |
|---|---|
| (581-594) | NO: |
| ATNHENTVSIAQTG | 183 |
| ATNHQTLFNSAQTG | 184 |
| ATNHNSTYLGAQTG | 185 |
| ATNHGSILTHAQTG | 186 |
| ATNHMMTTARAQTG | 187 |
| ATNHCSTSIRAQTG | 188 |
| ATNHEDNIRSAQTG | 189 |
| ATNHNNVISGAQTG | 190 |
| ATNHQGAYAQAQTG | 191 |
| ATNHQANYGQAQTG | 192 |
| ATNHNMNRVNAQTG | 193 |
| ATNHSNSVQSAQTG | 194 |
| ATNHSSTFQGAQTG | 195 |
| ATNHVSSFTSAQTG | 196 |
| ATNHSTTNFRAQTG | 197 |
| ATNHSSIFNSAQTG | 198 |
| ATNHAGNYNNAQTG | 199 |
| ATNHTSVISIAQTG | 200 |
| ATNHHSRVEIAQTG | 201 |
| ATNHSSIIYSAQTG | 202 |
| ATNHSGRDSYAQTG | 203 |
| ATNHSSSYNNAQTG | 204 |
| ATNHHNPSINAQTG | 205 |
| ATNHNRNGLLAQTG | 206 |
| ATNHESTSVRAQTG | 207 |
| ATNHNIRTEMAQTG | 208 |
| ATNHLSVSSIAQTG | 209 |
| ATNHEDIIRSAQTG | 210 |
| ATNRQTAQAQAQTG | 211 |
| ATNRQIAQAQAQTG | 212 |
| ATNHNTKLAIAQTG | 213 |
| ATNHTGTSIIAQTG | 214 |
| ATNHQWMSAQAQAQTG | 215 |
| ATNHQDARAQAQTG | 216 |
| ATNHQHYSAQAQAQTG | 217 |

In some embodiments, the rAAV virions of the disclosure comprise a capsid protein comprising an amino acid sequence of X₁DVQX₂X₃PGFX₄X₅X₆X₇X₈ (SEQ ID NO: 218) at the VR-VIII site, wherein each of X₁, X₂, X₃, X₄, X₅, X₆, X₇, and X₈ is any amino acid (e.g., wherein the rAAV virions are any one of: AAV9, AAV5, AAVrh.10, AAVrh.74, or a variant thereof).

In some embodiments, X₁ is alanine (A).

In some embodiments, X₂ is glutamine (Q).

In some embodiments, X₇ is threonine (T).

In some embodiments, X₅ is alanine (A) or proline (P).

In some embodiments, X₆ is glutamine (Q) or glutamic acid (E).

In some embodiments, X₄ is glutamine (Q), glycine (G), arginine (R), asparagine (N), histidine (H), methionine (M), proline (P), or serine (S).

In some embodiments, X₈ is glutamic acid (E), methionine (M), glutamine (Q), aspartic acid (D), leucine (L), alanine (A), cysteine (C), histidine (H), phenylalanine (F), tyrosine (Y), threonine (T), valine (V), isoleucine (I), serine (S), or asparagine (N). In some embodiments, X₈ is glutamic acid (E).

In some embodiments, X₃ is leucine (L), histidine (H), valine (V), cysteine (C), glutamine (Q), glycine (G), isoleucine (I), methionine (M), phenylalanine (F), proline (P), threonine (T), or tyrosine (Y).

In some embodiments, X₁ is A, X₂ is Q, X₇ is T, and/or the capsid protein comprises in the VR-VIII site an amino acid sequence of ADVQQX₃PGFX₄X₅X₆TX₈ (SEQ ID NO: 219), wherein each of X₃, X₄, X₅, X₆, and X₈ is any amino acid.

In some embodiments, X₅ is A or P, and X₆ is Q or E, and/or the capsid protein comprises in the VR-VIII site an amino acid sequence of X₁DVQX₂X₃PGFX₄AQX₇X₈ (SEQ ID NO: 220), X₁DVQX₂X₃PGFX₄AEX₇X₈ (SEQ ID NO: 221), X₁DVQX₂X₃PGFX₄PQX₇X₈ (SEQ ID NO: 222), X₁DVQX₂X₃PGFX₄PEX₇X₈ (SEQ ID NO: 223), ADVQQX₃PGFX₄AQTX₈ (SEQ ID NO: 224), ADVQQX₃PGFX₄AETX₈ (SEQ ID NO: 225), ADVQQX₃PGFX₄PQTX₈ (SEQ ID NO: 226), or ADVQQX₃PGFX₄PETX₈ (SEQ ID NO: 227), wherein each of X₁, X₂, X₃, X₄, X₇, and X₈ is any amino acid.

In some embodiments, X₄ is Q, X₅ is A, and X₆ is Q, and/or the capsid protein comprises in the VR-VIII site an amino acid sequence of X₁DVQX₂X₃PGFQAQX₇X₈ (SEQ ID NO: 228) or ADVQQX₃PGFQAQTX₈ (SEQ ID NO: 229), wherein each of X₁, X₂, X₃, X₇, and X₈ is any amino acid.

In some embodiments, X₃ is L, and X₈ is E, and/or the capsid protein comprises in the VR-VIII site an amino acid sequence of X₁DVQX₂LPGFX₄X₅X₆X₇E (SEQ ID NO: 230) or ADVQQLPGFX₄X₅X₆TE (SEQ ID NO: 231), wherein each of X₁, X₂, X₄, X₅, X₆, and X₇ is any amino acid.

In some embodiments, X₄ is Q, and/or the capsid protein comprises in the VR-VIII site an amino acid sequence of X₁DVQX₂X₃PGFQX₅X₆X₇X₈ (SEQ ID NO: 232) or ADVQQX₃PGFQX₅X₆TX₈ (SEQ ID NO: 233), wherein each of X₁, X₂, X₃, X₅, X₆, X₇, and X₈ is any amino acid.

In some embodiments, the capsid protein comprises, consists essentially of, or consists of a sequence having at least about 80% (e.g., at least 80%, 85%, 90%, 95%, 96%, 97%, 98%, 99%, or 100%) identity to any one of the following sequences provided in Table 7B at the VR-VIII site (positions 581-594 relative to reference sequence SEQ ID NO: 112), with up to 1, 2, or 3 substitutions:

TABLE 7B
Exemplary VR-VIII variant sequences

| VR-VIII Alignment | SEQ ID |
|---|---|
| (581-594) | NO: |
| ADVQQLPGFQAQTE | 234 |
| ADVQQHPGFQAQTE | 235 |
| ADVQQLPGFGAQTE | 236 |
| ADVQQLPGFRPETE | 237 |
| ADVQQLPGFNAQTE | 238 |
| ADVQQLPGFQAETE | 239 |
| ADVQQLPGFGAETE | 240 |
| ADVQQLPGFGPQTE | 24 |
| ADVQQLPGFHAQTE | 242 |
| ADVQQLPGFRAQTE | 243 |
| ADVQQLPGFRAETE | 244 |
| ADVQQLPGFMAQTE | 245 |
| ADVQQLPGFPAQTE | 246 |
| ADVQQLPGFSAQTE | 247 |
| ADVQQLPGFSAETE | 248 |
| ADVQQLPGFRPQTE | 249 |
| ADVQQVPGFQAQTM | 250 |
| ADVQQVPGFQAQTQ | 251 |
| ADVQQCPGFQAQTE | 252 |
| ADVQQQPGFQAQTE | 253 |
| ADVQQGPGFQAQTD | 254 |
| ADVQQHPGFQAQTD | 255 |
| ADVQQHPGFQAQTL | 256 |
| ADVQQIPGFQAQTA | 257 |
| ADVQQIPGFQAQTC | 258 |
| ADVQQIPGFQAQTQ | 259 |
| ADVQQIPGFQAQTH | 260 |
| ADVQQIPGFQAQTM | 261 |
| ADVQQLPGFQAQTD | 262 |
| ADVQQLPGFQAQTC | 263 |
| ADVQQLPGFQAQTQ | 264 |
| ADVQQLPGFQAQTL | 265 |
| ADVQQLPGFQAQTM | 266 |
| ADVQQMPGFQAQTD | 267 |
| ADVQQMPGFQAQTE | 268 |
| ADVQQMPGFQAQTL | 269 |
| ADVQQMPGFQAQTF | 270 |
| ADVQQMPGFQAQTY | 271 |
| ADVQQFPGFQAQTA | 272 |
| ADVQQFPGFQAQTQ | 273 |
| ADVQQFPGFQAQTH | 274 |
| ADVQQFPGFQAQTT | 275 |
| ADVQQFPGFQAQTV | 276 |
| ADVQQPPGFQAQTE | 277 |
| ADVQQTPGFQAQTE | 278 |
| ADVQQYPGFQAQTA | 279 |
| ADVQQYPGFQAQTC | 280 |
| ADVQQYPGFQAQTQ | 281 |
| ADVQQYPGFQAQTH | 282 |
| ADVQQYPGFQAQTI | 283 |
| ADVQQYPGFQAQTM | 284 |
| ADVQQYPGFQAQTS | 285 |
| ADVQQYPGFQAQTT | 286 |
| ADVQQYPGFQAQTV | 287 |
| ADVQQVPGFQAQTA | 288 |
| ADVQQVPGFQAQTN | 289 |
| ADVQQVPGFQAQTD | 290 |
| ADVQQVPGFQAQTC | 291 |
| ADVQQVPGFQAQTH | 292 |
| ADVQQVPGFQAQTL | 293 |

In some aspects, the rAAV capsid protein shares at least 80%, at least 85%, at least 90%, or at least 95% polypeptide sequence identity to an AAV9 VP3 reference sequence according to SEQ ID NO: 114, wherein the capsid protein comprises, relative to reference sequence SEQ ID NO: 112: (i) an amino acid sequence X₁DVQX₂X₃PGFX₄X₅X₆X₇X₈ (SEQ ID NO: 218) at the VR-VIII site or at positions 581 to 594, wherein each of X₁, X₂, X₃, X₄, X₅, X₆, X₇, and X₈ is any amino acid; or (ii) amino acid substitutions T582D, N583V, H584Q, A587P, Q588G, and A589F. In some embodiments, X₁ is alanine (A). In some embodiments, X₂ is glutamine (Q). In some embodiments, X₇ is threonine (T). In some embodiments, X₅ is alanine (A) or proline (P). In some embodiments, X₆ is glutamine (Q) or glutamic acid (E). In some embodiments, X₄ is glutamine (Q), glycine (G), arginine (R), asparagine (N), histidine (H), methionine (M), proline (P), or serine (S). In some embodiments, X₈ is glutamic acid (E), methionine (M), glutamine (Q), aspartic acid (D), leucine (L), alanine (A), cysteine (C), histidine (H), phenylalanine (F), tyrosine (Y), threonine (T), valine (V), isoleucine (I), serine (S), or asparagine (N). In some embodiments, X₃ is leucine (L), histidine (H), valine (V), cysteine (C), glutamine (Q), glycine (G), isoleucine (I), methionine (M), phenylalanine (F), proline (P), threonine (T), or tyrosine (Y).

In some embodiments, the capsid protein comprises any substitution and/or insertion motif described herein. In some embodiments, the capsid protein comprises a substitution motif having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 95%, 96%, 97%, 98%, 99%, or 100% sequence identity to any substitution motif described herein. In some embodiments, the capsid protein comprises an insertion motif having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 95%, 96%, 97%, 98%, 99%, or 100% sequence identity to any insertion motif described herein.

It should be noted that the above modified VR-VIII motifs are described in the context of AAV9 capsid proteins for illustrative purposes only and are not meant to be limited to AAV9 capsid proteins. Instead, any modified VR-VIII motif described herein can be applied to other AAV capsid proteins of a different serotype (e.g., AAV5, AAVrh.10, or AAVrh.74), for example, by replacing the wild type sequence at the VR-VIII site of the corresponding capsid protein (e.g., amino acid positions 570 to 583 of AAV5 VP1 capsid protein sequence according to SEQ ID NO: 294, amino acid positions 581 to 594 of AAV9 VP1 capsid protein sequence according to SEQ ID NO: 112, amino acid positions 583 to 596 of AAVrh.10 VP1 capsid protein sequence according to SEQ ID NO: 298, or amino acid positions 583 to 596 of AAVrh.74 VP1 capsid protein sequence according to SEQ ID NO: 302) with any of the modified VR-VIII motifs described herein to generate a variant of the capsid protein of a particular serotype. In some embodiments, the capsid protein is a variant of an AAV5, AAV9, AAVth.10, or AAVrh.74 capsid protein.

TABLE 8
Exemplary AAV5, AAVrh.10, and AAVrh.74
sequences

| | | SEQ |
|---|---|---|
| | | ID |
| Name | Sequence | NO: |
| AAV5 | MSFVDHPPDWLEEVGEGLREFLGLEAGPPKPKPNQ | 294 |
| capsid | QHQDQARGLVLPGYNYLGPGNGLDRGEPVNRADEV | |
| VP1 | AREHDISYNEQLEAGDNPYLKYNHADAEFQEKLAD | |
| | DTSFGGNLGKAVFQAKKRVLEPFGLVEEGAKTAPT | |
| | GKRIDDHFPKRKKARTEEDSKPSTSSDAEAGPSGS | |
| | QQLQIPAQPASSLGADTMSAGGGGPLGDNNQGADG | |
| | VGNASGDWHCDSTWMGDRVVTKSTRTWVLPSYNNH | |
| | QYREIKSGSVDGSNANAYFGYSTPWGYFDFNRFHS | |
| | HWSPRDWQRLINNYWGFRPRSLRVKIFNIQVKEVT | |
| | VQDSTTTIANNLTSTVQVFTDDDYQLPYVVGNGTE | |
| | GCLPAFPPQVFTLPQYGYATLNRDNTENPTERSSF | |
| | FCLEYFPSKMLRTGNNFEFTYNFEEVPFHSSFAPS | |
| | QNLFKLANPLVDQYLYRFVSTNNTGGVQFNKNLAG | |
| | RYANTYKNWFPGPMGRTQGWNLGSGVNRASVSAFA | |
| | TTNRMELEGASYQVPPQPNGMTNNLQGSNTYALEN | |
| | TMIFNSQPANPGTTATYLEGNMLITSESETQPVNR | |
| | VAYNVGGQMATNNQSSTTAPATGTYNLQEIVPGSV | |
| | WMERDVYLQGPIWAKIPETGAHFHPSPAMGGFGLK | |
| | HPPPMMLIKNTPVPGNITSFSDVPVSSFITQYSTG | |
| | QVTVEMEWELKKENSKRWNPEIQYTNNYNDPQFVD | |
| | FAPDSTGEYRTTRPIGTRYLTRPL | |
| AAV5 | TAPTGKRIDDHFPKRKKARTEEDSKPSTSSDAEAG | 295 |
| capsid | PSGSQQLQIPAQPASSLGADTMSAGGGGPLGDNNQ | |
| VP2 | GADGVGNASGDWHCDSTWMGDRVVTKSTRTWVLPS | |
| | YNNHQYREIKSGSVDGSNANAYFGYSTPWGYFDFN | |
| | RFHSHWSPRDWQRLINNYWGFRPRSLRVKIFNIQV | |
| | KEVTVQDSTTTIANNLTSTVQVFTDDDYQLPYVVG | |
| | NGTEGCLPAFPPQVFTLPQYGYATLNRDNTENPTE | |
| | RSSFFCLEYFPSKMLRTGNNFEFTYNFEEVPFHSS | |
| | FAPSQNLFKLANPLVDQYLYRFVSTNNTGGVQFNK | |
| | NLAGRYANTYKNWFPGPMGRTQGWNLGSGVNRASV | |
| | SAFATTNRMELEGASYQVPPQPNGMTNNLQGSNTY | |
| | ALENTMIFNSQPANPGTTATYLEGNMLITSESETQ | |
| | PVNRVAYNVGGQMATNNQSSTTAPATGTYNLQEIV | |
| | PGSVWMERDVYLQGPIWAKIPETGAHFHPSPAMGG | |
| | FGLKHPPPMMLIKNTPVPGNITSFSDVPVSSFITQ | |
| | YSTGQVTVEMEWELKKENSKRWNPEIQYTNNYNDP | |
| | QFVDFAPDSTGEYRTTRPIGTRYLTRPL | |
| AAV5 | MSAGGGGPLGDNNQGADGVGNASGDWHCDSTWMGD | 296 |
| capsid | RVVTKSTRTWVLPSYNNHQYREIKSGSVDGSNANA | |
| VP3 | YFGYSTPWGYFDFNRFHSHWSPRDWQRLINNYWGF | |
| | RPRSLRVKIFNIQVKEVTVQDSTTTIANNLTSTVQ | |
| | VFTDDDYQLPYVVGNGTEGCLPAFPPQVFTLPQYG | |
| | YATLNRDNTENPTERSSFFCLEYFPSKMLRTGNNF | |
| | EFTYNFEEVPFHSSFAPSQNLFKLANPLVDQYLYR | |
| | FVSTNNTGGVQFNKNLAGRYANTYKNWFPGPMGRT | |
| | QGWNLGSGVNRASVSAFATTNRMELEGASYQVPPQ | |
| | PNGMTNNLQGSNTYALENTMIFNSQPANPGTTATY | |
| | LEGNMLITSESETQPVNRVAYNVGGQMATNNQSST | |
| | TAPATGTYNLQEIVPGSVWMERDVYLQGPIWAKIP | |
| | ETGAHFHPSPAMGGFGLKHPPPMMLIKNTPVPGNI | |
| | TSFSDVPVSSFITQYSTGQVTVEMEWELKKENSKR | |
| | WNPEIQYTNNYNDPQFVDFAPDSTGEYRTTRPIGT | |
| | RYLTRPL | |
| AAV5 | ATNNQSSTTAPATG | 297 |
| VR-VIII | | |
| AAVrh.10 | MAADGYLPDWLEDNLSEGIREWWDLKPGAPKPKAN | 298 |
| capsid | QQKQDDGRGLVLPGYKYLGPFNGLDKGEPVNAADA | |
| VP1 | AALEHDKAYDQQLKAGDNPYLRYNHADAEFQERLQ | |
| | EDTSFGGNLGRAVFQAKKRVLEPLGLVEEGAKTAP | |
| | GKKRPVEPSPQRSPDSSTGIGKKGQQPAKKRLNFG | |
| | QTGDSESVPDPQPIGEPPAGPSGLGSGTMAAGGGA | |
| | PMADNNEGADGVGSSSGNWHCDSTWLGDRVITTST | |
| | RTWALPTYNNHLYKQISNGTSGGSTNDNTYFGYST | |
| | PWGYFDFNRFHCHFSPRDWQRLINNNWGFRPKRLN | |
| | FKLFNIQVKEVTQNEGTKTIANNLTSTIQVFTDSE | |
| | YQLPYVLGSAHQGCLPPFPADVFMIPQYGYLTLNN | |
| | GSQAVGRSSFYCLEYFPSQMLRTGNNFEFSYQFED | |
| | VPFHSSYAHSQSLDRLMNPLIDQYLYYLSRTQSTG | |
| | GTAGTQQLLFSQAGPNNMSAQAKNWLPGPCYRQQR | |
| | VSTTLSQNNNSNFAWTGATKYHLNGRDSLVNPGVA | |
| | MATHKDDEERFFPSSGVLMFGKQGAGKDNVDYSSV | |
| | MLTSEEEIKTTNPVATEQYGVVADNLQQQNAAPIV | |
| | GAVNSQGALPGMVWQNRDVYLQGPIWAKIPHTDGN | |
| | FHPSPLMGGFGLKHPPPQILIKNTPVPADPPTTFS | |
| | QAKLASFITQYSTGQVSVEIEWELQKENSKRWNPE | |
| | IQYTSNYYKSTNVDFAVNTDGTYSEPRPIGTRYLT | |
| | RNL | |
| AAVrh.10 | TAPGKKRPVEPSPQRSPDSSTGIGKKGQQPAKKRL | 299 |
| capsid | NFGQTGDSESVPDPQPIGEPPAGPSGLGSGTMAAG | |
| VP2 | GGAPMADNNEGADGVGSSSGNWHCDSTWLGDRVIT | |
| | TSTRTWALPTYNNHLYKQISNGTSGGSTNDNTYFG | |
| | YSTPWGYFDFNRFHCHFSPRDWQRLINNNWGFRPK | |
| | RLNFKLFNIQVKEVTQNEGTKTIANNLTSTIQVFT | |
| | DSEYQLPYVLGSAHQGCLPPFPADVFMIPQYGYLT | |
| | LNNGSQAVGRSSFYCLEYFPSQMLRTGNNFEFSYQ | |
| | FEDVPFHSSYAHSQSLDRLMNPLIDQYLYYLSRTQ | |
| | STGGTAGTQQLLFSQAGPNNMSAQAKNWLPGPCYR | |
| | QQRVSTTLSQNNNSNFAWTGATKYHLNGRDSLVNP | |
| | GVAMATHKDDEERFFPSSGVLMFGKQGAGKDNVDY | |
| | SSVMLTSEEEIKTTNPVATEQYGVVADNLQQQNAA | |
| | PIVGAVNSQGALPGMVWQNRDVYLQGPIWAKIPHT | |
| | DGNFHPSPLMGGFGLKHPPPQILIKNTPVPADPPT | |
| | TFSQAKLASFITQYSTGQVSVEIEWELQKENSKRW | |
| | NPEIQYTSNYYKSTNVDFAVNTDGTYSEPRPIGTR | |
| | YLTRNL | |
| AAVrh.10 | MAAGGGAPMADNNEGADGVGSSSGNWHCDSTWLGD | 300 |
| capsid | RVITTSTRTWALPTYNNHLYKQISNGTSGGSTNDN | |
| VP3 | TYFGYSTPWGYFDFNRFHCHFSPRDWQRLINNNWG | |
| | FRPKRLNFKLFNIQVKEVTQNEGTKTIANNLTSTI | |
| | QVFTDSEYQLPYVLGSAHQGCLPPFPADVFMIPQY | |
| | GYLTLNNGSQAVGRSSFYCLEYFPSQMLRTGNNFE | |
| | FSYQFEDVPFHSSYAHSQSLDRLMNPLIDQYLYYL | |
| | SRTQSTGGTAGTQQLLFSQAGPNNMSAQAKNWLPG | |
| | PCYRQQRVSTTLSQNNNSNFAWTGATKYHLNGRDS | |
| | LVNPGVAMATHKDDEERFFPSSGVLMFGKQGAGKD | |
| | NVDYSSVMLTSEEEIKTTNPVATEQYGVVADNLQQ | |
| | QNAAPIVGAVNSQGALPGMVWQNRDVYLQGPIWAK | |
| | IPHTDGNFHPSPLMGGFGLKHPPPQILIKNTPVPA | |
| | DPPTTFSQAKLASFITQYSTGQVSVEIEWELQKEN | |
| | SKRWNPEIQYTSNYYKSTNVDFAVNTDGTYSEPRP | |
| | IGTRYLTRNL | |
| AAVrh.10 | ADNLQQQNAAPIVG | 301 |
| VR-VIII | | |
| AAVrh.74 | MAADGYLPDWLEDNLSEGIREWWDLKPGAPKPKAN | 302 |
| capsid | QQKQDNGRGLVLPGYKYLGPFNGLDKGEPVNAADA | |
| VP1 | AALEHDKAYDQQLQAGDNPYLRYNHADAEFQERLQ | |
| | EDTSFGGNLGRAVFQAKKRVLEPLGLVESPVKTAP | |
| | GKKRPVEPSPQRSPDSSTGIGKKGQQPAKKRLNFG | |
| | QTGDSESVPDPQPIGEPPAGPSGLGSGTMAAGGGA | |
| | PMADNNEGADGVGSSSGNWHCDSTWLGDRVITTST | |
| | RTWALPTYNNHLYKQISNGTSGGSTNDNTYFGYST | |
| | PWGYFDFNRFHCHFSPRDWQRLINNNWGFRPKRLN | |
| | FKLFNIQVKEVTQNEGTKTIANNLTSTIQVFTDSE | |
| | YQLPYVLGSAHQGCLPPFPADVFMIPQYGYLTLNN | |
| | GSQAVGRSSFYCLEYFPSQMLRTGNNFEFSYNFED | |
| | VPFHSSYAHSQSLDRLMNPLIDQYLYYLSRTQSTG | |
| | GTAGTQQLLFSQAGPNNMSAQAKNWLPGPCYRQQR | |
| | VSTTLSQNNNSNFAWTGATKYHLNGRDSLVNPGVA | |
| | MATHKDDEERFFPSSGVLMFGKQGAGKDNVDYSSV | |
| | MLTSEEEIKTTNPVATEQYGVVADNLQQQNAAPIV | |
| | GAVNSQGALPGMVWQNRDVYLQGPIWAKIPHTDGN | |
| | FHPSPLMGGFGLKHPPPQILIKNTPVPADPPTTFN | |
| | QAKLASFITQYSTGQVSVEIEWELQKENSKRWNPE | |
| | IQYTSNYYKSTNVDFAVNTEGTYSEPRPIGTRYLT | |
| | RNL | |
| AAVrh.74 | TAPGKKRPVEPSPQRSPDSSTGIGKKGQQPAKKRL | 303 |
| capsid | NFGQTGDSESVPDPQPIGEPPAGPSGLGSGTMAAG | |
| VP2 | GGAPMADNNEGADGVGSSSGNWHCDSTWLGDRVIT | |
| | TSTRTWALPTYNNHLYKQISNGTSGGSTNDNTYFG | |
| | YSTPWGYFDFNRFHCHFSPRDWQRLINNNWGFRPK | |
| | RLNFKLFNIQVKEVTQNEGTKTIANNLTSTIQVFT | |
| | DSEYQLPYVLGSAHQGCLPPFPADVFMIPQYGYLT | |
| | LNNGSQAVGRSSFYCLEYFPSQMLRTGNNFEFSYN | |
| | FEDVPFHSSYAHSQSLDRLMNPLIDQYLYYLSRTQ | |
| | STGGTAGTQQLLFSQAGPNNMSAQAKNWLPGPCYR | |
| | QQRVSTTLSQNNNSNFAWTGATKYHLNGRDSLVNP | |
| | GVAMATHKDDEERFFPSSGVLMFGKQGAGKDNVDY | |
| | SSVMLTSEEEIKTTNPVATEQYGVVADNLQQQNAA | |
| | PIVGAVNSQGALPGMVWQNRDVYLQGPIWAKIPHT | |
| | DGNFHPSPLMGGFGLKHPPPQILIKNTPVPADPPT | |
| | TFNQAKLASFITQYSTGQVSVEIEWELQKENSKRW | |
| | NPEIQYTSNYYKSTNVDFAVNTEGTYSEPRPIGTR | |
| | YLTRNL | |
| AAVrh.74 | MAAGGGAPMADNNEGADGVGSSSGNWHCDSTWLGD | 304 |
| capsid | RVITTSTRTWALPTYNNHLYKQISNGTSGGSTNDN | |
| VP3 | TYFGYSTPWGYFDFNRFHCHFSPRDWQRLINNNWG | |
| | FRPKRLNFKLFNIQVKEVTQNEGTKTIANNLTSTI | |
| | QVFTDSEYQLPYVLGSAHQGCLPPFPADVFMIPQY | |
| | GYLTLNNGSQAVGRSSFYCLEYFPSQMLRTGNNFE | |
| | FSYNFEDVPFHSSYAHSQSLDRLMNPLIDQYLYYL | |
| | SRTQSTGGTAGTQQLLFSQAGPNNMSAQAKNWLPG | |
| | PCYRQQRVSTTLSQNNNSNFAWTGATKYHLNGRDS | |
| | LVNPGVAMATHKDDEERFFPSSGVLMFGKQGAGKD | |
| | NVDYSSVMLTSEEEIKTTNPVATEQYGVVADNLQQ | |
| | QNAAPIVGAVNSQGALPGMVWQNRDVYLQGPIWAK | |
| | IPHTDGNFHPSPLMGGFGLKHPPPQILIKNTPVPA | |
| | DPPTTFNQAKLASFITQYSTGQVSVEIEWELQKEN | |
| | SKRWNPEIQYTSNYYKSTNVDFAVNTEGTYSEPRP | |
| | IGTRYLTRNL | |
| AAVrh.74 | ADNLQQQNAAPIVG | 301 |
| VR- | | |
| VIII | | |

In some embodiments, the capsid protein comprises an insertion polypeptide or insertion motif compared to the wild-type or parental capsid protein. In some embodiments, the capsid protein additionally comprises one or more amino acid substitutions in the amino acid sequence of the wild-type or parental capsid protein sequence from which it is derived. In some embodiments, the insertion motif is inserted at a surface loop region of the capsid protein, for example, at a VR-I, VR-II, VR-IV, VR-V, VR-VII and/or VR-VIII site, as described.

In some embodiments, the insertion motif comprises or consists of an amino acid sequence of “RGDAARL” (SEQ ID NO: 373); and/or the engineered capsid protein comprises an amino acid sequence of “RGDAARL” (SEQ ID NO: 373).

In some embodiments, the insertion motif comprises or consists of an amino acid sequence of “SHVRGDL” (SEQ ID NO: 380); and/or the engineered capsid protein comprises an amino acid sequence of “SHVRGDL” (SEQ ID NO: 380).

In some embodiments, the insertion motif comprises or consists of an amino acid sequence of “VVSSGAR” (SEQ ID NO: 381); and/or the engineered capsid protein comprises an amino acid sequence of “VVSSGAR” (SEQ ID NO: 381).

In some embodiments, the insertion motif comprises or consists of an amino acid sequence of “VRGD” (SEQ ID NO: 397); and/or the engineered capsid protein comprises an amino acid sequence of “VRGD” (SEQ ID NO: 397).

The insertion motif can occur (e.g., be inserted) at any position of the capsid protein, for example, at a surface or an exposed region of the capsid protein. In some embodiments, the engineered AAV capsid protein comprises an insertion motif as described herein inserted at a surface loop region of the capsid protein, e.g., the VR-I, VR-II, VR-IV, VR-V, VR-VII, and/or VR-VIII site of the capsid protein. In some embodiments, the engineered AAV capsid protein comprises an insertion motif as described inserted at the VR-IV and/or the VR-VIII site of the capsid protein. In certain of these embodiments, the engineered capsid protein additionally comprises one or more amino acid substitutions in the same VR site as the insertion or at a different location from the insertion.

In some embodiments, the engineered AAV capsid protein is an engineered AAV9 capsid protein comprises an insertion motif as described herein inserted into the wild-type AAV9 VP1 (SEQ ID NO: 112), AAV9 VP2 (SEQ ID NO: 113), or AAV9 VP3 (SEQ ID NO: 114); and optionally further comprises one or more amino acid substitutions. In some embodiments, the insertion motif as described herein is inserted into anywhere within the VR-I site (between amino acids 262 and 269 of the parental sequence of SEQ ID NO: 112); anywhere with the VR-II site (between amino acids 328 and 332 of the parental sequence of SEQ ID NO: 112); anywhere within the VR-IV site (between amino acids 448 and 462 of the parental sequence of SEQ ID NO: 112); anywhere within the VR-V site (between amino acids 491 and 504 of the parental sequence of SEQ ID NO: 112); anywhere within the VR-VII site (between amino acids 547 and 557 of the parental sequence of SEQ ID NO: 112); and/or anywhere within the VR-VIII site (between amino acids 581 and 595 of the parental sequence of SEQ ID NO: 112). In some embodiments, the insertion motif as described herein is inserted into anywhere within the VR-IV site (between amino acids 448 and 462 of the parental sequence of SEQ ID NO: 112); and/or anywhere within the VR-VIII site (between amino acids 581 and 595 of the parental sequence of SEQ ID NO: 112). In some embodiments, the insertion motif as described herein is inserted into anywhere within the VR-IV site (between amino acids 448 and 462 of the parental sequence of SEQ ID NO: 112). In some embodiments, the insertion motif as described herein is inserted into anywhere within the VR-VIII site (between amino acids 581 and 595 of the parental sequence of SEQ ID NO: 112). In any of the above embodiments, the engineered AAV9 capsid protein additionally comprises one or more amino acid substitutions in the same VR site as or a different location from the insertion.

In some embodiments, the engineered AAV capsid protein is an engineered AAV5 capsid protein comprises an insertion motif as described herein inserted into the wild-type AAV5 VP1 (SEQ ID NO: 294), AAV5 VP2 (SEQ ID NO: 295), or AAV5 VP3 (SEQ ID NO: 296); and optionally further comprises one or more amino acid substitutions. In some embodiments, the insertion motif as described herein is inserted into anywhere within the VR-I site (between amino acids 252 and 256 of the parental sequence of SEQ ID NO: 294); anywhere with the VR-II site (between amino acids 317 and 321 of the parental sequence of SEQ ID NO: 294); anywhere within the VR-IV site (between amino acids 437 and 461 of the parental sequence of SEQ ID NO: 294); anywhere within the VR-V site (between amino acids 477 and 490 of the parental sequence of SEQ ID NO: 294); anywhere within the VR-VII site (between amino acids 533 and 546 of the parental sequence of SEQ ID NO: 294); and/or anywhere within the VR-VIII site (between amino acids 570 and 584 of the parental sequence of SEQ ID NO: 294). In some embodiments, the insertion motif as described herein is inserted into anywhere within the VR-IV site (between amino acids 437 and 461 of the parental sequence of SEQ ID NO: 294); and/or anywhere within the VR-VIII site (between amino acids 570 and 584 of the parental sequence of SEQ ID NO: 294). In some embodiments, the insertion motif as described herein is inserted into anywhere within the VR-IV site (between amino acids 437 and 461 of the parental sequence of SEQ ID NO: 294). In some embodiments, the insertion motif as described herein is inserted into anywhere within the VR-VIII site (between amino acids 570 and 584 of the parental sequence of SEQ ID NO: 294). In any of the above embodiments, the engineered AAV5 capsid protein additionally comprises one or more amino acid substitutions in the same VR site as or a different location from the insertion.

In some embodiments, the engineered AAV capsid protein is an engineered AAVrh.10 capsid protein comprises an insertion motif as described herein inserted into the wild-type AAVrh.10 VP1 (SEQ ID NO: 298), AAVrh.10 VP2 (SEQ ID NO: 299), or AAVrh.10 VP3 (SEQ ID NO: 300); and optionally further comprises one or more amino acid substitutions. In some embodiments, the insertion motif as described herein is inserted into anywhere within the VR-I site (between amino acids 263 and 267 of the parental sequence of SEQ ID NO: 298); anywhere with the VR-II site (between amino acids 329 and 333 of the parental sequence of SEQ ID NO: 298); anywhere within the VR-IV site (between amino acids 449 and 464 of the parental sequence of SEQ ID NO: 298); anywhere within the VR-V site (between amino acids 493 and 506 of the parental sequence of SEQ ID NO: 298); anywhere within the VR-VII site (between amino acids 549 and 559 of the parental sequence of SEQ ID NO: 298); and/or anywhere within the VR-VIII site (between amino acids 583 and 597 of the parental sequence of SEQ ID NO: 298). In some embodiments, the insertion motif as described herein is inserted into anywhere within the VR-IV site (between amino acids 449 and 464 of the parental sequence of SEQ ID NO: 298); and/or anywhere within the VR-VIII site (between amino acids 583 and 597 of the parental sequence of SEQ ID NO: 298). In some embodiments, the insertion motif as described herein is inserted into anywhere within the VR-IV site (between amino acids 449 and 464 of the parental sequence of SEQ ID NO: 298). In some embodiments, the insertion motif as described herein is inserted into anywhere within the VR-VIII site (between amino acids 583 and 597 of the parental sequence of SEQ ID NO: 298). In any of the above embodiments, the engineered AAVrh.10 capsid protein additionally comprises one or more amino acid substitutions in the same VR site as or a different location from the insertion.

In some embodiments, the engineered AAV capsid protein is an engineered AAVrh.74 capsid protein comprises an insertion motif as described herein inserted into the wild-type AAVrh.74 VP1 (SEQ ID NO: 302), AAVrh.74 VP2 (SEQ ID NO: 303), or AAVrh.74 VP3 (SEQ ID NO: 304); and optionally further comprises one or more amino acid substitutions. In some embodiments, the insertion motif as described herein is inserted into anywhere within the VR-I site (between amino acids 263 and 267 of the parental sequence of SEQ ID NO: 302); anywhere with the VR-II site (between amino acids 329 and 333 of the parental sequence of SEQ ID NO: 302); anywhere within the VR-IV site (between amino acids 449 and 464 of the parental sequence of SEQ ID NO: 302); anywhere within the VR-V site (between amino acids 493 and 506 of the parental sequence of SEQ ID NO: 302); anywhere within the VR-VII site (between amino acids 549 and 559 of the parental sequence of SEQ ID NO: 302); and/or anywhere within the VR-VIII site (between amino acids 583 and 597 of the parental sequence of SEQ ID NO: 302). In some embodiments, the insertion motif as described herein is inserted into anywhere within the VR-IV site (between amino acids 449 and 464 of the parental sequence of SEQ ID NO: 302); and/or anywhere within the VR-VIII site (between amino acids 583 and 597 of the parental sequence of SEQ ID NO: 302). In some embodiments, the insertion motif as described herein is inserted into anywhere within the VR-IV site (between amino acids 449 and 464 of the parental sequence of SEQ ID NO: 302). In some embodiments, the insertion motif as described herein is inserted into anywhere within the VR-VIII site (between amino acids 583 and 597 of the parental sequence of SEQ ID NO: 302). In any of the above embodiments, the engineered AAVrh.74 capsid protein additionally comprises one or more amino acid substitutions in the same VR site as or a different location from the insertion.

In some embodiments, the capsid protein is an engineered AAV9 capsid protein that comprises an insertion polypeptide or insertion motif at the VR-VIII site, e.g., between amino acids 588 (glutamine (Q)) and 589 (alanine (A)) within the VR-VIII site in reference to the wild-type full-length AAV9 capsid protein of SEQ ID NO: 112. In some embodiments, the engineered AAV9 capsid protein further comprises one or more amino acid substitutions within the VR-VIII site, including, for example, at one or more of amino acid positions 587-590 in reference to the wild-type full-length AAV9 capsid protein of SEQ ID NO: 112. In certain of these embodiments, the insertion motif comprises an amino acid sequence of “RGDAARL” (SEQ ID NO: 373), “RTDLKGL” (SEQ ID NO: 374), “YPSTGSG” (SEQ ID NO: 375), “FAGSLTRA” (SEQ ID NO: 376), “DRTLTTR” (SEQ ID NO: 377), “RIAGRDV” (SEQ ID NO: 378), or “SLGSGVR” (SEQ ID NO: 379).

In some embodiments, the capsid protein is an engineered AAV9 capsid protein that comprises an insertion polypeptide or insertion motif at the VR-IV site, e.g., between amino acids 453 (glycine (G)) and 454 (serine (S)), and/or between amino acids 456 (glutamine (Q)) and 457 (asparagine (N)), within the VR-IV site in reference to the wild-type full-length AAV9 capsid protein of SEQ ID NO: 112. In certain of these embodiments, the insertion motif comprises an amino acid sequence of “SHVRGDL” (SEQ ID NO: 380), “VVSSGAR” (SEQ ID NO: 381), “PQYGRGG” (SEQ ID NO: 382), “LQVSRVS” (SEQ ID NO: 383), “VRSYSSN” (SEQ ID NO: 384), “TMRVGSL” (SEQ ID NO: 385), “GAYSRGV” (SEQ ID NO: 386), “LRGGSLG” (SEQ ID NO: 387), or “VYGTGVR” (SEQ ID NO: 388).

In some embodiments, the capsid protein is an engineered AAV5 capsid protein that comprises an insertion polypeptide or insertion motif at the VR-VIII site, e.g., between amino acids 574 (glutamine (Q)) and 575 (serine (S)) within the VR-VIII site in reference to the wild-type full-length AAV5 capsid protein of SEQ ID NO: 294. In certain of these embodiments, the insertion motif comprises an amino acid sequence of “DKLIIVS” (SEQ ID NO: 389), “AEDRTKL” (SEQ ID NO: 390), “LSASASL” (SEQ ID NO: 391), “LADQTKL” (SEQ ID NO: 392), “LLLKLQE” (SEQ ID NO: 393), “ELPVKTG” (SEQ ID NO: 394), “LDLKVVG” (SEQ ID NO: 395), or “RDAVL” (SEQ ID NO: 396).

In some embodiments, the capsid protein shares, or comprises a sequence sharing, at least 80%, at least 85%, at least 90%, at least 95%, at least 96%, at least 97%, at least 98%, at least 99%, or 100% amino acid sequence identity to an AAV5 VP1 sequence according to SEQ ID NO: 294, an AAV5 VP2 sequence according to SEQ ID NO: 295, or an AAV5 VP3 sequence according to SEQ ID NO: 296, except for the specified modifications (e.g., the capsid protein has the specified modifications and, excluding the modified portion of the sequence from sequence comparison, the capsid protein shares the recited sequence identity to the AAV5 VP1, VP2, or VP3 sequence) described herein.

In some embodiments, the capsid protein shares, or comprises a sequence sharing, at least 80%, at least 85%, at least 90%, at least 95%, at least 96%, at least 97%, at least 98%, at least 99%, or 100% amino acid sequence identity to an AAV9 VP1 sequence according to SEQ ID NO: 112, an AAV9 VP2 sequence according to SEQ ID NO: 113, or an AAV9 VP3 sequence according to SEQ ID NO: 114, except for the specified modifications (e.g., the capsid protein has the specified modifications and, excluding the modified portion of the sequence from sequence comparison, the capsid protein shares the recited sequence identity to the AAV9 VP1, VP2, or VP3 sequence) described herein.

In some embodiments, the capsid protein shares, or comprises a sequence sharing, at least 80%, at least 85%, at least 90%, at least 95%, at least 96%, at least 97%, at least 98%, at least 99%, or 100% amino acid sequence identity to an AAVrh.10 VP1 sequence according to SEQ ID NO: 298, an AAVrh.10 VP2 sequence according to SEQ ID NO: 299, or an AAVrh.10 VP3 sequence according to SEQ ID NO: 300, except for the specified modifications (e.g., the capsid protein has the specified modifications and, excluding the modified portion of the sequence from sequence comparison, the capsid protein shares the recited sequence identity to the AAVrh.10 VP1, VP2, or VP3 sequence) described herein.

In some embodiments, the capsid protein shares, or comprises a sequence sharing, at least 80%, at least 85%, at least 90%, at least 95%, at least 96%, at least 97%, at least 98%, at least 99%, or 100% amino acid sequence identity to an AAVrh.74 VP1 sequence according to SEQ ID NO: 302, an AAVrh.74 VP2 sequence according to SEQ ID NO: 303, or an AAVrh.74 VP3 sequence according to SEQ ID NO: 304, except for the specified modifications (e.g., the capsid protein has the specified modifications and, excluding the modified portion of the sequence from sequence comparison, the capsid protein shares the recited sequence identity to the AAVrh.74 VP1, VP2, or VP3 sequence) described herein.

In some embodiments, the capsid protein comprises, consists essentially of, or consists of an amino acid sequence at least 80%, 85%, 90%, 95%, 96%, 97%, 98%, 99%, or 100% identical to any one of the modified capsid protein sequences disclosed herein (e.g., VP1, VP2, or VP3), or a functional fragment thereof.

In some embodiments, the capsid protein comprises, consists essentially of, or consists of a polypeptide sequence of any one of the modified capsid protein sequences disclosed herein (e.g., VP1, VP2, or VP3).

Exemplary capsid protein sequences are provided in Table 9A below. In some embodiments, the capsid protein comprises, consists essentially of, or consists of an amino acid sequence that shares at least 80%, 85%, 90%, 95%, 96%, 97%, 98%, 99%, or 100% sequence identity to any one of SEQ ID NOs: 317-326 and 351-360. In some embodiments, the capsid protein comprises, consists essentially of, or consists of any one of SEQ ID NOs: 317-326 and 351-360. In some embodiments, the capsid protein comprises, consists essentially of, or consists of an amino acid sequence that shares at least 80%, 85%, 90%, 95%, 96%, 97%, 98%, 99%, or 100% sequence identity to any one of SEQ ID NOs: 351. In some embodiments, the capsid protein comprises, consists essentially of, or consists of an amino acid sequence that shares at least 80%, 85%, 90%, 95%, 96%, 97%, 98%, 99%, or 100% sequence identity to any one of SEQ ID NOs: 352. In some embodiments, the capsid protein comprises, consists essentially of, or consists of an amino acid sequence that shares at least 80%, 85%, 90%, 95%, 96%, 97%, 98%, 99%, or 100% sequence identity to any one of SEQ ID NOs: 353. In some embodiments, the capsid protein comprises, consists essentially of, or consists of an amino acid sequence that shares at least 80%, 85%, 90%, 95%, 96%, 97%, 98%, 99%, or 100% sequence identity to any one of SEQ ID NOs: 354. In some embodiments, the capsid protein comprises, consists essentially of, or consists of an amino acid sequence that shares at least 80%, 85%, 90%, 95%, 96%, 97%, 98%, 99%, or 100% sequence identity to any one of SEQ ID NOs: 355. In some embodiments, the capsid protein comprises, consists essentially of, or consists of an amino acid sequence that shares at least 80%, 85%, 90%, 95%, 96%, 97%, 98%, 99%, or 100% sequence identity to any one of SEQ ID NOs: 356. In some embodiments, the capsid protein comprises, consists essentially of, or consists of an amino acid sequence that shares at least 80%, 85%, 90%, 95%, 96%, 97%, 98%, 99%, or 100% sequence identity to any one of SEQ ID NOs: 357. In some embodiments, the capsid protein comprises, consists essentially of, or consists of an amino acid sequence that shares at least 80%, 85%, 90%, 95%, 96%, 97%, 98%, 99%, or 100% sequence identity to any one of SEQ ID NOs: 358. In some embodiments, the capsid protein comprises, consists essentially of, or consists of an amino acid sequence that shares at least 80%, 85%, 90%, 95%, 96%, 97%, 98%, 99%, or 100% sequence identity to any one of SEQ ID NOs: 359. In some embodiments, the capsid protein comprises, consists essentially of, or consists of an amino acid sequence that shares at least 80%, 85%, 90%, 95%, 96%, 97%, 98%, 99%, or 100% sequence identity to any one of SEQ ID NOs: 360. In some embodiments, the modifications in the VR-IV and/or VR-VIII sites already present in any one of capsid proteins of SEQ ID NOs: 351-360 are not further modified, but amino acid substitutions or modifications can be present at other sites of a capsid protein. In some embodiments, the modifications in the VR-IV and/or VR-VIII sites already present in any one of capsid proteins of SEQ ID NOs: 317-326 are not further modified, but amino acid substitutions or modifications can be present at other sites of a capsid protein.

Additional exemplary capsid sequences are provided in the Sequence Listing document, which is incorporated by reference into the specification in its entirety, including SEQ ID NOs: 327-350 and 361-372. In some embodiments, the capsid protein comprises, consists essentially of, or consists of an amino acid sequence that shares at least 80%, 85%, 90%, 95%, 96%, 97%, 98%, 99% or 100% sequence identity to any one of SEQ ID NOs: 327-350 and 361-372. In some embodiments, the capsid protein comprises, consists essentially of, or consists of any one of SEQ ID NOs: 327-350 and 361-372. In some embodiments, the modifications in the VR-IV and/or VR-VIII sites already present in any one of capsid proteins of SEQ ID NOs: 327-350 and 361-372 are not further modified, but amino acid substitutions or modifications can be present at other sites of a capsid protein.

TABLE 9A
Exemplary AAV capsid sequences

| | | SEQ |
|---|---|---|
| | | ID |
| Name | Sequence | NO: |
| ACE10 | MAADGYLPDWLEDNLSEGIREWWALKPGAPQPKAN | 317 |
| | QQHQDNARGLVLPGYKYLGPGNGLDKGEPVNAADA | |
| | AALEHDKAYDQQLKAGDNPYLKYNHADAEFQERLK | |
| | EDTSFGGNLGRAVFQAKKRLLEPLGLVEEAAKTAP | |
| | GKKRPVEQSPQEPDSSAGIGKSGAQPAKKRLNFGQ | |
| | TGDTESVPDPQPIGEPPAAPSGVGSLTMASGGGAP | |
| | VADNNEGADGVGSSSGNWHCDSQWLGDRVITTSTR | |
| | TWALPTYNNHLYKQISNSTSGGSSNDNAYFGYSTP | |
| | WGYFDFNRFHCHFSPRDWQRLINNNWGFRPKRLNF | |
| | KLFNIQVKEVTDNNGVKTIANNLTSTVQVFTDSDY | |
| | QLPYVLGSAHEGCLPPFPADVFMIPQYGYLTLNDG | |
| | SQAVGRSSFYCLEYFPSQMLRTGNNFQFSYEFENV | |
| | PFHSSYAHSQSLDRLMNPLIDQYLYYLSKTINGSG | |
| | QNQQTLKFSVAGPSNMAVQGRNYIPGPSYRQQRVS | |
| | TTVTQNNNSEFAWPGASSWALNGRNSLMNPGPAMA | |
| | SHKEGEDRFFPLSGSLIFGKQGTGRDNVDADKVMI | |
| | TNEEEIKTTNPVATESYGQVATNHEDNIRSAQTGW | |
| | VQNQGILPGMVWQDRDVYLQGPIWAKIPHTDGNFH | |
| | PSPLMGGFGMKHPPPQILIKNTPVPADPPTAFNKD | |
| | KLNSFITQYSTGQVSVEIEWELQKENSKRWNPEIQ | |
| | YTSNYYKSNNVEFAVNTEGVYSEPRPIGTRYLTRN | |
| | L | |
| ZC373 | MAADGYLPDWLEDNLSEGIREWWALKPGAPQPKAN | 318 |
| | QQHQDNARGLVLPGYKYLGPGNGLDKGEPVNAADA | |
| | AALEHDKAYDQQLKAGDNPYLKYNHADAEFQERLK | |
| | EDTSFGGNLGRAVFQAKKRLLEPLGLVEEAAKTAP | |
| | GKKRPVEQSPQEPDSSAGIGKSGAQPAKKRLNFGQ | |
| | TGDTESVPDPQPIGEPPAAPSGVGSLTMASGGGAP | |
| | VADNNEGADGVGSSSGNWHCDSQWLGDRVITTSTR | |
| | TWALPTYNNHLYKQISNSTSGGSSNDNAYFGYSTP | |
| | WGYFDFNRFHCHFSPRDWQRLINNNWGFRPKRLNF | |
| | KLFNIQVKEVTDNNGVKTIANNLTSTVQVFTDSDY | |
| | QLPYVLGSAHEGCLPPFPADVFMIPQYGYLTLNDG | |
| | SQAVGRSSFYCLEYFPSQMLRTGNNFQFSYEFENV | |
| | PFHSSYAHSQSLDRLMNPLIDQYLYYLSKTIKGSG | |
| | QNQQTLKFSVAGPSNMAVQGRNYIPGPSYRQQRVS | |
| | TTVTQNNNSEFAWPGASSWALNGRNSLMNPGPAMA | |
| | SHKEGEDRFFPLSGSLIFGKQGTGRDNVDADKVMI | |
| | TNEEEIKTTNPVATESYGQVATNHENTVSIAQTGW | |
| | VQNQGILPGMVWQDRDVYLQGPIWAKIPHTDGNFH | |
| | PSPLMGGFGMKHPPPQILIKNTPVPADPPTAFNKD | |
| | KLNSFITQYSTGQVSVEIEWELQKENSKRWNPEIQ | |
| | YTSNYYKSNNVEFAVNTEGVYSEPRPIGTRYLTRN | |
| | L | |
| ZC375 | MAADGYLPDWLEDNLSEGIREWWALKPGAPQPKAN | 319 |
| | QQHQDNARGLVLPGYKYLGPGNGLDKGEPVNAADA | |
| | AALEHDKAYDQQLKAGDNPYLKYNHADAEFQERLK | |
| | EDTSFGGNLGRAVFQAKKRLLEPLGLVEEAAKTAP | |
| | GKKRPVEQSPQEPDSSAGIGKSGAQPAKKRLNFGQ | |
| | TGDTESVPDPQPIGEPPAAPSGVGSLTMASGGGAP | |
| | VADNNEGADGVGSSSGNWHCDSQWLGDRVITTSTR | |
| | TWALPTYNNHLYKQISNSTSGGSSNDNAYFGYSTP | |
| | WGYFDFNRFHCHFSPRDWQRLINNNWGFRPKRLNF | |
| | KLFNIQVKEVTDNNGVKTIANNLTSTVQVFTDSDY | |
| | QLPYVLGSAHEGCLPPFPADVFMIPQYGYLTLNDG | |
| | SQAVGRSSFYCLEYFPSQMLRTGNNFQFSYEFENV | |
| | PFHSSYAHSQSLDRLMNPLIDQYLYYLSKTIKGSG | |
| | QNQQTLKFSVAGPSNMAVQGRNYIPGPSYRQQRVS | |
| | TTVTQNNNSEFAWPGASSWALNGRNSLMNPGPAMA | |
| | SHKEGEDRFFPLSGSLIFGKQGTGRDNVDADKVMI | |
| | TNEEEIKTTNPVATESYGQVATNHNSTYLGAQTGW | |
| | VQNQGILPGMVWQDRDVYLQGPIWAKIPHTDGNFH | |
| | PSPLMGGFGMKHPPPQILIKNTPVPADPPTAFNKD | |
| | KLNSFITQYSTGQVSVEIEWELQKENSKRWNPEIQ | |
| | YTSNYYKSNNVEFAVNTEGVYSEPRPIGTRYLTRN | |
| | L | |
| ZC377 | MAADGYLPDWLEDNLSEGIREWWALKPGAPQPKAN | 320 |
| | QQHQDNARGLVLPGYKYLGPGNGLDKGEPVNAADA | |
| | AALEHDKAYDQQLKAGDNPYLKYNHADAEFQERLK | |
| | EDTSFGGNLGRAVFQAKKRLLEPLGLVEEAAKTAP | |
| | GKKRPVEQSPQEPDSSAGIGKSGAQPAKKRLNFGQ | |
| | TGDTESVPDPQPIGEPPAAPSGVGSLTMASGGGAP | |
| | VADNNEGADGVGSSSGNWHCDSQWLGDRVITTSTR | |
| | TWALPTYNNHLYKQISNSTSGGSSNDNAYFGYSTP | |
| | WGYFDFNRFHCHFSPRDWQRLINNNWGFRPKRLNF | |
| | KLFNIQVKEVTDNNGVKTIANNLTSTVQVFTDSDY | |
| | QLPYVLGSAHEGCLPPFPADVFMIPQYGYLTLNDG | |
| | SQAVGRSSFYCLEYFPSQMLRTGNNFQFSYEFENV | |
| | PFHSSYAHSQSLDRLMNPLIDQYLYYLSKTIKGSG | |
| | QNQQTLKFSVAGPSNMAVQGRNYIPGPSYRQQRVS | |
| | TTVTQNNNSEFAWPGASSWALNGRNSLMNPGPAMA | |
| | SHKEGEDRFFPLSGSLIFGKQGTGRDNVDADKVMI | |
| | TNEEEIKTTNPVATESYGQVATNHQANYGQAQTGW | |
| | VQNQGILPGMVWQDRDVYLQGPIWAKIPHTDGNFH | |
| | PSPLMGGFGMKHPPPQILIKNTPVPADPPTAFNKD | |
| | KLNSFITQYSTGQVSVEIEWELQKENSKRWNPEIQ | |
| | YTSNYYKSNNVEFAVNTEGVYSEPRPIGTRYLTRN | |
| | L | |
| ZC401 | MAADGYLPDWLEDNLSEGIREWWALKPGAPQPKAN | 321 |
| | QQHQDNARGLVLPGYKYLGPGNGLDKGEPVNAADA | |
| | AALEHDKAYDQQLKAGDNPYLKYNHADAEFQERLK | |
| | EDTSFGGNLGRAVFQAKKRLLEPLGLVEEAAKTAP | |
| | GKKRPVEQSPQEPDSSAGIGKSGAQPAKKRLNFGQ | |
| | TGDTESVPDPQPIGEPPAAPSGVGSLTMASGGGAP | |
| | VADNNEGADGVGSSSGNWHCDSQWLGDRVITTSTR | |
| | TWALPTYNNHLYKQISNSTSGGSSNDNAYFGYSTP | |
| | WGYFDFNRFHCHFSPRDWQRLINNNWGFRPKRLNF | |
| | KLFNIQVKEVTDNNGVKTIANNLTSTVQVFTDSDY | |
| | QLPYVLGSAHEGCLPPFPADVFMIPQYGYLTLNDG | |
| | SQAVGRSSFYCLEYFPSQMLRTGNNFQFSYEFENV | |
| | PFHSSYAHSQSLDRLMNPLIDQYLYYLSKTINGSG | |
| | QNQQTLKFSVAGPSNMAVQGRNYIPGPSYRQQRVS | |
| | TTVTQNNNSEFAWPGASSWALNGRNSLMNPGPAMA | |
| | SHKEGEDRFFPLSGSLIFGKQGTGRDNVDADKVMI | |
| | TNEEEIKTTNPVATESYGQVATNHMMTTARAQTGW | |
| | VQNQGILPGMVWQDRDVYLQGPIWAKIPHTDGNFH | |
| | PSPLMGGFGMKHPPPQILIKNTPVPADPPTAFNKD | |
| | KLNSFITQYSTGQVSVEIEWELQKENSKRWNPEIQ | |
| | YTSNYYKSNNVEFAVNTEGVYSEPRPIGTRYLTRN | |
| | L | |
| ZC402 | MAADGYLPDWLEDNLSEGIREWWALKPGAPQPKAN | 322 |
| | QQHQDNARGLVLPGYKYLGPGNGLDKGEPVNAADA | |
| | AALEHDKAYDQQLKAGDNPYLKYNHADAEFQERLK | |
| | EDTSFGGNLGRAVFQAKKRLLEPLGLVEEAAKTAP | |
| | GKKRPVEQSPQEPDSSAGIGKSGAQPAKKRLNFGQ | |
| | TGDTESVPDPQPIGEPPAAPSGVGSLTMASGGGAP | |
| | VADNNEGADGVGSSSGNWHCDSQWLGDRVITTSTR | |
| | TWALPTYNNHLYKQISNSTSGGSSNDNAYFGYSTP | |
| | WGYFDFNRFHCHFSPRDWQRLINNNWGFRPKRLNF | |
| | KLFNIQVKEVTDNNGVKTIANNLTSTVQVFTDSDY | |
| | QLPYVLGSAHEGCLPPFPADVFMIPQYGYLTLNDG | |
| | SQAVGRSSFYCLEYFPSQMLRTGNNFQFSYEFENV | |
| | PFHSSYAHSQSLDRLMNPLIDQYLYYLSKTINGSG | |
| | QNQQTLKFSVAGPSNMAVQGRNYIPGPSYRQQRVS | |
| | TTVTQNNNSEFAWPGASSWALNGRNSLMNPGPAMA | |
| | SHKEGEDRFFPLSGSLIFGKQGTGRDNVDADKVMI | |
| | TNEEEIKTTNPVATESYGQVATNHQGAYAQAQTGW | |
| | VQNQGILPGMVWQDRDVYLQGPIWAKIPHTDGNFH | |
| | PSPLMGGFGMKHPPPQILIKNTPVPADPPTAFNKD | |
| | KLNSFITQYSTGQVSVEIEWELQKENSKRWNPEIQ | |
| | YTSNYYKSNNVEFAVNTEGVYSEPRPIGTRYLTRN | |
| | L | |
| ZC428 | MAADGYLPDWLEDNLSEGIREWWALKPGAPQPKAN | 323 |
| | QQHQDNARGLVLPGYKYLGPGNGLDKGEPVNAADA | |
| | AALEHDKAYDQQLKAGDNPYLKYNHADAEFQERLK | |
| | EDTSFGGNLGRAVFQAKKRLLEPLGLVEEAAKTAP | |
| | GKKRPVEQSPQEPDSSAGIGKSGAQPAKKRLNFGQ | |
| | TGDTESVPDPQPIGEPPAAPSGVGSLTMASGGGAP | |
| | VADNNEGADGVGSSSGNWHCDSQWLGDRVITTSTR | |
| | TWALPTYNNHLYKQISNSTSGGSSNDNAYFGYSTP | |
| | WGYFDFNRFHCHFSPRDWQRLINNNWGFRPKRLNF | |
| | KLFNIQVKEVTDNNGVKTIANNLTSTVQVFTDSDY | |
| | QLPYVLGSAHEGCLPPFPADVFMIPQYGYLTLNDG | |
| | SQAVGRSSFYCLEYFPSQMLRTGNNFQFSYEFENV | |
| | PFHSSYAHSQSLDRLMNPLIDQYLYYLSKTINGSG | |
| | QNQQTLKFSVAGPSNMAVQGRNYIPGPSYRQQRVS | |
| | TTVTQNNNSEFAWPGASSWALNGRNSLMNPGPAMA | |
| | SHKEGEDRFFPLSGSLIFGKQGTGRDNVDADKVMI | |
| | TNEEEIKTTNPVATESYGQVATNHNSTYLGAQTGW | |
| | VQNQGILPGMVWQDRDVYLQGPIWAKIPHTDGNFH | |
| | PSPLMGGFGMKHPPPQILIKNTPVPADPPTAFNKD | |
| | KLNSFITQYSTGQVSVEIEWELQKENSKRWNPEIQ | |
| | YTSNYYKSNNVEFAVNTEGVYSEPRPIGTRYLTRN | |
| | L | |
| ZC478 | MAADGYLPDWLEDNLSEGIREWWALKPGAPQPKAN | 324 |
| | QQHQDNARGLVLPGYKYLGPGNGLDKGEPVNAADA | |
| | AALEHDKAYDQQLKAGDNPYLKYNHADAEFQERLK | |
| | EDTSFGGNLGRAVFQAKKRLLEPLGLVEEAAKTAP | |
| | GKKRPVEQSPQEPDSSAGIGKSGAQPAKKRLNFGQ | |
| | TGDTESVPDPQPIGEPPAAPSGVGSLTMASGGGAP | |
| | VADNNEGADGVGSSSGNWHCDSQWLGDRVITTSTR | |
| | TWALPTYNNHLYKQISNSTSGGSSNDNAYFGYSTP | |
| | WGYFDFNRFHCHFSPRDWQRLINNNWGFRPKRLNF | |
| | KLFNIQVKEVTDNNGVKTIANNLTSTVQVFTDSDY | |
| | QLPYVLGSAHEGCLPPFPADVFMIPQYGYLTLNDG | |
| | SQAVGRSSFYCLEYFPSQMLRTGNNFQFSYEFENV | |
| | PFHSSYAHSQSLDRLMNPLIDQYLYYLSKTINGSG | |
| | QNQQTLKFSVAGPSNMAVQGRNYIPGPSYRQQRVS | |
| | TTVTQNNNSEFAWPGASSWALNGRNSLMNPGPAMA | |
| | SHKEGEDRFFPLSGSLIFGKQGTGRDNVDADKVMI | |
| | TNEEEIKTTNPVATESYGQVATNHCSTSIRAQTGW | |
| | VQNQGILPGMVWQDRDVYLQGPIWAKIPHTDGNFH | |
| | PSPLMGGFGMKHPPPQILIKNTPVPADPPTAFNKD | |
| | KLNSFITQYSTGQVSVEIEWELQKENSKRWNPEIQ | |
| | YTSNYYKSNNVEFAVNTEGVYSEPRPIGTRYLTRN | |
| | L | |
| ZC536 | MAADGYLPDWLEDNLSEGIREWWALKPGAPQPKAN | 325 |
| | QQHQDNARGLVLPGYKYLGPGNGLDKGEPVNAADA | |
| | AALEHDKAYDQQLKAGDNPYLKYNHADAEFQERLK | |
| | EDTSFGGNLGRAVFQAKKRLLEPLGLVEEAAKTAP | |
| | GKKRPVEQSPQEPDSSAGIGKSGAQPAKKRLNFGQ | |
| | TGDTESVPDPQPIGEPPAAPSGVGSLTMASGGGAP | |
| | VADNNEGADGVGSSSGNWHCDSQWLGDRVITTSTR | |
| | TWALPTYNNHLYKQISNSTSGGSSNDNAYFGYSTP | |
| | WGYFDFNRFHCHFSPRDWQRLINNNWGFRPKRLNF | |
| | KLFNIQVKEVTDNNGVKTIANNLTSTVQVFTDSDY | |
| | QLPYVLGSAHEGCLPPFPADVFMIPQYGYLTLNDG | |
| | SQAVGRSSFYCLEYFPSQMLRTGNNFQFSYEFENV | |
| | PFHSSYAHSQSLDRLMNPLIDQYLYYLSKTIKGSG | |
| | QNQQTLKFSVAGPSNMAVQGRNYIPGPSYRQQRVS | |
| | TTVTQNNNSEFAWPGASSWALNGRNSLMNPGPAMA | |
| | SHKEGEDRFFPLSGSLIFGKQGTGRDNVDADKVMI | |
| | TNEEEIKTTNPVATESYGQVATNHEDNIRSAQTGW | |
| | VQNQGILPGMVWQDRDVYLQGPIWAKIPHTDGNFH | |
| | PSPLMGGFGMKHPPPQILIKNTPVPADPPTAFNKD | |
| | KLNSFITQYSTGQVSVEIEWELQKENSKRWNPEIQ | |
| | YTSNYYKSNNVEFAVNTEGVYSEPRPIGTRYLTRN | |
| | L | |
| ZC538 | MAADGYLPDWLEDNLSEGIREWWALKPGAPQPKAN | 326 |
| | QQHQDNARGLVLPGYKYLGPGNGLDKGEPVNAADA | |
| | AALEHDKAYDQQLKAGDNPYLKYNHADAEFQERLK | |
| | EDTSFGGNLGRAVFQAKKRLLEPLGLVEEAAKTAP | |
| | GKKRPVEQSPQEPDSSAGIGKSGAQPAKKRLNFGQ | |
| | TGDTESVPDPQPIGEPPAAPSGVGSLTMASGGGAP | |
| | VADNNEGADGVGSSSGNWHCDSQWLGDRVITTSTR | |
| | TWALPTYNNHLYKQISNSTSGGSSNDNAYFGYSTP | |
| | WGYFDFNRFHCHFSPRDWQRLINNNWGFRPKRLNF | |
| | KLFNIQVKEVTDNNGVKTIANNLTSTVQVFTDSDY | |
| | QLPYVLGSAHEGCLPPFPADVFMIPQYGYLTLNDG | |
| | SQAVGRSSFYCLEYFPSQMLRTGNNFQFSYEFENV | |
| | PFHSSYAHSQSLDRLMNPLIDQYLYYLSKTIKGSG | |
| | QNQQTLKFSVAGPSNMAVQGRNYIPGPSYRQQRVS | |
| | TTVTQNNNSEFAWPGASSWALNGRNSLMNPGPAMA | |
| | SHKEGEDRFFPLSGSLIFGKQGTGRDNVDADKVMI | |
| | TNEEEIKTTNPVATESYGQVATNHNNVISGAQTGW | |
| | VQNQGILPGMVWQDRDVYLQGPIWAKIPHTDGNFH | |
| | PSPLMGGFGMKHPPPQILIKNTPVPADPPTAFNKD | |
| | KLNSFITQYSTGQVSVEIEWELQKENSKRWNPEIQ | |
| | YTSNYYKSNNVEFAVNTEGVYSEPRPIGTRYLTRN | |
| | L | |
| ZC621 | MAADGYLPDWLEDNLSEGIREWWALKPGAPQPKAN | 351 |
| | QQHQDNARGLVLPGYKYLGPGNGLDKGEPVNAADA | |
| | AALEHDKAYDQQLKAGDNPYLKYNHADAEFQERLK | |
| | EDTSFGGNLGRAVFQAKKRLLEPLGLVEEAAKTAP | |
| | GKKRPVEQSPQEPDSSAGIGKSGAQPAKKRLNFGQ | |
| | TGDTESVPDPQPIGEPPAAPSGVGSLTMASGGGAP | |
| | VADNNEGADGVGSSSGNWHCDSQWLGDRVITTSTR | |
| | TWALPTYNNHLYKQISNSTSGGSSNDNAYFGYSTP | |
| | WGYFDFNRFHCHFSPRDWQRLINNNWGFRPKRLNF | |
| | KLFNIQVKEVTDNNGVKTIANNLTSTVQVFTDSDY | |
| | QLPYVLGSAHEGCLPPFPADVFMIPQYGYLTLNDG | |
| | SQAVGRSSFYCLEYFPSQMLRTGNNFQFSYEFENV | |
| | PFHSSYAHSQSLDRLMNPLIDQYLYYLSKTINGSG | |
| | QNQQTLKFSVAGPSNMAVQGRNYIPGPSYRQQRVS | |
| | TTVTQNNNSEFAWPGASSWALNGRNSLMNPGPAMA | |
| | SHKEGEDRFFPLSGSLIFGKQGTGRDNVDADKVMI | |
| | TNEEEIKTTNPVATESYGQVADVQQLPGFQAQTEW | |
| | VQNQGILPGMVWQDRDVYLQGPIWAKIPHTDGNFH | |
| | PSPLMGGFGMKHPPPQILIKNTPVPADPPTAFNKD | |
| | KLNSFITQYSTGQVSVEIEWELQKENSKRWNPEIQ | |
| | YTSNYYKSNNVEFAVNTEGVYSEPRPIGTRYLTRN | |
| | L | |
| ZC623 | MAADGYLPDWLEDNLSEGIREWWALKPGAPQPKAN | 352 |
| | QQHQDNARGLVLPGYKYLGPGNGLDKGEPVNAADA | |
| | AALEHDKAYDQQLKAGDNPYLKYNHADAEFQERLK | |
| | EDTSFGGNLGRAVFQAKKRLLEPLGLVEEAAKTAP | |
| | GKKRPVEQSPQEPDSSAGIGKSGAQPAKKRLNFGQ | |
| | TGDTESVPDPQPIGEPPAAPSGVGSLTMASGGGAP | |
| | VADNNEGADGVGSSSGNWHCDSQWLGDRVITTSTR | |
| | TWALPTYNNHLYKQISNSTSGGSSNDNAYFGYSTP | |
| | WGYFDFNRFHCHFSPRDWQRLINNNWGFRPKRLNF | |
| | KLFNIQVKEVTDNNGVKTIANNLTSTVQVFTDSDY | |
| | QLPYVLGSAHEGCLPPFPADVFMIPQYGYLTLNDG | |
| | SQAVGRSSFYCLEYFPSQMLRTGNNFQFSYEFENV | |
| | PFHSSYAHSQSLDRLMNPLIDQYLYYLSKTINGSG | |
| | QNQQTLKFSVAGPSNMAVQGRNYIPGPSYRQQRVS | |
| | TTVTQNNNSEFAWPGASSWALNGRNSLMNPGPAMA | |
| | SHKEGEDRFFPLSGSLIFGKQGTGRDNVDADKVMI | |
| | TNEEEIKTTNPVATESYGQVADVQQHPGFQAQTEW | |
| | VQNQGILPGMVWQDRDVYLQGPIWAKIPHTDGNFH | |
| | PSPLMGGFGMKHPPPQILIKNTPVPADPPTAFNKD | |
| | KLNSFITQYSTGQVSVEIEWELQKENSKRWNPEIQ | |
| | YTSNYYKSNNVEFAVNTEGVYSEPRPIGTRYLTRN | |
| | L | |
| ZC625 | MAADGYLPDWLEDNLSEGIREWWALKPGAPQPKAN | 353 |
| | QQHQDNARGLVLPGYKYLGPGNGLDKGEPVNAADA | |
| | AALEHDKAYDQQLKAGDNPYLKYNHADAEFQERLK | |
| | EDTSFGGNLGRAVFQAKKRLLEPLGLVEEAAKTAP | |
| | GKKRPVEQSPQEPDSSAGIGKSGAQPAKKRLNFGQ | |
| | TGDTESVPDPQPIGEPPAAPSGVGSLTMASGGGAP | |
| | VADNNEGADGVGSSSGNWHCDSQWLGDRVITTSTR | |
| | TWALPTYNNHLYKQISNSTSGGSSNDNAYFGYSTP | |
| | WGYFDFNRFHCHFSPRDWQRLINNNWGFRPKRLNF | |
| | KLFNIQVKEVTDNNGVKTIANNLTSTVQVFTDSDY | |
| | QLPYVLGSAHEGCLPPFPADVFMIPQYGYLTLNDG | |
| | SQAVGRSSFYCLEYFPSQMLRTGNNFQFSYEFENV | |
| | PFHSSYAHSQSLDRLMNPLIDQYLYYLSKTINGSH | |
| | VRGDLSGQNQQTLKFSVAGPSNMAVQGRNYIPGPS | |
| | YRQQRVSTTVTQNNNSEFAWPGASSWALNGRNSLM | |
| | NPGPAMASHKEGEDRFFPLSGSLIFGKQGTGRDNV | |
| | DADKVMITNEEEIKTTNPVATESYGQVATNHQSAQ | |
| | AQAQTGWVQNQGILPGMVWQDRDVYLQGPIWAKIP | |
| | HTDGNFHPSPLMGGFGMKHPPPQILIKNTPVPADP | |
| | PTAFNKDKLNSFITQYSTGQVSVEIEWELQKENSK | |
| | RWNPEIQYTSNYYKSNNVEFAVNTEGVYSEPRPIG | |
| | TRYLTRNL | |
| ZC626 | MAADGYLPDWLEDNLSEGIREWWALKPGAPQPKAN | 354 |
| | QQHQDNARGLVLPGYKYLGPGNGLDKGEPVNAADA | |
| | AALEHDKAYDQQLKAGDNPYLKYNHADAEFQERLK | |
| | EDTSFGGNLGRAVFQAKKRLLEPLGLVEEAAKTAP | |
| | GKKRPVEQSPQEPDSSAGIGKSGAQPAKKRLNFGQ | |
| | TGDTESVPDPQPIGEPPAAPSGVGSLTMASGGGAP | |
| | VADNNEGADGVGSSSGNWHCDSQWLGDRVITTSTR | |
| | TWALPTYNNHLYKQISNSTSGGSSNDNAYFGYSTP | |
| | WGYFDFNRFHCHFSPRDWQRLINNNWGFRPKRLNF | |
| | KLFNIQVKEVTDNNGVKTIANNLTSTVQVFTDSDY | |
| | QLPYVLGSAHEGCLPPFPADVFMIPQYGYLTLNDG | |
| | SQAVGRSSFYCLEYFPSQMLRTGNNFQFSYEFENV | |
| | PFHSSYAHSQSLDRLMNPLIDQYLYYLSKTINGVV | |
| | SSGARSGQNQQTLKFSVAGPSNMAVQGRNYIPGPS | |
| | YRQQRVSTTVTQNNNSEFAWPGASSWALNGRNSLM | |
| | NPGPAMASHKEGEDRFFPLSGSLIFGKQGTGRDNV | |
| | DADKVMITNEEEIKTTNPVATESYGQVATNHQSAQ | |
| | AQAQTGWVQNQGILPGMVWQDRDVYLQGPIWAKIP | |
| | HTDGNFHPSPLMGGFGMKHPPPQILIKNTPVPADP | |
| | PTAFNKDKLNSFITQYSTGQVSVEIEWELQKENSK | |
| | RWNPEIQYTSNYYKSNNVEFAVNTEGVYSEPRPIG | |
| | TRYLTRNL | |
| ZC630 | MAADGYLPDWLEDNLSEGIREWWALKPGAPQPKAN | 355 |
| | QQHQDNARGLVLPGYKYLGPGNGLDKGEPVNAADA | |
| | AALEHDKAYDQQLKAGDNPYLKYNHADAEFQERLK | |
| | EDTSFGGNLGRAVFQAKKRLLEPLGLVEEAAKTAP | |
| | GKKRPVEQSPQEPDSSAGIGKSGAQPAKKRLNFGQ | |
| | TGDTESVPDPQPIGEPPAAPSGVGSLTMASGGGAP | |
| | VADNNEGADGVGSSSGNWHCDSQWLGDRVITTSTR | |
| | TWALPTYNNHLYKQISNSTSGGSSNDNAYFGYSTP | |
| | WGYFDFNRFHCHFSPRDWQRLINNNWGFRPKRLNF | |
| | KLFNIQVKEVTDNNGVKTIANNLTSTVQVFTDSDY | |
| | QLPYVLGSAHEGCLPPFPADVFMIPQYGYLTLNDG | |
| | SQAVGRSSFYCLEYFPSQMLRTGNNFQFSYEFENV | |
| | PFHSSYAHSQSLDRLMNPLIDQYLYYLSKTINGSG | |
| | QNQQTLKFSVAGPSNMAVQGRNYIPGPSYRQQRVS | |
| | TTVTQNNNSEFAWPGASSWALNGRNSLMNPGPAMA | |
| | SHKEGEDRFFPLSGSLIFGKQGTGRDNVDADKVMI | |
| | TNEEEIKTTNPVATESYGQVATNHQSSVRGDAARL | |
| | AGAQTGWVQNQGILPGMVWQDRDVYLQGPIWAKIP | |
| | HTDGNFHPSPLMGGFGMKHPPPQILIKNTPVPADP | |
| | PTAFNKDKLNSFITQYSTGQVSVEIEWELQKENSK | |
| | RWNPEIQYTSNYYKSNNVEFAVNTEGVYSEPRPIG | |
| | TRYLTRNL | |
| ZC631 | MAADGYLPDWLEDNLSEGIREWWALKPGAPQPKAN | 356 |
| | QQHQDNARGLVLPGYKYLGPGNGLDKGEPVNAADA | |
| | AALEHDKAYDQQLKAGDNPYLKYNHADAEFQERLK | |
| | EDTSFGGNLGRAVFQAKKRLLEPLGLVEEAAKTAP | |
| | GKKRPVEQSPQEPDSSAGIGKSGAQPAKKRLNFGQ | |
| | TGDTESVPDPQPIGEPPAAPSGVGSLTMASGGGAP | |
| | VADNNEGADGVGSSSGNWHCDSQWLGDRVITTSTR | |
| | TWALPTYNNHLYKQISNSTSGGSSNDNAYFGYSTP | |
| | WGYFDFNRFHCHFSPRDWQRLINNNWGFRPKRLNF | |
| | KLFNIQVKEVTDNNGVKTIANNLTSTVQVFTDSDY | |
| | QLPYVLGSAHEGCLPPFPADVFMIPQYGYLTLNDG | |
| | SQAVGRSSFYCLEYFPSQMLRTGNNFQFSYEFENV | |
| | PFHSSYAHSQSLDRLMNPLIDQYLYYLSKTINGSG | |
| | QNQQTLKFSVAGPSNMAVQGRNYIPGPSYRQQRVS | |
| | TTVTQNNNSEFAWPGASSWALNGRNSLMNPGPAMA | |
| | SHKEGEDRFFPLSGSLIFGKQGTGRDNVDADKVMI | |
| | TNEEEIKTTNPVATESYGQVADVQQVPGFQAQTMW | |
| | VQNQGILPGMVWQDRDVYLQGPIWAKIPHTDGNFH | |
| | PSPLMGGFGMKHPPPQILIKNTPVPADPPTAFNKD | |
| | KLNSFITQYSTGQVSVEIEWELQKENSKRWNPEIQ | |
| | YTSNYYKSNNVEFAVNTEGVYSEPRPIGTRYLTRN | |
| | L | |
| ZC632 | MAADGYLPDWLEDNLSEGIREWWALKPGAPQPKAN | 357 |
| | QQHQDNARGLVLPGYKYLGPGNGLDKGEPVNAADA | |
| | AALEHDKAYDQQLKAGDNPYLKYNHADAEFQERLK | |
| | EDTSFGGNLGRAVFQAKKRLLEPLGLVEEAAKTAP | |
| | GKKRPVEQSPQEPDSSAGIGKSGAQPAKKRLNFGQ | |
| | TGDTESVPDPQPIGEPPAAPSGVGSLTMASGGGAP | |
| | VADNNEGADGVGSSSGNWHCDSQWLGDRVITTSTR | |
| | TWALPTYNNHLYKQISNSTSGGSSNDNAYFGYSTP | |
| | WGYFDFNRFHCHFSPRDWQRLINNNWGFRPKRLNF | |
| | KLFNIQVKEVTDNNGVKTIANNLTSTVQVFTDSDY | |
| | QLPYVLGSAHEGCLPPFPADVFMIPQYGYLTLNDG | |
| | SQAVGRSSFYCLEYFPSQMLRTGNNFQFSYEFENV | |
| | PFHSSYAHSQSLDRLMNPLIDQYLYYLSKTINGSG | |
| | QNQQTLKFSVAGPSNMAVQGRNYIPGPSYRQQRVS | |
| | TTVTQNNNSEFAWPGASSWALNGRNSLMNPGPAMA | |
| | SHKEGEDRFFPLSGSLIFGKQGTGRDNVDADKVMI | |
| | TNEEEIKTTNPVATESYGQVADVQQVPGFQAQTQW | |
| | VQNQGILPGMVWQDRDVYLQGPIWAKIPHTDGNFH | |
| | PSPLMGGFGMKHPPPQILIKNTPVPADPPTAFNKD | |
| | KLNSFITQYSTGQVSVEIEWELQKENSKRWNPEIQ | |
| | YTSNYYKSNNVEFAVNTEGVYSEPRPIGTRYLTRN | |
| | L | |
| ZC633 | MAADGYLPDWLEDNLSEGIREWWALKPGAPQPKAN | 358 |
| | QQHQDNARGLVLPGYKYLGPGNGLDKGEPVNAADA | |
| | AALEHDKAYDQQLKAGDNPYLKYNHADAEFQERLK | |
| | EDTSFGGNLGRAVFQAKKRLLEPLGLVEEAAKTAP | |
| | GKKRPVEQSPQEPDSSAGIGKSGAQPAKKRLNFGQ | |
| | TGDTESVPDPQPIGEPPAAPSGVGSLTMASGGGAP | |
| | VADNNEGADGVGSSSGNWHCDSQWLGDRVITTSTR | |
| | TWALPTYNNHLYKQISNSTSGGSSNDNAYFGYSTP | |
| | WGYFDFNRFHCHFSPRDWQRLINNNWGFRPKRLNF | |
| | KLFNIQVKEVTDNNGVKTIANNLTSTVQVFTDSDY | |
| | QLPYVLGSAHEGCLPPFPADVFMIPQYGYLTLNDG | |
| | SQAVGRSSFYCLEYFPSQMLRTGNNFQFSYEFENV | |
| | PFHSSYAHSQSLDRLMNPLIDQYLYYLSKTINGSG | |
| | QNQQTLKFSVAGPSNMAVQGRNYIPGPSYRQQRVS | |
| | TTVTQNNNSEFAWPGASSWALNGRNSLMNPGPAMA | |
| | SHKEGEDRFFPLSGSLIFGKQGTGRDNVDADKVMI | |
| | TNEEEIKTTNPVATESYGQVADVQQLPGFGAQTEW | |
| | VQNQGILPGMVWQDRDVYLQGPIWAKIPHTDGNFH | |
| | PSPLMGGFGMKHPPPQILIKNTPVPADPPTAFNKD | |
| | KLNSFITQYSTGQVSVEIEWELQKENSKRWNPEIQ | |
| | YTSNYYKSNNVEFAVNTEGVYSEPRPIGTRYLTRN | |
| | L | |
| ZC634 | MAADGYLPDWLEDNLSEGIREWWALKPGAPQPKAN | 359 |
| | QQHQDNARGLVLPGYKYLGPGNGLDKGEPVNAADA | |
| | AALEHDKAYDQQLKAGDNPYLKYNHADAEFQERLK | |
| | EDTSFGGNLGRAVFQAKKRLLEPLGLVEEAAKTAP | |
| | GKKRPVEQSPQEPDSSAGIGKSGAQPAKKRLNFGQ | |
| | TGDTESVPDPQPIGEPPAAPSGVGSLTMASGGGAP | |
| | VADNNEGADGVGSSSGNWHCDSQWLGDRVITTSTR | |
| | TWALPTYNNHLYKQISNSTSGGSSNDNAYFGYSTP | |
| | WGYFDFNRFHCHFSPRDWQRLINNNWGFRPKRLNF | |
| | KLFNIQVKEVTDNNGVKTIANNLTSTVQVFTDSDY | |
| | QLPYVLGSAHEGCLPPFPADVFMIPQYGYLTLNDG | |
| | SQAVGRSSFYCLEYFPSQMLRTGNNFQFSYEFENV | |
| | PFHSSYAHSQSLDRLMNPLIDQYLYYLSKTINGSG | |
| | QNQQTLKFSVAGPSNMAVQGRNYIPGPSYRQQRVS | |
| | TTVTQNNNSEFAWPGASSWALNGRNSLMNPGPAMA | |
| | SHKEGEDRFFPLSGSLIFGKQGTGRDNVDADKVMI | |
| | TNEEEIKTTNPVATESYGQVADVQQLPGFRPETEW | |
| | VQNQGILPGMVWQDRDVYLQGPIWAKIPHTDGNFH | |
| | PSPLMGGFGMKHPPPQILIKNTPVPADPPTAFNKD | |
| | KLNSFITQYSTGQVSVEIEWELQKENSKRWNPEIQ | |
| | YTSNYYKSNNVEFAVNTEGVYSEPRPIGTRYLTRN | |
| | L | |
| ZC635 | MAADGYLPDWLEDNLSEGIREWWALKPGAPQPKAN | 360 |
| | QQHQDNARGLVLPGYKYLGPGNGLDKGEPVNAADA | |
| | AALEHDKAYDQQLKAGDNPYLKYNHADAEFQERLK | |
| | EDTSFGGNLGRAVFQAKKRLLEPLGLVEEAAKTAP | |
| | GKKRPVEQSPQEPDSSAGIGKSGAQPAKKRLNFGQ | |
| | TGDTESVPDPQPIGEPPAAPSGVGSLTMASGGGAP | |
| | VADNNEGADGVGSSSGNWHCDSQWLGDRVITTSTR | |
| | TWALPTYNNHLYKQISNSTSGGSSNDNAYFGYSTP | |
| | WGYFDFNRFHCHFSPRDWQRLINNNWGFRPKRLNF | |
| | KLFNIQVKEVTDNNGVKTIANNLTSTVQVFTDSDY | |
| | QLPYVLGSAHEGCLPPFPADVFMIPQYGYLTLNDG | |
| | SQAVGRSSFYCLEYFPSQMLRTGNNFQFSYEFENV | |
| | PFHSSYAHSQSLDRLMNPLIDQYLYYLSKTINGSG | |
| | QNQQTLKFSVAGPSNMAVQGRNYIPGPSYRQQRVS | |
| | TTVTQNNNSEFAWPGASSWALNGRNSLMNPGPAMA | |
| | SHKEGEDRFFPLSGSLIFGKQGTGRDNVDADKVMI | |
| | TNEEEIKTTNPVATESYGQVADVQQLPGFNAQTEW | |
| | VQNQGILPGMVWQDRDVYLQGPIWAKIPHTDGNFH | |
| | PSPLMGGFGMKHPPPQILIKNTPVPADPPTAFNKD | |
| | KLNSFITQYSTGQVSVEIEWELQKENSKRWNPEIQ | |
| | YTSNYYKSNNVEFAVNTEGVYSEPRPIGTRYLTRN | |
| | L | |

In some embodiments, the capsid is not a chimeric capsid and/or not a combinatory capsid.

In some embodiments, a recombinant adeno-associated virus (rAAV) virion comprises a capsid protein (such as any described herein) and a vector genome. The vector genome may comprise an expression cassette flanked by inverted terminal repeats (ITRs), wherein the expression cassette is any one described herein for expression of cardioprotective gene products described herein.

In some embodiments, the rAAV virion specifically transduces heart cells.

In some embodiments, the rAAV virion specifically transduces cardiomyocytes.

In some embodiments, the rAAV virion traffics to the heart.

In some embodiments, the rAAV virion traffics to at least one organ other than the liver.

In some embodiments, the rAAV virion exhibits a higher heart transduction efficiency than an rAAV virion having an AAV9 VP1 capsid protein according to SEQ ID NO: 112. In some embodiments, the rAAV virion exhibits a higher (e.g., at least 2, 3, 4, 5, 6, 7, 8, 9 or 10 times higher) heart-to-liver transduction ratio than an rAAV virion having an AAV9 VP1 capsid protein according to SEQ ID NO: 112. In some embodiments, administration of the rAAV virion to a subject leads to a lower (e.g., at least 2, 3, 4, 5, 6, 7, 8, 9 or 10 times lower) liver viral load than administration of an rAAV virion having an AAV9 VP1 capsid protein according to SEQ ID NO: 112. In some embodiments, the rAAV virion exhibits a higher transduction efficiency, optionally higher heart transduction efficiency, than an rAAV virion having an AAV9 VP1 capsid protein according to SEQ ID NO: 112, assessed in a primate. In some embodiments, the rAAV virion exhibits a higher (e.g., at least 2, 3, 4, 5, 6, 7, 8, 9 or 10 times higher) heart-to-liver transduction ratio than an rAAV virion having an AAV9 VP1 capsid protein according to SEQ ID NO: 112, assessed in a primate. In some embodiments, administration of the rAAV virion to a subject leads to a lower (e.g., at least 2, 3, 4, 5, 6, 7, 8, 9 or 10 times lower) liver viral load than administration of an rAAV virion having an AAV9 VP1 capsid protein according to SEQ ID NO: 112, as assessed in a primate.

In some embodiments, the rAAV virion exhibits a higher heart transduction efficiency than an rAAV virion having an AAV5 VP1 capsid protein according to SEQ ID NO: 294. In some embodiments, the rAAV virion exhibits a higher (e.g., at least 2, 3, 4, 5, 6, 7, 8, 9 or 10 times higher) heart-to-liver transduction ratio than an rAAV virion having an AAV5 VP1 capsid protein according to SEQ ID NO: 294. In some embodiments, administration of the rAAV virion to a subject leads to a lower (e.g., at least 2, 3, 4, 5, 6, 7, 8, 9 or 10 times lower) liver viral load than administration of an rAAV virion having an AAV5 VP1 capsid protein according to SEQ ID NO: 294. In some embodiments, the rAAV virion exhibits a higher transduction efficiency, optionally higher heart transduction efficiency, than an rAAV virion having an AAV5 VP1 capsid protein according to SEQ ID NO: 294, assessed in a primate. In some embodiments, the rAAV virion exhibits a higher (e.g., at least 2, 3, 4, 5, 6, 7, 8, 9 or 10 times higher) heart-to-liver transduction ratio than an rAAV virion having an AAV5 VP1 capsid protein according to SEQ ID NO: 294, assessed in a primate. In some embodiments, administration of the rAAV virion to a subject leads to a lower (e.g., at least 2, 3, 4, 5, 6, 7, 8, 9 or 10 times lower) liver viral load than administration of an rAAV virion having an AAV5 VP1 capsid protein according to SEQ ID NO: 294, as assessed in a primate.

In some embodiments, the rAAV virion exhibits a higher heart transduction efficiency than an rAAV virion having an AAVrh.10 VP1 capsid protein according to SEQ ID NO: 298. In some embodiments, the rAAV virion exhibits a higher (e.g., at least 2, 3, 4, 5, 6, 7, 8, 9 or 10 times higher) heart-to-liver transduction ratio than an rAAV virion having an AAVrh.10 VP1 capsid protein according to SEQ ID NO: 298. In some embodiments, administration of the rAAV virion to a subject leads to a lower (e.g., at least 2, 3, 4, 5, 6, 7, 8, 9 or 10 times lower) liver viral load than administration of an rAAV virion having an AAVrh.10 VP1 capsid protein according to SEQ ID NO: 298. In some embodiments, the rAAV virion exhibits a higher transduction efficiency, optionally higher heart transduction efficiency, than an rAAV virion having an AAVrh.10 VP1 capsid protein according to SEQ ID NO: 298, assessed in a primate. In some embodiments, the rAAV virion exhibits a higher (e.g., at least 2, 3, 4, 5, 6, 7, 8, 9 or 10 times higher) heart-to-liver transduction ratio than an rAAV virion having an AAVrh.10 VP1 capsid protein according to SEQ ID NO: 298, assessed in a primate. In some embodiments, administration of the rAAV virion to a subject leads to a lower (e.g., at least 2, 3, 4, 5, 6, 7, 8, 9 or 10 times lower) liver viral load than administration of an rAAV virion having an AAVrh.10 VP1 capsid protein according to SEQ ID NO: 298, as assessed in a primate.

In some embodiments, the rAAV virion exhibits a higher heart transduction efficiency than an rAAV virion having an AAVrh.74 VP1 capsid protein according to SEQ ID NO: 302. In some embodiments, the rAAV virion exhibits a higher (e.g., at least 2, 3, 4, 5, 6, 7, 8, 9 or 10 times higher) heart-to-liver transduction ratio than an rAAV virion having an AAVrh.74 VP1 capsid protein according to SEQ ID NO: 302. In some embodiments, administration of the rAAV virion to a subject leads to a lower (e.g., at least 2, 3, 4, 5, 6, 7, 8, 9 or 10 times lower) liver viral load than administration of an rAAV virion having an AAVrh.74 VP1 capsid protein according to SEQ ID NO: 302. In some embodiments, the rAAV virion exhibits a higher transduction efficiency, optionally higher heart transduction efficiency, than an rAAV virion having an AAVrh.74 VP1 capsid protein according to SEQ ID NO: 302, assessed in a primate. In some embodiments, the rAAV virion exhibits a higher (e.g., at least 2, 3, 4, 5, 6, 7, 8, 9 or 10 times higher) heart-to-liver transduction ratio than an rAAV virion having an AAVrh.74 VP1 capsid protein according to SEQ ID NO: 302, assessed in a primate. In some embodiments, administration of the rAAV virion to a subject leads to a lower (e.g., at least 2, 3, 4, 5, 6, 7, 8, 9 or 10 times lower) liver viral load than administration of an rAAV virion having an AAVrh.74 VP1 capsid protein according to SEQ ID NO: 302, as assessed in a primate.

In some embodiments, the rAAV virions comprise an AAVrh.74 capsid protein or a variant thereof as known in the art. In some embodiments, the rAAV virions comprise an AAVrh.10 capsid protein or a variant thereof as known in the art. In some embodiments, the rAAV virions comprise an AAV-SLB101 capsid protein or a variant thereof as known in the art or described in, e.g., WO 2021/072197, which is incorporated by reference herein in its entirety. In some embodiments, the rAAV virions comprise an AAVmod capsid protein or a variant thereof as known in the art or described in, e.g., WO 2022/173847 or in Olivieri et al. (2021) 24^{th} Annual Meeting of the American Society of Gene & Cell Therapy available at https://www.affiniatx.com/pdf/asgct_2021_olivieri.pdf, both of which are incorporated by reference herein in their entirety. In some embodiments, the rAAV virions comprise the AAV^{mut1dec1}, AAV^{deco1}, and/or AAV^{mut1} capsid protein or a variant thereof as known in the art or described in, e.g., WO 2022/173847. In some embodiments, the rAAV virions comprise an AAVcc.47 capsid protein or a variant thereof as known in the art or described in, e.g., Gonzalez et al. Nature Communications 13:5947 (2022), which is incorporated by reference herein in its entirety. In some embodiments, the rAAV virions comprise an AAVHSC16 capsid protein or a variant thereof as known in the art or described in, e.g., Smith et al. Molecular Therapy Methods & Clinical Development 26:224-238 (2022), which is incorporated by reference herein in its entirety. In some embodiments, the rAAV virions comprise a MyoAAV capsid protein or variant thereof as known in the art or described in, e.g., Tabebordbar et al. Cell 184(19):4919-4938. (2021), which is incorporated by reference herein in its entirety. In some embodiments, the rAAV virions comprise the MyoAAV-4E, MyoAAV-3F, MyoAAV-4A, or MyoAAV-4D capsid protein or variant thereof as known in the art or described in, e.g., Tabebordbar et al. In some embodiments, the rAAV virions comprise the 4D-C102 or C102 capsid protein or a variant thereof as known in the art or described in, e.g., US2021/0380643. Exemplary sequences of some of these capsid proteins are provided in Table 9B below.

TABLE 9B
Exemplary AAV capsid sequences

| | | SEQ |
|---|---|---|
| | | ID |
| Name | Sequence | NO: |
| AAV- | MAADGYLPDWLEDNLSEGIREWWALKPGAPQPKAN | 305 |
| SLB101 | QQHQDNARGLVLPGYKYLGPGNGLDKGEPVNAADA | |
| | AALEHDKAYDQQLKAGDNPYLKYNHADAEFQERLK | |
| | EDTSFGGNLGRAVFQAKKRLLEPLGLVEEAAKTAP | |
| | GKKRPVEQSPQEPDSSAGIGKSGAQPAKKRLNFGQ | |
| | TGDTESVPDPQPIGEPPAAPSGVGSLTMASGGGAP | |
| | VADNNEGADGVGSSSGNWHCDSQWLGDRVITTSTR | |
| | TWALPTYNNHLYKQISNSTSGGSSNDNAYFGYSTP | |
| | WGYFDFNRFHCHFSPRDWQRLINNNWGFRPKRLNF | |
| | KLFNIQVKEVTDNNGVKTIANNLTSTVQVFTDSDY | |
| | QLPYVLGSAHEGCLPPFPADVFMIPQYGYLTLNDG | |
| | SQAVGRSSFYCLEYFPSQMLRTGNNFQFSYEFENV | |
| | PFHSSYAHSQSLDRLMNPLIDQYLYYLSKTINGSG | |
| | QNQQTLKFSVAGPSNMAVQGRNYIPGPSYRQQRVS | |
| | TTVTQNNNSEFAWPGASSWALNGRNSLMNPGPAMA | |
| | SHKEGEDRFFPLSGSLIFGKQGTGRDNVDADKVMI | |
| | TNEEEIKTTNPVATESYGQVATNHQSAQRGDLGLS | |
| | AQAQTGWVQNQGILPGMVWQDRDVYLQGPIWAKIP | |
| | HTDGNFHPSPLMGGFGMKHPPPQILIKNTPVPADP | |
| | PTAFNKDKLNSFITQYSTGQVSVEIEWELQKENSK | |
| | RWNPEIQYTSNYYKSNNVEFAVNTEGVYSEPRPIG | |
| | TRYLTRNL | |
| AAVmutl | MAADGYLPDWLEDNLSEGIREWWALKPGAPQPKAN | 306 |
| dec1 | QQHQDNARGLVLPGYKYLGPGNGLDKGEPVNAADA | |
| | AALEHDKAYDQQLKAGDNPYLKYNHADAEFQERLK | |
| | EDTSFGGNLGRAVFQAKKRLLEPLGLVEEAAKTAP | |
| | GKKRPVEQSPQEPDSSAGIGKSGAQPAKKRLNFGQ | |
| | TGDTESVPDPQPIGEPPAAPSGVGSLTMASGGGAP | |
| | VADNNEGADGVGSSSGNWHCDSQWLGDRVITTSTR | |
| | TWALPTYNNHLYKQISNSTSGASTNDNAYFGYSTP | |
| | WGYFDFNRFHCHFSPRDWQRLINNNWGFRPKRLNF | |
| | KLFNIQVKEVTDNNGVKTIANNLTSTVQVFTDSDY | |
| | QLPYVLGSAHEGCLPPFPADVFMIPQYGYLTLNDG | |
| | SQAVGRSSFYCLEYFPSQMLRTGNNFQFSYEFENV | |
| | PFHSSYAHSQSLDRLMNPLIDQYLYYLSKTINGSG | |
| | QNQQTLKFSVAGPSNMAVQGRNYIPGPSYRQQRVS | |
| | TTVTQNNNSEFAWPGASSWALNGRNSLMNPGPAMA | |
| | SHKEGEDRFFPLSGSLIFGKQGTGRDNVDADKVMI | |
| | TNEEEIKTTNPVATESYGQVATNHQSAQRGDLLLS | |
| | AQAQTGWVQNQGILPGMVWQDRDVYLQGPIWAKIP | |
| | HTDGNFHPSPLMGGFGMKHPPPQILIKNTPVPADP | |
| | PTAFNKDKLNSFITQYSTGQVSVEIEWELQKENSK | |
| | RWNPEIQYTSNYYKSNNVEFAVNTEGVYSEPRPIG | |
| | TRYLTRNL | |
| AAVdecol | MAADGYLPDWLEDNLSEGIREWWALKPGAPQPKAN | 307 |
| | QQHQDNARGLVLPGYKYLGPGNGLDKGEPVNAADA | |
| | AALEHDKAYDQQLKAGDNPYLKYNHADAEFQERLK | |
| | EDTSFGGNLGRAVFQAKKRLLEPLGLVEEAAKTAP | |
| | GKKRPVEQSPQEPDSSAGIGKSGAQPAKKRLNFGQ | |
| | TGDTESVPDPQPIGEPPAAPSGVGSLTMASGGGAP | |
| | VADNNEGADGVGSSSGNWHCDSQWLGDRVITTSTR | |
| | TWALPTYNNHLYKQISNSTSGGSSNDNAYFGYSTP | |
| | WGYFDFNRFHCHFSPRDWQRLINNNWGFRPKRLNF | |
| | KLFNIQVKEVTDNNGVKTIANNLTSTVQVFTDSDY | |
| | QLPYVLGSAHEGCLPPFPADVFMIPQYGYLTLNDG | |
| | SQAVGRSSFYCLEYFPSQMLRTGNNFQFSYEFENV | |
| | PFHSSYAHSQSLDRLMNPLIDQYLYYLSKTINGSG | |
| | QNQQTLKFSVAGPSNMAVQGRNYIPGPSYRQQRVS | |
| | TTVTQNNNSEFAWPGASSWALNGRNSLMNPGPAMA | |
| | SHKEGEDRFFPLSGSLIFGKQGTGRDNVDADKVMI | |
| | TNEEEIKTTNPVATESYGQVATNHQSAQRGDLLLS | |
| | AQAQTGWVQNQGILPGMVWQDRDVYLQGPIWAKIP | |
| | HTDGNFHPSPLMGGFGMKHPPPQILIKNTPVPADP | |
| | PTAFNKDKLNSFITQYSTGQVSVEIEWELQKENSK | |
| | RWNPEIQYTSNYYKSNNVEFAVNTEGVYSEPRPIG | |
| | TRYLTRNL | |
| AAVmut1 | MAADGYLPDWLEDNLSEGIREWWALKPGAPQPKAN | 308 |
| | QQHQDNARGLVLPGYKYLGPGNGLDKGEPVNAADA | |
| | AALEHDKAYDQQLKAGDNPYLKYNHADAEFQERLK | |
| | EDTSFGGNLGRAVFQAKKRLLEPLGLVEEAAKTAP | |
| | GKKRPVEQSPQEPDSSAGIGKSGAQPAKKRLNFGQ | |
| | TGDTESVPDPQPIGEPPAAPSGVGSLTMASGGGAP | |
| | VADNNEGADGVGSSSGNWHCDSQWLGDRVITTSTR | |
| | TWALPTYNNHLYKQISNSTSGASTNDNAYFGYSTP | |
| | WGYFDFNRFHCHFSPRDWQRLINNNWGFRPKRLNF | |
| | KLFNIQVKEVTDNNGVKTIANNLTSTVQVFTDSDY | |
| | QLPYVLGSAHEGCLPPFPADVFMIPQYGYLTLNDG | |
| | SQAVGRSSFYCLEYFPSQMLRTGNNFQFSYEFENV | |
| | PFHSSYAHSQSLDRLMNPLIDQYLYYLSKTINGSG | |
| | QNQQTLKFSVAGPSNMAVQGRNYIPGPSYRQQRVS | |
| | TTVTQNNNSEFAWPGASSWALNGRNSLMNPGPAMA | |
| | SHKEGEDRFFPLSGSLIFGKQGTGRDNVDADKVMI | |
| | TNEEEIKTTNPVATESYGQVATNHQSAQAQAQTGW | |
| | VQNQGILPGMVWQDRDVYLQGPIWAKIPHTDGNFH | |
| | PSPLMGGFGMKHPPPQILIKNTPVPADPPTAFNKD | |
| | KLNSFITQYSTGQVSVEIEWELQKENSKRWNPEIQ | |
| | YTSNYYKSNNVEFAVNTEGVYSEPRPIGTRYLTRN | |
| | L | |
| AAVcc.47 | MAADGYLPDWLEDNLSEGIREWWALKPGAPQPKAN | 309 |
| | QQHQDNARGLVLPGYKYLGPGNGLDKGEPVNAADA | |
| | AALEHDKAYDQQLKAGDNPYLKYNHADAEFQERLK | |
| | EDTSFGGNLGRAVFQAKKRLLEPLGLVEEAAKTAP | |
| | GKKRPVEQSPQEPDSSAGIGKSGAQPAKKRLNFGQ | |
| | TGDTESVPDPQPIGEPPAAPSGVGSLTMASGGGAP | |
| | VADNNEGADGVGSSSGNWHCDSQWLGDRVITTSTR | |
| | TWALPTYNNHLYKQISNSTSGGSSNDNAYFGYSTP | |
| | WGYFDFNRFHCHFSPRDWQRLINNNWGFRPKRLNF | |
| | KLFNIQVKEVTDNNGVKTIANNLTSTVQVFTDSDY | |
| | QLPYVLGSAHEGCLPPFPADVFMIPQYGYLTLNDG | |
| | SQAVGRSSFYCLEYFPSQMLRTGNNFQFSYEFENV | |
| | PFHSSYAHSQSLDRLMNPLIDQYLYYLSKTIGVSL | |
| | GGGQTLKFSVAGPSNMAVQGRNYIPGPSYRQQRVS | |
| | TTVTQNNNSEFAWPGASSWALNGRNSLMNPGPAMA | |
| | SHKEGEDRFFPLSGSLIFGKQGTGRDNVDADKVMI | |
| | TNEEEIKTTNPVATESYGQVATNHQSAQAQAQTGW | |
| | VQNQGILPGMVWQDRDVYLQGPIWAKIPHTDGNFH | |
| | PSPLMGGFGMKHPPPQILIKNTPVPADPPTAFNKD | |
| | KLNSFITQYSTGQVSVEIEWELQKENSKRWNPEIQ | |
| | YTSNYYKSNNVEFAVNTEGVYSEPRPIGTRYLTRN | |
| | L | |
| AAVHSC16 | MAADGYLPDWLEDNLSEGIREWWALKPGAPQPKAN | 310 |
| | QQHQDNARGLVLPGYKYLGPGNGLDKGEPVNAADA | |
| | AALEHDKAYDQQLKAGDNPYLKYNHADAEFQERLK | |
| | EDTSFGGNLGRAVFQAKKRLLEPLGLVEEAAKTAP | |
| | GKKRPVEQSPQEPDSSAGIGKSGAQPAKKRLNFGQ | |
| | TGDTESVPDPQPIGEPPAAPSGVGSLTMASGGGAP | |
| | VADNNEGADGVGSSSGNWHCDSQWLGDRVITTSTR | |
| | TWALPTYNNHLYKQISNSTSGGSSNDNAYFGYSTP | |
| | WGYFDFNRFHCHFSPRDWQRLINNNWGFRPKRLNF | |
| | KLFNIQVKEVTDNNGVKTIANNLTSTVQVFTDSDY | |
| | QLPYVLGSAHEGCLPPFPADVFMIPQYGYLTLNDG | |
| | SQAVGRSSFYCLEYFPSQMLRTGNNFQFSYEFENV | |
| | PFHSSYAHSQSLDRLMNPLIDQYLYYLSKTINGSG | |
| | QNQQTLKFSVAGPSNMAVQGRNYIPGPSYRQQRVS | |
| | TTVTQNNNSEIAWPRASSWALNGRNSLMNPGPAMA | |
| | SHKEGEDRFFPLSGSLIFGKQGTGRDNVDADKVMI | |
| | TNEEEIKTTNPVATESYGQVATNHQSAQAQAQTGW | |
| | VQNQGILPGMVWQDRDVYLQGPIWAKIPHTDGNFH | |
| | PSPLMGGFGMKHPPPQILIKNTPVPADPPTAFNKD | |
| | KLNSFITQYSTGQVSVEIEWELQKENSKRWNPEIQ | |
| | YTSNYCKSNNVEFAVNTEGVYSEPRPIGTRYLTRN | |
| | L | |
| MyoAAV- | MAADGYLPDWLEDNLSEGIREWWALKPGAPQPKAN | 311 |
| 4E | QQHQDNARGLVLPGYKYLGPGNGLDKGEPVNAADA | |
| | AALEHDKAYDQQLKAGDNPYLKYNHADAEFQERLK | |
| | EDTSFGGNLGRAVFQAKKRLLEPLGLVEEAAKTAP | |
| | GKKRPVEQSPQEPDSSAGIGKSGAQPAKKRLNFGQ | |
| | TGDTESVPDPQPIGEPPAAPSGVGSLTMASGGGAP | |
| | VADNNEGADGVGSSSGNWHCDSQWLGDRVITTSTR | |
| | TWALPTYNNHLYKQISNSTSGGSSNDNAYFGYSTP | |
| | WGYFDFNRFHCHFSPRDWQRLINNNWGFRPKRLNF | |
| | KLFNIQVKEVTDNNGVKTIANNLTSTVQVFTDSDY | |
| | QLPYVLGSAHEGCLPPFPADVFMIPQYGYLTLNDG | |
| | SQAVGRSSFYCLEYFPSQMLRTGNNFQFSYEFENV | |
| | PFHSSYAHSQSLDRLMNPLIDQYLYYLSKTINGSG | |
| | QNQQTLKFSVAGPSNMAVQGRNYIPGPSYRQQRVS | |
| | TTVTQNNNSEFAWPGASSWALNGRNSLMNPGPAMA | |
| | SHKEGEDRFFPLSGSLIFGKQGTGRDNVDADKVMI | |
| | TNEEEIKTTNPVATESYGQVATNHQENRRGDFNNT | |
| | AQAQTGWVQNQGILPGMVWQDRDVYLQGPIWAKIP | |
| | HTDGNFHPSPLMGGFGMKHPPPQILIKNTPVPADP | |
| | PTAFNKDKLNSFITQYSTGQVSVEIEWELQKENSK | |
| | RWNPEIQYTSNYYKSNNVEFAVNTEGVYSEPRPIG | |
| | TRYLTRNL | |
| MyoAAV- | MAADGYLPDWLEDNLSEGIREWWALKPGAPQPKAN | 312 |
| 3F | QQHQDNARGLVLPGYKYLGPGNGLDKGEPVNAADA | |
| | AALEHDKAYDQQLKAGDNPYLKYNHADAEFQERLK | |
| | EDTSFGGNLGRAVFQAKKRLLEPLGLVEEAAKTAP | |
| | GKKRPVEQSPQEPDSSAGIGKSGAQPAKKRLNFGQ | |
| | TGDTESVPDPQPIGEPPAAPSGVGSLTMASGGGAP | |
| | VADNNEGADGVGSSSGNWHCDSQWLGDRVITTSTR | |
| | TWALPTYNNHLYKQISNSTSGGSSNDNAYFGYSTP | |
| | WGYFDFNRFHCHFSPRDWQRLINNNWGFRPKRLNF | |
| | KLFNIQVKEVTDNNGVKTIANNLTSTVQVFTDSDY | |
| | QLPYVLGSAHEGCLPPFPADVFMIPQYGYLTLNDG | |
| | SQAVGRSSFYCLEYFPSQMLRTGNNFQFSYEFENV | |
| | PFHSSYAHSQSLDRLMNPLIDQYLYYLSKTINGSG | |
| | QNQQTLKFSVAGPSNMAVQGRNYIPGPSYRQQRVS | |
| | TTVTQNNNSEFAWPGASSWALNGRNSLMNPGPAMA | |
| | SHKEGEDRFFPLSGSLIFGKQGTGRDNVDADKVMI | |
| | TNEEEIKTTNPVATESYGQVATNHQSAQRGDHASW | |
| | AQAQTGWVQNQGILPGMVWQDRDVYLQGPIWAKIP | |
| | HTDGNFHPSPLMGGFGMKHPPPQILIKNTPVPADP | |
| | PTAFNKDKLNSFITQYSTGQVSVEIEWELQKENSK | |
| | RWNPEIQYTSNYYKSNNVEFAVNTEGVYSEPRPIG | |
| | TRYLTRNL | |
| MyoAAV- | MAADGYLPDWLEDNLSEGIREWWALKPGAPQPKAN | 313 |
| 4A | QQHQDNARGLVLPGYKYLGPGNGLDKGEPVNAADA | |
| | AALEHDKAYDQQLKAGDNPYLKYNHADAEFQERLK | |
| | EDTSFGGNLGRAVFQAKKRLLEPLGLVEEAAKTAP | |
| | GKKRPVEQSPQEPDSSAGIGKSGAQPAKKRLNFGQ | |
| | TGDTESVPDPQPIGEPPAAPSGVGSLTMASGGGAP | |
| | VADNNEGADGVGSSSGNWHCDSQWLGDRVITTSTR | |
| | TWALPTYNNHLYKQISNSTSGGSSNDNAYFGYSTP | |
| | WGYFDFNRFHCHFSPRDWQRLINNNWGFRPKRLNF | |
| | KLFNIQVKEVTDNNGVKTIANNLTSTVQVFTDSDY | |
| | QLPYVLGSAHEGCLPPFPADVFMIPQYGYLTLNDG | |
| | SQAVGRSSFYCLEYFPSQMLRTGNNFQFSYEFENV | |
| | PFHSSYAHSQSLDRLMNPLIDQYLYYLSKTINGSG | |
| | QNQQTLKFSVAGPSNMAVQGRNYIPGPSYRQQRVS | |
| | TTVTQNNNSEFAWPGASSWALNGRNSLMNPGPAMA | |
| | SHKEGEDRFFPLSGSLIFGKQGTGRDNVDADKVMI | |
| | TNEEEIKTTNPVATESYGQVATNHQSNSRGDYNSL | |
| | AQAQTGWVQNQGILPGMVWQDRDVYLQGPIWAKIP | |
| | HTDGNFHPSPLMGGFGMKHPPPQILIKNTPVPADP | |
| | PTAFNKDKLNSFITQYSTGQVSVEIEWELQKENSK | |
| | RWNPEIQYTSNYYKSNNVEFAVNTEGVYSEPRPIG | |
| | TRYLTRNL | |
| MyoAAV- | MAADGYLPDWLEDNLSEGIREWWALKPGAPQPKAN | 314 |
| 4D | QQHQDNARGLVLPGYKYLGPGNGLDKGEPVNAADA | |
| | AALEHDKAYDQQLKAGDNPYLKYNHADAEFQERLK | |
| | EDTSFGGNLGRAVFQAKKRLLEPLGLVEEAAKTAP | |
| | GKKRPVEQSPQEPDSSAGIGKSGAQPAKKRLNFGQ | |
| | TGDTESVPDPQPIGEPPAAPSGVGSLTMASGGGAP | |
| | VADNNEGADGVGSSSGNWHCDSQWLGDRVITTSTR | |
| | TWALPTYNNHLYKQISNSTSGGSSNDNAYFGYSTP | |
| | WGYFDFNRFHCHFSPRDWQRLINNNWGFRPKRLNF | |
| | KLFNIQVKEVTDNNGVKTIANNLTSTVQVFTDSDY | |
| | QLPYVLGSAHEGCLPPFPADVFMIPQYGYLTLNDG | |
| | SQAVGRSSFYCLEYFPSQMLRTGNNFQFSYEFENV | |
| | PFHSSYAHSQSLDRLMNPLIDQYLYYLSKTINGSG | |
| | QNQQTLKFSVAGPSNMAVQGRNYIPGPSYRQQRVS | |
| | TTVTQNNNSEFAWPGASSWALNGRNSLMNPGPAMA | |
| | SHKEGEDRFFPLSGSLIFGKQGTGRDNVDADKVMI | |
| | TNEEEIKTTNPVATESYGQVATNHQASTRGDHGVL | |
| | AQAQTGWVQNQGILPGMVWQDRDVYLQGPIWAKIP | |
| | HTDGNFHPSPLMGGFGMKHPPPQILIKNTPVPADP | |
| | PTAFNKDKLNSFITQYSTGQVSVEIEWELQKENSK | |
| | RWNPEIQYTSNYYKSNNVEFAVNTEGVYSEPRPIG | |
| | TRYLTRNL | |
| 4D-C102 | MAADGYLPDWLEDTLSEGIRQWWKLKPGPPPPKPA | 315 |
| (variant | ERHKDDSRGLVLPGYKYLGPFNGLDKGEPVNEADA | |
| sequence- | AALEHDKAYDRQLDSGDNPYLKYNHADAEFQERLK | |
| 1) | EDTSFGGNLGRAVFQAKKRVLEPLGLVEEPVKTAP | |
| | GKKRPVEHSPVEPDSSSGTGKAGQQPARKRLNFGQ | |
| | TGDADSVPDPQPLGQPPAAPSGLGTNTMATGSGAP | |
| | MADNNEGADGVGNSSGNWHCDSTWMGDRVITTSTR | |
| | TWALPTYNNHLYKQISSQSGASNDNHYFGYSTPWG | |
| | YFDFNRFHCHFSPRDWQRLINNNWGFRPKRLNFKL | |
| | FNIQVKEVTQNDGTTTIANNLTSTVQVFTDSEYQL | |
| | PYVLGSAHQGCLPPFPADVFMVPQYGYLTLNNGSQ | |
| | AVGRSSFYCLEYFPSQMLRTGNNFTFSYTFEDVPF | |
| | HSSYAHSQSLDRLMNPLIDQYLYYLSRTNTPSGTT | |
| | TQSRLQFSQAGASDIRDQSRNWLPGPCYRQQRVSK | |
| | TSADNNNSEYSWTGATKYHLNGRDSLVNPGPAMAS | |
| | HKDDEEKFFPQSGVLIFGKQGSEKTNVDIEKVMIT | |
| | DEEEIRTTNPVATEQYGSVSTNLQRGNLANKTTNK | |
| | DARQAATADVNTQGVLPGMVWQDRDVYLQGPIWAK | |
| | IPHTDGHFHPSPLMGGFGLKHPPPQILIKNTPVPA | |
| | NPSTTFSAAKFASFITQYSTGQVSVEIEWELQKEN | |
| | SKRWNPEIQYTSNYNKSINVDFTVDTNGVYSEPRP | |
| | IGTRYLTRNL | |
| 4D-C102 | MAADGYLPDWLEDTLSEGIRQWWKLKPGPPPPKPA | 316 |
| (variant_ | ERHKDDSRGLVLPGYKYLGPFNGLDKGEPVNEADA | |
| sequence- | AALEHDKAYDRQLDSGDNPYLKYNHADAEFQERLK | |
| 2) | EDTSFGGNLGRAVFQAKKRVLEPLGLVEEPVKTAP | |
| | GKKRPVEHSPVEPDSSSGTGKAGQQPARKRLNFGQ | |
| | TGDADSVPDPQPLGQPPAAPSGLGTNTMATGSGAP | |
| | MADNNEGADGVGNSSGNWHCDSTWMGDRVITTSTR | |
| | TWALPTYNNHLYKQISSQSGASNDNHYFGYSTPWG | |
| | YFDFNRFHCHFSPRDWQRLINNNWGFRPKRLNFKL | |
| | FNIQVKEVTQNDGTTTIANNLTSTVQVFTDSEYQL | |
| | PYVLGSAHQGCLPPFPADVFMVPQYGYLTLNNGSQ | |
| | AVGRSSFYCLEYFPSQMLRTGNNFTFSYTFEDVPF | |
| | HSSYAHSQSLDRLMNPLIDQYLYYLSRTNTPSGTT | |
| | TQSRLQFSQAGASDIRDQSRNWLPGPCYRQQRVSK | |
| | TSADNNNSEYSWTGATKYHLNGRDSLVNPGPAMAS | |
| | HKDDEEKFFPQSGVLIFGKQGSEKTNVDIEKVMIT | |
| | DEEEIRTTNPVATEQYGSVSTNLQRGNLANKIQRT | |
| | DARQAATADVNTQGVLPGMVWQDRDVYLQGPIWAK | |
| | IPHTDGHFHPSPLMGGFGLKHPPPQILIKNTPVPA | |
| | NPSTTFSAAKFASFITQYSTGQVSVEIEWELQKEN | |
| | SKRWNPEIQYTSNYNKSINVDFTVDTNGVYSEPRP | |
| | IGTRYLTRNL | |

In some embodiments, the rAAV is replication defective, in that the rAAV virion cannot independently further replicate and package its genome. For example, when a cardiac cell is targeted with rAAV virions, the transgene is expressed in the targeted cardiac cell, however, since the targeted cardiac cell lacks AAV rep and cap genes and accessory function genes, the rAAV is not able to replicate.

In some embodiments, rAAV virions of the present disclosure encapsulating the expression cassettes as described herein, can be produced using helper-free production. rAAVs are replication-deficient viruses and normally require components from a live helper virus, such as adenovirus, in a host cell for packaging of infectious rAAV virions. rAAV helper-free production systems allow the production of infectious rAAV virions without the use of a live helper virus. In the helper-free system, a host packaging cell line is co-transfected with three plasmids. A first plasmid may contain adenovirus gene products (e.g., E2A, E4, and VA RNA genes) needed for the packaging of rAAV virions. A second plasmid may contain required AAV genes (e.g., REP and CAP genes). A third plasmid contains the polynucleotide sequence encoding the transgene of interest and a promoter flanked by ITRs. A host packaging cell line can be, for example, AAV-293 host cells. Suitable host cells contain additional components required for packaging infectious rAAV virions that are not supplied by the plasmids. In some embodiments, the CAP genes can encode, for example, AAV capsid proteins as described herein.

### Inhibitory RNA

In some embodiments, the inhibitory RNA provided herein can be, without limitation, a small interfering RNA (siRNA), a short hairpin RNA (shRNA), or an antisense RNA. The inhibitory RNA can be any RNA that regulates expression of genes by RNA interference (RNAi).

An siRNA can be a single stranded RNA or a double stranded RNA (dsRNA). A dsRNA comprises an antisense strand complementary to a target mRNA sequence, and a sense strand complementary to the antisense strand.

In some embodiments, the siRNA is any siRNA described in Dana et al., 2017, Int J Biomed Sci Vol. 13 No. 2: 48-57, incorporated herein by reference in its entirety. In some embodiments, the siRNA can be delivered using any viral and non-viral delivery methods described in Dana et al., 2017, Int J Biomed Sci Vol. 13 No. 2: 48-57, incorporated herein by reference in its entirety.

In some embodiments, the siRNA is any single stranded siRNA or double stranded siRNA described in Elsner, 2012, Nature Biotechnology Vol. 30 No. 11: 1063, incorporated herein by reference in its entirety. In some embodiments, the siRNA can be delivered using any delivery methods described in Elsner, 2012, Nature Biotechnology Vol. 30 No. 11: 1063, incorporated herein by reference in its entirety.

In some embodiments, the siRNA or shRNA are any of those described in Moore et al, 2010, Methods Mol Biol. 629: 141-158, incorporated herein by reference in its entirety. In some embodiments, the siRNA or shRNA can be delivered using any delivery methods, such as vector delivery, described in Moore et al, 2010, Methods Mol Biol. 629: 141-158, incorporated herein by reference in its entirety.

In some embodiments, the siRNA or shRNA are any of those described in Kurreck et al, 2017, J RNAi Gene Silencing Vol 13, 545-547, incorporated herein by reference in its entirety. In some embodiments, the siRNA or shRNA can be delivered using any delivery methods, such as viral vector delivery, e.g., by use of AAV, described in Kurreck et al, 2017, J RNAi Gene Silencing Vol 13, 545-547, incorporated herein by reference in its entirety.

In some embodiments, inhibitory RNA (e.g. siRNA or shRNA) can be delivered to a subject using delivery systems that include, but are not limited to lipid formulations, nanoparticles, functional groups covalently coupled to the RNA, or viral vectors such as AAV (see, e.g., Kurreck et al, J RNAi Gene Silencing, 2017, Vol 13, 545-547; Moore et al, Methods Mol Biol., 629: 141-158, 2010). In some embodiments, inhibitory RNA is delivered as a naked RNA.

In some embodiments, shRNA can be delivered using a DNA vector. In some embodiments shRNA can be delivered using a lentiviral vector.

In some embodiments, the inhibitory RNA provided herein inhibits expression of any gene conferring further cardiac risk in a cardiac cell or patient having a deleterious TTN mutation. In some embodiments, the inhibitory RNA provided herein confers a cardioprotective effect in a cardiac cell or patient having a deleterious TTN mutation. In some embodiments, the inhibitory RNA provided herein protects against and/or ameliorates sarcomere dysfunction and/or disarray in a cardiac cell or patient having a TTN mutation.

In some embodiments, the inhibitory RNA provided herein inhibits expression of any gene conferring further cardiac risk in a cardiac cell or patient having a deleterious MLP/CSRP3 mutation. In some embodiments, the inhibitory RNA provided herein confers a cardioprotective effect in a cardiac cell or patient having a deleterious MLP/CSRP3 mutation. In some embodiments, the inhibitory RNA provided herein protects against and/or ameliorates sarcomere dysfunction and/or disarray in a cardiac cell or patient having an MLP/CSRP3 mutation.

In some embodiments, the inhibitory RNA provided herein inhibits expression of MTSS1 gene. In some embodiments, the inhibitory RNA provided herein interferes with MTSS1 mRNA. In some embodiments, such inhibitory RNA is siRNA. In some embodiments, such inhibitory RNA is shRNA. In some embodiments, such inhibitory RNA regulates expression of MTSS1 by RNAi. In some embodiments, such inhibitory RNA is an antisense RNA.

In some embodiments, an inhibitory RNA inhibiting expression of MTSS1 has at least 15 contiguous nucleotides of any one of the sequences of SEQ ID NOs: 94-100. In some embodiments, an inhibitory RNA inhibiting expression of MTSS1 has at least 16 contiguous nucleotides of any one of the sequences of SEQ ID NOs: 94-100. In some embodiments, an inhibitory RNA inhibiting expression of MTSS1 has at least 17 contiguous nucleotides of any one of the sequences of SEQ ID NOs: 94-100. In some embodiments, an inhibitory RNA inhibiting expression of MTSS1 has at least 18 contiguous nucleotides of any one of the sequences of SEQ ID NOs: 94-100. In some embodiments, an inhibitory RNA inhibiting expression of MTSS1 has at least 19 contiguous nucleotides of any one of the sequences of SEQ ID NOs: 94-100.

In some embodiments, an inhibitory RNA inhibiting expression of MTSS1 has at least 15 contiguous nucleotides of any one of the sequences of SEQ ID NOs: 94-100, with up to 3 mismatches (e.g., 1, 2, or 3 mismatches). In some embodiments, an inhibitory RNA inhibiting expression of MTSS1 has at least 16 contiguous nucleotides of any one of the sequences of SEQ ID NOs: 94-100, with up to 3 mismatches (e.g., 1, 2, or 3 mismatches). In some embodiments, an inhibitory RNA inhibiting expression of MTSS1 has at least 17 contiguous nucleotides of any one of the sequences of SEQ ID NOs: 94-100, with up to 3 mismatches (e.g., 1, 2, or 3 mismatches). In some embodiments, an inhibitory RNA inhibiting expression of MTSS1 has at least 18 contiguous nucleotides of any one of the sequences of SEQ ID NOs: 94-100, with up to 3 mismatches (e.g., 1, 2, or 3 mismatches). In some embodiments, an inhibitory RNA inhibiting expression of MTSS1 has at least 19 contiguous nucleotides of any one of the sequences of SEQ ID NOs: 94-100, with up to 3 mismatches (e.g., 1, 2, or 3 mismatches).

In some embodiments, an inhibitory RNA inhibiting expression of MTSS1 has at least 15, at least 16, at least 17, at least 18, or at least 19 contiguous nucleotides of SEQ ID NO: 94. In some embodiments, an inhibitory RNA inhibiting expression of MTSS1 has at least 15, at least 16, at least 17, at least 18, or at least 19 contiguous nucleotides of SEQ ID NO: 95. In some embodiments, an inhibitory RNA inhibiting expression of MTSS1 has at least 15, at least 16, at least 17, at least 18, or at least 19 contiguous nucleotides of SEQ ID NO: 96. In some embodiments, an inhibitory RNA inhibiting expression of MTSS1 has at least 15, at least 16, at least 17, at least 18, or at least 19 contiguous nucleotides of SEQ ID NO: 97. In some embodiments, an inhibitory RNA inhibiting expression of MTSS1 has at least 15, at least 16, at least 17, at least 18, or at least 19 contiguous nucleotides of SEQ ID NO: 98. In some embodiments, an inhibitory RNA inhibiting expression of MTSS1 has at least 15, at least 16, at least 17, at least 18, or at least 19 contiguous nucleotides of SEQ ID NO: 99. In some embodiments, an inhibitory RNA inhibiting expression of MTSS1 has at least 15, at least 16, at least 17, at least 18, or at least 19 contiguous nucleotides of SEQ ID NO: 100.

In some embodiments, an inhibitory RNA inhibiting expression of MTSS1 shares at least 90%, 95%, 96%, 97%, 98%, 99%, or 100% identity to any one of the sequences of SEQ ID NOs: 94-100. In some embodiments, an inhibitory RNA inhibiting expression of MTSS1 comprises SEQ ID NO: 94. In some embodiments, an inhibitory RNA inhibiting expression of MTSS1 comprises SEQ ID NO: 95. In some embodiments, an inhibitory RNA inhibiting expression of MTSS1 comprises SEQ ID NO: 96. In some embodiments, an inhibitory RNA inhibiting expression of MTSS1 comprises SEQ ID NO: 97. In some embodiments, an inhibitory RNA inhibiting expression of MTSS1 comprises SEQ ID NO: 98. In some embodiments, an inhibitory RNA inhibiting expression of MTSS1 comprises SEQ ID NO: 99. In some embodiments, an inhibitory RNA inhibiting expression of MTSS1 comprises SEQ ID NO: 100.

### Gene Editing Constructs

In some embodiments, provided herein is a gene editing system targeting MTSS1 expression.

In some embodiments, provided herein is a vector comprising a polynucleotide sequence that encodes gene editing component(s) targeting MTSS1 expression, operatively linked to a promoter.

Gene editing approaches known to a person skilled in the art, e.g., by clustered regularly interspaced short palindromic repeat (CRISPR) and CRISPR-associated (Cas) proteins. The CRISPR/Cas system was originally discovered in prokaryotic organisms as a system involved in defense against invading phages and plasmids and has been adapted and used as a popular gene editing tool in research and clinical applications. CRISPR/Cas systems generally comprise at least two components: one or more non-coding RNAs referred to as guide RNAs (gRNAs) and a Cas protein with nuclease functionality. Cas9, originally derived from S. pyogenes (SpCas9) or S. aureus (SaCas9), is the most widely used Cas protein. One or more elements of a CRISPR/Cas system can derive from a type I, type II, or type III CRISPR system, e.g., derived from a particular organism comprising an endogenous CRISPR system, such as S. pyogenes. The gRNA guides the Cas protein to a particular genomic sequence to make a double-strand break (DSB) in the target DNA, thereby utilizing the cells' mechanisms for DSB repair and resulting in site-specific insertion, deletion, or other modifications of the target genomic sequence.

The components of a CRISPR system can be implemented in any suitable manner, meaning that the components of such systems including the RNA-guided nuclease (e.g., a Cas protein) and gRNA can be delivered, formulated, or administered in any suitable form to the cells. For example, the RNA-guided nuclease may be delivered to a cell complexed with a gRNA (e.g., as a ribonucleoprotein (RNP) complex), the RNA-guided nuclease may be delivered to a cell separate (e.g., uncomplexed) to a gRNA, the RNA-guided nuclease may be delivered to a cell as a polynucleotide (e.g., DNA or RNA) encoding the nuclease that is separate from a gRNA, or both the RNA-guided nuclease and the gRNA molecule may be delivered as polynucleotides encoding each component. Additionally, one or more vectors driving expression of one or more elements of the CRISPR system can be introduced into the cell such that expression of the elements of the CRISPR system direct formation of the CRISPR complex at one or more target sites.

In some embodiments, a Cas protein and a gRNA are introduced into the cell, for example, by transfecting or transducing the cell using one or more vectors encoding the Cas protein and gRNA. In general, target sites at the 5′ end of the gRNA direct the Cas protein to the target site, e.g., the gene, using complementary base pairing. The target site may be selected based on its location immediately 5′ of a protospacer adjacent motif (PAM) sequence, typically NGG or NAG. In this respect, the gRNA is targeted to the desired sequence by modifying the first 20, 19, 18, 17, 16, 15, 14, 13, 12, 11, or 10 nucleotides of the guide RNA to correspond to the target DNA sequence. In general, a CRISPR system is characterized by elements that promote the formation of a CRISPR complex at the site of a target sequence. Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex.

In some embodiments, the present disclosure provides polynucleotide sequences, expression cassettes, and/or vectors comprising a first polynucleotide encoding a Cas9 protein operably linked to a first promoter and a second polynucleotide encoding a gRNA (e.g., a gRNA targeting the MTSS1 gene) operably linked to a second promoter. In some embodiments, the first polynucleotide encoding a Cas9 protein and the second polynucleotide encoding a gRNA are in the same vector. In some embodiments, the first polynucleotide encoding a Cas9 protein and the second polynucleotide encoding a gRNA are in separate vectors.

In some embodiments, the first promoter is any promoter suitable for protein expression described herein. In some embodiments, the first promoter is any muscle-specific promoter or a cardiac-specific promoter described herein or known in the art. In some embodiments, the first promoter is a TNNT2 promoter, such as any TNNT2 promoter described herein. In some embodiments, the first promoter is a human TNNT2 promoter. In some embodiments, the first promoter comprises, essentially consists of, or consists of any one of SEQ ID NOs: 1-85. In some embodiments of the expression cassettes and vectors described herein, the TNNT2 promoter of SEQ ID NO: 1 or the TNNT2 promoter of SEQ ID NO: 3 is used for cardiac-specific Cas endonuclease expression.

In some embodiments, the second promoter is any promoter suitable for expression of RNA. In some embodiments, the second promoter is any Pol III promoter (e.g., human Pol III promoter) described herein or known in the art. In some embodiments, the second promoter is a U6 promoter. In some embodiments, the second promoter is a human U6 promoter. In some embodiments, the second promoter comprises, essentially consists of, or consists of SEQ ID NO: 101.

In some embodiments, the first polynucleotide sequence further comprises a polyadenylation (poly(A)) signal, and optionally a transcription termination signal.

In some embodiments, provided herein are expression cassettes and vectors comprising a Cas endonuclease protein (e.g., Cas9 or saCas9) operably linked to a protein expression-driving promoter (e.g., a cardiac-specific promoter such as a human TNNT2 promoter), and/or a second polynucleotide encoding a guide RNA complementary to a sequence of the MTSS1 gene or a non-coding sequence promoting expression of the MTSS1 gene (e.g., specifically promoting expression in muscle cells or cardiac cells) linked to an RNA expression-driving promoter (e.g., a U6 promoter such as human U6 promoter). In some embodiments, the first polynucleotide and the second polynucleotide are in the same expression cassette or vector. In some embodiments, the vector is a DNA-based vector, an mRNA-based vector, an AAV-based vector, a retrovirus-based vector, or a lentivirus-based vector. In some embodiments, the vector is an adenoviral vector or a lentiviral vector. In some embodiments, the vector is an AAV9 vector or a variant thereof. In some embodiments, provide herein is an rAAV virion comprising an AAV vector comprising a gene editing system described herein and capsid proteins, wherein the capsid proteins can be wild-type or engineered capsid proteins (such as any described herein),

In some embodiments, the polynucleotides, expression cassettes, and/or vectors described herein comprise, consist essentially of, or consist of (i) a first polynucleotide sequence encoding a Cas protein (e.g., a SpCas9 or SaCAs9) operably linked to a first promoter (e.g., a human TNNT2 promoter) and further comprising a poly(A) sequence, and (ii) a second polynucleotide encoding a gRNA (such as targeting MTSS1 as described herein) operably linked to a second promoter (e.g., a human U6 promoter). In some embodiments, the first and second polynucleotide sequences are present in a single vector. In some embodiments, the first and second polynucleotide sequences are present in separate vectors.

In some embodiments, a Cas endonuclease is any Cas endonuclease (e.g., Cas9) encoded by a gene equal to or less than 3.3 kb, 3.2 kb, 3.1 kb, 3 kb, 2.9 kb or 2.8 kb in size. In some embodiments, a Cas endonuclease is any Cas endonuclease (e.g., Cas9) which has the protein size of equal to or less than 1,100 amino acids, 1,075 amino acids, 1,060 amino acids, 1,050 amino acids, 1,000 amino acids, 950 amino acids, or 900 amino acids.

In some embodiments, Cas9 endonuclease is a Staphylococcus aureus Cas9 (SaCas9).

In some embodiments, the Cas endonuclease protein is any Cas endonuclease known in the art, including, for example, Cas1, Cas1B, Cas2, Cas3, Cas4, Cas5, Cas6, Cas7, Cas8, Cas9 (also known as Csn1 and Csxl2), Cas10, CasX, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csxl7, Csxl4, CsxlO, Csxl6, CsaX, Csx3, Csxl, Csxl5, Csf1, Csf2, Csf3, Csf4, homologs thereof, or modified versions thereof. These enzymes are known, for example, the amino acid sequence of S. pyogenes Cas9 protein may be found in the SwissProt database under accession number Q99ZW2. Illustrative examples of Cas enzymes that can be used are provided in Table 10A below (where N denotes any nucleotide).

TABLE 10A
Exemplary Cas enzymes and associated PAM sequences

| | | PAM | Size |
|---|---|---|---|
| Name | Species | Sequence | (amino acids) |
| SpCas9 | Streptococcus pyogenes | NGG | 1,368 |
| SaCas9 | Staphylococcus aureus | NNGRRT | 1,053 |
| FnCas9 | Francisella novicida | NGG | 1,244 |
| NmCas9 | Neisseria meningitidis | NNNNGATT | 1,055 |
| StCas9 | Streptococcus. Thermophilus | NGG | 1,364 |
| AaCas9 | Acidaminococcus sp | NNNNRYAC | 984 |
| GstCas9 | Geobacillus | NGG | 1,245 |
| | stearothermophilus | | |
| NmuCas9 | Neisseria mucosa | NNAGAAW | 1,054 |
| ScCas9 | Streptococcus canis | NGG | 1,342 |
| LbCas9 | Lachnospiraceae bacterium | TTTA | 1,305 |
| SsCas9 | Streptococcus sanguinis | NNGRRT | 1,365 |
| NsuCas9 | Neisseria subflava | NNNNGMTT | 1,056 |
| SeCas9 | Staphylococcus epidermidis | NNGS | 1,054 |
| SagalCas9 | Streptococcus agalactiae | NNGRRT | 1,363 |
| LmCas9 | Listeria monocytogenes | NGG | 1,359 |
| AspCas9 | Actinomyces sp. | NNAGAAW | 1,045 |
| SangCas9 | Streptococcus anginosus | NNNNGATT | 1,056 |
| SdCas9 | Streptococcus dysgalactiae | NNGRRT | 1,341 |
| CjCa9 | Campylobacter jejuni | NNNNRYAC | 1,235 |
| BtCas9 | Bacillus thuringiensis | NGG | 1,243 |
| CdCas9 | Clostridium difficile | NGG | 1,406 |
| ScristCas9 | Streptococcus cristatus | NNNNGATT | 1,056 |
| SmCas9 | Streptococcus mutans | NNGG | 1,333 |
| AsCas12a | Acidaminococcus sp. | TTTV | 1,278 |
| LbCas12b | Lachnospiraceae bacterium | TYCV | 1,187 |
| MboCas 12a | Moraxella bovoculi | TTTV | 1,286 |
| RspCas12b | Ruminococcus sp | TYCV | 1,185 |
| AccCas12a | Acidothermus cellulolyticus | TTTV | 1,285 |
| PspCas12a | Prevotella sp. | TTTV | 1,264 |
| PgiCas12b | Porphyromonas gingivalis | TYCV | 1,167 |
| SmaCas12a | Streptococcus mutans | TTTV | 1,259 |
| PinCas12b | Prevotella intermedia | TYCV | 1,188 |
| AspCas12a | Acidovorax sp. | TTTV | 1,284 |
| EspCas12b | Enterobacter sp. | TYCV | 1,296 |
| SpyCas12b | Streptococcus pneumoniae | TYCV | 1,157 |
| AacCas12a | Alicyclobacillus | TTTV | 1,285 |
| | acidocaldarius | | |
| AcbCas12b | Acidobacterium sp. | TYCV | 1,174 |
| PaeCas12a | Pseudomonas aeruginosa | TTTV | 1,284 |
| BfrCas12b | Bacteroides fragilis | TYCV | 1,181 |
| LcrCas12a | Lactobacillus crispatus | TTTV | 1,259 |
| SmaCas12b | Serratia marcescens | TYCV | 1,188 |
| MolCas 12a | Moraxella osloensis | TTTV | 1,284 |
| BunCas12b | Bacteroides uniformis | TYCV | 1,197 |
| KoxCas12a | Klebsiella oxytoca | TTTV | 1,292 |
| BthCas12b | Bacillus thuringiensis | TYCV | 1,182 |
| RspCasX | Ruminococcus sp. | TTN | 926 |
| PspCasX | Pseudomonas sp. | TTN | 918 |
| SepCasX | Staphylococcus epidermidis | TTN | 927 |

In some embodiments, the Cas endonuclease is Cas9. In some embodiments, the Cas9 is derived from S. aureus, S. pyogenes, F. novicida, N. meningitidis, S. thermophilus, Acidaminococcus sp., G. stearothermophilus, N. mucosa, S. canis, Lachnospiraceae bacterium, S. sanguinis, N. subflava, S. epidermidis, S. agalactiae, L. monocytogenes, Actinomyces sp., S. anginosus, S. dysgalactiae, C. jejuni, B. thuringiensis, C. difficile, S. cristatus, S. mutans, or S. pneumonia. In some embodiments, the Cas endonuclease is Cas9 derived from S. pyogenes (SpCas9) or S. aureus (SaCas9).

In some embodiments, the Cas endonuclease is Casl2a. In some embodiments, the Cas12a is derived from Acidaminococcus sp., M. bovoculi, A. cellulolyticus, Prevotella sp., S. mutans, Acidovorax sp., A. acidocaldarius, P. aeruginosa, L. crispatus, M. osloensis, or K. oxytoca.

In some embodiments, the Cas endonuclease is Casl2b. In some embodiments, the Cas12b is derived from Lachnospiraceae bacterium, Ruminococcus sp., P. gingivalis, P. intermedia, Enterobacter sp., S. pneumoniae, Acidobacterium sp., B. fragilis, S. marcescens, B. uniformis, or B. thuringiensis.

In some embodiments, the Cas endonuclease is CasX. In some embodiments, the CasX is derived from Ruminococcus sp., Pseudomonas sp., or S. epidermidis.

The protospacer adjacent motif (PAM), the sequence adjacent to the target sequence, is an essential targeting component for the design of CRISPR/Cas9-mediated gene editing. In some embodiments, the SaCas9 PAM sequence is NNGRR or for optimal on-target cutting is NNGRRT, wherein N comprises any of Adenine, Guanine, Cytosine, or Thymine, and R comprises any of Guanine or Adenine. PAMs for use with different Cas endonucleases are known in the art. Illustrative examples of PAMs for use with their respective Cas endonuclease are provided in Table 10A above.

In some embodiments, provided is a polynucleotide encoding a Cas (e.g., a Cas9) endonuclease. In some embodiments, the polynucleotide encoding a Cas endonuclease comprises or consists of a sequence that shares at least 60%, at least 65%, at least 70%, at least 75%, at least 80%, at least 85%, at least 90%, at least 95%, at least 96%, at least 97%, at least 98%, at least 99%, or 100% identity to SEQ ID NO: 398. In some embodiments, the Cas endonuclease comprises or consists of an amino acid sequence that shares at least 60%, at least 65%, at least 70%, at least 75%, at least 80%, at least 85%, at least 90%, at least 95%, at least 96%, at least 97%, at least 98%, at least 99%, or 100% identity to SEQ ID NO: 399.

TABLE 10B
Exemplary Cas endonuclease sequences

| | | SEQ |
|---|---|---|
| | | ID |
| Name | Sequence | NO: |
| SaCas9 | atggccccaaagaagaagcggaaggtcggt | 398 |
| DNA | atccacggagtcccagcagccaagcggaac | |
| sequence | tacatcctgggcctggacatcggcatcacc | |
| | agcgtgggctacggcatcatcgactacgag | |
| | acacgggacgtgatcgatgccggcgtgcgg | |
| | ctgttcaaagaggccaacgtggaaaacaac | |
| | gagggcaggcggagcaagagaggcgccaga | |
| | aggctgaagcggcggaggcggcatagaatc | |
| | cagagagtgaagaagctgctgttcgactac | |
| | aacctgctgaccgaccacagcgagctgagc | |
| | ggcatcaacccctacgaggccagagtgaag | |
| | ggcctgagccagaagctgagcgaggaagag | |
| | ttctctgccgccctgctgcacctggccaag | |
| | agaagaggcgtgcacaacgtgaacgaggtg | |
| | gaagaggacaccggcaacgagctgtccacc | |
| | aaagagcagatcagccggaacagcaaggcc | |
| | ctggaagagaaatacgtggccgaactgcag | |
| | ctggaacggctgaagaaagacggcgaagtg | |
| | cggggcagcatcaacagattcaagaccagc | |
| | gactacgtgaaagaagccaaacagctgctg | |
| | aaggtgcagaaggcctaccaccagctggac | |
| | cagagcttcatcgacacctacatcgacctg | |
| | ctggaaacccggcggacctactatgaggga | |
| | cctggcgagggcagccccttcggctggaag | |
| | gacatcaaagaatggtacgagatgctgatg | |
| | ggccactgcacctacttccccgaggaactg | |
| | cggagcgtgaagtacgcctacaacgccgac | |
| | ctgtacaacgccctgaacgacctgaacaat | |
| | ctcgtgatcaccagggacgagaacgagaag | |
| | ctggaatattacgagaagttccagatcatc | |
| | gagaacgtgttcaagcagaagaagaagccc | |
| | accctgaagcagatcgccaaagaaatcctc | |
| | gtgaacgaagaggatattaagggctacaga | |
| | gtgaccagcaccggcaagcccgagttcacc | |
| | aacctgaaggtgtaccacgacatcaaggac | |
| | attaccgcccggaaagagattattgagaac | |
| | gccgagctgctggatcagattgccaagatc | |
| | ctgaccatctaccagagcagcgaggacatc | |
| | caggaagaactgaccaatctgaactccgag | |
| | ctgacccaggaagagatcgagcagatctct | |
| | aatctgaagggctataccggcacccacaac | |
| | ctgagcctgaaggccatcaacctgatcctg | |
| | gacgagctgtggcacaccaacgacaaccag | |
| | atcgctatcttcaaccggctgaagctggtg | |
| | cccaagaaggtggacctgtcccagcagaaa | |
| | gagatccccaccaccctggtggacgacttc | |
| | atcctgagccccgtcgtgaagagaagcttc | |
| | atccagagcatcaaagtgatcaacgccatc | |
| | atcaagaagtacggcctgcccaacgacatc | |
| | attatcgagctggcccgcgagaagaactcc | |
| | aaggacgcccagaaaatgatcaacgagatg | |
| | cagaagcggaaccggcagaccaacgagcgg | |
| | atcgaggaaatcatccggaccaccggcaaa | |
| | gagaacgccaagtacctgatcgagaagatc | |
| | aagctgcacgacatgcaggaaggcaagtgc | |
| | ctgtacagcctggaagccatccctctggaa | |
| | gatctgctgaacaaccccttcaactatgag | |
| | gtggaccacatcatccccagaagcgtgtcc | |
| | ttcgacaacagcttcaacaacaaggtgctc | |
| | gtgaagcaggaagaaaacagcaagaagggc | |
| | aaccggaccccattccagtacctgagcagc | |
| | agcgacagcaagatcagctacgaaaccttc | |
| | aagaagcacatcctgaatctggccaagggc | |
| | aagggcagaatcagcaagaccaagaaagag | |
| | tatctgctggaagaacgggacatcaacagg | |
| | ttctccgtgcagaaagacttcatcaaccgg | |
| | aacctggtggataccagatacgccaccaga | |
| | ggcctgatgaacctgctgcggagctacttc | |
| | agagtgaacaacctggacgtgaaagtgaag | |
| | tccatcaatggcggcttcaccagctttctg | |
| | cggcggaagtggaagtttaagaaagagcgg | |
| | aacaaggggtacaagcaccacgccgaggac | |
| | gccctgatcattgccaacgccgatttcatc | |
| | ttcaaagagtggaagaaactggacaaggcc | |
| | aaaaaagtgatggaaaaccagatgttcgag | |
| | gaaaagcaggccgagagcatgcccgagatc | |
| | gaaaccgagcaggagtacaaagagatcttc | |
| | atcaccccccaccagatcaagcacattaag | |
| | gacttcaaggactacaagtacagccaccgg | |
| | gtggacaagaagcctaatagagagctgatt | |
| | aacgacaccctgtactccacccggaaggac | |
| | gacaagggcaacaccctgatcgtgaacaat | |
| | ctgaacggcctgtacgacaaggacaatgac | |
| | aagctgaaaaagctgatcaacaagagcccc | |
| | gaaaagctgctgatgtaccaccacgacccc | |
| | cagacctaccagaaactgaagctgattatg | |
| | gaacagtacggcgacgagaagaatcccctg | |
| | tacaagtactacgaggaaaccgggaactac | |
| | ctgaccaagtactccaaaaaggacaacggc | |
| | cccgtgatcaagaagattaagtattacggc | |
| | aacaaactgaacgcccatctggacatcacc | |
| | gacgactaccccaacagcagaaacaaggtc | |
| | gtgaagctgtccctgaagccctacagattc | |
| | gacgtgtacctggacaatggcgtgtacaag | |
| | ttcgtgaccgtgaagaatctggatgtgatc | |
| | aaaaaagaaaactactacgaagtgaatagc | |
| | aagtgctatgaggaagctaagaagctgaag | |
| | aagatcagcaaccaggccgagtttatcgcc | |
| | tccttctacaacaacgatctgatcaagatc | |
| | aacggcgagctgtatagagtgatcggcgtg | |
| | aacaacgacctgctgaaccggatcgaagtg | |
| | aacatgatcgacatcacctaccgcgagtac | |
| | ctggaaaacatgaacgacaagaggcccccc | |
| | aggatcattaagacaatcgcctccaagacc | |
| | cagagcattaagaagtacagcacagacatt | |
| | ctgggcaacctgtatgaagtgaaatctaag | |
| | aagcaccctcagatcatcaaaaagggcaaa | |
| | aggccggcggccacgaaaaaggccggccag | |
| | gcaaaaaagaaaaagtaa | |
| SaCas9 | MAPKKKRKVGIHGVPAAKRNYILGLDIGIT | 399 |
| protein | SVGYGIIDYETRDVIDAGVRLFKEANVENN | |
| sequence | EGRRSKRGARRLKRRRRHRIQRVKKLLFDY | |
| | NLLTDHSELSGINPYEARVKGLSQKLSEEE | |
| | FSAALLHLAKRRGVHNVNEVEEDTGNELST | |
| | KEQISRNSKALEEKYVAELQLERLKKDGEV | |
| | RGSINRFKTSDYVKEAKQLLKVQKAYHQLD | |
| | QSFIDTYIDLLETRRTYYEGPGEGSPFGWK | |
| | DIKEWYEMLMGHCTYFPEELRSVKYAYNAD | |
| | LYNALNDLNNLVITRDENEKLEYYEKFQII | |
| | ENVFKQKKKPTLKQIAKEILVNEEDIKGYR | |
| | VTSTGKPEFTNLKVYHDIKDITARKEIIEN | |
| | AELLDQIAKILTIYQSSEDIQEELTNLNSE | |
| | LTQEEIEQISNLKGYTGTHNLSLKAINLIL | |
| | DELWHTNDNQIAIFNRLKLVPKKVDLSQQK | |
| | EIPTTLVDDFILSPVVKRSFIQSIKVINAI | |
| | IKKYGLPNDIIIELAREKNSKDAQKMINEM | |
| | QKRNRQTNERIEEIIRTTGKENAKYLIEKI | |
| | KLHDMQEGKCLYSLEAIPLEDLLNNPFNYE | |
| | VDHIIPRSVSFDNSFNNKVLVKQEENSKKG | |
| | NRTPFQYLSSSDSKISYETFKKHILNLAKG | |
| | KGRISKTKKEYLLEERDINRFSVQKDFINR | |
| | NLVDTRYATRGLMNLLRSYFRVNNLDVKVK | |
| | SINGGFTSFLRRKWKFKKERNKGYKHHAED | |
| | ALIIANADFIFKEWKKLDKAKKVMENQMFE | |
| | EKQAESMPEIETEQEYKEIFITPHQIKHIK | |
| | DFKDYKYSHRVDKKPNRELINDTLYSTRKD | |
| | DKGNTLIVNNLNGLYDKDNDKLKKLINKSP | |
| | EKLLMYHHDPQTYQKLKLIMEQYGDEKNPL | |
| | YKYYEETGNYLTKYSKKDNGPVIKKIKYYG | |
| | NKLNAHLDITDDYPNSRNKVVKLSLKPYRF | |
| | DVYLDNGVYKFVTVKNLDVIKKENYYEVNS | |
| | KCYEEAKKLKKISNQAEFIASFYNNDLIKI | |
| | NGELYRVIGVNNDLLNRIEVNMIDITYREY | |
| | LENMNDKRPPRIIKTIASKTQSIKKYSTDI | |
| | LGNLYEVKSKKHPQIIKKGKRPAATKKAGQ | |
| | AKKKK | |

In some embodiments, the polynucleotide sequence encoding a Cas endonuclease is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

In some embodiments, the gene editing system provided herein inhibits expression of any gene conferring further cardiac risk in a cardiac cell or patient having a deleterious TTN mutation. In some embodiments, the gene editing system provided herein confers a cardioprotective effect in a cardiac cell or patient having a deleterious TTN mutation. In some embodiments, the gene editing system provided herein protects against and/or ameliorates sarcomere dysfunction and/or disarray in a cardiac cell or patient having a TTN mutation.

In some embodiments, the gene editing system provided herein inhibits expression of any gene conferring further cardiac risk in a cardiac cell or patient having a deleterious MLP/CSRP3 mutation. In some embodiments, the gene editing system provided herein confers a cardioprotective effect in a cardiac cell or patient having a deleterious MLP/CSRP3 mutation. In some embodiments, the gene editing system provided herein protects against and/or ameliorates sarcomere dysfunction and/or disarray in a cardiac cell or patient having an MLP/CSRP3 mutation.

### Effects of Administration of Vectors, Inhibitory RNA and Gene Editing Constructs

In some embodiments, administration of the vectors described herein causes specific expression of MMP11, SYNPO2LA, or SYNPO2LB, and/or any combination thereof, in cardiomyocytes and/or the heart of the subject. In some embodiments, administration of the vectors described herein causes specific expression of MMP11 in cardiomyocytes and/or the heart of the subject. In some embodiments, administration of the vectors described herein causes specific expression of SYNPO2LA in cardiomyocytes and/or the heart of the subject. In some embodiments, administration of the vectors described herein causes specific expression of SYNPO2LB in cardiomyocytes and/or the heart of the subject. In some embodiments, the specific expression is at least or more than 1.25 fold, 1.3 fold, 1.4 fold, 1.5 fold, 1.6 fold, 1.7 fold, 1.8 fold, 1.9 fold, 2 fold, 2.5 fold, 3 fold, 3.5 fold, 4 fold, 4.5 fold, 5 fold, 6 old, 7 fold, 8 fold, 9 fold or 10 fold (or any range between these any of these values) over the wild-type level of expression of these proteins or the level of expression of these proteins before administration of the vectors. In some embodiments, administration of the vectors described herein leads to low or undetectable overexpression of MMP11, SYNPO2LA, and/or SYNPO2LB in non-cardiac cells and/or the skeletal tissue, brain, and/or liver of the subject (e.g., overexpression by less than that in cardiac cells, e.g., less than 1.5 fold, or less than 1.25 fold over the wild-type level of expression of these proteins or the level of expression of these proteins before administration of the vectors). The expression differential between cardiac and non-cardiac cells and/or tissues can be at least 2 fold, 5 fold, 10 fold, 15 fold, 20 fold, 50 fold, 100 fold, 150 fold, or 200 fold or higher.

In some embodiments, administration of inhibitory RNA or gene editing systems described herein (either using a viral vector or by other means of delivery described herein) causes specific inhibition of expression of MTSS1 in cardiomyocytes and/or the heart of the subject. In some embodiments, the specific inhibition inhibits the expression of MTSS1 by at least or more than 10%, 20%, 30%, 40%, 50%, 60%, 70%, 80%, 90% or 95% (or any range between any of these values) of the wild-type level of expression of MTSS1 or the level of expression of MTSS1 before administration of the inhibitory RNA. In some embodiments, administration of the inhibitory RNA or gene editing system described herein leads to low or undetectable inhibition MTSS1 in non-cardiac cells and/or the skeletal tissue, brain, and/or liver of the subject (e.g., inhibition by less than that in cardiac cells, e.g., less than 30%, less than 20%, or less than 10% of the wild-type level of expression of MTSS1 or the level of expression of MTSS1 before administration of the inhibitory RNA). The inhibition differential between cardiac and non-cardiac cells and/or tissues can be at least 2-fold, 5 fold, 10 fold, 15 fold, 20 fold, 50 fold, 100 fold, 150 fold, or 200 fold or higher.

In some embodiments, the administration of the therapies described herein to a subject restores or improves cardiac function the subject. In some embodiments, the administration of the therapies described herein to a subject restores or improves contractile function of the heart in the subject. In some embodiments, the administration of the therapies described herein to a subject restores or improves sarcomere function in a cardiac cell, e.g., cardiomyocyte, in the subject. In some embodiments, the administration of the therapies described herein to a subject restores or improves sarcomere organization in a cardiac cell, e.g., cardiomyocyte, in the subject. For example, the improvement in sarcomere function or organization can be demonstrated by an increase in sarcomere number (sarcomere count), increase in sarcomere length, increase in length uniformity (length variation), and/or increase in orientation uniformity (angle variation). In some embodiments, the improvement in sarcomere function or organization is as assessed by any one, two, three, four or more of these parameters.

### Cells and Cell Therapies

Also provided herein is an isolated cell or population of cells comprising any vector described herein. Also provided herein is an isolated cell or population of cells comprising any inhibitory RNA (e.g., siRNA) described herein. Also provided herein is an isolated cell or population of cells comprising two or more vectors described herein. Also provided herein is an isolated cell or population of cells comprising one, two or more vectors described herein and one or more inhibitory RNA (e.g., siRNA) described herein.

In some embodiments, the cell is a cardiac cell.

As used herein the term “cardiac cell” refers to any cell present in the heart that provides a cardiac function, such as heart contraction or blood supply, or otherwise serves to maintain the structure of the heart. Cardiac cells as used herein encompass cells that exist in the epicardium, myocardium or endocardium of the heart. Cardiac cells also include, for example, cardiac muscle cells or cardiomyocytes, and cells of the cardiac vasculatures, such as cells of a coronary artery or vein. Other non-limiting examples of cardiac cells include epithelial cells, endothelial cells, fibroblasts, cardiac stem or progenitor cells, cardiac conducting cells and cardiac pacemaking cells that constitute the cardiac muscle, blood vessels and cardiac cell supporting structure. Cardiac cells may be derived from stem cells, including, for example, embryonic stem cells or induced pluripotent stem cells.

In some embodiments, the cell is a cardiomyocyte.

In some embodiments, the cell is an induced pluripotent stem cell.

The disclosure provides methods of manipulating polypeptide expression in a cell comprising contacting the cell with any vector or virion (e.g., rAAV virion), inhibitory RNA or gene editing system described herein. In some embodiments, the cell is a cardiac cell. In some embodiments, the cell is a cardiomyocyte. In some embodiments, the contacting is in vitro. In some embodiments, the contacting is in vivo. In some embodiments, the polypeptide is any polypeptide for use in treating or preventing a heart disease. In some embodiments, the polypeptide is any polypeptide described herein. In some embodiments, the polypeptide is encoded by any transgene described herein.

The disclosure provides methods of manipulating polypeptide expression in a tissue comprising contacting the tissue with any vector or virion (e.g., rAAV virion), inhibitory RNA or gene editing system described herein. In some embodiments, the tissue is cardiac tissue. In some embodiments, the contacting is in vitro. In some embodiments, the contacting is in vivo.

The disclosure provides methods of manipulating polypeptide expression in an organ comprising contacting the organ with any vector or virion (e.g., rAAV virion), inhibitory RNA or gene editing system described herein. In some embodiments, the organ is a heart. In some embodiments, the heart is diseased or at risk of disease. In some embodiments, the heart has borderline or reduced ejection fraction. In some embodiments, the heart has a normal ejection fraction. In some embodiments, the heart comprises a genetic mutation associated with a heart disease. In some embodiments, the genetic mutation is a TTN mutation (e.g., TTNtv). In some embodiments, the genetic mutation is a MLP/CSRP3 gene mutation. In some embodiments, the contacting is in vitro. In some embodiments, the contacting is in vivo.

Also provided herein is a cell therapy composition comprising any cell described herein.

The disclosure provides methods for expressing a polynucleotide a cell. The method may comprise, for example, transducing a target cell with the rAAV virions, rAAV vector genomes, or expression cassettes described herein. A target cell can be, for example and without limitation, a cardiac cell, a muscle cell, an induced pluripotent stem cell-derived cardiomyocyte (iPSC-CM), a cardiomyocyte, a TTN P22353X^{+/-} cell. In one embodiment, the cell comprises a mutation of the endogenous TTN gene, e.g., resulting in truncating variants in the TTN gene. In one embodiment, the truncating variants in the TTN gene cause dilated cardiomyopathy. (Akhtar et. al, Circ Heart Fail. 2020; 13:e006832). In one embodiment, the cell is a TTN P22353X^{+/-} cell. In one embodiment, the cell comprises a mutation of the endogenous MLP/CSRP3 gene.

### Pharmaceutical Compositions and Kits

Also provided herein are pharmaceutical compositions comprising at least one vector described herein or at least one inhibitory RNA described herein. Also provided herein are pharmaceutical compositions comprising two or more vectors described herein (e.g., one expressing at least one of MMP11 and a SYNPO2L, and another expressing at least one of an inhibitory RNA for MTSS1 or CRISPR/Cas MTSS1, or any other combination). Also provided herein are pharmaceutical compositions comprising two or more inhibitory RNA described herein (e.g., two different siRNA inhibiting MTSS1). Also provided herein are pharmaceutical compositions comprising one or more vectors described herein (e.g., expressing MMP11, SYNPO2L, siMTSS1, and/or CRISPR/Cas MTSS1) and one or more inhibitory RNA described herein (e.g., siMTSS1).

The present disclosure provides pharmaceutical compositions for treating and/or preventing heart disease (e.g., heart disease associated with truncating variants in the TTN gene). The present disclosure further provides pharmaceutical compositions comprising a vector (e.g., an rAAV vector genome or rAAV virion) described herein, and/or an inhibitory RNA described herein, and one or more pharmaceutically acceptable carriers, diluents or excipients for parenteral delivery. In some embodiments, the vector genome comprises a gene encoding MMP11, SYNPO2LA, SYNPO2LB, a Cas endonuclease/guide RNA targeting MTSS1, and/or inhibitory RNA targeting MTSS1.

Further, the present disclosure also provides pharmaceutical compositions comprising a naked inhibitory RNA or inhibitory RNA formulated in a non-viral-vector delivery vehicle (e.g., lipid nanoparticle). In some embodiments, the inhibitory RNA inhibits expression of MTSS1.

In various embodiments, the pharmaceutical compositions described herein contain one or more pharmaceutically acceptable excipients. Pharmaceutically acceptable excipients can include vehicles (e.g., carriers, diluents and excipients) that are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. Illustrative pharmaceutical forms suitable for injectable use include, e.g., sterile aqueous solutions or dispersions; formulations including sesame oil, peanut oil or aqueous propylene glycol; and sterile powders for the extemporaneous preparation of sterile injectable solutions or dispersions.

In some embodiments, the pharmaceutical compositions of the disclosure comprise about 1×10⁸ genome copies per milliliter (GC/mL), about 5×10⁸ GC/mL, about 1×10⁹ GC/mL, about 5×10⁹ GC/mL, about 1×10¹⁰ GC/mL, about 5×10¹⁰ GC/mL, about 1×10¹¹ GC/mL, about 5×10¹¹ GC/mL, about 1×10¹² GC/mL, about 5×10¹² GC/mL, about 5×10¹³ GC/mL, about 1×10¹⁴ GC/mL, or about 5×10¹⁴ GC/mL of the viral vector (e.g. rAAV virion).

In some embodiments, the pharmaceutical compositions of the disclosure comprise about 1×10⁸ viral genomes per milliliter (vg/mL), about 5×10⁸ vg/mL, about 1×10⁹ vg/mL, about 5×10⁹ vg/mL, about 1×10¹⁰ vg/mL, about 5×10¹⁰ vg/mL, about 1×10¹¹ vg/mL, about 5×10¹¹ vg/mL, about 1×10¹² vg/mL, about 5×10¹² vg/mL, about 5×10¹³ vg/mL, about 1×10¹⁴ vg/mL, or about 5×10¹⁴ vg/mL of the viral vector (e.g., rAAV virion).

In some embodiments, the pharmaceutical compositions of the disclosure are administered in a total volume of about 1 mL, 5 mL, 10 mL, about 20 mL, about 25 mL, about 30 mL, about 35 mL, about 40 mL, about 45 mL, about 50 mL, about 55 mL, about 60 mL, 65 mL, about 70 mL, about 75 mL, about 80 mL, about 85 mL, about 90 mL, about 95 mL, about 100 mL, about 105 mL, about 110 mL, about 115 mL, about 120 mL, about 125 mL, about 130 mL, about 135 mL, about 140 mL, about 145 mL, about 150 mL, about 155 mL, about 160 mL, about 165 mL, about 170 mL, about 175 mL, about 180 mL, about 185 mL, about 190 mL, about 200 mL, about 205 mL, about 210 mL, about 215 mL, or about 220 mL.

In some embodiments, the present disclosure provides a kit comprising a container housing a pharmaceutical composition as described herein.

### Patient Populations

Subjects who are suitable for treatment using the compositions and methods of the present disclosure include individuals (e.g., mammalian subjects, such as humans, non-human primates, domestic mammals, experimental non-human mammalian subjects such as mice, rats, etc.) having a cardiac condition.

In some embodiments, the subject to be treated in accordance with the methods described herein is a mammal. In some embodiments, the subject to be treated in accordance with the methods described herein is a human.

In some embodiments, the subject to be treated does not have a deleterious mutation in or deletion of MMP11. In some embodiments, the subject to be treated does not have a deleterious mutation in or deletion of SYNPO2L (e.g., SYNPO2LA, SYNPO2LB). In some embodiments, the subject to be treated does not have a mutation in (e.g., a gain of function mutation) or overexpression of MTSS1.

In some embodiments, the subject to be treated has one or more deleterious mutations in a gene associated with cardiac disease or dysfunction. In some embodiments, the subject to be treated has one or more deleterious mutations (e.g., a truncating mutation) in the TNN gene and/or the MLP/CSRP3 gene.

Truncating variants in TTN (TTNtv) account for 15 to 25% of DCM cases (Herman et al. NEJM 2012, Mazzarotto et al. Circulation 2020, Fang et al. Herz 2020). Truncating variants in the TTN gene (TTNtv) are the common cause of heritable dilated cardiomyopathy. Patient populations with heart disease associated with truncating variants in the TTN gene (TTNtv), include, but are not limited to, patient populations with dilated cardiomyopathy, left ventricular systolic dysfunction, atrial and/or ventricular arrhythmia, and/or malignant ventricular arrhythmia (Circ Heart Fail. 2020; 13:e006832).

In some embodiments, the subject to be treated has a mutation in TTN. In some embodiments, the subject to be treated has any mutation in TTN known in the art or described herein. In some embodiments, the subject to be treated has a truncating variant of the TTN gene (TTNtv). In some embodiments, the subject to be treated has a truncating variant of the TTN gene Akhtar et. al, Circ Heart Fail. 2020; 13:e006832, which is incorporated by reference herein in its entirety.

In some embodiments, the subject to be treated has a mutation in MLP/CSRP3. In some embodiments, the subject to be treated has any mutation in MLP/CSRP3 known in the art or described herein.

In some embodiments, the subject to be treated has a heart disease. In some embodiments, the subject to be treated is at risk of heart disease.

In some embodiments, the subject to be treated has a TTN-associated heart disease. In some embodiments, the subject to be treated is at risk of TTN-associated heart disease. The heart disease can be an acquired form of heart disease or a genetic (e.g., polygenic) form of heart disease.

In some embodiments, the subject to be treated has an MLP/CSPR3-associated heart disease. In some embodiments, the subject to be treated is at risk of MLP/CSPR3-associated heart disease.

In some embodiments, the subject to be treated has cardiomyopathy, e.g., DCM (dilated cardiomyopathy). In some embodiments, the subject to be treated is at risk of cardiomyopathy, e.g., DCM. In some embodiments, the subject to be treated has hypertrophic cardiomyopathy (HCM). In some embodiments, the subject to be treated is at risk of hypertrophic cardiomyopathy. The cardiomyopathy, such as DCM or hypertrophic cardiomyopathy, can be an acquired form of cardiomyopathy or a genetic (e.g., polygenic) form of cardiomyopathy.

In some embodiments, the subject to be treated has a TTN-associated cardiomyopathy, e.g., TTN-associated HCM or TTN-associated DCM. In some embodiments, the subject to be treated is at risk of TTN-associated cardiomyopathy, e.g., TTN-associated HCM or TTN-associated DCM.

In some embodiments, the subject to be treated has an MLP/CSPR3-associated cardiomyopathy, e.g., MLP/CSPR3-associated HCM or MLP/CSPR3-associated DCM. In some embodiments, the subject to be treated is at risk of MLP/CSPR3-associated cardiomyopathy, e.g., e.g., MLP/CSPR3-associated HCM or MLP/CSPR3-associated DCM.

In some embodiments, the subject to be treated has an idiopathic DCM. In some embodiments, the subject to be treated is at risk of an idiopathic DCM.

In some embodiments, the subject to be treated has a TTN-associated idiopathic DCM. In some embodiments, the subject to be treated is at risk of a TTN-associated idiopathic DCM.

In some embodiments, the subject to be treated has an MLP/CSPR3-associated idiopathic DCM. In some embodiments, the subject to be treated is at risk of an MLP/CSPR3-associated idiopathic DCM. In some embodiments, the subject to be treated has a heart failure (e.g., heart failure with reduced ejection fraction). In some embodiments, the subject to be treated is at risk of heart failure (e.g., heart failure with reduced ejection fraction). The heart failure, such as heart failure with reduced ejection fraction, can be an acquired form of heart failure or a genetic (e.g., polygenic) form of heart failure.

In some embodiments, the subject to be treated has a TTN-associated heart failure. In some embodiments, the subject to be treated is at risk of TTN-associated heart failure.

In some embodiments, the subject to be treated has an MLP/CSPR3-associated heart failure. In some embodiments, the subject to be treated is at risk of MLP/CSPR3-associated heart failure.

In some embodiments, the subject to be treated has a systolic dysfunction, e.g., left ventricular systolic dysfunction. In some embodiments, the subject to be treated is at risk of a systolic dysfunction, e.g., left ventricular systolic dysfunction. In some embodiments, the subject to be treated has a genetic or polygenic form of systolic dysfunction, e.g., left ventricular systolic dysfunction.

In some embodiments, the subject to be treated has a TTN-associated systolic dysfunction, e.g., left ventricular systolic dysfunction. In some embodiments, the subject to be treated is at risk of TTN-associated systolic dysfunction, e.g., left ventricular systolic dysfunction.

In some embodiments, the subject to be treated has a MLP/CSPR3-associated systolic dysfunction, e.g., left ventricular systolic dysfunction. In some embodiments, the subject to be treated is at risk of MLP/CSPR3-associated systolic dysfunction, e.g., left ventricular systolic dysfunction.

In some embodiments, the subject to be treated has an arrhythmia, e.g., atrial and/or ventricular arrhythmia, and/or malignant ventricular arrhythmia. In some embodiments, the subject to be treated is at risk of an arrhythmia, e.g., atrial and/or ventricular arrhythmia, and/or malignant ventricular arrhythmia. In some embodiments, the subject to be treated has a genetic or polygenic form of arrhythmia, e.g., atrial and/or ventricular arrhythmia, and/or malignant ventricular arrhythmia.

In some embodiments, the subject to be treated has a TTN-associated an arrhythmia, e.g., atrial and/or ventricular arrhythmia, and/or malignant ventricular arrhythmia. In some embodiments, the subject to be treated is at risk of TTN-associated an arrhythmia, e.g. atrial and/or ventricular arrhythmia, and/or malignant ventricular arrhythmia.

In some embodiments, the subject to be treated has an MLP/CSPR3-associated an arrhythmia, e.g., atrial and/or ventricular arrhythmia, and/or malignant ventricular arrhythmia. In some embodiments, the subject to be treated is at risk of MLP/CSPR3-associated an arrhythmia, e.g., atrial and/or ventricular arrhythmia, and/or malignant ventricular arrhythmia. Other subjects that can be treated using the compositions and methods of the present disclosure include, but are not limited to, individuals having a congenital heart defect, individuals suffering from a degenerative muscle disease, individuals suffering from a condition that results in ischemic heart tissue (e.g., individuals with coronary artery disease), and the like. In some examples, a method is useful to treat a degenerative muscle disease or condition (e.g., familial cardiomyopathy, dilated cardiomyopathy, hypertrophic cardiomyopathy, restrictive cardiomyopathy, or coronary artery disease with resultant ischemic cardiomyopathy). In some examples, a subject method is useful to treat individuals having a cardiac or cardiovascular disease or disorder, for example, cardiovascular disease, angina, arrhythmia, congenital heart disease, congestive heart failure, myocarditis, valve disease coronary, artery disease dilated, diastolic dysfunction, cardiomyopathy, hypertrophic cardiomyopathy, restrictive cardiomyopathy, coronary artery disease with resultant ischemic cardiomyopathy, mitral valve prolapse, or myocardial infarction (heart attack).

In some embodiments, the compositions and methods disclosed herein can be used for the prevention and/or treatment of cardiomyopathies in a subject. In some embodiments, the compositions and methods disclosed herein can be used for the prevention and/or treatment of genetic or polygenic forms of cardiomyopathies in a subject. In some embodiments, the compositions and methods described herein can be used to treat cardiomyopathies affiliated with mutations in TTN gene, such as dilated cardiomyopathy and other heart diseases associated with truncating variants in the TTN gene. In some embodiments, the mutations in TTN gene include, but are not limited to those described in Circ Heart Fail. 2020; 13:e006832. In some embodiments, the mutations in TTN gene is any pathogenic mutation in the TTN gene.

In some embodiments, the compositions and methods described herein can be used to treat cardiomyopathies affiliated with mutations in MLP/CSPR3 gene, such as dilated cardiomyopathy and other heart diseases associated with truncating variants in the MLP/CSPR3 gene.

### Methods of Treatment

### Heart Diseases to be Treated

In some embodiments, the heart disease to be treated or prevented in accordance with the methods described herein is any heart disease.

In some embodiments, the heart disease to be treated or prevented in accordance with the methods described herein is an acquired (such as not caused by a known mutation) form of a heart disease. In some embodiments, the heart disease to be treated or prevented in accordance with the methods described herein is a genetic (e.g., polygenic) form of a heart disease.

In some embodiments, the heart disease to be treated or prevented in accordance with the methods described herein is a TTN-associated heart disease.

In some embodiments, the heart disease to be treated or prevented in accordance with the methods described herein is an MLP/CSPR3-associated heart disease.

In some embodiments, the heart disease to be treated or prevented in accordance with the methods described herein is associated with a mutation in TTN, e.g., any mutation in TTN known in the art or described herein. In some embodiments, the heart disease to be treated or prevented in accordance with the methods described herein is associated with a truncating variant of the TTN gene (TTNtv).

In some embodiments, the heart disease to be treated or prevented in accordance with the methods described herein is associated with a mutation in MLP/CSPR3, e.g., any mutation in MLP/CSPR3 known in the art or described herein. In some embodiments, the heart disease to be treated or prevented in accordance with the methods described herein is associated with a truncating variant of the MLP/CSPR3 gene.

In some embodiments, the heart disease to be treated or prevented in accordance with the methods described herein is cardiomyopathy, e.g., dilated cardiomyopathy (DCM). In some embodiments, the heart disease to be treated or prevented in accordance with the methods described herein is an idiopathic DCM. In some embodiments, the heart disease to be treated or prevented in accordance with the methods described herein is an acquired form of cardiomyopathy, e.g., acquired dilated cardiomyopathy (DCM). In some embodiments, the heart disease to be treated or prevented in accordance with the methods described herein is a genetic form of cardiomyopathy, e.g., genetic dilated cardiomyopathy (DCM). In some embodiments, the heart disease to be treated or prevented in accordance with the methods described herein is hypertrophic cardiomyopathy. In some embodiments, the heart disease to be treated or prevented in accordance with the methods described herein is an acquired hypertrophic cardiomyopathy. In some embodiments, the heart disease to be treated or prevented in accordance with the methods described herein is a genetic hypertrophic cardiomyopathy.

In some embodiments, the heart disease to be treated or prevented in accordance with the methods described herein is TTN-associated cardiomyopathy, e.g., TTN-associated DCM. In some embodiments, the heart disease to be treated or prevented in accordance with the methods described herein is a TTN-associated idiopathic DCM. In some embodiments, the heart disease to be treated or prevented in accordance with the methods described herein is a TTN-associated hypertrophic cardiomyopathy.

In some embodiments, the heart disease to be treated or prevented in accordance with the methods described herein is MLP/CSPR3-associated cardiomyopathy, e.g., MLP/CSPR3-associated DCM. In some embodiments, the heart disease to be treated or prevented in accordance with the methods described herein is an MLP/CSPR3-associated idiopathic DCM. In some embodiments, the heart disease to be treated or prevented in accordance with the methods described herein is an MLP/CSPR3-associated hypertrophic cardiomyopathy. In some embodiments, the heart disease to be treated or prevented in accordance with the methods described herein is a heart failure. In some embodiments, the heart disease to be treated or prevented in accordance with the methods described herein is an acquired form of heart failure. In some embodiments, the heart disease to be treated or prevented in accordance with the methods described herein is a genetic (e.g., polygenic) form of heart failure. In some embodiments, the heart disease to be treated or prevented in accordance with the methods described herein is a TTN-associated heart failure. In some embodiments, the heart disease to be treated or prevented in accordance with the methods described herein is an MLP/CSPR3-associated heart failure.

In some embodiments, the heart disease to be treated or prevented in accordance with the methods described herein is a heart failure with reduced ejection fraction (e.g., an acquired form or a genetic or polygenic form). In some embodiments, the heart disease to be treated or prevented in accordance with the methods described herein is a TTN-associated heart failure with reduced ejection fraction. In some embodiments, the heart disease to be treated or prevented in accordance with the methods described herein is a MLP/CSPR3-associated heart failure with reduced ejection fraction.

In some embodiments, the heart disease is selected from cardiomyopathy, systolic dysfunction, and arrhythmia, e.g., TTN-associated cardiomyopathy, TTN-associated systolic dysfunction, TTN-associated arrhythmia, MLP/CSPR3-associated cardiomyopathy, MLP/CSPR3-associated systolic dysfunction, MLP/CSPR3-associated arrhythmia. In some embodiments, the heart disease is selected from acquired forms of cardiomyopathy, systolic dysfunction, and arrhythmia. In some embodiments, the heart disease is selected from genetic (e.g., polygenic) forms of cardiomyopathy, systolic dysfunction, and arrhythmia.

In some embodiments, the heart disease is selected from dilated cardiomyopathy, left ventricular systolic dysfunction, atrial and/or ventricular arrhythmia, and malignant ventricular arrhythmia, e.g., when these conditions are associated with a TTN mutation. In some embodiments, the heart disease is selected from dilated cardiomyopathy, left ventricular systolic dysfunction, atrial and/or ventricular arrhythmia, and malignant ventricular arrhythmia, e.g., when these conditions are associated with an MLP/CSPR3 mutation. In some embodiments, the heart disease is selected from acquired forms of dilated cardiomyopathy, left ventricular systolic dysfunction, atrial and/or ventricular arrhythmia, and malignant ventricular arrhythmia. In some embodiments, the heart disease is selected from genetic (e.g., polygenic) forms of dilated cardiomyopathy, left ventricular systolic dysfunction, atrial and/or ventricular arrhythmia, and malignant ventricular arrhythmia.

In some embodiments, the heart disease to be treated or prevented in accordance with the methods described herein is a systolic dysfunction, e.g., left ventricular systolic dysfunction. In some embodiments, the heart disease to be treated or prevented in accordance with the methods described herein is an acquired systolic dysfunction. In some embodiments, the heart disease to be treated or prevented in accordance with the methods described herein is a genetic (e.g., polygenic) systolic dysfunction In some embodiments, the heart disease to be treated or prevented in accordance with the methods described herein is a TTN-associated systolic dysfunction, e.g., left ventricular systolic dysfunction. In some embodiments, the heart disease to be treated or prevented in accordance with the methods described herein is an MLP/CSRP3-associated systolic dysfunction, e.g., left ventricular systolic dysfunction.

In some embodiments, the heart disease to be treated or prevented in accordance with the methods described herein is an arrhythmia, e.g., atrial and/or ventricular arrhythmia, and/or malignant ventricular arrhythmia. In some embodiments, the heart disease to be treated or prevented in accordance with the methods described herein is an acquired arrhythmia. In some embodiments, the heart disease to be treated or prevented in accordance with the methods described herein is a genetic (e.g., polygenic) arrhythmia. In some embodiments, the heart disease to be treated or prevented in accordance with the methods described herein is a TTN-associated an arrhythmia, e.g., atrial and/or ventricular arrhythmia, and/or malignant ventricular arrhythmia. In some embodiments, the heart disease to be treated or prevented in accordance with the methods described herein is an MLP/CSPR3-associated an arrhythmia, e.g., atrial and/or ventricular arrhythmia, and/or malignant ventricular arrhythmia.

The cardiomyopathy treated or prevented by the compositions and methods described herein can also include cardiomyopathies associated with a pulmonary embolus, a venous thrombosis, a myocardial infarction, a transient ischemic attack, a peripheral vascular disorder, atherosclerosis, ischemic cardiac disease and/or other myocardial injury or vascular disease. In certain embodiments, the cardiomyopathies treated by the compositions and methods described herein can include cardiac diseases associated with myocardial tissue hypercontractility, such as heart failure related to left ventricular hypercontractility.

In some embodiments, the cardiomyopathy is hypertrophic cardiomyopathy.

### Methods of Treating Heart Disease

The compositions that are described herein can be employed in a method of treating a subject with a cardiac disease or condition. “Treating” or “treatment of a condition or subject in need thereof” refers to (1) taking steps to obtain beneficial or desired results, including clinical results such as the reduction of symptoms; (2) inhibiting the disease, for example, arresting or reducing the development of the disease or its clinical symptoms; (3) relieving the disease, for example, causing regression of the disease or its clinical symptoms; and/or (4) delaying the disease. For purposes of this invention, beneficial or desired clinical results include, but are not limited to, promoting cardiac sarcomere contraction.

In some embodiments, the compositions and methods described herein can induce detectable expression of a therapeutic protein or nucleic acid (e.g., MMP11, SYNPO2LA, and/or SYNPO2LB protein), or a mutant, variant, or fragment thereof, to modulate sarcomere architecture and/or contractile function of the myocardial tissue in a subject in need thereof. In some embodiments, the compositions and methods described herein can decrease the expression of a protein or nucleic acid (e.g., MTSS1 protein), or a mutant, variant, or fragment thereof, to modulate sarcomere architecture and/or contractile function of the myocardial tissue in a subject in need thereof.

In some embodiments, the compositions and methods described herein improve sarcomere architecture (e.g., as measured by assessment of sarcomere count, sarcomere length, sarcomere angle and/or sarcomere fit, using any methods known in the art or described herein).

In some embodiments, the compositions and methods described herein improve contractile function of the myocardial tissue (e.g., in a subject being treated).

### Routes and Modes of Administration

The vectors and virions (e.g., AAV) and/or inhibitory RNA of the present disclosure can be administered to a subject in need thereof by systemic application (such as parenteral application), e.g., by intravenous, intra-arterial or intraperitoneal delivery of a vector in analogy to what has been shown in animal models (Katz et al., 2012, Gene Ther. 19:659-669). In some embodiments, the vectors and virions (e.g., AAV) and/or inhibitory RNA of the present disclosure treats or prevents hypertrophic cardiomyopathy, wherein the vector is administered or parenterally. In some embodiments, the vectors and virions (e.g., AAV) and/or inhibitory RNA of the present disclosure are to be administered intravenously (e.g., by IV infusion).

In some embodiments, the vectors and virions (e.g., AAV) and/or inhibitory RNA of the present disclosure can be delivered by direct administration to the heart tissue.

In some embodiments, the vectors and virions (e.g., AAV) and/or inhibitory RNA of the present disclosure can be delivered by intracoronary administration. In some embodiments, the administration is by antegrade epicardial coronary artery infusion, e.g., a single infusion over a 10-minute period in a cardiac catheterization laboratory after angiography (percutaneous intracoronary delivery without vessel balloon occlusion) with the use of standard 5F or 6F guide or diagnostic catheters (Jaski et al., 2009, J Card Fail. 15: 171-181).

In some embodiments, the vectors and virions (e.g., AAV) and/or inhibitory RNA of the present disclosure can be delivered by direct injection into the heart or cardiac catheterization. In some embodiments, the vectors and virions (e.g., AAV) and/or inhibitory RNA of the present disclosure can be delivered by intracardiac catheter delivery via retrograde coronary sinus infusion (RCSI).

When direct injection is used, it may be performed either by open-heart surgery or by minimally invasive surgery. In some cases, the vectors and virions (e.g., AAV) and/or inhibitory RNA can be delivered to the pericardial space by injection or infusion.

In some embodiments, the amount, concentration, and volume of the composition that modulates contractile function in myocardial tissue administered to a subject can be controlled and/or optimized to substantially improve the functional parameters of the heart while mitigating adverse side effects.

The amount of the composition that modulates contractile function administered to myocardial tissue can also be an amount required to result in the detectable expression of a therapeutic protein or nucleic acid (e.g., MMP11, SYNPO2LA, and/or SYNPO2LB protein) or a mutant, variant, or fragment thereof in the heart; preserve and/or improve contractile function; delay the emergence of cardiomyopathy or reverse the pathological course of the disease; increase myocyte viability; improve myofilament function; inhibit left ventricular hypertrophy; cardiac hypertrophy regression, normalize systolic and diastolic function in heart; and restore normal cross-bridge behavior at the myofilament level.

The amount of the composition that modulates contractile function administered to myocardial tissue can also be an amount required to result in the decrease of the expression of a protein (e.g., MTSS1 protein) or a mutant, variant, or fragment thereof in the heart; preserve and/or improve contractile function; delay the emergence of cardiomyopathy or reverse the pathological course of the disease; increase myocyte viability; improve myofilament function; inhibit left ventricular hypertrophy; cardiac hypertrophy regression, normalize systolic and diastolic function in heart; and restore normal cross-bridge behavior at the myofilament level.

In some embodiments, the compositions and methods disclosed herein result in detectable expression (or detectable overexpression, over the wild-type level of expression) of MMP11, SYNPO2LA, and/or SYNPO2LB protein, or a mutant, variant, or fragment thereof, in a cardiac cell of the subject being treated. In some embodiments, the administration of the compositions and methods described herein (e.g., an rAAV vector genome or rAAV virion) causes specific expression of MMP11, SYNPO2LA, and/or SYNPO2LB protein in the heart of the subject.

In some embodiments, the compositions and methods disclosed herein result in decreased expression of MTSS1 protein in a cardiac cell of the subject being treated. In some embodiments, the administration of the compositions and methods described herein (e.g., an rAAV vector genome or rAAV virion) causes decreased expression of MTSS1 in the heart of the subject.

“Detectable expression” typically refers to expression at least 5%, 10%, 15%, 20% or more compared to a control subject or tissue not treated with the vector. In some embodiments, detectable expression means expression at 1.5-fold, 2-fold, 2.5-fold, or 3-fold greater than a no-vector control. Expression can be assess by Western blot, as described in the example that follows, or enzyme-linked immunosorbent assay (ELISA), or other methods known in the art. In some cases, expression is measured quantitatively using a standard curve. Standard curves can be generated using purified protein by methods described in the examples or known in the art. Alternatively, expression of the therapeutic gene product can be assessed by quantification of the corresponding mRNA.

In some embodiments, the detectable expression of the therapeutic gene product in heart tissue occurs at doses, in vector genomes (vg) per kilogram weight of subject (kg), of 3×10¹⁴ vg/kg or less, 2×10¹⁴ vg/kg or less, 1×10¹⁴ vg/kg or less, 9×10¹³ vg/kg or less, 8×10¹³ vg/kg or less, 7×10¹³ vg/kg or less, 6×10¹³ vg/kg or less, 5×10¹³ vg/kg or less, 4×10¹³ vg/kg or less, 3×10¹³ vg/kg or less, 2×10¹³ vg/kg or less, or 1×10¹³ vg/kg or less.

In some embodiments, the methods of the disclosure comprise administering an rAAV virion encoding MMP11, SYNPO2LA, SYNPO2LB, Cas endonuclease/guide RNA targeting MTSS1, and/or an inhibitory RNA inhibiting expression of MTSS1 at a dose of about 1×10⁸ genome copies per milliliter (GC/mL), about 5×10⁸ GC/mL, about 1×10⁹ GC/mL, about 5×10⁹ GC/mL, about 1×10¹⁰ GC/mL, about 5×10¹⁰ GC/mL, about 1×10¹¹ GC/mL, about 5×10¹¹ GC/mL, about 1×10¹² GC/mL, about 5×10¹² GC/mL, about 5×10¹³ GC/mL, about 1×10¹⁴ GC/mL, or about 5×10¹⁴ GC/mL of the rAAV virion.

In some embodiments, the methods of the disclosure comprise intravenously administering an rAAV virion encoding MMP11, SYNPO2LA, SYNPO2LB, Cas endonuclease/guide RNA targeting MTSS1, and/or an inhibitory RNA inhibiting expression of MTSS1 at a dose of about 3×10¹² GC/mL, about 3×10¹³ GC/mL, about 1×10¹⁴ GC/mL, or about 3×10¹⁴ GC/mL of the rAAV virion.

In some embodiments, the methods of the disclosure comprise administering, by localized delivery to the heart, an rAAV virion encoding MMP11, SYNPO2LA, SYNPO2LB, Cas endonuclease/guide RNA targeting MTSS1, and/or siRNA MTSS1 at a dose of about 3×10¹¹ GC/mL, about 3×10¹² GC/mL, about 1×10¹³ GC/mL, or about 3×10¹³ GC/mL of the rAAV virion.

In some embodiments, the methods of the disclosure comprise administering an rAAV virion encoding MMP11, SYNPO2LA, SYNPO2LB, Cas endonuclease/guide RNA targeting MTSS1, and/or an inhibitory RNA inhibiting expression of MTSS1 at a dose of about 1×10⁸ viral genomes per milliliter (vg/mL), about 5×10⁸ vg/mL, about 1×10⁹ vg/mL, about 5×10⁹ vg/mL, about 1×10¹⁰ vg/mL, about 5×10¹⁰ vg/mL, about 1×10¹¹ vg/mL, about 5×10¹¹ vg/mL, about 1×10¹² vg/mL, about 5×10¹² vg/mL, about 5×10¹³ vg/mL, about 1×10¹⁴ vg/mL, or about 5×10¹⁴ vg/mL of the rAAV virion.

In some embodiments, the methods of the disclosure comprise intravenously administering an rAAV virion encoding MMP11, SYNPO2LA, SYNPO2LB, Cas endonuclease/guide RNA targeting MTSS1, and/or an inhibitory RNA inhibiting expression of MTSS1 at a dose of about 3×10¹² vg/mL, about 3×10¹³ vg/mL, about 1×10¹⁴ vg/mL, or about 3×10¹⁴ vg/mL of the rAAV virion.

In some embodiments, the methods of the disclosure comprise administering, by localized delivery to the heart, an rAAV virion encoding MMP11, SYNPO2LA, SYNPO2LB, Cas endonuclease/guide RNA targeting MTSS1, and/or an inhibitory RNA inhibiting expression of MTSS1 at a dose of about 3×10¹¹ vg/mL, about 3×10¹² vg/mL, about 1×10¹³ vg/mL, or about 3×10¹³ vg/mL of the rAAV virion.

Genome copies per milliliter can be determined by quantitative polymerase change reaction (qPCR) using a standard curve generated with a reference sample having a known concentration of the polynucleotide genome of the virus. For AAV, the reference sample used is often the transfer plasmid used in generation of the rAAV virion but other reference samples may be used.

Alternatively, or in addition, the concentration of a viral vector can be determined by measuring the titer of the vector on a cell line. Viral titer is typically expressed as viral particles (vp) per unit volume (e.g., vp/mL). In various embodiments, the pharmaceutical compositions of the disclosure comprise about 1×10⁸ viral particles per milliliter (vp/mL), about 5×10⁸ vp/mL, about 1×10⁹ vp/mL, about 5×10⁹ vp/mL, about 1×10¹⁰ vp/mL, about 5×10¹⁰ vp/mL, about 1×10¹¹ vp/mL, about 5×10¹¹ vp/mL, about 1×10¹² vp/mL, about 5×10¹² vp/mL, about 5×10¹³ vp/mL, or about 1×10¹⁴ vp/mL, or about 5×10¹⁴ of the viral vector (e.g., rAAV virion).

The viral vector administered to the subject can be traced by a variety of methods. For example, recombinant viruses labeled with or expressing a marker (such as green fluorescent protein, or beta-galactosidase) can readily be detected. The recombinant viruses may be engineered to cause the target cell to express a marker protein, such as a surface-expressed protein or a fluorescent protein. Alternatively, the infection of target cells with recombinant viruses can be detected by their expression of a cell marker that is not expressed by the animal employed for testing (for example, a human-specific antigen when injecting cells into an experimental animal). The presence and phenotype of the target cells can be assessed by fluorescence microscopy (e.g., for green fluorescent protein, or beta-galactosidase), by immunohistochemistry (e.g., using an antibody against a human antigen), by ELISA (using an antibody against a human antigen), or by RT-PCR analysis using primers and hybridization conditions that cause amplification to be specific for RNA indicative of a cardiac phenotype.

### Exemplary Embodiments

In some aspects, provided herein is a vector comprising a polynucleotide encoding one or more gene products, operably linked to one or more promoters. In some embodiments, the gene product has a cardioprotective effect when the vector is administered to a cell or a mammal having a deleterious mutation in the TTN gene. In some embodiments, the deleterious mutation in the TTN gene is a truncating variant mutation in the TTN gene. In some embodiments, the cardioprotective effect is protection against and/or amelioration of sarcomere dysfunction or disarray observed in cells (e.g., cardiomyocytes) having a deleterious mutation in the TTN gene.

In some embodiments of the vectors described herein, the gene product is not a TTN polypeptide. In some embodiments, the gene product is an MMP11 polypeptide, and optionally wherein the promoter is a cardiac-specific promoter (e.g., a TNNT2 promoter). In some embodiments, the gene product is a SYNPO2LA polypeptide or a SYNPO2LB polypeptide, and optionally wherein the promoter is a cardiac-specific promoter (e.g., a TNNT2 promoter). In some embodiments, the gene product is an MTSS1 inhibitor that inhibits MTSS1 (e.g., inhibits the expression of MTSS1 gene or inhibits the protein level or activity of MTSS1 gene product), and optionally wherein the promoter is a cardiac-specific promoter (e.g., a TNNT2 promoter). In some embodiments, the gene product is an MTSS1 inhibitor that inhibits the expression of MTSS1. In some embodiments, the MTSS1 inhibitor is an inhibitory RNA inhibiting the expression of MTSS1, and the promoter is a short RNA-specific promoter (e.g. a U6 promoter). In some embodiments, the inhibitory RNA is an siRNA. In some embodiments, the inhibitory RNA is an shRNA. In some embodiments, the MTSS1 inhibitor is a CRISPR/Cas system-based inhibitor. In some embodiments, the MTSS1 inhibitor comprises a Cas endonuclease protein operably linked to a promoter, optionally wherein the promoter is a cardiac-specific promoter (e.g., TNNT2 promoter), and/or a guide RNA (gRNA) optionally linked to a short RNA-specific promoter (e.g., a U6 promoter). In some embodiments, the gRNA is complementary to a sequence of the MTSS1 gene (such as a coding sequence). In some embodiments, the gRNA is complementary to a non-coding sequence regulating expression of the MTSS1 gene. In some embodiments, the gRNA is complementary to a sequence of an enhancer of the MTSS1 gene. In some embodiments, the enhancer is a cardiac-specific enhancer of the MTSS1 gene.

In some aspects, provided herein is a vector comprising a polynucleotide encoding one or more gene products, operably linked to one or more promoters. In some embodiments, the gene product has a cardioprotective effect when the vector is administered to a cell or a mammal having a deleterious mutation in the MLP/CSRP3 gene. In some embodiments, the cardioprotective effect is protection against and/or amelioration of sarcomere dysfunction or disarray observed in cells (e.g., cardiomyocytes) having a deleterious mutation in the MLP/CSRP3 gene. In some embodiments of the vectors described herein, the gene product is not an MLP/CSRP3 polypeptide. In some embodiments, the gene product is an MMP11 polypeptide, and optionally wherein the promoter is a cardiac-specific promoter (e.g., a TNNT2 promoter). In some embodiments, the gene product is a SYNPO2LA polypeptide or a SYNPO2LB polypeptide, and optionally wherein the promoter is a cardiac-specific promoter (e.g., a TNNT2 promoter). In some embodiments, the gene product is an MTSS1 inhibitor that inhibits MTSS1 (e.g., inhibits the expression of MTSS1 gene or inhibits the protein level or activity of MTSS1 gene product), and optionally wherein the promoter is a cardiac-specific promoter (e.g., a TNNT2 promoter). In some embodiments, the gene product is an MTSS1 inhibitor that inhibits the expression of MTSS1. In some embodiments, the MTSS1 inhibitor is an inhibitory RNA inhibiting the expression of MTSS1, and the promoter is a short RNA-specific promoter (e.g., a U6 promoter). In some embodiments, the inhibitory RNA is an siRNA. In some embodiments, the inhibitory RNA is an shRNA. In some embodiments, the MTSS1 inhibitor is a CRISPR/Cas system-based inhibitor. In some embodiments, the MTSS1 inhibitor comprises a Cas endonuclease protein operably linked to a promoter, optionally wherein the promoter is a cardiac-specific promoter (e.g., TNNT2 promoter), and/or a guide RNA (gRNA) optionally linked to a short RNA-specific promoter (e.g., a U6 promoter). In some embodiments, the gRNA is complementary to a sequence of the MTSS1 gene (such as a coding sequence, e.g., the sequence of exon 1 of MTSS1). In some embodiments, the gRNA is complementary to a non-coding sequence regulating the expression of the MTSS1 gene. In some embodiments, the gRNA is complementary to a non-coding sequence promoting the expression of MTSS1 in muscle cells, cardiac cells, cardiomyocytes or the heart, and not substantially promoting the expression or promoting a lower level of expression of MTSS1 in other cells or tissues, e.g., liver, kidney or brain cells and tissues. In some embodiments, the gRNA is complementary to a sequence of an enhancer of the MTSS1 gene. In some embodiments, the enhancer is a cardiac-specific enhancer of the MTSS1 gene. In some embodiments, the gRNA is complementary to a sequence of one or more transcription binding sites (such as any one or more specified herein) in an enhancer of the MTSS1 gene. In some embodiments, the targeted enhancer sequence or the transcription binding sites in the enhancer are cardiac-specific, or promote the expression of MTSS1 in muscle cells, cardiac cells, cardiomyocytes or the heart, and do not substantially promote the expression or promote a lower level of expression of MTSS1 in, e.g., liver, kidney or brain cells and tissues. In some embodiments, the enhancer is the native cardiac-specific enhancer of the MTSS1 gene. In some embodiments, the gRNA is complementary to a sequence of a promoter of the MTSS1 gene. In some embodiments, the promoter is a native cardiac-specific promoter of the MTSS1 gene. In some embodiments, the gRNA inhibits the expression of the MTSS1 gene by targeting a muscle-specific or cardiac-specific non-coding sequence regulating the expression of the MTSS1 gene (e.g., by targeting a muscle-specific or cardiac-specific promoter or enhancer region of the MTSS1 gene).

In some aspects, provided herein is a vector comprising a polynucleotide encoding one or more gene products, operably linked to one or more promoters, wherein the gene product is selected from the group consisting of:
(a) the gene product is an MMP11 polypeptide (e.g., wherein the promoter is a cardiac-specific promoter, such as a TNNT2 promoter);
(b) the gene product is a SYNPO2LA polypeptide (e.g., wherein the promoter is a cardiac-specific promoter, such as a TNNT2 promoter);
(c) the gene product is a SYNPO2LB polypeptide (e.g., wherein the promoter is a cardiac-specific promoter, such as aTNNT2 promoter); and
(d) the gene product is an MTSS1 inhibitor, e.g., an inhibitory RNA (e.g., an siRNA or shRNA) inhibiting the expression of MTSS1 (e.g., wherein the promoter is a short RNA-specific promoter, such as a U6 promoter), or a Cas endonuclease protein (e.g., wherein the promotor is a cardiac-specific promoter, such as a TNNT2 promoter) and/or a guide RNA (gRNA) (e.g., wherein the promoter is a short RNA-specific promoter, such as a U6 promoter).

In some embodiments of the vectors described herein, the polynucleotide encodes two, three, or more gene products. In some embodiments, two, three, or more gene products are selected from the gene products (a), (b), (c) and (d) specified above.

In some embodiments of the vectors described herein, the vector is a viral vector. In some embodiments, the viral vector is an adeno-associated virus vector (AAV). In some embodiments, the AAV is AAV9. However, other vectors, including other viral vectors and other AAV serotypes are also contemplated for use herein as described herein. In some embodiments of the vectors described herein, the vector is a lentiviral vector.

In some embodiments of the vectors described herein, the vector genome has a size equal to or less than 5.6 kB. In some embodiments, the vector genome has a size equal to or less than 5 kB. In some embodiments, the vector genome has a size equal to or less than 4.8 kB. In some embodiments, the vector genome has a size of between 4 kB and 5.6 kB, 4 kB and 5 kB, or 4 kB and 4.8 kB. In some of these embodiments, the vector is an AAV (e.g., AAV9-based) vector.

In some embodiments of the vectors described herein, the polynucleotide encoding an MMP11 polypeptide shares at least 80%, 85%, 90%, 95%, 96%, 97%, 98%, 99%, or 100% identity to SEQ ID NO: 86, and/or the MMP11 polypeptide shares at least 80%, 85%, 90%, 95%, 96%, 97%, 98%, 99%, or 100% identity to SEQ ID NO: 87.

In some embodiments of the vectors described herein, the polynucleotide encoding a SYNPO2LA polypeptide shares at least 80%, 85%, 90%, 95%, 96%, 97%, 98%, 99%, or 100% identity to SEQ ID NO: 88, and/or the SYNPO2LA polypeptide shares at least 80%, 85%, 90%, 95%, 96%, 97%, 98%, 99%, or 100% identity to SEQ ID NO: 89.

In some embodiments of the vectors described herein, the polynucleotide encoding SYNPO2LB polypeptide shares at least 80%, 85%, 90%, 95%, 96%, 97%, 98%, 99%, or 100% identity to SEQ ID NO: 90, and/or the SYNPO2LB polypeptide shares at least 80%, 85%, 90%, 95%, 96%, 97%, 98%, 99%, or 100% identity to SEQ ID NO: 91.

In some embodiments of the vectors described herein, the inhibitory RNA inhibiting the expression of MTSS1, comprises at least 15, 16, 17, 18, or 19 contiguous nucleotides, with 0, 1, 2 or 3 mismatches, optionally with 0 mismatch, of any one of the following sequences: SEQ ID NOs: 94-100. In some embodiments, the inhibitory RNA inhibiting the expression of MTSS1, comprises at least 15, 16, 17, 18, or 19 contiguous nucleotides of any one of the following sequences: SEQ ID NOs: 94-100. In some embodiments, the inhibitory RNA targets any region of MTSS1 mRNA of SEQ ID NO: 92. In some embodiments, the inhibitory RNA inhibits the expression of MTSS1 protein of SEQ ID NO: 93. In some embodiments, the inhibitory RNA is an siRNA. In some embodiments, the inhibitory RNA is an shRNA.

In some aspects, provided herein is a recombinant AAV (rAAV) virion comprising one or more vectors described herein, and an AAV capsid protein. In some embodiments, the rAAV virion is a serotype AAV9 virion or a variant thereof, and/or the AAV capsid protein is an AAV9 capsid protein or a variant thereof. In some embodiments, the rAAV virion comprises any of the AAV capsid proteins described herein, including without limitation any of the engineered capsid proteins (e.g., engineered AAV9 capsid proteins comprising one or more substitutions or insertions) described herein.

In some aspects, provided herein is a method of expressing a gene product in a cell, comprising transducing the cell with any vector or virion described herein. In some aspects, provided herein is an ex vivo method of expressing a gene product in a cell, comprising transducing the cell with any vector or virion described herein. In some aspects, provided herein is an in vitro method of expressing a gene product in a cell, comprising transducing the cell with any vector or virion described herein.

In some aspects, provided herein is an isolated cell comprising any of the vectors described herein. In some aspects, provided herein is an isolated cell comprising any of the virions described herein. In some embodiments, the isolated cell is an induced pluripotent stem cell. In some embodiments, the isolated cell is an isolated cardiac cell. In some embodiments, the isolated cell is an isolated cardiomyocyte.

In some aspects, provided herein is a cell therapy composition comprising any cell or isolated cell, or population of cells, described herein.

In some aspects, provided herein is a pharmaceutical composition comprising at least one of the vectors described herein. In some aspects, provided herein is a pharmaceutical composition comprising two, three, or more of the vectors described herein.

In some aspects, provided herein is a pharmaceutical composition comprising at least one of the virions described herein.

In some aspects, provided herein is a method of treating and/or preventing a heart disease in a subject, comprising administering to the subject one or more vectors described herein. In some aspects, provided herein is a method of treating and/or preventing a heart disease in a subject, comprising administering to the subject one or more virions described herein. In some aspects, provided herein is a method of treating and/or preventing a heart disease in a subject, comprising administering to the subject a cell therapy described herein. In some aspects, provided herein is a method of treating and/or preventing a heart disease in a subject, comprising administering to the subject a pharmaceutical composition described herein. In some aspects, provided herein is a method of treating and/or preventing a heart disease in a subject, comprising administering to the subject any inhibitory RNA inhibiting the expression of MTSS1 described herein. In some embodiments, the inhibitory RNA is an siRNA or an shRNA.

In some embodiments, provided herein is a method of treating and/or preventing a heart disease in a subject, comprising administering to the subject two, three, or more vectors described herein. In some embodiments, provided herein is a method of treating and/or preventing a heart disease in a subject, comprising administering to the subject two, three, or more inhibitory RNAs described herein. In some embodiments, provided herein is a method of treating and/or preventing a heart disease in a subject, comprising administering to the subject at least one (e.g., two or more) vectors described herein and at least one (e.g., two or more) inhibitory RNA described herein.

In some embodiments of the methods described herein, the heart disease is an acquired or genetic form of heart failure or cardiomyopathy. In some embodiments of the methods described herein, the heart disease is an acquired or genetic form of heart failure with reduced ejection fraction or dilated cardiomyopathy.

In some embodiments of the methods described herein, the subject has a genetic mutation associated with heart failure or cardiomyopathy. In some embodiments of the methods described herein, the subject has a genetic mutation associated with heart failure with reduced ejection fraction or dilated cardiomyopathy.

In some embodiments of the methods described herein, the heart disease is associated with a deleterious mutation in the TTN gene. In some embodiments, the deleterious mutation in the TTN gene is a truncating variant mutation in the TTN gene. In some embodiments, the subject has a deleterious mutation in the TTN gene.

In some embodiments of the methods described herein, the heart disease is associated with a deleterious mutation in the MLP/CSRP3 gene. In some embodiments, the subject has a deleterious mutation in the MLP/CSRP3 gene.

In some embodiments, the subject is a mammal (e.g., a human).

In some embodiments of the methods described herein, the heart disease is cardiomyopathy. In some embodiments, the cardiomyopathy is dilated cardiomyopathy. In some embodiments, the heart disease is heart failure (e.g., heart failure with reduced ejection fraction). In some embodiments, the administering to the subject improves cardiac function and/or ameliorates sarcomere dysfunction in cardiac cells of the subject.

In some embodiments of the methods described herein, the administering is by systemic administration, e.g., parenteral administration. In some embodiments, the systemic administration is intravenous administration (e.g., an IV infusion).

In some embodiments of the methods described herein, the administering is by local administration to the heart. In some embodiments, the local administration is by direct injection into the heart or cardiac tissue. In some embodiments, the local administration is intracoronary administration. In some embodiments, the local administration is retrograde coronary sinus infusion.

In some aspects, provided herein is any MTSS1 inhibitor that inhibits MTSS1 (e.g., inhibits the expression of MTSS1 gene or inhibits the protein level or activity of MTSS1 gene product). In some embodiments, the MTSS1 inhibitor is cardiac-specific. In some embodiments, the MTSS1 inhibitor is not a vector or is not comprised within a vector. In some embodiments, the MTSS1 inhibitor is a small molecule. In some aspects, provided herein is a pharmaceutical composition comprising any MTSS1 inhibitor. In some aspects, provided herein is a method of treating and/or preventing a heart disease in a subject, comprising administering to the subject any MTSS1 inhibitor.

### Enumerated Embodiments

The present technology includes, but is not limited to, the following specific embodiments:

1. A vector comprising a polynucleotide encoding one or more gene products, operably linked to one or more promoters, wherein the gene product has a cardioprotective effect when the vector is administered to a cell or a mammal having a deleterious mutation in TTN gene, optionally wherein the gene product is not a TTN polypeptide.

2. The vector of embodiment 1, wherein the gene product is an MMP11 polypeptide, and optionally wherein the promoter is a TNNT2 promoter.

3. The vector of embodiment 1, wherein the gene product is a SYNPO2LA polypeptide or a SYNPO2LB polypeptide, and optionally wherein the promoter is a TNNT2 promoter.

4. The vector of embodiment 1, wherein the gene product is an inhibitory RNA inhibiting the expression of MTSS1, optionally wherein the inhibitory RNA is an siRNA or an shRNA, and optionally wherein the promoter is a U6 promoter.

5. The vector of any one of embodiments 1-4, wherein the deleterious mutation in the TTN gene is a truncating variant mutation in the TTN gene.

6. The vector of any one of embodiments 1-5, wherein the cardioprotective effect is protection against and/or amelioration of sarcomere dysfunction or disarray observed in cells having a deleterious mutation in the TTN gene, optionally wherein the cells are cardiomyocytes.

7. A vector comprising a polynucleotide encoding one or more gene products, operably linked to one or more promoters, wherein the gene product is selected from the group consisting of:
(a) an MMP11 polypeptide, and optionally wherein the promoter is a TNNT2 promoter;
(b) a SYNPO2LA polypeptide or a SYNPO2LB polypeptide, and optionally wherein the promoter is a TNNT2 promoter; and
(c) an inhibitory RNA, optionally wherein the inhibitory RNA is an siRNA or an shRNA, inhibiting the expression of MTSS1, and optionally wherein the promoter is a U6 promoter.

8. The vector of any one of embodiments 1-7, wherein the polynucleotide encodes two or more gene products, and wherein two or more gene products are selected from the gene products (a), (b) and (c) specified in embodiment 7.

9. The vector of any one of embodiments 1-8, wherein the vector is a viral vector.

10. The vector of embodiment 9, wherein the viral vector is an adeno-associated virus vector (AAV).

11. The vector of embodiment 10, wherein the AAV is AAV9.

12. The vector of any one of embodiments 9-11, wherein the vector has a size equal to or less than 5.6 kB.

13. The vector of any one of embodiments 2-11, wherein, if used:
the polynucleotide encoding the MMP11 polypeptide shares at least 80%, at least 85%, at least 90%, at least 95%, at least 96%, at least 97%, at least 98%, at least 99%, or 100% identity to SEQ ID NO: 86, and/or the MMP11 polypeptide shares at least 80%, at least 85%, at least 90%, at least 95%, at least 96%, at least 97%, at least 98%, at least 99%, or 100% identity to SEQ ID NO: 87;
the polynucleotide encoding the SYNPO2LA polypeptide shares at least 80%, at least 85%, at least 90%, at least 95%, at least 96%, at least 97%, at least 98%, at least 99%, or 100% identity to SEQ ID NO: 88, and/or the SYNPO2LA polypeptide shares at least 80%, at least 85%, at least 90%, at least 95%, at least 96%, at least 97%, at least 98%, at least 99%, or 100% identity to SEQ ID NO: 89;
the polynucleotide encoding the SYNPO2LB polypeptide shares at least 80%, at least 85%, at least 90%, at least 95%, at least 96%, at least 97%, at least 98%, at least 99%, or 100% identity to SEQ ID NO: 90, and/or the SYNPO2LB polypeptide shares at least 80%, at least 85%, at least 90%, at least 95%, at least 96%, at least 97%, at least 98%, at least 99%, or 100% identity to SEQ ID NO: 91; or
the inhibitory RNA, optionally siRNA or shRNA, inhibiting the expression of MTSS1: comprises at least 15, 16, 17, 18, or 19 contiguous nucleotides, with 0, 1, 2, or 3 mismatches, optionally with 0 mismatches, of any one of the following sequences: SEQ ID NOs: 94-100; targets any region of MTSS1 mRNA of SEQ ID NO: 92; or inhibits the expression of MTSS1 protein of SEQ ID NO: 93.

14. An isolated cell comprising the vector of any one of embodiments 1-13.

15. The isolated cell of embodiment 14, wherein the isolated cell is an induced pluripotent stem cell or an isolated cardiomyocyte.

16. A cell therapy composition comprising the cell of embodiment 14 or embodiment 15.

17. A pharmaceutical composition comprising at least one vector of any one of embodiments 1-13.

18. A method of treating and/or preventing heart disease in a subject, comprising administering to a subject the vector of any one of embodiments 1-13, the cell therapy of embodiment 16, the pharmaceutical composition of embodiment 17, the virion of embodiment 34 or 35, or an inhibitory RNA inhibiting the expression of MTSS1, optionally wherein the inhibitory RNA is an siRNA.

19. The method of embodiment 18, wherein the heart disease is an acquired or genetic form of heart failure or cardiomyopathy, optionally wherein the heart disease is an acquired or genetic form of heart failure with reduced ejection fraction or dilated cardiomyopathy.

20. The method of embodiment 18 or 19, wherein the subject has a genetic mutation associated with heart failure or cardiomyopathy, optionally wherein the subject has a genetic mutation associated with heart failure with reduced ejection fraction or dilated cardiomyopathy.

21. The method of embodiment 18, wherein the heart disease is associated with a deleterious mutation in the TTN gene or the subject has a deleterious mutation in the TTN gene.

22. The method of embodiment 21, wherein the deleterious mutation in the TTN gene is a truncating variant mutation in the TTN gene.

23. The method of embodiment 18, wherein the heart disease is associated with a deleterious mutation in the MLP/CSRP3 gene or the subject has a deleterious mutation in the MLP/CSRP3 gene.

24. The method of any one of embodiments 18-23, wherein the heart disease is cardiomyopathy.

25. The method of embodiment 24, wherein the cardiomyopathy is dilated cardiomyopathy.

26. The method of any one of embodiments 18-23, wherein the heart disease is heart failure.

27. The method of embodiment 26, wherein the heart failure is heart failure with reduced ejection fraction.

28. The method of any one of embodiments 18-27, wherein the administering improves cardiac function and/or ameliorates sarcomere dysfunction in cardiac cells of the subject.

29. The method of any one of embodiments 18-28, wherein the administering is systemic administration or local administration to the heart.

30. The method of embodiment 29, wherein the systemic administration is intravenous administration.

31. The method of embodiment 29, wherein the local administration is by direct injection into the heart or cardiac tissue, intracoronary administration or retrograde coronary sinus infusion.

32. A method of expressing a gene product in a cell, comprising transducing the cell with the vector of any one of embodiments 1-13.

33. The method of embodiment 32, which is an in vitro or ex vivo method.

34. A recombinant AAV (rAAV) virion, comprising the vector of any one of embodiments 1-13, and an AAV capsid protein, optionally wherein the rAAV virion is a serotype AAV9 virion, or a variant thereof, and/or the AAV capsid protein is an AAV9 capsid protein or variant thereof.

35. A recombinant AAV (rAAV) virion, comprising the vector of any one of embodiments 1-13, and any one of the AAV capsid proteins described herein.

### EXAMPLES

### Example 1: High Content Screening to Identify Cardioprotective Genes and siRNAs

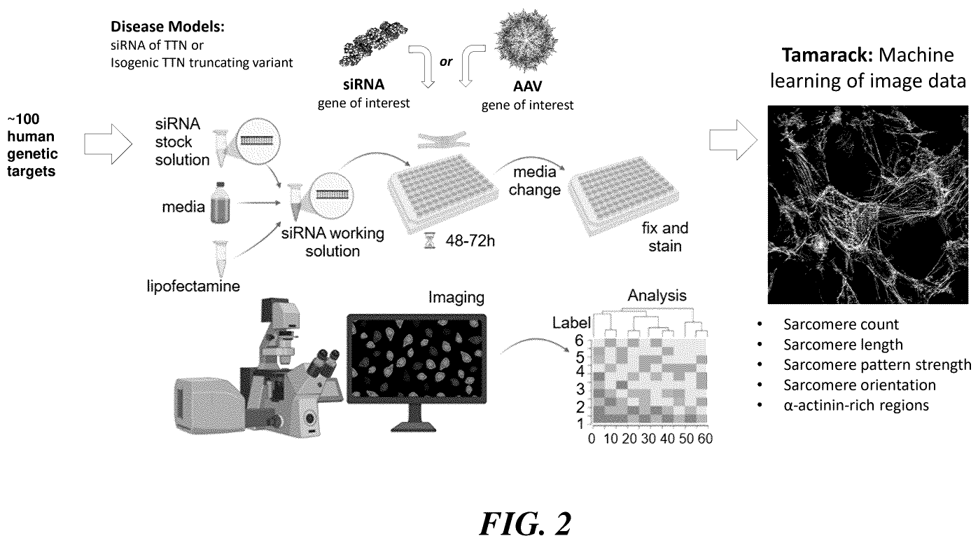

Internal analyses were undertaken using available genetic data from human heart failure and contractility phenotypes to prioritize a set of 104 genetic targets for screening. Of the 104, 96 targets were amenable to genetic manipulation by overexpression from either packaging in an AAV9 vector under the control of the TNNT2 promoter, or inhibition using siRNA molecules. Knockdown and/or overexpression was performed systematically (with technical replicates) in induced pluripotent cardiomyocytes (iPSCs) with compromised TTN, either treated with an siTTN siRNA or containing a known TTN truncating variant P22353X^{+/-}. Cellular and sarcomere structure were quantified by automated quantification of three-channel imaging (DAPI, MYBPC3, ACTN2) of high-throughput microscopy. Comparisons between conditions were performed by linear modeling accounting for technical replicates. High throughput imaging and quantification of cardiomyocytes captured cellular phenotypes and disturbances in sarcomere structure due to mutation or knockdown of cardiomyopathy gene TTN. FIG. 2 shows an illustration of the performed screening and cardioprotective target identification.

### Materials & Methods:

The day prior to the experiment start, 384-well plates were coated with Matrigel and stored in a 37° C. CO₂ incubator overnight. After overnight incubation, plates were flicked clean of excess solution. iCell Cardiomyocytes (‘CDI cells’) or an isogenic mutant line (TTN P22353X^{+/-} or a BAG3 KO^{+/-}) were thawed and seeded on 384-well pre-coated plates at a density of 10 k/well. Cells were allowed to recover in plating media for 4 hours and switched to maintenance media for 7 days, with media changes every other day. After 7 days, cells were treated with one of three conditions: a) an siRNA library consisting of 98 individual genes of interest (GOI) for silencing; b) siTTN across the entire plate(s) to induce sarcomere disarray and the same siRNA library, or c) siTTN across the entire plate(s) and, 48 hours later, an AAV library to overexpress the same GOIs. 7 days after the initial siRNA treatment, cells were fixed with 4% PFA and stained with a nuclear stain and florescence antibodies for ACTN2 and cMYBPC.

After fixation, plates were imaged in a Z-stack using an Image Xpress Micro confocal microscope in three separate channels (DAPI, ACTN2, DAPI). 5 images were taken per well in a cross (‘+’) pattern to capture the images across the entire well. Images were then analyzed using a MATLAB analysis software ‘Tamarack’ which outputs nuclear count, sarcomere count, sarcomere length, sarcomere angle, and sarcomere fit score for each image. Nuclei were detected using a straight-forward circle-identifying algorithm. Sarcomere parameters included count, length, angle, and fit-score and were quantified from the imaging data for each treatment. Output of images were averaged for each well, and wells were averaged for each condition. This analysis allowed measuring the impact of knocking down or overexpressing a specific gene on the sarcomere structure in wild-type and a cardiomyopathy background.

### Results

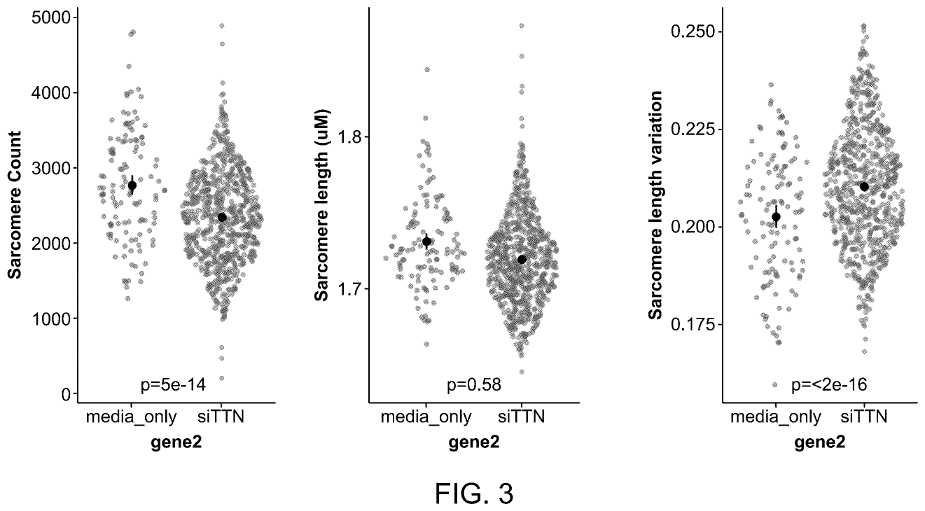

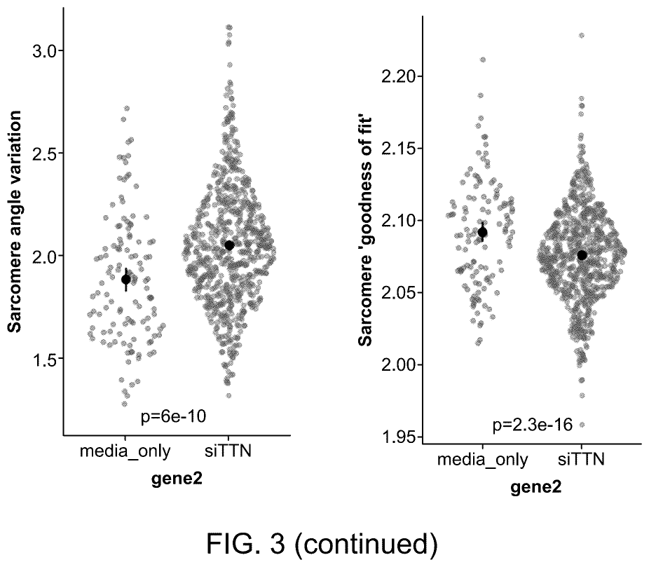

As shown in FIG. 3, siRNA knockdown of TTN in wild-type (CDI) IPS cardiomyocytes disturbed sarcomere architecture, decreasing sarcomere count, length, and algorithmic ‘goodness of fit’, while increasing the variability in sarcomere length and orientation. For example, treatment of iPSCs with siTTN decreased sarcomere count from 17% to 33%.

As a result of this screening, SYNPO2L genes were identified as having cardioprotective effect in the background of compromised TTN, and MTSS1 was identified as a risk-carrying gene while siRNA inhibition of MTSS1 conferred a cardioprotective effect.

### SYNPO2L

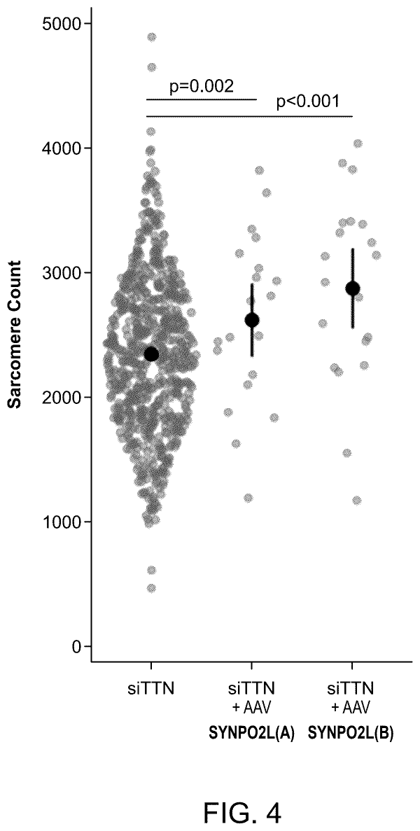

As shown in FIG. 4, high-content microscopy of an induced pluripotent stem cell (iPSC) line treated with siTTN to mimic sarcomere defects revealed an increase in sarcomere count when treated with AAV delivery of the longer isoform of SYNPO2L_A (p=0.002) or the shorter isoform of SYNPO2L_B (p<0.001). Further, sarcomere density in TTN backgrounds was increased by AAV delivery of SYNPO2L isoforms.

These results show that supplementation or overexpression of SYNPO2LA and/or SYNPO2LB using AAV can restore aspects of sarcomere structure and have a beneficial therapeutic effect in individuals with a TTN cardiomyopathy and more broadly with DCM or heart failure.

### MTSS1

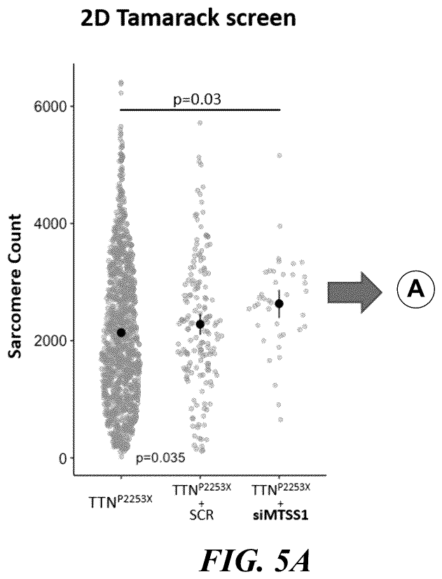

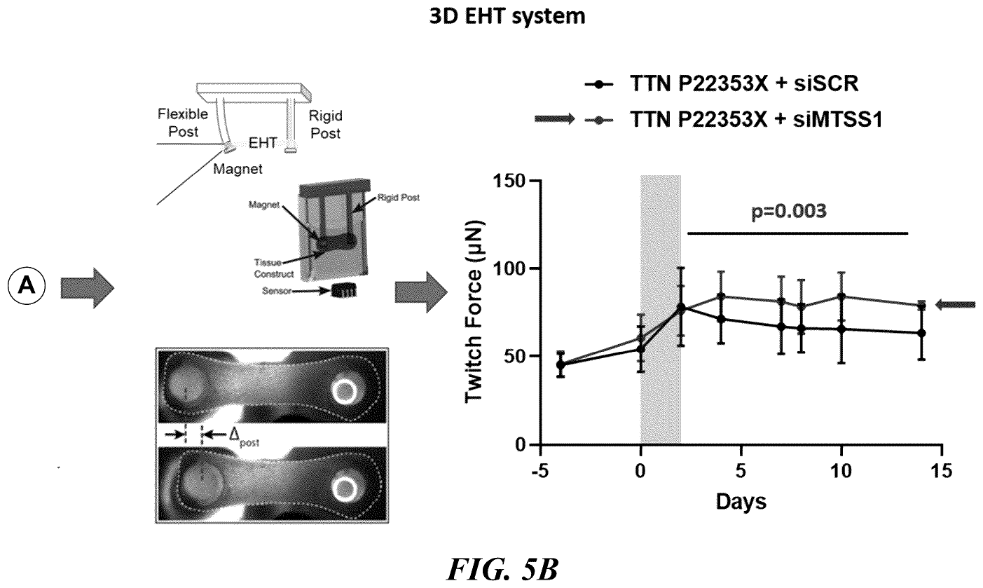

As shown in FIG. 5A, high-content microscopy of an induced pluripotent stem cell (iPSC) line which carries a heterozygous TTN truncating mutation (P22353X^{+/-}) revealed an increase in total sarcomere count with siRNA knockdown of MTSS1 treatment (p=0.03) relative to untreated cells. MTSS1 siRNA used in the experiments was siRNA having SEQ ID NO: 94. Inhibition of MTSS using siRNA technology improved 2D sarcomere architecture in TTN deficient iPSC cardiomyocytes. Engineered heart tissues derived from the same TTN P22353X^{+/-} iPSC line revealed a significant and sustained improvement in force produced when treated with siMTSS1 at 5 nM concentration for 48 hours (22.1 PN, SE 7.1 SE, p=0.003) relative to untreated cells.

These results show that inhibition of MTSS1 can have a beneficial therapeutic effect in individuals with a TTN cardiomyopathy and more broadly with DCM or heart failure.

### Example 2: Engineered Heart Tissue Validation with siRNA Targeting MTSS1

Engineered heart tissue (EHT) studies were conducted using the CuriBio Mantarray platform, and tissues were prepared as per the manufacturer's protocols. hiPSC-derived cardiomyocytes (hiPSC-CMs) were generated from an isogenic mutant line (TTN P22353X^{+/-}) and were mixed with primary human cardiac fibroblasts at a 6.67:1 ratio. 5 mg/mL fibrinogen solution was added to the cardiomyocyte-cardiac fibroblast cell suspension and mixed thoroughly. This mixture was then added to an ice-cold 6 U/mL solution of thrombin that had already been added to each well of the casting plate. Polydimethylsiloxane (PDMS) posts were inserted into the casting wells, and the casted tissues were allowed to crosslink and form around the posts for 90 minutes in a 37° C. CO₂ incubator before being lifted out of the casting plate and transferred to a fresh 24-well plate filled with 2 mL of media per well. After 7 days of culture, culture media was switched from RPMI supplemented with B-27 to a maturation media formulation. After an additional 14 days of culture, baseline contractile properties of EHTs were measured and then tissues were treated with 5 nM of siMTSS1 (siRNA targeting MTSS1) for 48 hours. Contractility measurements were then conducted every 2-3 days for a further 10-14 days of culture and the difference in contractility after siMTSS1 treatment was assessed with standard linear modeling.

As shown in FIG. 5B, the engineered heart tissues derived from the TTN P22353X^{+/-} iPSC line revealed a significant and sustained improvement in force produced when treated with siMTSS1 at 5 nM concentration for 48 hours (22.1 μN, SE 7.1 SE, p=0.003) relative to untreated cells. Inhibition of MTSS using siRNA technology improved contractility in 3D engineered heart tissues in TTN deficient iPSC cardiomyocytes.

These results further show that inhibition of MTSS1 can have a beneficial therapeutic effect in individuals with a TTN cardiomyopathy and more broadly with DCM or heart failure.

### Example 3: Clinical Studies

A pharmaceutical composition comprising rAAV virions encoding MMP11, SYNPO2LA, SYNPO2LB, and/or siRNA/shRNA targeting MTSS1, as described herein, and/or siRNA/shRNA targeting MTSS1 delivered without a viral vector (e.g., as a naked or in a lipid based nanocarrier), is administered intravenously or by retrograde coronary sinus infusion (RCSI). Functional efficacy is determined by cardiac functional status assessments (e.g., New York Heart Association Functional Classification, NYHA; Cardiopulmonary exercise test, CPET), quality of life questionnaires (e.g., Kansas City Cardiomyopathy Questionnaire Clinical Quality Score, KCCQ-CSS), cardiac imaging (e.g., echocardiography), cardiac biomarkers (e.g. troponin and NT-proBNP), cardiac rhythm and immunologic assessments, cardiac functional status assessments (e.g., Pediatric Interagency Registry for Mechanically Assisted Circulatory Support, PEDIMACS; Ross classifications), and/or Major Adverse Cardiac Events (MACE) (total death, cardiac transplantation, initiation of inotropes, initiation of ventilatory, or mechanical circulatory support). Clinical studies may include monitoring safety and continued efficacy (e.g., adverse events, severe adverse events, electrocardiogram, cardiac enzymes, biomarkers, functional status, left ventricular (LV) function/mass, quality of life, serum chemistries, liver function tests) on an annual basis for up to 10 years.

### Example 4: Screening for siMTSS1 in a Different Genetic DCM Background

### Methods:

### Plate Preparation

The day prior to experiment start 384-well plates were pre-coated with Matrigel and stored in a 37° C. CO₂ incubator overnight. After overnight incubation, the plates were flicked clean of excess solution. iCell Cardiomyocytes (‘CDI cells’) were thawed and seeded on the 384-well pre-coated plates at a density of 10 k/well. Cells were allowed to recover in plating media for 4 hours and then switched to maintenance media for 7 days, with media changes every other day. After 7 days, cells were treated with a) an siRNA library with the MLP/CSRP3 gDCM background, alongside b) siMTSS1 at 0, 5 nM, or 10 nM concentrations (siMTSS1 used in the experiments was siRNA having SEQ ID NO: 94). Seven days after initial siRNA treatment cells were fixed with 4% PFA and stained with a nuclear stain and florescence antibodies for ACTN2 and cMYBPC.

### Imaging and Analysis

After fixation, plates were imaged in a Z-stack using an Image Xpress Micro confocal microscope in three separate channels. 5 images were taken per well in a cross (‘+’) pattern to capture the images across the entire well. Images were then analyzed using the Tamarack software, described in Example 1, which outputs nuclear count, sarcomere count, sarcomere length, sarcomere angle, and sarcomere fit score for each image. Nuclei were detected using a straight-forward circle-identifying algorithm while sarcomere parameters were including count, length, angle, and fit-score were identified using a custom which employs a characteristic 3-element ‘wavelet’ pattern to identify and place sarcomeres within the image. Output of images were averaged for each well, and wells were averaged for each condition.

Using the data from Tamarack, a random forest classification algorithm was trained to distinguish wells with scrambled control (SCR) siRNA from the MLP/CSRP3 siRNA with excellent sensitivity and specificity, and subsequently applied to the siMTSS1 treated samples to assess the likelihood that the knockdown of MLP/CSRP3 appeared to be rescued by treatment with siMTSS1.

### Results

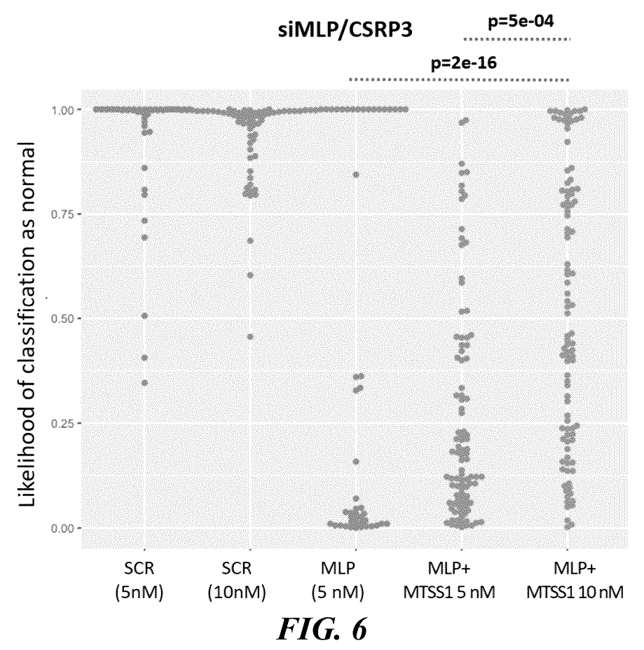

FIG. 6 shows the probabilities of ‘rescue’ or classification as SCR treatment alone. The SCR control samples treated at 5 nM (median probability 0.99) or 10 nM (median probability 0.99) conditions were primarily classified correctly as rescued. The siMLP knockdown alone were primarily classified as abnormal (median probability 0.02). In comparison to the siMLP knockdown alone, the 5 nM siMTSS1+siMLP displayed improved likelihood of normal appearance (median probability 0.16, p=5e-04) and the 10 nM siMTSS1+siMLP displayed even higher likelihood of normal appearance (median probability 0.48, p=2e-16). These results indicate that inhibition of MTSS1 improved sarcomere architecture or appearance in MLP/CSRP3 deficient cardiomyocytes.

These results show that inhibition of MTSS1 can have a beneficial therapeutic effect in the context of another mutant background associated with heart failure and cardiomyopathy.

### Example 5: Engineered Heart Tissue Validation (MTSS1 Only)

### Methods:

Engineered heart tissue (EHT) studies were conducted using the CuriBio Mantarray platform, and tissues were prepared as per the manufacturer's protocols. hiPSC-derived cardiomyocytes (hiPSC-CMs) generated from a wild-type cell line were mixed with primary human cardiac fibroblasts at a 6.67:1 ratio. 5 mg/mL fibrinogen solution was added to the cardiomyocyte-cardiac fibroblast cell suspension and mixed thoroughly. This mixture was then added to an ice-cold 6 U/mL solution of thrombin which had already been added to each well of the casting plate. Polydimethylsiloxane (PDMS) posts were inserted into the casting wells, and the casted tissues were allowed to crosslink and form around the posts for 90 minutes in a 37° C. CO₂ incubator before being lifted out of the casting plate and transferred to a fresh 24-well plate filled with 2 mL of media per well. After 7 days of culture, culture media was switched from RPMI supplemented with B-27 to an in-house maturation media formulation. After an additional 14 days of culture, baseline contractile properties of EHTs were measured and then tissues were treated with 10 nM scrambled control, 5 nM and 10 nM of siMTSS1 with siMLP at 5 nM, or siMLP 5 nM alone for 48 hours. Contractility measurements were then conducted every day for 7 days of culture and the difference in contractility after siMTSS1 treatment in comparison to siMLP and siSCR controls was assessed with standard linear modeling after normalization to baseline values.

### Results

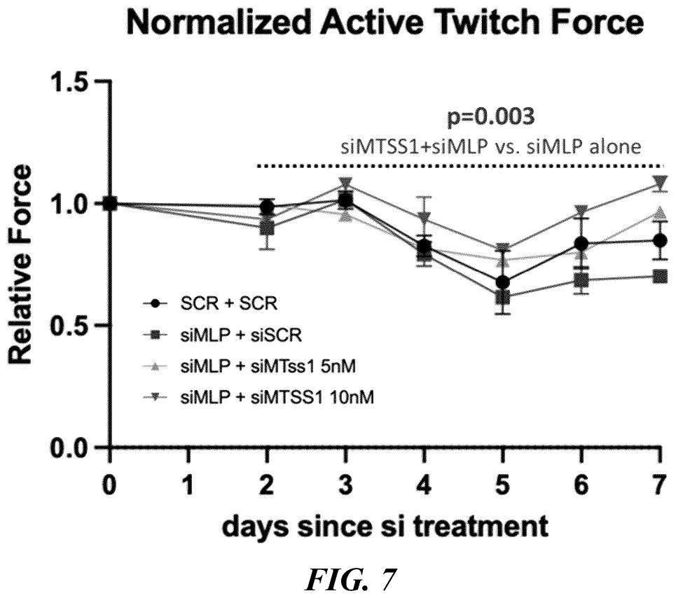

Results are shown in FIG. 7. The wild-type iPSCs treated with siMLP revealed a significant and sustained improvement in force produced when treated with siMTSS1 at 5 nM or 10 nM concentration for 48 hours (average 13% improvement over baseline, SE 3.7%, p=0.001) over 7 days. These data indicate that inhibition of MTSS1 improved contractility in 3D engineered heart tissues in MLP/CSPR3 deficient iPSC cardiomyocytes.

These results show that inhibition of MTSS1 can have a beneficial therapeutic effect in the context of another mutant background associated with heart failure and cardiomyopathy.

### Example 6: Carriers of MTSS1/rs12541595 Allele Show Significantly Improved Event Free Survival in TTN DCM

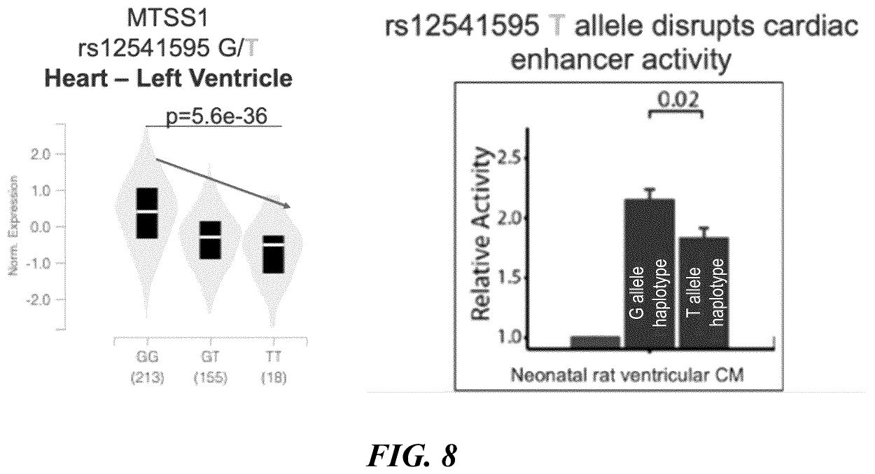

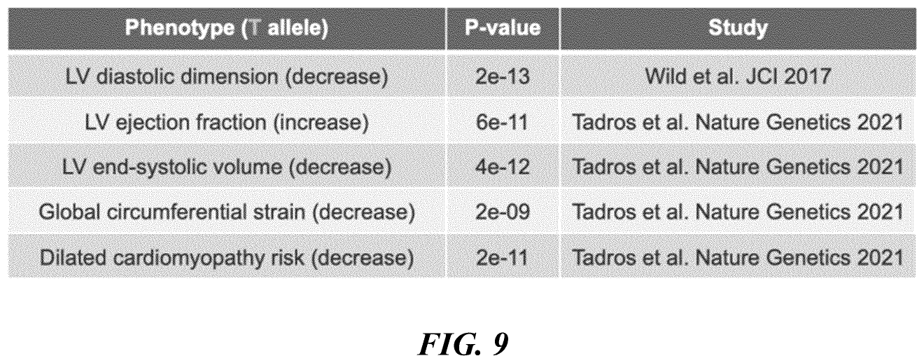

Inter-individual variability among the genetic composition of cardiac structure and function is common. A single-nucleotide polymorphism (SNP) mutation of G to T near the MTSS1 gene locus (rs12541595), within the cardiac-specific enhancer region of MTSS1, disrupts the activities of the enhancer and results in decreased MTSS1 expression in the heart (see FIG. 8). Based on published data (e.g., Wild et al., J. Clin. Invest. (2017) 127(5):1798-1812; Tadros et al., Nat. Genet. (2021) 53(2):128-134), it has also been found that the T allele is associated with several phenotypes including decreased left ventricle (LV) diastolic dimension, increased LV ejection fraction, decreased LV end-systolic volume, decreased global circumferential strain, and decreased dilated cardiomyopathy risks (FIG. 9).

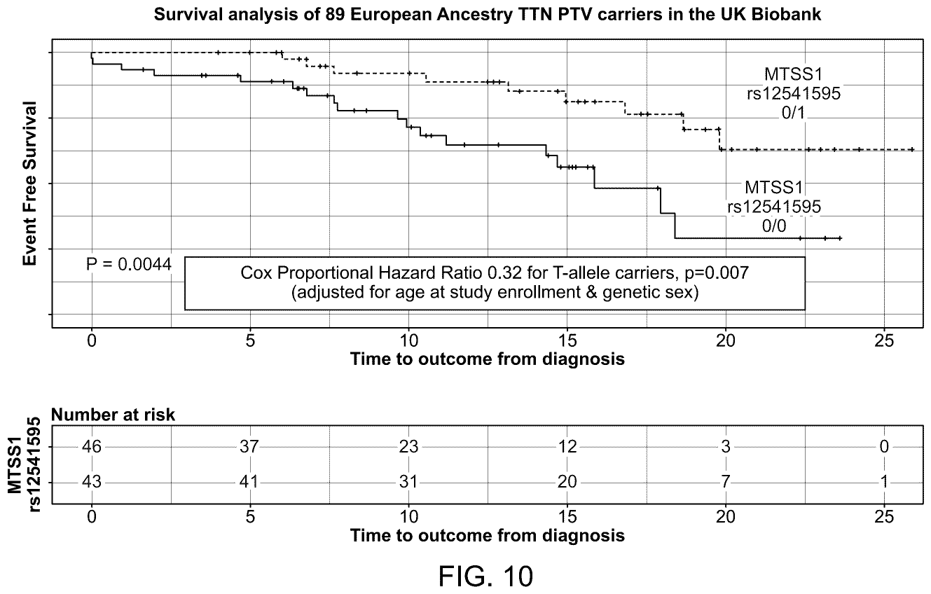

Based on the cardioprotective effect of MTSS1 knockdown by siRNA as observed in the previous examples, it was assessed whether the rs12541595 T allele, which is known to decrease MTSS1 expression levels by inhibiting its cardiac-specific enhancer activity, would also have an effect on cardiac outcome in patients with TTN-associated dilated cardiomyopathy (DCM). To this end, survival analysis was performed on 89 individuals of European ancestry from the UK Biobank study who carry a TTN protein-truncating variant (PTV) and have been diagnosed with DCM any time after enrollment (enrollment ages for the entire 500,000-person study ranged from ages 39 years to 89 years). Those 89 individuals included 46 with “wild-type” rs12541595 G alleles (MTSS1 rs12541595 0/0) and 43 with one copy of the rs12541595 T allele (MTSS1 rs12541595 0/1). Outcome or events of the study included death or heart transplant. As shown in FIG. 10, carriers of the rs12541595 T allele showed significantly improved event-free survival in TTN DCM by a Hazard ratio of 0.32 with p-value of 0.007, even after adjustment for age and genetic sex. These data strongly support reducing MTSS1 levels or activity as a therapy for TTNptv DCM and possibly other forms of genetic dilated cardiomyopathy and non-genetic heart failure.

### INCORPORATION BY REFERENCE

Various references such as patents, patent applications, and publications are cited herein, the disclosures of which are hereby incorporated herein by reference in their entireties. Also, all references mentioned herein are specifically incorporated by reference to disclose and describe the methods and/or materials in connection with which the publications are cited.

1-68. (canceled)

69. An inhibitory siRNA or shRNA inhibiting the expression of MTSS1, optionally wherein the MTSS1 is human MTSS1.

70. The inhibitory siRNA or shRNA of claim 69, which comprises at least 15, 16, 17, 18, or 19 contiguous nucleotides, with 0, 1, 2, or 3 mismatches, of any one of SEQ ID NOs: 94-100.

71-87. (canceled)

88. The inhibitory siRNA or shRNA of claim 69, which comprises at least 15, 16, 17, 18, or 19 contiguous nucleotides, with 0 mismatches, of any one of SEQ ID NOs: 94-100.

89. The inhibitory siRNA or shRNA of claim 69, which comprises at least 15, 16, 17, 18, or 19 contiguous nucleotides, with 0 mismatches, of SEQ ID NO: 94.

90. The inhibitory siRNA or shRNA of claim 69, which targets any region of MTSS1 mRNA of SEQ ID NO: 92, or inhibits the expression of MTSS1 protein of SEQ ID NO: 93.

91. The inhibitory siRNA or shRNA of claim 69, which is an siRNA.

92. The inhibitory siRNA or shRNA of claim 69, which is an shRNA.

93. A pharmaceutical composition comprising the inhibitory siRNA or shRNA of claim 69, and a pharmaceutically acceptable carrier.

94. A method of treating and/or preventing heart disease in a subject, comprising administering to a subject the inhibitory siRNA or shRNA of claim 69.

95. The method of claim 94, wherein the heart disease is an acquired or genetic form of heart failure or cardiomyopathy.

96. The method of claim 94, wherein the subject has a genetic mutation associated with heart failure or cardiomyopathy.

97. The method of claim 96, wherein the heart disease is associated with a deleterious mutation in the TTN gene, and/or the subject has a deleterious mutation in the TTN gene.

98. The method of claim 97, wherein the deleterious mutation in the TTN gene is a truncating variant mutation in the TTN gene.

99. The method of claim 96, wherein the heart disease is associated with a deleterious mutation in the MLP/CSRP3 gene, and/or the subject has a deleterious mutation in the MLP/CSRP3 gene.

100. The method of claim 95, wherein the cardiomyopathy is dilated cardiomyopathy.

101. The method of claim 95, wherein the heart disease is an acquired or genetic form of heart failure.

102. The method of claim 101, wherein the heart failure is heart failure with reduced ejection fraction.

103. The method of claim 94, wherein the administering improves cardiac function and/or ameliorates sarcomere dysfunction in cardiac cells of the subject.

104. A vector comprising a polynucleotide encoding the inhibitory siRNA or shRNA of claim 69, operably linked to a promoter.

105. A method of inhibiting the expression of MTSS1 in a cell, comprising transducing the cell with the vector of claim 104.

106. The method of claim 105, wherein the transducing of the cell is in vitro or ex vivo.

107. A vector comprising one or more polynucleotides encoding one or more gene products, operably linked to one or more promoters, wherein the one or more gene products are selected from the group consisting of:
a SYNPO2LA polypeptide, optionally wherein the promoter is cardiac-specific promoter, optionally wherein the cardiac-specific promoter is a TNNT2 promoter, optionally wherein the SYNPO2LA polypeptide is human SYNPO2LA polypeptide; and
a SYNPO2LB polypeptide, optionally wherein the promoter is cardiac-specific promoter, optionally wherein the cardiac-specific promoter is a TNNT2 promoter, optionally wherein the SYNPO2LB polypeptide is human SYNPO2LB polypeptide
